SEQUENCE LISTING (183 sequences)

SEQ ID NO: 1 - DNA, 795 bp, Thlaspi arvense
  source 1..795: mol_type = genomic DNA
atggggaagagaacaagtaacagtttgaagaaagatgagttaaacagaggagcttggactgatcaagaagacaagatccttaaagattatatcatgatccatggcgaaggcaaatggagcactctcccaaaccaagccggtctcaagaggtgtggcaaaagctgcagactcaggtggaagaactacttgagaccggggataaagcgcggaaacatctcatctgatgaagaagaactcataatccgcctccataatctccttggaaacagatggtctctgatagctgggaggcttccggggcgaacagacaatgaaataaagaaccattggaactcaaacctccgcaaaagacttcccaaaacaaaaaccaaccaaccaaaacttcgaaaacattcaaccaacatcaagaacaatgtctgtgttatacgcacaaaggcgattaggtgctcaaaggctctgacttttcagaaccagagtagtagtggtagtaccaatcttcttcctatgaaagaacaagtgagctctatgatggatcatgaagctggttcttcgttgttggaagatcttgaaattgattttgataaaatccaatcagagtttctcttccctgatctgatggatttggaaggtttgggttgtggaaacgtaacatcacttgtttcatctgaggagattttaggagattatgttcgtgctgatgaatcttctcagggtaatcttgatctcaatagacctttcactccttgtcttcatcgtggcaacgatgaagattggctccgagttttcaattgttag

SEQ ID NO: 2 - protein, 264 aa, Thlaspi arvense
MGKRTSNSLKKDELNRGAWTDQEDKILKDYIMIHGEGKWSTLPNQAGLKRCGKSCRLRWKNYLRPGIKRGNISSDEEELIIRLHNLLGNRWSLIAGRLPGRTDNEIKNHWNSNLRKRLPKTKTNQPKLRKHSTNIKNNVCVIRTKAIRCSKALTFQNQSSSGSTNLLPMKEQVSSMMDHEAGSSLLEDLEIDFDKIQSEFLFPDLMDLEGLGCGNVTSLVSSEEILGDYVRADESSQGNLDLNRPFTPCLHRGNDEDWLRVFNC

SEQ ID NO: 3 - DNA, 3512 bp, Thlaspi arvense
  source 1..3512: mol_type = genomic DNA
tctcagccaaattgacaaataaaaatcacatttagaaaacatttcacggttttgtatgtaaactagtaaaaaaaaaaaaagagaacatgttatttcaatatataacattggaaagtatagtatattgattctctcactaatctaaaaatttatgcatcaataatttttaatagattaaaaatctaaagttaatcttattggaaattaaatataattattaagtttcaatatatatagacatgttatctctttcatttttaaccaactagtgatatgggacaaagtacaaaatgttagttacatcagttttaatctgatataaaatttgtatcaatttttttaatgttaacaaaattattgattacattgattatttaaatagtgtaaatttataattttatatcagttttgtttaactgatgtaaactaatataagtttgtatcaattttattgaacggaagtcaaataatcaataaaaatatcaattttcagaactgatgttaaatcacatattttatatcatttattaataagtaattcaccttttttgtagtgtgatatgagttattggcgtttaataataattttaaaaaattatagtttcatgttaaatcttacaaaattatataaatcttttaaaatcaaaaattatttatttgagcatttgcaataatgaacagttgcgaatgaataattaataaaatatttaaaaattaaaaatgtaaataacaaaatgtatttaaactctgaatatatgtatataataacataaattgacttaaaaaataataaatcatatgtacaaagttgacgccttcttaaatactgtttttataatttctaatataataattattcaaatggaaaaattaaacttgaatatgaaccttttaactctttgattgtcaaaaataaaaagttttcatgaaatagagttccgaacttaaaaaagagacatttgaaacttgggaccccaaatttatgtaacactgaaagatcttaaggagttttattttttgtttgttcatgacgcaggactatgtgtactgtgtaggcatgaagaccatgtgttaccgtcttaactgtatcaaatgatgattgcttacatactagaatgtggctagcaatgactattttcactctgtatacatacttataccccatatgcaatacaataatgatcaaggtcgttttaggacctatatgattggagtctaaggcacgagagactagactagcgatcatcgtgtgaagtgagttcaaaggatttcagatccttagtaataagtacaaaagtatattcttgatgacaaaattcggtagagttaaatctcaccaaaactgatacactattattaccaggtgagaaaaggattgatatttcaaagtgtgtgtgtgtgacagaagttttggaaagcgacatgtgtacggacattgcaacaactccatataaagttactttgttaacacaaaaagaatctattctctactcaacactaaagacaattgtaccaaccaaacaaccacaagagagagaaagagagtatggggaagagaacaagtaacagtttgaagaaagatgagttaaacagaggagcttggactgatcaagaagacaagatccttaaagattatatcatgatccatggcgaaggcaaatggagcactctcccaaaccaagccggtacatatccatctatctattgatctatccgtctttaaatgcaaatcttcatctacttacaataattgcacataaaggtctcaagaggtgtggcaaaagctgcagactcaggtggaagaactacttgagaccggggataaagcgcggaaacatctcatctgatgaagaagaactcataatccgcctccataatctccttggaaacaggttaattaacttcctctttcaccgaaacacagattgttttcttttcaagttaagtttttccataatttttgcggcgtaatgcagatggtctctgatagctgggaggcttccggggcgaacagacaatgaaataaagaaccattggaactcaaacctccgcaaaagacttcccaaaacaaaaaccaaccaaccaaaacttcgaaaacattcaaccaacatcaagaacaatgtctgtgttatacgcacaaaggcgattaggtgctcaaaggctctgacttttcagaaccagagtagtagtggtagtaccaatcttcttcctatgaaagaacaagtgagctctatgatggatcatgaagctggttcttcgttgttggaagatcttgaaattgattttgataaaatccaatcagagtttctcttccctgatctgatggatttggaaggtttgggttgtggaaacgtaacatcacttgtttcatctgaggagattttaggagattatgttcgtgctgatgaatcttctcagggtaatcttgatctcaatagacctttcactccttgtcttcatcgtggcaacgatgaagattggctccgagttttcaattgttagagcgtatcactagtctacataattacatacatatatatctgtacgaacaaaattatatttgtattttgtttgaaagcttctaatttctaattactaattttgttgtgttttctaatccatagattatcaattttgcactctttttcaattaaagacttatggaaatggcttttgtctttagaataaaatacaaattgcaaaatatcgcaaatacgcaatgaagattatttattttcgatagattttattcaattattatgtgcatgcataaatgatagagcttttattatagcctaactaccgcttaactaccctaccaataaattatacgagggtcattgttcttggtaatacgaaaaaaatctttgcaatattgtcttaacgaaattgaattttaatatacacacatatccatttataggcagacacggtcatacatctaaggatttattgttttttgtttcttatcattcttcggaatcattgtataaatggttattgttgtaacaagagaaaggacatcgggttataattttatagaggaaacctaaatttgtgattgtcgactgtggccgctgacggcggttcagtgtcactagtttatggtcttttatctatgctattgtgggtgcgaatatgtccttttgttaaccttcttaaatattgaaataaggttgttttcggattgtcaatgttattaaacaaatccattctcaatagtttgacaaaaaaaaaaaaaaaatccattctcaataaatagctgatgcaggaaagactattggtttttctgtattttaagttatcggatcttttatatgatataatccattacaaatagacactaattagacaagtgttaaaaatagttcttattatcaatttggcagtgatacataaataaacctattacacctacctattaggtgattatctgcgtatacatacatacacacggatttacattttattatagattttaaaatgtataaataatataccatattttattt

SEQ ID NO: 4 - DNA, 795 bp, Thlaspi arvense
  source 1..795: mol_type = genomic DNA
atggggaagagaacaagtaacagtttgaagaaagatgagttaaacagaggagcttggactgatcaagaagacaagatccttaaagattatatcatgatccatggcgaaggcaaatggagcactctcccaaaccaagccggtctcaagaggtgtggcaaaagctgcagactcaggtggaagaactacttgagaccggggataaagcgcggaaacatctcatctgatgaagaagaactcataatccgcctccataatctccttggaaacagatggtctctgatagctgggaggcttccggggcgaacagacaatgaaataaagaaccattgaaactcaaacctccgcaaaagacttcccaaaacaaaaaccaaccaaccaaaacttcgaaaacattcaaccaacatcaagaacaatgtctgtgttatacgcacaaaggcgattaggtgctcaaaggctctgacttttcagaaccagagtagtagtggtagtaccaatcttcttcctatgaaagaacaagtgagctctatgatggatcatgaagctggttcttcgttgttggaagatcttgaaattgattttgataaaatccaatcagagtttctcttccctgatctgatggatttggaaggtttgggttgtggaaacgtaacatcacttgtttcatctgaggagattttaggagattatgttcgtgctgatgaatcttctcagggtaatcttgatctcaatagacctttcactccttgtcttcatcgtggcaacgatgaagattggctccgagttttcaattgttag

SEQ ID NO: 5 - protein, 109 aa, Thlaspi arvense
MGKRTSNSLKKDELNRGAWTDQEDKILKDYIMIHGEGKWSTLPNQAGLKRCGKSCRLRWKNYLRPGIKRGNISSDEEELIIRLHNLLGNRWSLIAGRLPGRTDNEIKNH

SEQ ID NO: 6 - DNA, 813 bp, Thlaspi arvense
  source 1..813: mol_type = genomic DNA
atgagagattcaaactcaatccttgatctgtctcttcagatcagccttcctaactctcacgccggagaacttcttcacggcggtgaccgaagctccaccacaagtagtgattctggaagcagtctcagtgagttgggccatgagaacaacttcttcaacaaacctctcctaagcttaggttttgatcatcattatcgttatcaaaggcactcaaacatgatccaaccacaaatctacggtcgagatttcaagagaagctcatcatcaatggtttgtcttaaacgaagcattcgagctccaagaatgagatggacttctactctccatgcgcactttgtccatgctgttcagcttcttggcggccatgaaagagctacgcctaaatcagtgttggagttgatgaatgtgaaggatctaaccctagctcatgtcaagagtcacttgcagatgtatagaacagtgaaatgcactgataaaggatcatcaggagaaggaaaggtagagaaagatacagagctgatgacagaggacaataataataatgaagaagctgacgaaggaactgacacaaattcgccaaactcatcatctgtgcaaaagacccaaagggcttcttgttcatccaaaaagagagtgtgtatgaacatatctacacaagcagaacctcacttgggatcaactcgtcacactaatgatgatgggaagaaagaggcgatcaacgctcatctcaatttggaattcacattaggccggcaaagttgggggatggactattcggaacccttaagagatctaactcttctcaagtgctaa

SEQ ID NO: 7 - protein, 270 aa, Thlaspi arvense
MRDSNSILDLSLQISLPNSHAGELLHGGDRSSTTSSDSGSSLSELGHENNFFNKPLLSLGFDHHYRYQRHSNMIQPQIYGRDFKRSSSSMVCLKRSIRAPRMRWTSTLHAHFVHAVQLLGGHERATPKSVLELMNVKDLTLAHVKSHLQMYRTVKCTDKGSSGEGKVEKDTELMTEDNNNNEEADEGTDTNSPNSSSVQKTQRASCSSKKRVCMNISTQAEPHLGSTRHTNDDGKKEAINAHLNLEFTLGRQSWGMDYSEPLRDLTLLKC

SEQ ID NO: 8 - DNA, 4752 bp, Thlaspi arvense
  source 1..4752: mol_type = genomic DNA
gaggtcggtcgattcaaacttcttttccctttttgtcttttgtgtatatttccatatctatcctttattctccgagtaataaggaaattcttctttatcttttgaattagtccgatgtttccggtcttcaaaggttatccttaagagatttccttaaatccgaactaggtcggcacggtcttcctagtcttgatgggcttaagttattgcgagggaaaaccttttaggaaatgtattcattttccctctaacaatgttttaagtatgtattaacaaaataaaaattaatatatgaaaacaattaataaaaataagtaagatgcttatgtgaagcagaatattcccgaccgtgtgtctacttaccgtggccaaagacatcgcatgaaaaaacaaaccaaagagtaaggtaaattaaatagaatgtatttaactagattgaacttggagtaattagttgtctttctctttttgcacttttgaaaagataaacaaaatttccttttggagctactttgtggttcaaacaagaatccccatggcaaaacggcagttactttgtcagtcaacttttgaaatctgaggctttcaaggattgtaagaaatattaaatacaaaactctgttgtattttccatcgaccctaagagacaggccttttcaagcctatctttttagcattggaaattttaaagtgttttattttatttatttaaaatagtaaaaacatgcaaatcatagggtactcattatctaattatcacactcactctctttcctcggcttggtttaacctttttaaccactgatcaaataatacgtagccaatttgtccaaaatcttccaacttcttttcctcctttcttacttctttaaattattttagtattcgtcataaatcaaaagatacgttgtcttgaacatgtacacaggttgtagtatttgtgtgaaacacaaaagtgtatactagtattttagttaggaaatctacaccatatatttacttttcttataaattaagtatatgactttgacattattttaatgatacataatcttttgattactttcatcagttattttttttgctaaaaattaatttaaatgttgtttttaacctactaaagatttcatgcgtacgtcagagactattttttgtttctcatatcatgatatgtaatcagaaaagtgaaaacaggttgccatgtacattcatttatgatacacaattgattttttttttctttttaccaaataacaggttattcatcagttgccacattggcgaaagatgaaaatactattttcattgatccgcataaataaaaaatcctttgattttaccactttgttatggtagtcatttacgtagttttaattcggttatctacccgaattacgttttttttgcttaaaagttgaaaatgttattctacgtagggactaacatctggtaatacagtactacgataagagtgaataattaagagttttataaataggttatatatttaaatcatttgtacacgtagaaaataaaacatattattcttaaaaaaagacgatagactatgtttattgagataaagacaatagttacgtcgttgcttttcttgctctctctctctccatttctttctctctccgctatttaaatagacaaaggattaatgaaaaagtagagagatgatgatgttagactccagaagcagtatgagagattcaaactcaatccttgatctgtctcttcagatcagccttcctaactctcacgccggagaacttcttcacggcggtgaccgaagctccaccacaagtagtgattctggaagcagtctcagtgagttgggccatgagaacaacttcttcaacaaacctctcctaagcttaggttttgatcatcattatcgttatcaaaggcactcaaacatgatccaaccacaaatctacggtcgagatttcaagagaagctcatcatcaatggtttgtcttaaacgaagcattcgagctccaagaatgagatggacttctactctccatgcgcactttgtccatgctgttcagcttcttggcggccatgaaagtattttgctttcacattttcctttttaaaagaaataatcaagatttatgtcaagtaaaatgttttgaaaccaaagtactaagatttgttgcttatttgctggtctcaggagctacgcctaaatcagtgttggagttgatgaatgtgaaggatctaaccctagctcatgtcaagagtcacttgcaggtccatctctattttaatttatttttcttatatattttattataaaaatagtaatggaatctcataagaatgcaataggtttaagtaaacatttatgaaagtgtatctatttggtccactagctaacacatgattgcgtagttgaaataaacttgtattagctatactttttttattttttatttggttttgatttttaaaggatgtgtgtttgttgatgcagatgtatagaacagtgaaatgcactgataaaggatcatcaggtatgcattcaaaccggattggctgatttttgaattcctactggatgtaaatgtaactatctcatttttttttttttgttttaatttgtatttgtttcttcttgggaataacaggagaaggaaaggtagagaaagatacagagctgatgacagaggacaataataataatgaagaagctgacgaaggaactgacacaaattcgccaaactcatcatctgtgcaaaagacccaaaggttactcttttatctattctattttgtaaaaactttgcatttaatctctttgcctttgttatgcgcgaattttgatgaatccctcttctagtcgtatgtgaccctgcttgtccatttttataaatagaaaaaatttctatgatctcagttaaattgtgtgtattcactgttgatagtaaataagtaggttcaaataaaacaatatatgaatatgaatgtccaacaatatgaattattattagtagatttctcaaatgtgttttttatcatttaaatttgaacagggcttcttgttcatccaaaaagagagtgtgtatgaacatatctacacaagcagaacctcacttgggatcaactcgtcacactaatgtacattctttaacaaattgttacaatgttattatagatgtgcactctcacatatacgtacatatacccttctgtatactgtttctaaaatgtgaaatctttggaaataggatgatgggaagaaagaggcgatcaacgctcatctcaatttggaattcacattaggccggcaaagttgggggatggactattcggaacccttaagagatctaactcttctcaagtgctaatcgtttagcttggagaactacaacaaataagtcagcttaggttatcaatttaacataattaacttgtttgatcgtaatagacattggaagaatcattatcatcatatatgaatttcttacaaaaaatgttctcatttttttttttgggatgtaaacaagagattcggattaagtagtagctatgttatggttgtagtggatgagaagggagtgcaagttcaagtagagatgaacttatgattggttgccaccaaatatgcatggtggatttgttggtagtttttttttatgttttcattgactgctattcatattgtcgcagtgaatatatatttaaatgattttgttttgattctttgtcttgatttggattagaaagtagaattcggcaaaactagaggtttttttactgatattctgcatgacaataaaactctcagttctatttaaatttttcgctttgttattgatacatataccgtttaaaaggatacattaaacaatactgaatagtactctcaaattctgtttttcgaaggatatgagttggtagatatttcacaacatcaaaaaacgaaggtgaaaataccttcttcagattgcaaagaattacagaaattttgtgccaggtttcgcagaccgcctgttgtaagagatcgcaacatatatcttgaaccaatccctcgaaagcataaataaacaaagtagacactaagaaatgattatataaataatataatatgtttaatttattaattatatgtagctgcaattgcttcatgaatattatgtggtagagactgacaaaaataaatggaaagagacacttcatcacaacatggtaatcataactcttcatccagaaaacttccatacctcttcatttatatgagaatttaatgttataggcaatctaataaatactctccctttgaaggtgcatccaactgaaaagcagggaccgatgccttccctttctaggaaacttgtcgcgccttaccagaacagtactctgccaagtccaatctagacttcatattgtcctttgtcttcctttgacattgagaacggtgttgattatgttgtctataaaattcttcgtattgtgcataacatctagattatgccttatcaaatggtccttccaaaaagacaattcccaaaatatactcatcttatacaaactatgttggtcaccacaagcgttcacatgataatgataaccttccatgcattggccatcagaataacatgccatatgctggaaaaaggctaatagtccacattaatactgaccccatgacaatattctgctgacaagaaacatgaaatgcctgaacactatgctcctataatatttgcagctcataaaacaatgattggagaaatt

SEQ ID NO: 9 - DNA, 1041 bp, Thlaspi arvense
  source 1..1041: mol_type = genomic DNA
atgacaatcttggatcagaccgttgtaacaaccggatcgaagaaggcttgtgtcatcggtggcacaggaaacttagcttctactctcatcaagcatttgcttcaaagtggctacaaagttaacactacagttagagatccagagaatgagaagaaaatggctcacttaagggtacttcaagaacttggggacctcaagatcttcaaggcggaatttactgatgaagagagtttcgattcaccagtttcgggctgtgattacgttttccatgtcgcaacgcctatcaactttacatctgaagatcccgagaaagacatgatcaagccagggatacaaggagtgaccaatgtgttgaaatcttgcttaaaatcgaaatcagtcaagcgtgtgatctacacttcttcagctgctgcggtttccatcaacaatctttctggacctggacttgtgatgaacgaagaaaactggactgaccttgattatctcacaaaggagaagccgtttaactggggctacccagtgtcaaagatactagcagaaaaggcagcttgtaaatttgcggaagagaacaagatcgatctagttaccgtgattccggcactcatatccggaaaatctctcctctcggatcctcctccgagcagctcatttctctctatgtctttaatcaccgggaatgaaatgtatctgaaaggtctcaaggaaatgcagaagcaatctggctccatctcgttcagccacgtgaaggatttggctcgtgcccatttgtttcttgcggagaaagaaactgcgtctggtcgttacatttgctgtacttacaacacaagtgttccggagattgcagattttctcaggcagagatatcctaagtacaatgtgctgtctgaattcgaagagtgcttatcaagtgcgaagctgacgctatcttcggaaaaactcatcaatgaaggctttcgattcgaatatgggattaatgagatctatgatgagatgatagagcacttcgagtccaaaggattaatcaaagctaaagaatcttga

SEQ ID NO: 10 - protein, 346 aa, Thlaspi arvense
MTILDQTVVTTGSKKACVIGGTGNLASTLIKHLLQSGYKVNTTVRDPENEKKMAHLRVLQELGDLKIFKAEFTDEESFDSPVSGCDYVFHVATPINFTSEDPEKDMIKPGIQGVTNVLKSCLKSKSVKRVIYTSSAAAVSINNLSGPGLVMNEENWTDLDYLTKEKPFNWGYPVSKILAEKAACKFAEENKIDLVTVIPALISGKSLLSDPPPSSSFLSMSLITGNEMYLKGLKEMQKQSGSISFSHVKDLARAHLFLAEKETASGRYICCTYNTSVPEIADFLRQRYPKYNVLSEFEECLSSAKLTLSSEKLINEGFRFEYGINEIYDEMIEHFESKGLIKAKES

SEQ ID NO: 11 - DNA, 4124 bp, Thlaspi arvense
  source 1..4124: mol_type = genomic DNA
gccgccgccgccgcgaaggccaagaagaaggatgtggaggctccgctgacaattgctttaggcttacaggcgccgaaatcgggaacgaagcggccgagtgaagggacttcaagggaggctaattcaaagcgagctaagaaggttacttcaggggacgacgagaagaagatcggcgaagactcaaagaaacctgcttttcagagactgtggtctgaggaagacgaaatcactgtgcttcaaggtatgatcgatttcaatgctgatacaggcaagtctccttacgaagacacgaatgtgtattacgatttcatcaagaaaaagattagctttgaggttagcaagaaccagttcatggataagattaggagcttgaagaagaagtatataggcaaaggaaagactgccttcacgaaacctcacgatcagagatctttcaagctgtgccaacacatatggggacctgaaggaatggctctcgagtcagcggttaagtccaatggcgtatcgagaaagagccagaagaagaagaagcttgactctgtgaagcaagagctgtcttttgcttcttcccctaatggcaaaacggttgatgatgataaaaaagtgttgatccatggaggagatgtggagtcttcggttgctgcgaagaagcatgattggttcgagagctcgtttcttgttcgcgccattgccggtttgggagttgatgagtatactgtgaaacagaggtggagcttggtgcctgttgagacgaagaagaaggttgaagagaagatgaagatgttgcaggccaaggagattgattttgtgttggagaagacacagtttttgcatgaggttacatcgatgatcgctgaagcatctaagaagaagacattagatatatagatttgatccgaaaatgccaatgccaatgcctctctcttttgttttttgaatcttaggaattatctcttttacttcccttttatgatctatcaatctatgtaatttttctggtttttctggtggtttttaatatggaactctctctcttttggatttgttgcttatacataaaagcaccatggaagtaaattctacatggtgaaatatgaagaagatccaaacaaactgttttttttttttttatgaaatgggtttattcaggatgaattttgttgaattgaggctaaaaaattttggtgttctctaaaacaaaaagttaaatgaaactttcagctcaaagatcaaattggttgaagaaagcaaaactcttttcacaagtagatgatatcaaatcagcaagtcaattaaaagtttgactctttttttgattcttaggtgagagagttagttattaaagaagaggaaggtaacacaaaattagttgcttcaaaactcacgtgcttaccttctaaaaagactttttgatcaatggttgtaccaaatgtgcaagaccataagctttgccactataaaaacgagtgctaaggccataaactcataacagtcagatctaaatatctgtgtttaagaactagtatcagatgacaatcttggatcagaccgttgtaacaaccggatcgaagaaggcttgtgtcatcggtggcacaggaaacttagcttctactctcatcaagcatttgcttcaaagtggctacaaagttaacactacagttagagatccaggttcttcatttcttctttctttcttctcttgaggttctttgagtgtttagtgactcttttattctgaatttgcagagaatgagaagaaaatggctcacttaagggtacttcaagaacttggggacctcaagatcttcaaggcggaatttactgatgaagagagtttcgattcaccagtttcgggctgtgattacgttttccatgtcgcaacgcctatcaactttacatctgaagatcccgaggtctgattttcaacagattgtgtatgtttttttttctctgactcttaagagaaacgttttcagctatcctttgtttgtgtttccatgtacagaaagacatgatcaagccagggatacaaggagtgaccaatgtgttgaaatcttgcttaaaatcgaaatcagtcaagcgtgtgatctacacttcttcagctgctgcggtttccatcaacaatctttctggacctggacttgtgatgaacgaagaaaactggactgaccttgattatctcacaaaggagaagccgtttaactgggtaatttacaatttcttgcgagccaagatagggtttacttggaccagtttacttactacatctctgttcttttctagggctacccagtgtcaaagatactagcagaaaaggcagcttgtaaatttgcggaagagaacaagatcgatctagttaccgtgattccggcactcatatccggaaaatctctcctctcggatcctcctccgagcagctcatttctctctatgtctttaatcaccggtaaacaccaattttactgtttgactccttctgttaaagtttcacaataagaaagtcaaagatgaatggtttttttttgttagggaatgaaatgtatctgaaaggtctcaaggaaatgcagaagcaatctggctccatctcgttcagccacgtgaaggatttggctcgtgcccatttgtttcttgcggagaaagaaactgcgtctggtcgttacatttgctgtacttacaacacaagtgttccggagattgcagattttctcaggcagagatatcctaagtacaatgtgctgtctgagtaagcatttatatccacagaaactgaaaatcttaatggaaaattctgaattttctcgttaattttcgctgtgaaatttggcagattcgaagagtgcttatcaagtgcgaagctgacgctatcttcggaaaaactcatcaatgaaggctttcgattcgaatatgggattaatgagatctatgatgagatgatagagcacttcgagtccaaaggattaatcaaagctaaagaatcttgaaatttataatgtgaagatatggatttatgagtatatgagtctttgttctcattctcattctataaatggcattaaataataagttggttgatttgatatgtattttgatatacacacctagaaaatgaaaaacaagatttttcaagctttatattactctacgaagctgattagtaattttacttctaagtttctcacatttcacaactaccttttttctttcccatgttataaatatataacttttttttcagcataaaaagaagttgtggaaagaatgcgacgaactattttaccaaaaaaaaaggtgacaatctacatggaaaaaagtaaataaaatgtgttaataaactttcgatttaaacaagttgtgtatttacatacaattgatatatatatatataatactttataatactgtattcttttatcaaaaggatatatactgtaatatttagcaaattatacataaaactatcaaattaaagagtatgatgtattccactgaaaacataaaaaatgatatatgtagaaaccaacctgagtgtatgcgcgtggatattcgtttagtaataatgataatgatatgagtagactatattacctaaagccaatcaacatatgaccaatctacaagctcgagctttattagatcaaaacataaaaattggtatatgtagaaatgttgtctaaattaaaccaagatgaaaaacatgagaaaacattaaaacatcactttttattcttggtgatttgttattagaactccgagcaaagaacgaatctgtgaggacatcatgcatgtatgtcttactcagcgtaatagggacattgtgcaagtcactgcaagagatctccaatgatgatctaacggatccaagaaagtaaaggaaaagaagaaggaagagcagattggtgagactcggatccaagaattagaggaagatctgaaggatctgaagcggatgtgcactgttctagaaacctttctagggaaagaaaaggcaaatgtgtttgaagccagagcccctgctatgacgttagatgagttgtataatgacttaggttctgcatgatgcgttgtggtattgtatgttcagggccggctctaagattatgggagtttgaaacaaaataaaaaat

SEQ ID NO: 12 - DNA, 1461 bp, Thlaspi arvense
  source 1..1461: mol_type = genomic DNA
atggatccgacgacgccgtttctcggtggcgaggtcgaagaggattatgctccggcgaggacatggagcgacgtcaagcgagtttttgctacggagtcggccaaaatgtggatgatcgccgctcccattggtttcaacatcatctgtcagtacggagttacctccatcaccaatattttcgtcggccatatcggcgaggtcgagctctccgccgtcgccatctccctctccgtcatcggcaccttttccttcggcttcctgcttggcatgggaagtgcacttgaaacactctgtggacaagcatttggagctggtcaagtccatatgttaggcgtttacatgcagagatcttggattatcttattcgtctcctgcatctttctccttcctatttacatattcgccacgccgattctgagactcctcggccaagcagaggagatcgccgttccagctggagaattcactcttttaaccatccctcagctattctcactcgccatcaacttcccaacctccaagttccttcaagcgcagagcaaagtcatcgcgattgcttggatcgggttcatcgctttcgtcctacacgtcggtatgctctggctgtttataatcgtgtttggttggggaacaaacggtgctgccttggcgtttaatctcaccaactggggaacagcgatctctcaagtcgtttatgtgattggttggtgtaatgaaggctggtctggtttgtcttggttggcatttaaagagatttgggctttcgttagactctccatagcatctgctgttatgctttgtcttgagatctggtacatgatgagtatcatcgtccttactggtcgccttgacaacgctgttatcgctgttgattccctttccatatgcatgaatctcaatggtctggaggccatgttgttcatcggaataaacgctgctataagtgtccgtgtctccaatgagcttggcttaggccgtccacgagcagcgaaatactctgtctatgtcacggtgttcgagtctctcctcatcggtcttgtctttatggtggctatcatcataggcagagaccattttgcgatcatcttcacgagcagcaaagtacttcaacgcgcagtgtctaagctagcttatcttcttggtataaccatggttctcaacagcgtgcagccagtcatttccggtgtggctgttggaggaggttggcaaagtttggtggcttatataaacttgggttgttactacattttcggccttccctttggatatcttcttggttacaaagcaaacttaggagtgatgggactttggtcgggaatgatagccgggacagcgcttcaaacgttgctactgatgtttgtcttgtacaagacaaactggaataaagaggttgaagagacgatggaacgtatgaagaaatggggagggagcgagacgacatcgaatgatgtaactgcgtga

SEQ ID NO: 13 - protein, 486 aa, Thlaspi arvense
MDPTTPFLGGEVEEDYAPARTWSDVKRVFATESAKMWMIAAPIGFNIICQYGVTSITNIFVGHIGEVELSAVAISLSVIGTFSFGFLLGMGSALETLCGQAFGAGQVHMLGVYMQRSWIILFVSCIFLLPIYIFATPILRLLGQAEEIAVPAGEFTLLTIPQLFSLAINFPTSKFLQAQSKVIAIAWIGFIAFVLHVGMLWLFIIVFGWGTNGAALAFNLTNWGTAISQVVYVIGWCNEGWSGLSWLAFKEIWAFVRLSIASAVMLCLEIWYMMSIIVLTGRLDNAVIAVDSLSICMNLNGLEAMLFIGINAAISVRVSNELGLGRPRAAKYSVYVTVFESLLIGLVFMVAIIIGRDHFAIIFTSSKVLQRAVSKLAYLLGITMVLNSVQPVISGVAVGGGWQSLVAYINLGCYYIFGLPFGYLLGYKANLGVMGLWSGMIAGTALQTLLLMFVLYKTNWNKEVEETMERMKKWGGSETTSNDVTA

SEQ ID NO: 14 - DNA, 4540 bp, Thlaspi arvense
  source 1..4540: mol_type = genomic DNA
gcagttcacatttaaatagtccaatatgttattattggcaagcaacccgaattgaaatgagacagtggaaacattagcaatccaaaacatcatacgaacctacttctacttataccttagagaagaataatcagattacgagtaacaagaatgaagaacctgagtcgcttagccttaatgttctaaatcaaataactaatctgagctcatttgcaaaacattggcccattttaaaattcttggcactgatacgcagcacaaaaggaggttactgtaaaagcccaatgtgcgtacattcattattcacaaaaaaaaatcttgtttgacttaaaattgtggccccctttttgggtttgtcttttaatttcatgtattttttaattaattgttttctattttggcattaatatggctctattaattataatttatgatacaaaatcagaatggaccgggctgggcctttggtgaagttggtgggctcggtttaagaattttatttttttcattattttgacttgcattaaataaaacaagtcaatgggaaatgtcatcatttatctgactggtttaatgacaaaggtttaatgatgaaacagggaaataaatacaaacgatgacgtaaactgaaaggggaccacaggaccctgtcgtttccgatgaaaaagaagaagaaaattccctctccgattcttcttcctgttcatattcattttacaaacggtacgtaactcactttttaatacttcacttaaggacatcaacctattttcggaaacgacaacttttccactcttggttcgagcttagattgattcaacatttttttcctaattaacttacatatactgtattcattttactgtctaacagtaaactcgagctatcctggcaagtttaatcttctcgacaacttactatatagttgatactatatcactaggatcaattttataaaaaaaaaaacaaattaatcacgtgaaaataattctcacacatgataaataaactaggagtaacaagtttgatgtgacattacactaacaaactttcacaacactaccctatcaatattaaatgtcaaatgatggaaaagagaaaagcagttttatgcttttgaataatcttgtatgcatatttctgatatgtcaaagtacgtggtgtctcatgcaccacccgttagcctcacctacctaatctacttctcaattccatgccgaattcggtaataaatttctcgtacgttttgtttttcctttccgctaattacccaaaaactaaaataggctaataactccttcaatttttattattgttattatttgattagtatggcaaaacaaggggagagagaagtgcgtgattcaacggttttgttatataaaccaaaaccccataattccgcaacaaacattgactcagagtgagagagagagagagagagagagagaagtctgtctgtgtgagctttgggtttaacattttgagactcgcacggagatggatccgacgacgccgtttctcggtggcgaggtcgaagaggattatgctccggcgaggacatggagcgacgtcaagcgagtttttgctacggagtcggccaaaatgtggatgatcgccgctcccattggtttcaacatcatctgtcagtacggagttacctccatcaccaatattttcgtcggccatatcggcgaggtcgagctctccgccgtcgccatctccctctccgtcatcggcaccttttccttcggcttcctggtatgcttttctcttcttctaactgatatctttcatgtaactctgctatgtatcaatcaagagcatcttcttgtgacaaatcgacaagaaacagagtctaatttaggtttgaaaactgcagcttggcatgggaagtgcacttgaaacactctgtggacaagcatttggagctggtcaagtccatatgttaggcgtttacatgcagagatcttggattatcttattcgtctcctgcatctttctccttcctatttacatattcgccacgccgattctgagactcctcggccaagcagaggagatcgccgttccagctggagaattcactcttttaaccatccctcagctattctcactcgccatcaacttcccaacctccaagttccttcaagcgcagagcaaagtcatcgcgattgcttggatcgggttcatcgctttcgtcctacacgtcggtatgctctggctgtttataatcgtgtttggttggggaacaaacggtgctgccttggcgtttaatctcaccaactggggaacagcgatctctcaagtcgtttatgtgattggttggtgtaatgaaggctggtctggtttgtcttggttggcatttaaagagatttgggctttcgttagactctccatagcatctgctgttatgctttgtcttgagatctggtacatgatgagtatcatcgtccttactggtcgccttgacaacgctgttatcgctgttgattccctttccatatggtgagttttgatcaacattttacaatgcttcttgtatcatcaacatctttcccactctttttttttgcatttcatttcagcatgaatctcaatggtctggaggccatgttgttcatcggaataaacgctgctataaggtagtactatcattatctgacacaacttttgtttaacccattgttcagcttgtaaactgatcatgtgtctacctacttgtgtactatatattagtgtccgtgtctccaatgagcttggcttaggccgtccacgagcagcgaaatactctgtctatgtcacggtgttcgagtctctcctcatcggtcttgtctttatggtggctatcatcataggcagagaccattttgcgatcatcttcacgagcagcaaagtacttcaacgcgcagtgtctaagctagcttatcttcttggtataaccatggttctcaacagcgtgcagccagtcatttccggtaacaaaaatctaaaattctcatgttccatcaaaacaaaaaccagaggtctcataagaccataaccgtcccggtttgttcatcttgttttaggtgtggctgttggaggaggttggcaaagtttggtggcttatataaacttgggttgttactacattttcggccttccctttggatatcttcttggttacaaagcaaacttaggagtgatggtaagcgaatacaaagaatgacatgaataatgtagatctttcggacatgcatgtttcataaactgatacgtgagagattataacgttttcagggactttggtcgggaatgatagccgggacagcgcttcaaacgttgctactgatgtttgtcttgtacaagacaaactggaataaagaggtaaagaacacctgagacacaaaaccgtagtatatacactggttcattgagatcgattgacttatggaaatataaaattgtaaaaactgtgatgaacaggttgaagagacgatggaacgtatgaagaaatggggagggagcgagacgacatcgaatgatgtaactgcgtgactatttcttttgttaatattaattaattaattattgtttagctttatatatatgaacatgtaggtctcagcttttttgtttgttttccattggtttggcagcaccagtaactctctatttactatttacgctgtaggaaacttttcattcagtgatgtaacgatgcatgcttttgtcactttgtttctcttggagtaaactaaatgttaggtacattttctcgtgtaacacaaattttattagacggcttttagtcttcaatgcaaattcaagtgacttgtacatagattcctgtcttctacttccatttccttcagaagtcaattgtatgtttccattttattcttctaccggtggaaaataatatagtatcagtacgtaatttttagcgtgcttgtaacacaggatggccgagcggtctaagccacgagactcaagttcttgtcctcctataggaggatatggattcaaatccctcttgtgacataataatttctttaaaaaaaaaatcattttgctatactttgcatatttttctttttctttttaagaatgcatgcatgtcttactttctcgtttctgcttgataggtcgtagtgtcctaataagctcttctgagaaatctcttatttttcttcttgaacgttggattcagcatttcttcattgtgatttttgacttgcgaagatacgcacacttatacattgattagggtcatcctcgtctcaagtgttattgcaatgtctaggtttggacatagcacactagtctttgctatttcatcacccaaaatccattaaatcatggtgatgatttgatttaaagctaatggttttgctaatttgattttttttatgtgtattttttgttctgtaataggtggcgtgagtggaacaacagaagggtggaacacaaacacttattaggtctccctgttttcaacatgatgcatttgattttgttggttatatctcctcaaatgcttgttgcattgcctaaa

SEQ ID NO: 15 - DNA, 2322 bp, Thlaspi arvense
  source 1..2322: mol_type = genomic DNA
atgtcaatggccgtcgaaatgtcatcgaaacaacccaccaaagatttcttctcctctccagccctctccctctctctcgctggtatattccggaatgcatcctccggcagcaggaaccctgaggaggactttttggggagaagggtagttgacgatgaggatcggacggtggagatgagcagcgagaactcgggacccacgagatccagatcagaggaagacttggaggatcaagaggaggaggaggaggatgaggaggaagacggagcaggaaacaagggcaacaagaggaagaggaagaagtatcaccgccacaccaccgatcagattagacacatggaagcgctgttcaaagagacgccccatccagacgagaagcaaagacagcagctgagcaagcaattagggctggctcctcgccaggtcaaattctggttccaaaaccgccgcacccagatcaaggctattcaagaacggcacgagaactcgctgctgaaagcggaactagagaagctgagggaggaaaacaaaggcatgagagagtcttttgccaaggctaattcttgctgcccaaactgcggaggaggcaccgatgatgtccacatcgagaactccaaactgaaggcggagctggataagcttcgtgcggctctcggacgcactccctacccactccaggcctcatgctccgatgatcaaccacaccgtctcgacttctacacgggcgtctttgccctcgacaagtcccgcatcgtggagattgccagccgagccacccttgagctccagaagatggcctcctccggccaacctctttggctccgcagccttgagactggccgtgacattctcaactacgacgagtatctcaaggacttccctcaagctcaggcctctcccctccatgcaaggagatccatcgaagcatccagggatgtggggatcgtgtttatggacgcacacaaacttgctcagagttttatggacgtggggcaatggaaagagatgtttgcgtgcttgatctcaaaggcggcgacggttgatgtaatccggcagggtgaagggccttcaaggatcgacggtgcgattcagttgatgtttggggagatgcaactgctcactccggttgtccccacaagagaagtgtacttcgtgagaagctgccggcagcttagccctgagaaatgggccatcgtggacgtatcagtctctctggaggaagacgacaacaacaacaacacggaggacaaggaggcttcgctgcttaaatgccggaaacgcccctcaggttgcatcatcgaggacacctccaacggccactccaaggtcacctgggtggagcacctcgacttgtctgcctccaccgttcagcctctcttccgctcctttgtcaacaccggtttggcctttggggctcgacactgggtcgccaccctccagctccactgcgaacgcctcgtcttcttcatggctaccaacgtccctaccaaggactctctcggtccgtccattatttacactctctctctccctctctctctttctccctctcaccttttcttaaccccaatcctcctctcaggagttacaacgcttgccgggagaaagagcgtgctcaagatggcccagaggatgacacaaagcttctaccgcgccattgctgcttccagctaccaccaatggaccaaaatcaccaccaaaactggacaagacatgagggtttcttccaggaagaacctccatgatcctggtgagcccaccggagtcatcgtctgcgcttcttcctccctctggttacctgtttctcccactctcctcttcgatttctttagagatgaagctcgtcgccatgagtgggatgctttgtcaaacggagctcatgttcagtctatcgcaagcttatccaagggacaagacagaggcaactcagtgtctatccagacagtgaaatcgagagaaaagagcatatgggtgctgcaggacagcagcacaaactcatatgagtcggtggtcgtatacgctcccgtagatataaacacgacacagctggtgattgcaggacatgatccaagcaacatccaaatcctgccttgtggattctcaatcatacccgatggagtagaatcaagaccactggtaatcacgtctgcacaagaggacagaaacagccaaggagggtctctgctcacactggccctccaaacgctcatcaacacttctcctgcagcaaagctgaatatggagtccgtggaatccgtcacaaacctcgtctctctcaccctccacaacattaagagaagcctccaaatcgaagattgttga

SEQ ID NO: 16 - protein, 773 aa, Thlaspi arvense
MSMAVEMSSKQPTKDFFSSPALSLSLAGIFRNASSGSRNPEEDFLGRRVVDDEDRTVEMSSENSGPTRSRSEEDLEDQEEEEEDEEEDGAGNKGNKRKRKKYHRHTTDQIRHMEALFKETPHPDEKQRQQLSKQLGLAPRQVKFWFQNRRTQIKAIQERHENSLLKAELEKLREENKGMRESFAKANSCCPNCGGGTDDVHIENSKLKAELDKLRAALGRTPYPLQASCSDDQPHRLDFYTGVFALDKSRIVEIASRATLELQKMASSGQPLWLRSLETGRDILNYDEYLKDFPQAQASPLHARRSIEASRDVGIVFMDAHKLAQSFMDVGQWKEMFACLISKAATVDVIRQGEGPSRIDGAIQLMFGEMQLLTPVVPTREVYFVRSCRQLSPEKWAIVDVSVSLEEDDNNNNTEDKEASLLKCRKRPSGCIIEDTSNGHSKVTWVEHLDLSASTVQPLFRSFVNTGLAFGARHWVATLQLHCERLVFFMATNVPTKDSLGPSIIYTLSLPLSLSPSHLFLTPILLSGVTTLAGRKSVLKMAQRMTQSFYRAIAASSYHQWTKITTKTGQDMRVSSRKNLHDPGEPTGVIVCASSSLWLPVSPTLLFDFFRDEARRHEWDALSNGAHVQSIASLSKGQDRGNSVSIQTVKSREKSIWVLQDSSTNSYESVVVYAPVDINTTQLVIAGHDPSNIQILPCGFSIIPDGVESRPLVITSAQEDRNSQGGSLLTLALQTLINTSPAAKLNMESVESVTNLVSLTLHNIKRSLQIEDC

SEQ ID NO: 17 - DNA, 5982 bp, Thlaspi arvense
  source 1..5982: mol_type = genomic DNA
aacccataaagagcattcatttccttttaaggtcgctgaaattaatgagtaacgatcagtcaatgcgtctcgttgaccttccaaaacatccgacgtctctttccgttgcatatccctcggctctgtccctgcagctcccacgtcatccatatttcccctctatccaatttttaactttctaacttattcaacaactcttcctccatgcatttacctcattatctaatcgtattgtttactagtagtaatatagcatactagagtagtagattcggattcccggaataatatatattagcataagttattggagcacaaggcatttcaggtttccatgtagctccaataatatcctcttcactctctcttaccaatgcttttccactttcctatgtcacgcaattactagcttgcttgctttacaaccaagttttattaaccaacataaaatatagagatttaatgttgcattttgtaatcataagttactaattgcttgaagaaagagatcacaataacagacaatacgtacacattacatcataaccaggtaagtatatagtatataataaataaataaatagaagtcataataagagaaatgatgatgataatcaaggaggaaagaagaaagcagaaaatgcggttggagaattaggtgcttaaagttagttgagtccatcccagtatctaacggtcaactctctcctctttcgtccttatttgtattttattttgtttttgaaacaataaactgacatacaatatgtctttctctcactactctctctctctctctctctatacacaaattcaattaaagaagagacagagaagttcgccttttgtctgtatacccttaaatcatgcaacccccttattctcattccttctctctgcctacagtaaactctaggaacgacattatgtggtttaaaccgattcaattcatgattagtggcaaaattctatatgattttcaaccacatgatatatgattttcaacagtaatattgtgcggaaaaataatgcacagcattttattctatatacaaaaggtaaattggaaaggagaattttccagatgcaatagcgaatttgctcgaatatttttataaaaaaaaacgaagtcgatatcgacgcctgcaaataatgtagctggccatgtttgcaaagtaatcaagtgtgaataaaattgtaaaactaatggagtatatatagatattaaaaagaagaaggggttggttaaatataattaaatgaagcacctaaaagtcaagcaatagagaaatatctgaaaaacgaccgtttgtatgtataattagaattgaagggctagcttagctatatagccatatacagtatatgtgaaagagtacattttggtacacacacacacaaaaaaaaagaaataaggaaaatatatagagatatataaatagcacaaggaagaaggaggagagggaagataaagcaaataaaaaaattgggagcgttcgtatgtcaatggccgtcgaaatgtcatcgaaacaacccaccaaagatttcttctcctctccagccctctccctctctctcgtacgcgtactctctgttctactactctcgtaaactcatctaaataaaaatttactgtattgtatatatgtatctatttgtgaatggtactgatataaatacaggctggtatattccggaatgcatcctccggcagcaggaaccctgaggaggactttttggggagaagggtagttgacgatgaggatcggacggtggagatgagcagcgagaactcgggacccacgagatccagatcagaggaagacttggaggatcaagaggaggaggaggaggatgaggaggaagacggagcaggaaacaagggcaacaagaggaagaggaagaagtatcaccgccacaccaccgatcagattagacacatggaagcgtacgttcttccttccttaattattattctttatatttcatctgtagctctaatggattaccatttaatcaaattagggtttcaaaacatggaacatttcgattaattggtcttgatgatatatgaatttgatgacctcaactcctccatatatacttcaattatgactagtcatcatcatcatgccctacacatagggattagagtatatagtagtcagtctgtgctgaacagtaggcctcttcatttctactgccatataaaaaggaagaatgcatatttcatgtatttattagtagtgatgaacaaattaatatatattgcagatcgatgatccagcaatagttaattatttgaatgcgtttaattacatatcttgctctttaattttctctcgtactccctagtatatacatgtgtttatactataacataggacgggacaagtgtttccattcaatcaccctatgattaaactaaaaccaagttctttcaaaacaaaaaactaaaaccaagagcactaatgtgtatagctagttagggctcatataattaagagtttaattcatactatgttttcgaataggaggataaaaaaaagatatgaaagaattaattaaatatgaagaatgatcatggagctatgaatgagaaaactgagggttgcattgcaggctgttcaaagagacgccccatccagacgagaagcaaagacagcagctgagcaagcaattagggctggctcctcgccaggtcaaattctggttccaaaaccgccgcacccagatcaaggtatgtatatcgacgtacgtgatgatgatgatatatatggatttcttaagacaaaaagactattcgcttggagctacatatgtatatagttatagtgaaatactgaaatgtaactgaacaggctattcaagaacggcacgagaactcgctgctgaaagcggaactagagaagctgagggaggaaaacaaaggcatgagagagtcttttgccaaggctaattcttgctgcccaaactgcggaggaggcaccgatgatgtccacatcgagaactccaaactgaaggcggagctggataagcttcgtgcggctctcggacgcactccctacccactccaggcctcatgctccgatgatcaaccacaccgtctcgacttctacacgggcgtctttgccctcgacaagtcccgcatcgtggagattgccagccgagccacccttgagctccagaagatggcctcctccggccaacctctttggctccgcagccttgagactggccgtgacattctcaactacgacgagtatctcaaggacttccctcaagctcaggcctctcccctccatgcaaggagatccatcgaagcatccagggatgtggggatcgtgtttatggacgcacacaaacttgctcagagttttatggacgtggtactctttttctttcatttcctccaaatatttacgaatatacttttgtgtatcacatgtaggcaatataaagatatatatacaacgtgcaggggcaatggaaagagatgtttgcgtgcttgatctcaaaggcggcgacggttgatgtaatccggcagggtgaagggccttcaaggatcgacggtgcgattcagttggttagtgaatcaccttttgatagatagtgatgacacgcgagagatgagagctaatataatacattgatggacatattggcagatgtttggggagatgcaactgctcactccggttgtccccacaagagaagtgtacttcgtgagaagctgccggcagcttagccctgagaaatgggccatcgtggacgtatcagtctctctggaggaagacgacaacaacaacaacacggaggacaaggaggcttcgctgcttaaatgccggaaacgcccctcaggttgcatcatcgaggacacctccaacggccactccaaggtcacctgggtggagcacctcgacttgtctgcctccaccgttcagcctctcttccgctcctttgtcaacaccggtttggcctttggggctcgacactgggtcgccaccctccagctccactgcgaacgcctcgtcttcttcatggctaccaacgtccctaccaaggactctctcggtccgtccattatttacactctctctctccctctctctctttctccctctcaccttttcttaaccccaatcctcctctcaggagttacaacgcttgccgggagaaagagcgtgctcaagatggcccagaggatgacacaaagcttctaccgcgccattgctgcttccagctaccaccaatggaccaaaatcaccaccaaaactggacaagacatgagggtttcttccaggaagaacctccatgatcctggtgagcccaccggagtcatcgtctgcgcttcttcctccctctggttacctgtttctcccactctcctcttcgatttctttagagatgaagctcgtcgccatgaggtaccttgtggcatacacttccttcgcacacacagaaccaataaaaccgttttaaatctaactatacatacgtattattatcatcagtgggatgctttgtcaaacggagctcatgttcagtctatcgcaagcttatccaagggacaagacagaggcaactcagtgtctatccagacagtgaaatcgagagaaaagagcatatgggtgctgcaggacagcagcacaaactcatatgagtcggtggtcgtatacgctcccgtagatataaacacgacacagctggtgattgcaggacatgatccaagcaacatccaaatcctgccttgtggattctcaatcatacccgatggagtagaatcaagaccactggtaatcacgtctgcacaagaggacagaaacagccaaggagggtctctgctcacactggccctccaaacgctcatcaacacttctcctgcagcaaagctgaatatggagtccgtggaatccgtcacaaacctcgtctctctcaccctccacaacattaagagaagcctccaaatcgaagattgttgatgacaactcacagcaggtgtgatgattgttgttacatatataagtatatcataatgtatagcagtgcattaagttttgtacaaaaacaaccctctctctctctcccgcaatcatactgttatccaacactttttgcagcattaagagagcatcatactcgactaagaacccaatacacaaaactttaccgaaacaaaagtgggatcagatgaaccaaatgcataaccctgatttagatacacagtaattggatcagaaacgaaggcctgatcaaattccacaacttaaacaatacgcaattcgagcaggaaaacaaaatgcattccataaacagagagaattacacttgattgcaatacacaacaaacagaaacgaaaaacagtaaaaaagacagctttttttttttaccaaaacaaacaaacactcagtgagtcgcattctcctctacgccggctgctgagattccaagggaatgccgaggtcgttcccagcttctcctccgccttccgctttcctgctgctccttgagacgacagcaagcgccacgaatgacttagtcttactgatgggtctgctcttctccagcctcaccacatcgcccaccttgaactgattatccgggtcatgcgcctggtacttcttcttcttcctcacgcgcctcttgtacttgggatgcggagccagcctcaccacctccaccgccacggtcttgtcgttggttgcgcacaccacgcgcccctgcatcgttttcatggctctgatgaccggaaccaatgccggccttcgagggttcgggaataaattgggcttagagagagatgagagtggagtcgagccatgggcgaatggagacgaaagcttcagagcttgcagagatgaggttattagcgacgtcatcgttatccccctcctctatgggatcgatttggaagacgatgaagaagataaggcgatagatcatgttattataatattggcttttgctaaatgggttaacaa

SEQ ID NO: 18 - DNA, 2007 bp, Thlaspi arvense
  source 1..2007: mol_type = genomic DNA
atggctactacgtataagcctaagaacattctcatcactggagctgctggattcattgcctctcatgttgccaacagattaatccgtagctatccagattacaagatcgttgtgttggacaagcttgattactgttcagatctgaagaatctcgatccttcgttttcttctccaaatttcaagtttgtgaaaggagatatcgcgagtgatgacctcgttaactacctcctcatcactgaaaacatcgacaccatcatgcactttgctgctcaaacccatgttgataactcctttggtaatagctttgagttcaccaagaacaacatttatggtactcatgtcctcttggaagcctgtaaagtaacaggacagatcaggaggtttattcatgtgagcaccgatgaagtctatggagaaaccgatgaggatgctgctgtaggaaaccatgaggcgtctcagcttttacccacaaacccatactcagccactaaggctggtgctgagatgcttgtgatggcttacggtagatcatacggactgccggttattacaactcgtgggaacaatgtttacggtcctaaccagtttcccgaaaaactgattcctaagttcatcttgttggctatgagtgggaagccgcttccgatccacggagatggatctaacgtaaggagttacttatactgtgaagacgttgctgaagcgtttgaggttgttcttcacaaaggagaagtcggtcacgtctacaacatcgggacaaagagagaaagaagagtgatcgatgtggcgacagacatctgcaaacttttcgggaaagatcctgagtcgagcatcgagttcgtggagaatcgaccgtttaacgatcaaaggtacttccttgatgatcagaagctgaagaatctgggttggtcagagcgaaccgcgtgggaagatggactgaagaagacaatggaatggtacattcagaatcctgagtggtggggtgatgtttctggagctttgcttcctcatccgagaatgcttatgatgcccggaggaagagttcctgacggctccgatgagaagaatgactcgtcaagcaacgcggtccagacattcacggttgtaacaccgaacaacaagactggtggttctagtgacaaagctttcttgaagtttttgatctacggtaagactggttggatcggtggtctcttagggaaactatgtgagaagcaagggattacttatgagtatgggaaaggacgtttagaagatagagcctctctcgtcgcggatattcgcagtatcaaaccgacccatgtgtttaacgctgctggtttaactggcagaccaaacgttgactggtgtgaatctcacaaaccagagaccattcgtgtcaatgtcgctggtactttgactctcgctgatgtttgcagagagaatgatctcttgatgatgaacttcgccaccggttgcatattcgagtacgatgctgcacatccagagcgttcgggtattggtttcaaggaagaagacaagccgaatttcactggctctttctactcaaaaaccaaagccatggtcgaggagcttctgagagaatttgacaatgtgtgtaccttgagagtgcggatgccgatctcatcagacctaaacaacccgagaaacttcatcacgaagatctcgcggtacaacaaagtggtgaacatcccgaacagcatgaccatactggacgagcttctgccgatctccatcgagatggcgaagagaaacctaagagggatatggaacttcaccaacccaggggtggtgagccacaacgagatactggagatgtacaagagttacatcgagccagggtttaaatggtccaacttcacagtggaagaacaagcaaaggtcattgttgctcctcgaagcaacaatgaaatggatgggactaaactaagcaaggagttcccagagatgctaccaattaaagaggcgctgatcaaatacgtcttcgaacctaacaagagaacctga

SEQ ID NO: 19 - protein, 668 aa, Thlaspi arvense
MATTYKPKNILITGAAGFIASHVANRLIRSYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKLIPKFILLAMSGKPLPIHGDGSNVRSYLYCEDVAEAFEVVLHKGEVGHVYNIGTKRERRVIDVATDICKLFGKDPESSIEFVENRPFNDQRYFLDDQKLKNLGWSERTAWEDGLKKTMEWYIQNPEWWGDVSGALLPHPRMLMMPGGRVPDGSDEKNDSSSNAVQTFTVVTPNNKTGGSSDKAFLKFLIYGKTGWIGGLLGKLCEKQGITYEYGKGRLEDRASLVADIRSIKPTHVFNAAGLTGRPNVDWCESHKPETIRVNVAGTLTLADVCRENDLLMMNFATGCIFEYDAAHPERSGIGFKEEDKPNFTGSFYSKTKAMVEELLREFDNVCTLRVRMPISSDLNNPRNFITKISRYNKVVNIPNSMTILDELLPISIEMAKRNLRGIWNFTNPGVVSHNEILEMYKSYIEPGFKWSNFTVEEQAKVIVAPRSNNEMDGTKLSKEFPEMLPIKEALIKYVFEPNKRT

SEQ ID NO: 20 - DNA, 5240 bp, Thlaspi arvense
  source 1..5240: mol_type = genomic DNA
acccttattttcaatcacagctccccaagatagatcagttaagcaacagcaacacacatatggccattgatgctaagctgttgcaggcgcaatcacagttcggtccagcgggagcagcagcggttgcggtagcggcacaccggaacattggcacaattagttacggcgcagcatcgtagactcatcaaacaaacagcattttcagacacatctttaacttacatcaccctccaaatccaggcaacgcgaaaccaggtgattccggtctcggtctctttatgtaagtattattcagccaggctctgatttaaacaactttgcagtgatattaataaaagactttcagttgtaactcaccagaacaagatcgcttagatcagtctgatcccgggttatcaccggtgattgtttcggttatcttgtttgctccatagtctttgaatttgttataagcgtagttcatcgaagagaagaaaaccgtcgtgattatatacttcttaaacttcaagatcggtcccattactccttaattgttcttcaacaatgatgaatctctttgtctatcagaaaattgattgatctatttggctatatagagagaatcattaggccgaaaattgtggaagtcaacaaaagtcattggtccgtagatctttctcatccgtaaatcaatcttttgcacattataattagccattttattcaatggttaccagtaaaaaagatatgtaaaagataatttcgaattcttgacgccattaaagtgtctatataatttacccgccaatttaagataaccggttttatgggcctgaactcagcacattaaaggttaacatttactataataattcaatgaacttaactccttttcttcaattacatgaaaaagtgcatttttgtctacttgattttttaattgatgaaaaaggaagtaaattaattattccgttacgtgaccttaagcatcagcattgaatcatctgttatgtttaatttcaggtcttttcttgttgaactgatgattgatatcaggttgacatgtgatatttctattcagtctagaattatttgtttttctaactgaacatatgtacgttttatcataaacaaatcagggattttattaaatcccaagtttaaccttccaataaaagaaatcatatttttattcagtttaaatcattttaagaaatatatatcagacaaaaatgcaaaatattttgtttacttcttccacaattaattaatctccttaattatacaaattttattctctttttattttttggaattttcttctattagtatagattttgtgtgaaaaaataaacaaaaaaaagacatcaaatttttatgtcggacgctaagctcacgtaaaatccgtaatatctaagacaagtaagggtaaatttgtcaattgacccctacggttctccgtaactgtctcaggatatatgaggcgcactgttcagagacgctccgtagaccgtagattccctcttcttcctcctctctctctctctctctctctctctctctcttctcatagaagtgtctctctctctctcttaggattcttctctgattctaacctgaattgtgtcggtacgtgatatgttgctctctttttgttgctgctgaaatttccataatgctttaagttaattttgcgaaatgctatacgtgagtaacggacaagtgtacaacatcagacaactgatcttgtttttgttcttttttttagatttctgtgttgtcggtttatgatcccatatgaaaagttcattatttttgttcgtataagatctggctactgaatttattcggttttggcgattggatctggatctgcctttattctgatttcctttcatatgatccgttctgaattttcgaatgatactgtgacgttattgttgttgttgttgcagatacttgatttcaaatggctactacgtataagcctaagaacattctcatcactggagctgctggattcattgcctctcatgttgccaacagattaatccgtagctatccagattacaagatcgttgtgttggacaagcttgattactgttcagatctgaagaatctcgatccttcgttttcttctccaaatttcaagtttgtgaaaggagatatcgcgagtgatgacctcgttaactacctcctcatcactgaaaacatcgacaccatcatgcactttgctgctcaaacccatgttgataactcctttggtaatagctttgagttcaccaagaacaacatttatggtactcatgtcctcttggaagcctgtaaagtaacaggacagatcaggaggtttattcatgtgagcaccgatgaagtctatggagaaaccgatgaggatgctgctgtaggaaaccatgaggcgtctcagcttttacccacaaacccatactcagccactaaggctggtgctgagatgcttgtgatggcttacggtagatcatacggactgccggttattacaactcgtgggaacaatgtttacggtcctaaccagtttcccgaaaaactgattcctaagttcatcttgttggctatgagtgggaagccgcttccgatccacggagatggatctaacgtaaggagttacttatactgtgaagacgttgctgaagcgtttgaggttgttcttcacaaaggagaagtcggtcacgtctacaacatcgggacaaagagagaaagaagagtgatcgatgtggcgacagacatctgcaaacttttcgggaaagatcctgagtcgagcatcgagttcgtggagaatcgaccgtttaacgatcaaaggtacttccttgatgatcagaagctgaagaatctgggttggtcagagcgaaccgcgtgggaagatggactgaagaagacaatggaatggtacattcagaatcctgagtggtggggtgatgtttctggagctttgcttcctcatccgagaatgcttatgatgcccggaggaagagttcctgacggctccgatgagaagaatgactcgtcaagcaacgcggtccagacattcacggttgtaacaccgaacaacaagactggtggttctagtgacaaagctttcttgaagtttttgatctacggtaagactggttggatcggtggtctcttagggaaactatgtgagaagcaagggattacttatgagtatgggaaaggacgtttagaagatagagcctctctcgtcgcggatattcgcagtatcaaaccgacccatgtgtttaacgctgctggtttaactggcagaccaaacgttgactggtgtgaatctcacaaaccagagaccattcgtgtcaatgtcgctggtactttgactctcgctgatgtttgcagagagaatgatctcttgatgatgaacttcgccaccggttgcatattcgagtacgatgctgcacatccagagcgttcgggtattggtttcaaggaagaagacaagccgaatttcactggctctttctactcaaaaaccaaagccatggtaaaatactaatatctataactcatgacagtctcgaaacaaagaagagtgtctttgtttataatctggttctgcttttttctcttcttctttgttcaccaggtcgaggagcttctgagagaatttgacaatgtgtgtaccttgagagtgcggatgccgatctcatcagacctaaacaacccgagaaacttcatcacgaagatctcgcggtacaacaaagtggtgaacatcccgaacagcatgaccatactggacgagcttctgccgatctccatcgagatggcgaagagaaacctaagagggatatggaacttcaccaacccaggggtggtgagccacaacgagatactggagatgtacaagagttacatcgagccagggtttaaatggtccaacttcacagtggaagaacaagcaaaggtcattgttgctcctcgaagcaacaatgaaatggatgggactaaactaagcaaggagttcccagagatgctaccaattaaagaggcgctgatcaaatacgtcttcgaacctaacaagagaacctgagacctgaacagcactcttcgccacaagtgcttacctataactgtttccttttctttctttctctttcaacagaaatgtctttttatgtttactcatcgtcttatgatccttgtaggtagtttcactatattgattctactaaaatatgatttatcattattatttatagagactaaagttttttttttctttctcaaattctacatttcccctcggattttacttactaagagacagtagtacacttataaaaatgaagagccacttactggttgtgatcattagggattccaccaaggcaggttcaaaacacttgaacttgattattaagctatagcctgagaagagaagcaaacaacaagtgttgatttgaaataaaccgaagtatatgatgtgaatttggtcaatattcagggttatagttttcttacgtgaacagttgagggcgggaacgagagagctgagagcgtttaataggggagagaataaggtttcgtagagttgactggggattagcagcgccaccaccaccgccaaatacaggatgtcctcctcctcctgctcttctgtttccaaggtctgttggaattgcgggtgagtcgttggagatgtcgaatggacctgagtttgaactgttctgtcttgctggccaaatctcatctttaggtcctggtgttgcaggagagtaaaacgaccggtctgcgccaaataagtcaacttgatttggagatattaaccaacaatagacttggaatcaaaagaagcactaggctcaaggtagttcattaacctttgcgagggggctctctgttctccatcatgttaactggtgggtttgagaaaaagtcaggttcgtggtgtcgtgggtagaaattgtttgctggttggatggctgtacgtttacatgactcgtattcacttcttagctggtcatacatctcatcgagcttcctcttctgtctggtgtttgaaacaataattacagctgagttagagataaggaacagaaccaggcaaacaaagcgtgtgttaaatcactgaccttgatttctcggagaacttctcttggagctcttgtttatccttggtcaagttttctacctcttgctccatcatctgacacctcttgcccatcttctggtatgctgtatgcacctgctccattttctcgctaaactttgct

SEQ ID NO: 21 - DNA, 1995 bp, Thlaspi arvense
  source 1..1995: mol_type = genomic DNA
atggctacatataagcccaagaacatcctcatcactggggctgctggattcatagcgtcccatgtcgccaacaggcttgttcgaagctaccctgactacaaaatcgttgtgcttgacaagcttgattactgctcaaacctgaaaaacctcaatccttcgaaatcctctccaaacttcaagtttgtcaagggagacatcgccagtgctgacctcgtcaactaccttctcatcactgaaaacatcgacacgattatgcactttgctgctcagacccatgtcgacaattccttcggtaacagcttcgagtttaccaagaacaatatttacggcacccatgtccttctggaagcttgcaaagtcactgggcagatcaggaggttcatccatgtgagtaccgatgaggtctatggagagactgatgaggatgcttcagtgggaaaccatgaggcttctcagttgctccctacaaacccttactctgccactaaagctggagctgagatgcttgtgatggcatacggtagatcatacgggttgccagttataacgactcgcgggaacaatgtctatggtccgaatcagtttcctgaaaagttgatccctaagttcattctgttggctatgagtgggaagccactgccgatccacggagatggatctaacgtgaggagttacctctactgcgaagatgttgctgaggcgtttgaggttgttctacacaaaggggaagtcaaccatgtctacaatatagggacaacgagagaaaggagagtgattgatgttgccaacgacatctcaaaactctttggaatagaccctgactccaccattcagtttgttgagaaccggccttttaatgaccagaggtacttcctcgatgaccagaagctgaagaagttgggatggtctgaacgaaccacttgggaagaaggactgaaaaagacaatggattggtacactgcgaaccctgagtggtggggagatgtctccggagctctgctgcctcatccacggatgttgatgatgcctggtgatagactctctgatggctctgacgagcgcaaggatgcagacggtaatcagacattcacggtggttactcccaccaaggccggtggttccgcagacaaaacatccttaaagttcctcatctatggcaaaactgggtggctcggtggtctcctaggaaagatatgtgagaagcaagggattccgtacgagtatggaaaagggagactagaagacagagcttcaatcatggcggatattcgcagcgtcaaacctacccatgtcttcaatgccgcgggcttaacaggcaggcctaatgttgactggtgtgagtctcacaaaaccgagactatccgagtcaacgtcgctggtactttgactctggcagatgtttgcagagagaatgatctgttgatgatgaactttgccaccggttgtatatttgagtatgacgctgcacatccagaaggttcagggattggcttcaaggaagaagacaaaccgaatttcaccggttccttctactcaaagacaaaggcaatggtcgaagagcttctaagagagtttgacaacgtatgcaccttgagagtgaggatgccaatctcatccgacttgaacaacccgcgaaacttcatcacaaagatctcgcgttacaacaaagtggtgaacatcccaaacagcatgaccatactcgacgagctcttaccaatctcaatcgagatggcgaagaggaacctacgggggatttggaatttcaccaacccaggagtggtgagtcacaacgagatactagagatgtacaagagctacatcgagccaggtttcaaatggtccaacttcactttagaggaacaggctaaggtcattgtcgcaccacggagcaacaacgagatggatggttttaagctcagcaaggagtttccagagatgctttccatcaaagattcgttgatcaaatacgtcttcgaacccaacaagagaacgtaa

SEQ ID NO: 22 - protein, 664 aa, Thlaspi arvense
MATYKPKNILITGAAGFIASHVANRLVRSYPDYKIVVLDKLDYCSNLKNLNPSKSSPNFKFVKGDIASADLVNYLLITENIDTIMHFAAQTHVDNSFGNSFEFTKNNIYGTHVLLEACKVTGQIRRFIHVSTDEVYGETDEDASVGNHEASQLLPTNPYSATKAGAEMLVMAYGRSYGLPVITTRGNNVYGPNQFPEKLIPKFILLAMSGKPLPIHGDGSNVRSYLYCEDVAEAFEVVLHKGEVNHVYNIGTTRERRVIDVANDISKLFGIDPDSTIQFVENRPFNDQRYFLDDQKLKKLGWSERTTWEEGLKKTMDWYTANPEWWGDVSGALLPHPRMLMMPGDRLSDGSDERKDADGNQTFTVVTPTKAGGSADKTSLKFLIYGKTGWLGGLLGKICEKQGIPYEYGKGRLEDRASIMADIRSVKPTHVFNAAGLTGRPNVDWCESHKTETIRVNVAGTLTLADVCRENDLLMMNFATGCIFEYDAAHPEGSGIGFKEEDKPNFTGSFYSKTKAMVEELLREFDNVCTLRVRMPISSDLNNPRNFITKISRYNKVVNIPNSMTILDELLPISIEMAKRNLRGIWNFTNPGVVSHNEILEMYKSYIEPGFKWSNFTLEEQAKVIVAPRSNNEMDGFKLSKEFPEMLSIKDSLIKYVFEPNKRT

SEQ ID NO: 23 - DNA, 5650 bp, Thlaspi arvense
  source 1..5650: mol_type = genomic DNA
cctttcgtttgcatttttgtgttacggtgtgatatctggtgaggatgtacatatgtggtaaagtggcaataatagtagctatttataaaattttatcataaatattattttttacatgaaactatatataatacaaaatttttaggcaaacaaaaattattagtcaaaaagtataattcaaaactcacttgattcaaatatcggaatcagcgcgctagatggggtgataagatttaccatctcaactaaatgaccatatatatgactccatataaatataatcaaatagttttgatttaataaaacgtttactattatatacgtttaataaaaaagtactttaccagcttttacgtgagttatggaaaaattatgaaatccaagatgtaatcaagggttgctgtccaaaaaaataaaaaaaaaacattagttaagggttgataagacgtagaaataaatagatcgttacgtaaaacatctgcaggaagtgtattgatttgtaaacgttaaaatacttctctctgaccatttattatatgtcagtcgttataagagaaaacaaaacgcacatgttatattgacatgtggacgataatgtatattgacatgtgaagaagccaactccgtacgtaaagcgccatttgcgctgcaaatactgatcctcaactacagtggattcttttctcacaaaaacatagaccgtttcaaatatatgttcccaaacacttgtgagacattctatacaaagtttatcatgcacccttcctttttttttaacgtcaaaatgatcatgtacttctgtcacattatttttgtacattatgaatatatagacatttgtattctctctttcgccgaatacacacatctatatatttgtaaatgtagcaggtttaaattttggagtaaaagaatagttgattttgaccacatagagaactaatggatcaattctcactaatacaaatatttggtgaaagccttagaggtgagaaagtccaaggcccataaaggcctattacggccacccccggtcacgtaaaaccaagcctcgttctcgttgatgaggccgaatataaaaggcatcatcaaatcaaacatgtgcgtaacgtgatatttaatttagcatttgcaagtaatgatcacttctgttttttcttttagtatttagttcttcacttaagttttccacgttatttattaatatacaggaactgctttttcttattttattggaaaaatcatgttttatgtttgttcaattaaaatttcagcacaatttctttttcatttttttatatgaaaactttaaggtagaaatgggaaattgcgaaaccgaatcatgcggacaaaacgaaatttctgcgttgcctatattttttgttggtaatttttaaaacaataaacaattaatttgacgtgataaaaacaaagggacatgaaaaaactctaaaagcgtagtaatggtaatttggtaatttcaaagtccgctggttctccgtaactgtctcaaattaaaaagagggcttactgttttctgacgctcgttagattccctctctttcttcttcattttctcctcttcgtcttcactgctaattcccttgatctctctccctgtcctccaattttccctctctacaaatatcgactccttcttcaatattagattgatcctgatccgattcgagccggtacgtacctgaactgtttctgtttctctgagctaattcgtgaatgtacgaggttctgctgcgttttattttttttgatggtatagttgacatgttttcaggattttttgttgtatactctgatctttgatgcttgtttatcgttttgccattggatctgctgctcgatctgggcaatttctcgcatttgattgctacatttgtcataacttcgggatctggatctaccttttttttctgatcatttagaatttcgctaacagctttagattttcgtacttatcgcgtggatttggcttcccgctacttgttcagctgtatctgttaaaatctcacaaatttttattgaactgaatcatggggttttcaataggatagatctggttgagttgttctcctcagtagctgtttccttccacatttgagcttaatcgatttcatggagtaatacgggtgatcgtgatttcgttaatcacatctgatccgatgacgttcatgatatatatactagccattatttacctgtttgctggatcttttgtccttcgattaaatctggctgtttcgtcattgacgagtgtactgaaagcttttccgaatgcaaatataatgttttgtgttgttgcagattatatatttatcatcacatggctacatataagcccaagaacatcctcatcactggggctgctggattcatagcgtcccatgtcgccaacaggcttgttcgaagctaccctgactacaaaatcgttgtgcttgacaagcttgattactgctcaaacctgaaaaacctcaatccttcgaaatcctctccaaacttcaagtttgtcaagggagacatcgccagtgctgacctcgtcaactaccttctcatcactgaaaacatcgacacgattatgcactttgctgctcagacccatgtcgacaattccttcggtaacagcttcgagtttaccaagaacaatatttacggcacccatgtccttctggaagcttgcaaagtcactgggcagatcaggaggttcatccatgtgagtaccgatgaggtctatggagagactgatgaggatgcttcagtgggaaaccatgaggcttctcagttgctccctacaaacccttactctgccactaaagctggagctgagatgcttgtgatggcatacggtagatcatacgggttgccagttataacgactcgcgggaacaatgtctatggtccgaatcagtttcctgaaaagttgatccctaagttcattctgttggctatgagtgggaagccactgccgatccacggagatggatctaacgtgaggagttacctctactgcgaagatgttgctgaggcgtttgaggttgttctacacaaaggggaagtcaaccatgtctacaatatagggacaacgagagaaaggagagtgattgatgttgccaacgacatctcaaaactctttggaatagaccctgactccaccattcagtttgttgagaaccggccttttaatgaccagaggtacttcctcgatgaccagaagctgaagaagttgggatggtctgaacgaaccacttgggaagaaggactgaaaaagacaatggattggtacactgcgaaccctgagtggtggggagatgtctccggagctctgctgcctcatccacggatgttgatgatgcctggtgatagactctctgatggctctgacgagcgcaaggatgcagacggtaatcagacattcacggtggttactcccaccaaggccggtggttccgcagacaaaacatccttaaagttcctcatctatggcaaaactgggtggctcggtggtctcctaggaaagatatgtgagaagcaagggattccgtacgagtatggaaaagggagactagaagacagagcttcaatcatggcggatattcgcagcgtcaaacctacccatgtcttcaatgccgcgggcttaacaggcaggcctaatgttgactggtgtgagtctcacaaaaccgagactatccgagtcaacgtcgctggtactttgactctggcagatgtttgcagagagaatgatctgttgatgatgaactttgccaccggttgtatatttgagtatgacgctgcacatccagaaggttcagggattggcttcaaggaagaagacaaaccgaatttcaccggttccttctactcaaagacaaaggcaatggtaatatactactacatcacataaccaaatcacatgtgatcctcaaactgtgcaactctgttcataaatcctctgtttctattttcgccaggtcgaagagcttctaagagagtttgacaacgtatgcaccttgagagtgaggatgccaatctcatccgacttgaacaacccgcgaaacttcatcacaaagatctcgcgttacaacaaagtggtgaacatcccaaacagcatgaccatactcgacgagctcttaccaatctcaatcgagatggcgaagaggaacctacgggggatttggaatttcaccaacccaggagtggtgagtcacaacgagatactagagatgtacaagagctacatcgagccaggtttcaaatggtccaacttcactttagaggaacaggctaaggtcattgtcgcaccacggagcaacaacgagatggatggttttaagctcagcaaggagtttccagagatgctttccatcaaagattcgttgatcaaatacgtcttcgaacccaacaagagaacgtaataccctcagtaacacatacacacacacacacttcctctttatctgttcattgttatattattatcttgcaggtagtttcaccactataatgattctactataattttattttgtcatatgattcatttactctttttttggctttctacgtcgaacatgtagctctcttgacgcattctacgtcacaacacattttgttttcattaatattatttattaaacgataaaattgcttgaagtttattactttcactattgattcctccttttagttttcatctcaaaagagcagaggacagagattaagaacgaaacagcattgactgataacttatttccctatgcttttaaacttgaggtttctatttgttacaaaacaatgtctttcataaagttaaatctttctattgcataaatcgaaactgcaaactcaaaatgtaatataaattaaaccttctgtacttcctgtccccaaatcttcaccatacgaccatctaaaagcagaatttttcacactacgaacaacattccataatctgatcatccttgcaacaaagcataccgattcgtaagccttcaatttgaatgaagacgtaaaatgatgaataaaaggtaagagagaggaacagaaaaagctcagcaccttgaaatttgttgttctcttcacatagcttgagcgtattgttgtctagacaatggtggaatttcttgtcccgtcataaaatttaaagtcaaaagtcagtagtagttggtgattgattggctcctgagttcttacaaaggaggagaacacttaaaaaacagagtatgagaatataccttctgcctctgtagacaaccaatcatgtgcacagaacagagcctgtctaatatcagggctcaaggaactctggctatcatcgatcaccggtctcccggagttgaacgttgaatcaggcgcaaggatcgacatcggcgtccccaagatatcgcgtgccatcatagagaggatagggtatctcggcgtatggaccttccagtaattcagaatgttgaactcgccactacgaggaaagattggttcagacaagtacttgtccaagtccgatgttgtgttctggctctgagatgtctcgtggagaaacttgtcgaatcctttcaacctgtctctagtgtccatacctgctctacctaagccagagccagagtaggaagagtcgtccccaacaatggctgagcatatc

SEQ ID NO: 24 - DNA, 1092 bp, Thlaspi arvense
  source 1..1092: mol_type = genomic DNA
atgggtagacattcttgctgttacaaacaaaagctgaggaaagggctttggtctcctgaagaagacgagaagcttctcaatcacatcacaaatcacggccatggctgctggagctctgtccctaaactcgctggtttgcagagatgcggaaagagttgcagactgagatggatcaattacttgagacctgatttaaagagaggagctttctctccagaggaagaaaatctcatcgtcgagcttcacgctgtcctcggaaacagatggtcacagattgcagcaaggcttccgggaagaaccgataacgagatcaagaatctgtggaattcaagtatcaagaagaaactgaaacaaagaggcattgatccaaacacacacaaacccatctctgaagttgagggctttagcgacaaagacaaaccagcaacaagcgacaataaaagaagcagcaacgatcataagtctccgagttcctcctctgcaaccaaccaagacttcttcctagaaaggccatctgatttctccgactacttcggttttcagaagcttaacttcaactccaacctcggactctctgctgcaactgattcttcactctgctccatcattccggcgcagtttagccccggaaacatgtctggttctgtcttccagactccggtatgcgtaaagccttcaattagtcttcctccggacaacagttcgagcaccgtctccggaggagatcatgtgaaactggctgcacctaattgggaatttcagacaaacaacgcctccagtttcttcgacaatggcggattctcatggtcaatcccaaattcttcttcttcactagtcaaacccaatcataacttcgaagaaatgaaatggtcagagtatttgaacacaccgttcttcaatgggagcactgtacagagtcaaagctcacaaccgatctacatcaaatcagaggcagattacttagccaatgtttcgaacatgacagatccttggagccaaacccagaacgagaatttgggcacaaatgaagctactgacgtgttctccaaggatcttcagagaatggccgtctcctttggtcagtccctttag

SEQ ID NO: 25 - protein, 363 aa, Thlaspi arvense
MGRHSCCYKQKLRKGLWSPEEDEKLLNHITNHGHGCWSSVPKLAGLQRCGKSCRLRWINYLRPDLKRGAFSPEEENLIVELHAVLGNRWSQIAARLPGRTDNEIKNLWNSSIKKKLKQRGIDPNTHKPISEVEGFSDKDKPATSDNKRSSNDHKSPSSSSATNQDFFLERPSDFSDYFGFQKLNFNSNLGLSAATDSSLCSIIPAQFSPGNMSGSVFQTPVCVKPSISLPPDNSSSTVSGGDHVKLAAPNWEFQTNNASSFFDNGGFSWSIPNSSSSLVKPNHNFEEMKWSEYLNTPFFNGSTVQSQSSQPIYIKSEADYLANVSNMTDPWSQTQNENLGTNEATDVFSKDLQRMAVSFGQSL

SEQ ID NO: 26 - DNA, 4198 bp, Thlaspi arvense
  source 1..4198: mol_type = genomic DNA
aaaataattaattctaacttatgacttaaaaaccttgtatacgtctagctaaatcttattttcgacgatcttttttaggattgggtcccaataaacaaatggataaacggtaagagtctaattacgagaggccatgtccagataacgtaagaaaactacaaaaattcaacatgtgaatatttgagggaaagtttgggtaaatggacaaagaatataccctctctattatgactggcttaaaggcaaaaaaagggagaaaattgccaaagagaagtgagtccatttaaatttaaaaatgtataaagcaaacaaaagagagacttaattattctcattcatctgcaaaagaaaccatgtatttaaagcaaaatttctaaattccgttatgtagacatttaattccatttaatcatttctggaaacattaaccaatgatgacaatttcttgctggttataaacctttcccattatttcctttaattgatgcgttcatcttcacaaagatttagtccacatttagcatttgcttttaattcattgcagaagtctttcatgacgacaacaatcaactcttctttgtccacattcttatctagctatacgatctacctcgtgtgttattttaattcttccgttctcactgtaaatcatttttgtatagcgcttccaccgctaactattgaatatcacttttcaatacattaatcgttacaataagtttgtagctgcaagaaaaaatttagactcggatgcatttggtaacatagatatcatataaaacttttaaactttttctcgtcatgcatcactcaaagagtgctatatccttgtcacataccaaaaacgttttaggagcacgcattgcttcaaactctcgtctatgtgcgatttcgatacatttttctaattactctcaagtctcaactaagcacacttaattatggatttcttttttaaaaatagcctatttctcgatttacaccactgtaaatatatttccataaattctatatcgaaccatatactacatgagtatttttaagtgaaaactaattattttgtgacataaaaaacctgatatacgtctaccaaaaaaggttttttttgtaaaccatgacttacgtgaatctccgaaataaaaatctttagaaaagttgttgaggtcacgctggcttttggctgcgtacgtatagagtggtccgtaacatcttcgaggacaaactgaacataaatgagcatgtctccactacttagaaccacacttactattaattttaaaagaagtttttttttttttttatcaattgttgcatttgtatgtatgatcataatgggtccatttggagatgacgaatgtatgatgattagagtgatgtaaaggtcaaaactacatgaactccatctcttaaccctgattattttaccattggtgtgaaccaatctgatgtggaccaatcatattgtttttacaaaagctaaaaaagatgtacatactttttgctatttaatctttaaagagagtatagaatttcaacaaacccttccgaagaaagtgacttttcttagtcctattaatactctctctctctctggagatctgttatttcgttttctccacctttctcttcagattccattaaccttcaaaagttttaaatacatgctctctgctcaaagcgtttttctttcgttacttgtaaagtaaaacacagagctctcttctctttcatcgttcctttgctcagcattgcagctttaataaaccaaaatgggtagacattcttgctgttacaaacaaaagctgaggaaagggctttggtctcctgaagaagacgagaagcttctcaatcacatcacaaatcacggccatggctgctggagctctgtccctaaactcgctggtaacattttcttccttaattcatgggatacaaaacatgaacttaaaaaggctaccttttttgtaataacaattcacaaatctgagtctactacaggtttgcagagatgcggaaagagttgcagactgagatggatcaattacttgagacctgatttaaagagaggagctttctctccagaggaagaaaatctcatcgtcgagcttcacgctgtcctcggaaacaggtaataattcatagaaaacagaggatcatgtttgtgtcttaagcttcctctgtttttaacagaggatcatgtatatcttttaaccttcctctgtttttttatcagatggtcacagattgcagcaaggcttccgggaagaaccgataacgagatcaagaatctgtggaattcaagtatcaagaagaaactgaaacaaagaggcattgatccaaacacacacaaacccatctctgaagttgagggctttagcgacaaagacaaaccagcaacaagcgacaataaaagaagcagcaacgatcataagtctccgagttcctcctctgcaaccaaccaagacttcttcctagaaaggccatctgatttctccgactacttcggttttcagaagcttaacttcaactccaacctcggactctctgctgcaactgattcttcactctgctccatcattccggcgcagtttagccccggaaacatgtctggttctgtcttccagactccggtatgcgtaaagccttcaattagtcttcctccggacaacagttcgagcaccgtctccggaggagatcatgtgaaactggctgcacctaattgggaatttcagacaaacaacgcctccagtttcttcgacaatggcggattctcatggtcaatcccaaattcttcttcttcactagtcaaacccaatcataacttcgaagaaatgaaatggtcagagtatttgaacacaccgttcttcaatgggagcactgtacagagtcaaagctcacaaccgatctacatcaaatcagaggcagattacttagccaatgtttcgaacatgacagatccttggagccaaacccagaacgagaatttgggcacaaatgaagctactgacgtgttctccaaggatcttcagagaatggccgtctcctttggtcagtccctttagctttttttttttcttcttttctttattctaacagatgtagagaacaaaaaaatatacaatacatacatacgtacagtggatttaagtctgtatattccatgagctgtctttatttttacattttttaagtgtgttttatacgtcaaatattcttctattttttgcaaacctttcagtctcatactatatatacacttttatttcttttgttttgttataaagattatgatgcatagaattctcgatgatttcagaggcctgtgtttgcaattaatctgttgaagaacaatgatgaaagtaataaatcagtttctgtctgtttccgtgaacgagttgtaatccagagtcacatccttcaccatttttcagacctgtaatgaattaaaagacaaaaaaggacaaaaacataaaactaaagcaaatcataataaataaactaaagtgggaattagctcaactaataagaagggacacttttttcgaaaggacccgagttttaatacgatgtaaattttttaaaaaaaaagatttctcttttctgaaatcaatgactgatcacatatttgagagattagaaatcgaatacccctaatttcttcgggctattataaactttgaaaaaggtacgtatgtcgtttttgcttagtcttatcagagaactggaattgtccccattgacggcgtcggaatattctttagcggtagaaaattcggaacagttcgagaattttctatttttttgtctgttaaataaaatagttatatgatttgaccttaataaacatgaaggaggtgatgactgatgagaaggtgacttcatttttaatcctaaaaagtcaattgatttattttataacccatatttttttttttagattctaatccaaaacaattacttattacttttttagaaataacctaacgcaataactcgactgataggtaaaactaaggtactataatatgtttgttttgactttaaatttaaagacgatgctatatttttttgaaaatgaaaagaaattgatctatcaaacacataagtctagattcaacagtaattataaaaatagaataaagagagaaaagttaatattttcttggcattatgagaaatcaaatatatttaacctata

SEQ ID NO: 27 - DNA, 939 bp, Thlaspi arvense
  source 1..939: mol_type = genomic DNA
atgttctcatcactctccaaccacaattcaccacaatcaatggagtcaccacaatacgagatctcttcaagctcttcttctgaaaaacctagatactactttcaatcatctgatatattccccaacctcactcaaaaccctagtaacagcaatacccaaatcgagtcgttaccgcttatcgatgggataaatttaaactcaaaccttaacctaaaccttaagccaccgtatgttgaaaaagaagaggaagagaaagaagaggaggaagatcacgttgatctgagcttatacatcggccttcctagctctgataattcgagaaacggtgttaaactgccgaagaagaaaaatgggaaggacatcatcactcatgacgctggaaaacagatggagaatgaactttccggcaaagcatactggattccggcggtggagcaaattataataggcttcactcatttttcttgccatgtatgcttcaagacattcaatcgctacaacaatcttcagatgcacatgtggggccacggttcacaatacaggaaaggaccagagtcacttaaagggacgcagccacgagcaatgctagggatcccttgttactgctgcgtcgaagggtgtaggaaccacattgaccatcctcgatccaagccgcttaaagacttccgaacgctccaaacacactacaaacgcaaacacggccaaaagccctatgcgtgtcgcatctgcggtaagctgttggctgtcaaaggcgattggcgcacacacgagaagaattgtgggaaacgctgggtttgcgtttgcggttctgactttaaacacaaacgctcccttaaggaccatgttaaggcttttgggcctggtcatgggtcttatctaaccggtttgcttgacgagcaggcctctcattcttctctctctgagaatttgttcttttaa

SEQ ID NO: 28 - protein, 312 aa, Thlaspi arvense
MFSSLSNHNSPQSMESPQYEISSSSSSEKPRYYFQSSDIFPNLTQNPSNSNTQIESLPLIDGINLNSNLNLNLKPPYVEKEEEEKEEEEDHVDLSLYIGLPSSDNSRNGVKLPKKKNGKDIITHDAGKQMENELSGKAYWIPAVEQIIIGFTHFSCHVCFKTFNRYNNLQMHMWGHGSQYRKGPESLKGTQPRAMLGIPCYCCVEGCRNHIDHPRSKPLKDFRTLQTHYKRKHGQKPYACRICGKLLAVKGDWRTHEKNCGKRWVCVCGSDFKHKRSLKDHVKAFGPGHGSYLTGLLDEQASHSSLSENLFF

SEQ ID NO: 29 - DNA, 4025 bp, Thlaspi arvense
  source 1..4025: mol_type = genomic DNA
ttattgtttatcaccagtttggttgtcattaattaataagtaagatttgtgtttagtgacggctgattccgtttctagtgagtcagatgcgagatgattgaaaaccgagaagacgatgactccacaaaataagattttgtttaagttttataattgagttttagcgaaattcgttttcaatttttttttttagctttattttttttattgagaatttaagaattataaaagttttttaggtaaaatttgaagaaattttgattaataaattatttaatgaaatagctttaaattattggttgtagaagatgacataacagttttgaactctactattgattaattatttttgctgatgtggcatattgttttattcttattagttagttttttcataatttaatgatgtggtaatttgttatattcttattaactgattattttgatgattatttttgcttatgtgacaccttttagagagcaggaaatctatccttttatataaaggagattagattttaccgatcatgtattatttagtagccaattccatacgtaaaaaaaaagatttttacaatttggttataaatagcctgcatgttttatcggtcctatcttgattgtatatcgtaaaaaaagatgctgactctaagaaaacgaatttcatatggaccgtagtaacagggcccacgagtaattagggttttaacggctgacattttgatctctcatacatctaagtatctaactagaagtgttgaccgttagagaagaattaaataacttacatacatacaaaaacataagtcgaagaataatcataacagtgcatcttctacaataatgataacaaaaaaagtaaattagaaattagcatttattccaattaatacccatattcgaaaatgcacatggtagaaaagcaaaaaaagcttacgtaaattccaaatattgtcaaacccctgcaatgacaaagttgccaaaaattgaagaggttggtccacacaatttaaggttctttgcttcacactctatttataggcaaagagatgaaacagaggagattaaattgcttcttaacaaaggttgttttcactcaaccacatgccttctcaagtgtctgctgctcacattccccgagattctcatttactttttcctctatttggtacgtactttatattacaattctatttgtttctttgattattcgttttgttcatacttagttatcataaacatacatagttgatcttatattatacagtaaatttgataaatgtttctcttaattaatttactcacgcaatttaaaggagacgattgatacgcgggcctacttacgcacctgcatgattattagttataaaagttattgcaaacattaaattactttgatagctagagagcaattattatataaagctaatttttaattgtagatatacatttagtcgaaagttaaaaagaaaaatgaaatccctaaacaaacgttcccaaatgttctcatcactctccaaccacaattcaccacaatcaatggagtcaccacaatacgagatctcttcaagctcttcttctgaaaaacctagatactactttcaatcatctgatatattccccaacctcactcaaaaccctagtaacagcaatacccaaatcgagtcgttaccgcttatcgatgggataaatttaaactcaaaccttaacctaaaccttaagccaccgtatgttgaaaaagaagaggaagagaaagaagaggaggaagatcacgttgatctgagcttatacatcggccttcctagctctgataattcgagaaacggtgttaaactgccgaagaagaaaaatgggaaggacatcatcactcatgacgctggaaaacagatggagaatgaactttccggcaaagcatactggattccggcggtggagcaaattataataggcttcactcatttttcttgccatgtatgcttcaagacattcaatcgctacaacaatcttcaggtacaaaccaatgttatctcatgcgcattacgtgcatgcataaccaaataaaatcttaattcatatttgaatttgtttgttagagaacaaacggagaaaatcctattcgatttatatgtttataacccccttatctaatgttatgagttagaatgaatttatacatacataatcaaaccctaagttgactaactatatattattttgtagaataattgttaatatagatctgtttggatgcatggttgagatttaattaaaatatactgttaaataaacccaaatttaaaatgaatttaatagtttttctccatataattaattaacgtttcctttaaaatttcatccaaattaataattaatagattacttcagtgaaacaatatgatcgatcaacaataagattgtaatgattaatgaaccattttattagttgttaaacttatatttgattaaaaaaaggtggggggtatttgttatgaaaatttatcttgaaatcgtcagtctaaccattgttatccatttcattttttgttattacatatactgatatacaaatctttgttgacaccaaatatgaattcagatgcacatgtggggccacggttcacaatacaggaaaggaccagagtcacttaaagggacgcagccacgagcaatgctagggatcccttgttactgctgcgtcgaagggtgtaggaaccacattgaccatcctcgatccaagccgcttaaagacttccgaacgctccaaacacactacaaacgcaaacacggccaaaagccctatgcgtgtcgcatctgcggtaagctgttggctgtcaaaggcgattggcgcacacacgagaagaattgtgggaaacgctgggtttgcgtttgcggttctgactttaaacacaaacgctcccttaaggaccatgttaaggcttttgggcctggtcatgggtcttatctaaccggtttgcttgacgagcaggcctctcattcttctctctctgagaatttgttcttttaaaactttgtgcatctatcagtcttttgacgtgtgttgtccgtttatttagtctagaataatgtggtatctaataattctcgtagtgaataatatcataatctacttgatatgagaggtcaacatatttagtcccgcagttaataatatcataatttggtgcaccaactaagggaaatgactatcagccacacttagggattactcattaatcgtagactcataaaagctatatgtttaaaaatgctagtttgtatacatatcattagccacacttagggattacgcataaaccgtagactcataaaatctatatgtttaaaaatgttagtttgtataaaaggagatatatatgaaatgagtaaggaagtcacattcaagagcaagtgtagtgtgctttccgagaaatttcctccacatgtccagacggtacgaaatgccagttaattccaattctatcattattgatctgaatgtgatcgcaaaacatgatttgattagtttcctctctttataatttactaaaataaaatgacgaccatataacatcatttttcagaaggaaattaaaacatatcaagaatattcatgtaaaatagttaaaatatcagttaaaaatgttaccattctatacatattatattttatatacatggactgaattcgacaatattggtaaatatcaataaatccaaaatcctgattatttgtaaatcaataaaatagtattatgatacaaaccaattcatatacggtgattcgaaatttcagtacatcatgaacatggaacaatggattttatgaactgatgagttgtccaacgttcgttacgtgcatgcattcaatgcctcggtgccacattactcaccttttatgtatcctctctccattgtttttttttttttatctaacttattgggtatccaaattcatttttgatccttattttagtgaaaccatgcatgtcctacgtactatcgctcgtcatatttaaaggaacagg

SEQ ID NO: 30 - DNA, 966 bp, Thlaspi arvense
  source 1..966: mol_type = genomic DNA
atgttctcatcactctccaaccacgattcaccacattcaatggagtcaccagactcaccacaatatgagatctcttcatgctcttcttctgaaaaacctagatactactttcaatcccttgatctcttccccaacctcactcaaaacgcttgtaacaacaataacctaatcgagcctttaccacttatcgatcggatagactcaagctcaaaccttaaccttaaccgtaagccaaaggcattgtatgttgaggaaggagaggaagaggaagaacaggaggaagaggacgtggatgtgagcttacgcatcggccttcctggctctgataatttgagcaacggtgctaaattttcgaagaaaaatgggaaggagatcatcactcacgatgccggaaaacaatcagagaatgaactttccggcaaggcatactggattccagcagtggagcaaattataataggcttcactcatttttcttgtcacgtctgtttcaagacattcaaccgctacaacaatcttcagatgcacatgtggggccacggttcacagtacaggaaaggaccagattcacttaaagggacgcagccacgagccatgctagggatcccttgttactgctgcgtcgaaggctgtaagaaccacattgaccatcctcgctccaagccgctcaaagactttcgaacgctccaaacgcactacaaacgcaaacacggccaaaagccctatgcgtgtcgcatctgcggtaagcttttggctgtcaaaggtgattggcgtacgcatgagaagaattgcgggaaacggtgggtttgcgtttgcggttccgattttaaacacaaacgctccctgaaggatcacgttaaagcttttgggcctggtcatgggtcttatccgaccggtttgtttgatgagcaggcctctaattcttctgtctttgaaagtttgttctgtgtgatcggtgagtattag

SEQ ID NO: 31 - protein, 321 aa, Thlaspi arvense
MFSSLSNHDSPHSMESPDSPQYEISSCSSSEKPRYYFQSLDLFPNLTQNACNNNNLIEPLPLIDRIDSSSNLNLNRKPKALYVEEGEEEEEQEEEDVDVSLRIGLPGSDNLSNGAKFSKKNGKEIITHDAGKQSENELSGKAYWIPAVEQIIIGFTHFSCHVCFKTFNRYNNLQMHMWGHGSQYRKGPDSLKGTQPRAMLGIPCYCCVEGCKNHIDHPRSKPLKDFRTLQTHYKRKHGQKPYACRICGKLLAVKGDWRTHEKNCGKRWVCVCGSDFKHKRSLKDHVKAFGPGHGSYPTGLFDEQASNSSVFESLFCVIGEY

SEQ ID NO: 32 - DNA, 3923 bp, Thlaspi arvense
  source 1..3923: mol_type = genomic DNA
cacgaaacaagttcaattaaatttaacggaaataaatggtaattaatgaaagtttgaattgtattaattactgcttccacgaaacaaccaaaaaaatcatttgacaaagtaatttgtgtacggtattagccacatggggccaatcctctatgtatatcggagttgttttgccaaatgcccaattcttactctagtaaatattaaaccattttgtagatcctaatcttgagtcaactcaaagtcctatgtttggaaactaaaaataagtttaaatcctctagactacgactaagttgtaaaaagaaatgttcaaaattccttagacattgaaattgaatacttcaattaaagaaaattttaccagcatgttcatagtagtagaccaccacagaacaaaattaatagtttttacattgcattcttatataataattttggagtataattgtaattataaacaaaataactaaaagaaaaggaaagtattcttgacatatttataggtactaatttgctagtacgactattaattatggagattaaatctagtatttgactaaccagaaacattttaaaggtaagtgataagttgataacctcgcaattatttttgaaagctattaatcactttagcagaatgtatacaagttctatattaacaagtttatctcaaaatcttgagatcctactaataaatcataacttttctcttattggtatgtcgaatctagtgtttaccaaactagaggtgttgaccgttagagacaattaaacaacttacatacatacaaaagtacaagccgaagaataatagtaataacagtgcattttctacaataattaaaacaaaaaagtatattaaaaattagcatttattccaattaataccaatattcgaaaattaatatggtagaaaagcaaaaagcttacgtaaattccacagattgtcaaaaccctgcaatgaaaaagttgccaaaaattgacgaggttggtccacaaaatttaaggttctttgcttcacactctatttataggcgaagagatgaaacagaggaaattaattactccttaacaaaggttgttttcactcaaccacatgccttctcaagtgtctgctgctcacattccccgagattctcatttacttactcctctatttggtacgtcctttatattacaattctagtattttttttttcattattcgttttgttcatacttcgtttcataaacatatatagttaatcttatattatacagtaactttgataaatatgtatcttaatttactcacgcagtttagagagacgattgatacgtggggactacttacgtacctgcatgattatataagttataaaagttattgcagaacattaaattactttgatagctcgagagcaatcattatataaagctatatttttaatcacaaatatacatctactcgaaagttttttttttaaaaaaaagatgaaatccctaaacaaacgttcccaaatgttctcatcactctccaaccacgattcaccacattcaatggagtcaccagactcaccacaatatgagatctcttcatgctcttcttctgaaaaacctagatactactttcaatcccttgatctcttccccaacctcactcaaaacgcttgtaacaacaataacctaatcgagcctttaccacttatcgatcggatagactcaagctcaaaccttaaccttaaccgtaagccaaaggcattgtatgttgaggaaggagaggaagaggaagaacaggaggaagaggacgtggatgtgagcttacgcatcggccttcctggctctgataatttgagcaacggtgctaaattttcgaagaaaaatgggaaggagatcatcactcacgatgccggaaaacaatcagagaatgaactttccggcaaggcatactggattccagcagtggagcaaattataataggcttcactcatttttcttgtcacgtctgtttcaagacattcaaccgctacaacaatcttcaggtaccaatgttatctcaaccaaattaaaaccagaattcaaattcgtttttttgtttctttatttaataaaaatagactttaatatatatatgtttggatgcatggtaaagaaaatatttattgaatagaccatggtttaaattcaaattacaagtttgattggctatcgttttaatagtttttcctcttataattaactcttcctttaatagttattcaaagagatttcaaccaaattaatacacagatttcttcaatgaaacagtatgatcgatctctaacaagattgtaatgattgatgaaccatgttattattagttaaaataaaattggagtcaaagatatgtgtatttgctgtgaacatttatcttgaatacggcagtcaaaccattttttatcaatttcattttttgtttttacgtatgcaaatctttgttgactccaaatatgaatgcagatgcacatgtggggccacggttcacagtacaggaaaggaccagattcacttaaagggacgcagccacgagccatgctagggatcccttgttactgctgcgtcgaaggctgtaagaaccacattgaccatcctcgctccaagccgctcaaagactttcgaacgctccaaacgcactacaaacgcaaacacggccaaaagccctatgcgtgtcgcatctgcggtaagcttttggctgtcaaaggtgattggcgtacgcatgagaagaattgcgggaaacggtgggtttgcgtttgcggttccgattttaaacacaaacgctccctgaaggatcacgttaaagcttttgggcctggtcatgggtcttatccgaccggtttgtttgatgagcaggcctctaattcttctgtctttgaaagtttgttctgtgtgatcggtgagtattagagtaactgagaggaatgtaactgagagaaaaaagaaaagtaataattaaaataattgaaaataataagtaagagcatctagatagtccttaccaaaaaaaagatcatctagatagtctttccatagtatctgatttatataatactaatataatgtatttatataattaaattataaattaataaagagatgaaccaattcttaacagacatttgcctagagacatatctttattcttaacaaacatttggctagaaacatatctaatagtttcttgtttcaaaaatgtcttcgctttttttctgtttttctcccttcctttttccttattttttttatttttcttattcactaaatatactcccataatatctgctatataatattaatataatatatttatataattaaattaaaaattcataaagagatgaaccaattcttaatagatatttggctaaatacatatttaatatttttttgtttgaaagatatctttgctatttttttctgtttccttctcttcttttcttcttatatttcttattttttttattcaataaatatattttaaaaaacaccaattttcttataagaatatctgaaaataattgagaataattaaattaaatattttcttttagtaaaacttataattatatatatgctaaaaagatatgtgatatttattattctatctttaaagatttttttatcgtgaatcactattttcagttatccaacctaaaatgaatactcagagagcaaccacatcgaaagtgttagagattgaatgaatacagttcttaaaaggtttatttcacattaatgaagtctatcaaaacaaaatttatctaattacatctctataacaaaaatgataattctaaagttaaatgaaaaaaaaaaacaaaaaaatagagagaatttttctttaaaaaaaagataagaaagatcaataaaaccgacttcctcacattataaatagaaaagtcttcat

SEQ ID NO: 33 - DNA, 1707 bp, Thlaspi arvense
  source 1..1707: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggaccagtgttaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 34 - protein, 568 aa, Thlaspi arvense
MSQYSFFYFFLISLFLYENCIAYRYTFTVIEAPYSKLCSTKKILTVNGQFPGPVLRAYKGDTIYVNVRNQASENITLHWHGVEQPRNPWSDGPEYITQCPIQPGSDFTYKILLSIEDATVWWHAHSSWTRATVHGLIFVYPRPPDTLPFPEPDYEVPLVFGEWWKRDVREVVEDFMRNGGEPNVSDALTINGHPGFLYPCSQSDTFKLVVEKGKTYRIRMVNAAMNLILFFAIANHKLTVVAADGHYTKPLTASYITISPGQTLDLLLYADQSPESTYNMAARAYHSNPNVGFNNSTTVGILRYYSSNDAGTSSSERYPYLPGYNDTSAAFDFFTKIKGLYSRVAPAKVSRRIITTVSINLLKCPNDSCAGPNGSRLAASMNNISFVTPSHVDILRAYYLHINGVYGTRFPEFPPRIFNFTADDQPLFLQTPRLATEVKKFQYGETVEIVIQGTSLVGGGIDHPMHLHGFSFYVVGLGFGNFNARKDPSNYNLDDPPYRNTATVPRNGWIAIRFVADNPGVWFMHCHFDRHQTWGMNVVFIVKNGIKPNQKILPPPPGLPPCDQFENL

SEQ ID NO: 35 - DNA, 5054 bp, Thlaspi arvense
  source 1..5054: mol_type = genomic DNA
catgcacgtaagaaactaaatcaacacataaggtaacgaggctcggaatagttgggtttcggatttttatttttaaaaagaaagcatcacctaaccaaaactcataagggcatctccatattaaaaaatacaaattaatagaatggaaaaagaaagatagagacgtatctcgtttgaactttgaaatatgtttagcaacgtatctaattaagtgtcatattctgattagtttatattatttttagatactctatttaagatatgcaccgttaaaggtgttctaaagcatcttcatttactctgatagtatatccaaaacttattataataatataatttatttgtgtaataaaatattaaattattaaaagtgtgatgcatatattaaaaatgacagatattgagaaacatatctcatgtcatgctcttttctttgaaaaacagttcgtaatttctctcattctctctctttctttctaattaatttttttattatccagatattctctgaaataccgttgttgcaaatacttttagaacacctttaacagtgtataatctcaaatagaatatctaagatttttttttattaatataaaaaaattaaactaactaatacttgacacttggtcagatacatttctagataggattctctatctctctccatcctttccattattttgtacattttaattattataaattggtcatatatgactttcagatattattctcgaattttccatcacttaccaacatttgttaaaataattttccgtttaaaatagtttaatagtatatattaacaggttacaggtatgaaaatgactttagtatgagatagggagtcgagagtttttaaaatattcgaatactttagacttctcaaattacccatttaaaaatattcaataacttagacttctcgcattacccaaaataccaaatttgtctgaaaccaagctatatctgaattcttttgtcctgtattttaattactatacttcgaacagtatattcagatatctgaacaattcgttctcacctcttaccttctattactggtttcaatactcgaaataaatgctattgctaaaaagtctgtttgttacttttacactataaatttagaatgttatttcccttaagttgaaagttacgtttgaccgagagatgtcaagtattttcaagagtattacgattcttttcagacatagatattctaattaacttttaaagcttacgatacgaagacaggatttgtgtatgaacatcgatggcataacaacatatcttgaatcttgtccaccagagttatctggggggcagttttgaaaatataaaagatctgtaaggttaataaaaaaaacattatcttgtaattccgtagttcttatatcgaaactgttactaatttgagaaagacgtggagagctagaggcgtctataaatatatattcctctctcagaaatcataatatacctaatttattaaaaaagtctctcaaaatgtcacaatattccttcttctatttcttcctaatctctcttttcctctacgaaaattgcattgcgtatcgctacacattcacggtgagttcaaaattaattaatcaatttagtcaggcgtgagtaaactaacttaaaactgatggtatataggttattgaagctccatatagcaaactgtgtagcacgaagaagattttgaccgttaatggtcagtttcctggaccagtgttaagggcttacaaaggtgacaccatttacgttaacgttcgtaaccaagctagtgaaaatatcacattgcattggtatttcatccaaaaccctttttttcgtcttcttaattatgtaaccattttaaatacaagaacataaacgaaatttcgatggttgaataaaaacaggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggtacaaaccaggacatataacaaaagatcagtgaagttgcttttttttttttttttttttttatctctcttaaattaaaattaatttcataaaaacttgacaggagagtggtggaagagggatgtgagagaagtagtggaggatttcatgaggaacggaggtgaacctaatgtgtccgatgctttgactatcaatgggcatcctggtttcttgtatccttgctctcaatcaggtttcttttactttttccctttctcctattaatttcttattttgtttttaatatacagttgtgggataaaaactaattgtggagatgaaataaaatttagggatattgatcattgaaacaaattgtgaaatgattcaaacgagcattttttttttcaagtcaactgtttttaatttgtttttcctcgttttttcaaaagtttcttactaatttagttagttaaacataattaatgtttgatcaaattgtccaaaaattagttaagaacttacgagtggttgataaaaaaaaaatcacgaggcatattaattcaaaccaaaatagttgtgtttgtatttattagttcttaaaaattgtgctcataaatttgaaactagcatttgcatgcatatttatgtttaaaacctagttagtaaagtccgttattgacatgcatacgttacgtatacgtatgtttaacagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggtacgtacacttacataaacctctaactttgatttggttcaaatctctttttaatattttgtgtttttgtaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataaatgattttatagcatgttgcagtattgtgtttgatgtaataaactactgcatatttacaaaaaaaaatataatcttaaattttgtttttatttaattaattttcacaagtttttctatcgtagcagtcataataaggaaagatactcccaagacaaaacagtgtctgaagtcattattttaaccctgagcctttctttctcataatatcttcaaaagctatagatgtggtgaagtttacctcgctcacactgataagtccatgtcttgtaccatatcatcataatcttgagggacctctgcgtgatggaaacgccctgtgcaagtggagtgatgcatctgtgtaatgtgactctgttgaagtgctatagccttgtggcttctactccagtctcttcggctccaaaggtataacggtggcggcatattgttccactgctctatcgtttgatcatttacatctatcgatccaggcaagtagaacgactacagagcaaaaaaacaaaggaatgtgattgaaatgtattggttatactaaacgagagtttgattcattagtgaacgtacttaccatgccagaaagtaatctcgtgtcttcccaaatcaagtcatactcagctatctcgtcgaccctgtgattgtagaggaaaacaaaggttatgaaaagtttgtaactgacactctatatttgctatttacctttttgtttcacttggtacgatgaggattagaatcttgggtctaaactcgagggccttcctgatgaaaatgttagcaagactggatttgtaaccgaaaggcgggtttaatcccattatctgtttttactcacgaccaagtaatatctcagttagtgaattcaaaaagtgtatttcacctttaggatgttggttgcgttttttaccagtcgagagccatcgggtaactcttgtttcttcacactcaaccaatctctcttctcaaagttgaagttatactgccatggaaacatagtgtttcagaaacatcttcaaacacaacaataaaagagtagtg

SEQ ID NO: 36 - DNA, 1524 bp, Thlaspi arvense
  source 1..1524: mol_type = genomic DNA
atgagctccacggagacatatgagcctctattgagacggctccactcagattctcagttaaccgtaggttcttcaccggagatagaggagtttctcggccgtcgtagatccacggtgacgccacggtggtggctaaggctggccgtatgggaatcaaagcttctttggacgctctctggagcctccatagtggtctctgtgctgaattacatgctcagcttcgtcaccgttatgttcatcggccatcttggctctcttcagctcgccggcgcatccatcgccaccgtcggtatccaaggcctcgcttacggtatcatgttgggaatggcgagcgcggtccagacagtgtgtggtcaagcgtacggcgcgaggcagtactcatcaatgggaataatttgccaacgagccatggtcttgcacctcgcagctgcggtcctcctcacgttcctctactggtactcgggtccgatcctaaaggcgatgggccaatccgcagccatcgcacgcgagggtcaggtctttgcacgtgggattattccgcagatttatgcttttgccctcgcttgccctatgcagaggttcctccaggctcaaaaaattgtaaaccctttggcttacatgtcactaggagttttcgtgctacacacgctactaacctggctggtaaccaacgtcctgcatttcggcttgctcggtgcagctctggtgctgagtttttcgtggtggcttctcgcggctgtgaatggtctgtatatcgtgatgagcccgagttgcaaggaaacttggaccgggttctcagctagggctttaagagggatttggccttacttcaagctcacgatagcttcagcagtcatgctatgtttggagatatggtacgtccaagggctagtgattatttccggtttactcaccaatcccacaattgccctagacgcaatttcgatttgcatgtattactggaattgggatatgcagttcatgcttggtctaagtgcggcaatcactgtccgagtgagcaacgagctaggagcgggaaacccacgagtggctaagttatcagtggtagtggttaacatcacgacggttgtcatcagcttattcctctgtgtcgttgtgctcgtgttccgcattggccttagtaaagccttcaccagcgacgcagaggttatagctgcagtctctgatctctttcccctgctcgccgtttccattttcttaaacggaatccaaccaattctctctggtgttgccattggaagtgggtggcaagcagtggtggcttatgtgaatcttgttacttactatgtcattggtcttcctattggctgtgttcttggcttcaaaaccagtcttggagttgcggggatctggtgggggatgattgcaggagttatacttcaaaccctaactttgattgttcttacactcagaactaactggaattccgaggtggagaatgcagctcataggttaaaagcttcagcaaatgagagtcaagaaatggctaccgaaggaatctaa

SEQ ID NO: 37 - protein, 507 aa, Thlaspi arvense
MSSTETYEPLLRRLHSDSQLTVGSSPEIEEFLGRRRSTVTPRWWLRLAVWESKLLWTLSGASIVVSVLNYMLSFVTVMFIGHLGSLQLAGASIATVGIQGLAYGIMLGMASAVQTVCGQAYGARQYSSMGIICQRAMVLHLAAAVLLTFLYWYSGPILKAMGQSAAIAREGQVFARGIIPQIYAFALACPMQRFLQAQKIVNPLAYMSLGVFVLHTLLTWLVTNVLHFGLLGAALVLSFSWWLLAAVNGLYIVMSPSCKETWTGFSARALRGIWPYFKLTIASAVMLCLEIWYVQGLVIISGLLTNPTIALDAISICMYYWNWDMQFMLGLSAAITVRVSNELGAGNPRVAKLSVVVVNITTVVISLFLCVVVLVFRIGLSKAFTSDAEVIAAVSDLFPLLAVSIFLNGIQPILSGVAIGSGWQAVVAYVNLVTYYVIGLPIGCVLGFKTSLGVAGIWWGMIAGVILQTLTLIVLTLRTNWNSEVENAAHRLKASANESQEMATEGI

SEQ ID NO: 38 - DNA, 4858 bp, Thlaspi arvense
  source 1..4858: mol_type = genomic DNA
accgtttctttttgttgcgtgtcgagaataatagtcatctatgtgttttatatatagagagtcatagactatagaaaaaatgttataagaggacaaaatttcaataaattgaattagtttaaatttgatcaacaatttttttatcatcttatttaattcttagatgattatagtaaacaactaagatatcccagtatcaatatattcatgcataaatagaatgaaattgtattgggggggggggggaatatacattatttaattatatagattgtcatcaagtaattaaataaaataccaaaatgacattaaccataacaaagtgaatatgctgctgcatatttgtaagaaatcaaaagaaactttgcagcatataattactcgatgactaatacaagtatattgtgttgaaatttgatgtagtgtggaatagttaagattatttagaaaataaaaccacttttaataattacctgaaaatgagtaataatcttctaatttggtggggccatccgacctaatagacaactcattgatttcatcttacgtcagggacagttcataaaatgcaaataattgtctttttttttggtaatgattgtctttaatctttattttgtagattaacagattatattattcctttttctttcttactgttcttcagattatttggatggtttcagttgggcctcaggagcagaccggtaaatgggttggaggactattggtgggccacatttctgaaaagcatttcttcctctcaaaagctcattattatgagtatctttttatacggtagaaaggagtacataagtggtaacataaaatatattctgccaaaaaagttagttgattcttcttaatcactactccttttcttgataattatctacgtgggaaacactatttgtcctccccccatttaatttggttgaaagtcttgagactagaggttggctcaatccaaaatcgttgacgaacgttaggtatcgttatcaactacaactctttttttcactgaggtaccgttatatttatatacctacctagttttactcttgtgcacaaggatatttcggttcggtttgatatctttctcgatcttgaagaaatattaccaatttggagcttatacattcctcggtctggtatcattcaacaaagtttttggtttggtttgtttaacaaataatatgaatgtgtgtttagtcgtcccttaggtatttctcaatctctttcttttaagcttcactttcttaataagccattaagtaacgtaactagagaatggttgttacactatacactaacaaaaatcagaccaaacagtcatgaaagatcttaaccatcaattccgtttcggtcaacgactcgaaccctcagcataaccaaaacaaacagtcgtaatataaacacgcccggtctataactataacaaaataacggcaaaagaaagaaaatacgagaaaaagaagaagacgaacaaacaaacggaccatgagctccacggagacatatgagcctctattgagacggctccactcagattctcagttaaccgtaggttcttcaccggagatagaggagtttctcggccgtcgtagatccacggtgacgccacggtggtggctaaggctggccgtatgggaatcaaagcttctttggacgctctctggagcctccatagtggtctctgtgctgaattacatgctcagcttcgtcaccgttatgttcatcggccatcttggctctcttcagctcgccggcgcatccatcgccaccgtcggtatccaaggcctcgcttacggtatcatggtatgtgctttagagagtaaacgacgttgacgtcgttcgatgttatagcttcacattcatttgcttatcattttgtaatatagtaaatccgggttttgaattaggtttctcaatattttgaccattttatgaataccagcaaccataataaaaatacaaactgtcggatcagttttgttctccatttgagatccgattttggtctttcagatgcaaaccatattaggatacagttcggttttcatgtcggctattttggtccggtttatcatttttgatcaatcttagccttaggcatctcccaactgtttttatgtatcaaatgtactagttgggaatggcgagcgcggtccagacagtgtgtggtcaagcgtacggcgcgaggcagtactcatcaatgggaataatttgccaacgagccatggtcttgcacctcgcagctgcggtcctcctcacgttcctctactggtactcgggtccgatcctaaaggcgatgggccaatccgcagccatcgcacgcgagggtcaggtctttgcacgtgggattattccgcagatttatgcttttgccctcgcttgccctatgcagaggttcctccaggctcaaaaaattgtaaaccctttggcttacatgtcactaggagttttcgtgctacacacgctactaacctggctggtaaccaacgtcctgcatttcggcttgctcggtgcagctctggtgctgagtttttcgtggtggcttctcgcggctgtgaatggtctgtatatcgtgatgagcccgagttgcaaggaaacttggaccgggttctcagctagggctttaagagggatttggccttacttcaagctcacgatagcttcagcagtcatgctatggtaaatgtctttaatctaaacacaaatctgtatgatttgaccggttaaaatttatggtttgaagaaagctgatatataccaatttaaaatgaaatatctgcagtttggagatatggtacgtccaagggctagtgattatttccggtttactcaccaatcccacaattgccctagacgcaatttcgatttggtactcctttcacccgagatttatcatatgccgacgaacctcaacggttacgtcttaacccggttaagtgattttgcagcatgtattactggaattgggatatgcagttcatgcttggtctaagtgcggcaatcacgtaacaaaacaaaattaaaaatggcatgtagtttttaatttaaatgatccatttgcaaaaaccgtgccgtttttgtgtgttttgtgttagtgtccgagtgagcaacgagctaggagcgggaaacccacgagtggctaagttatcagtggtagtggttaacatcacgacggttgtcatcagcttattcctctgtgtcgttgtgctcgtgttccgcattggccttagtaaagccttcaccagcgacgcagaggttatagctgcagtctctgatctctttcccctgctcgccgtttccattttcttaaacggaatccaaccaattctctctggtaaaaacacacacaagaaaaagaggctcttttgtgaattttggtttgttgattgatctttgctctatggattcaaaggtgttgccattggaagtgggtggcaagcagtggtggcttatgtgaatcttgttacttactatgtcattggtcttcctattggctgtgttcttggcttcaaaaccagtcttggagttgcggtataatctcttttactctttctttgtgtttatatgtattgtatgcatcataagagatgggaggtatttcattacaggggatctggtgggggatgattgcaggagttatacttcaaaccctaactttgattgttcttacactcagaactaactggaattccgaggtaaaacaatcaccaaatctctctttttttctctaaatactccaaagtccttacctttactctgcatctcttacaggtggagaatgcagctcataggttaaaagcttcagcaaatgagagtcaagaaatggctaccgaaggaatctaacagcaactctgttcttttctcctctcttttgttggcaagagatatgaaataaataccttctgtttattaggattcagtaacatatttcatttcgttaaactacagacaaaaaataatggacaaactacttaatcctcttctgcaaaatctttcttctgcctttagatgattacaaaaatcaaaactttctcgttgttttcataaccccacaagtttttccgtctgtgagctaacataagccaaagacttttcttgtgttttatcatacaacacagtttctcttgcatcatcatcaccatctccttcatagtcctcctcatcctcatcctcatcctcatcatcactactcacatcgacatcatcatcatcatcatcatcatcatcagatctgtagactccagcaatgatggttgtagtctcatctctgagaccagtaagttctttagcttcctctagctcatcttgagttgaagccaatgacaaaagcacattcttcgctttctgatcaggtggtaccccacaactctccatctccttgtaccaactaagagcgctcccgaaatccttgcacctcccagacgcatccatgatagtcgttatgatggtttgattcgctttgatgccacttaacctcattttctcatatacctccataattttatcaagatcgtttgctttcgcatagccttttatcattgtcccataggtgactatattcggctcaaaaccatctacctttatcctcttgaagaatttctcagcaccctccatgtccgaagcattcacatacgctgataacatagttgtgtaagaccagagatccgggaaaaatctgcaaagggaattatgttttgttaccaatctaaaacagagaagcaacacagttcacatgatagaattattctttacctgtcacgtcgcatgctttgaaaaacggtctttgcttgatccaccattccagaaatagcgaatgcatcaagcaaaatgttataagctttat

SEQ ID NO: 39 - DNA, 2214 bp, Thlaspi arvense
  source 1..2214: mol_type = genomic DNA
atgcttccattaatggcgataccacttgcgacttgcaggagcatcaactggtcagccacggaaaggattcctgtttcgcttctgttccggagtattcttctccaagacgacgaagtttgtagcgctgtgccactataccggatcctcgatcagaatgacgggcaacttggtcctataagtatggccgaggaatcagacaaaccattgctggatcctgatactctcaacagagaaggaattgacttgggtctgttgccattggaggaggtttttgaatacctaagaacatctccacgggggcttttatctggagatgctgaagaaagattgacgatatttggtcctaacagccttgaagagaaacgggagaacaagtttctgaagttcctaggttttatgtggaatcctttgtcatgggttatggaagctgcagcattgatggccatcgccctagcagatagtgaagtagagactatcagtcttttgctataccatttctgctcagtgctgaccggagaatcgctacctgtgaccaagaagaagggtgagcaagtcttctctggctctacttgtaagcaaggtgagatagaagctgttgtgatagccaccggttcgagcactttctttggtaaaacagcatctttggtggacagcacagatgcaactggacattttcagcaggttctaagcttgtgccagcagaaaaatgagattgcgcaaagagtttatgccatcataaatagatttgcagaaaaaggtttgaggtctcttgctgttgcttatcaggaaattccagagagaagcagcaacagtcctggaggaccatggttgttctgtggtctgttgccactgtttgatcctccaaggcatgacagtgctgaaaccatactgagagctcttaaccttggagtttgtgttaagatgatcaccggtgatcagttggcgattgcaaaggagacaggaaggcgacttgggatgggaaccaatatgtatccttcttcctctttgttaggccacaacaacgatgatcacgaagccattccattggatgagcttattgaaatggcagatggatttgctggagtgttccctgaacacaagtatgagattgtaaagatattacaagaaaagaagcatgtggttggaatgaccggagatggtgtgaatgatgctcctgctctgaaaaaggctgacattggaatagctgtcgctgatgcaacagatgccgcaagaagttctgctgacattgtactaactgagcctggcttaagtgtaattatcagtgctgtcttgaccagcagagccattttccagcgtatgaagaactatacagtatatgcagtctcgatcaccatacgaatagtgctcggttttacacttttagcgttgatatgggaatacgactttccacctttcatggttttgataatcgcaatactcaatgacgggaccatcatgactatctctaaagatcgagtaaggccatctcctacacccgagagttggaagctcaaccagatatttgcgactggaattgtcattggaacataccttgcattggtcactgtcctattctactggatcattgtctctaccaccttcttcgagaaacacttccatgtaaaatcaatcggcaacaacagtgaacaagtctcatccgctctgtatctccaagtaagcatcatcagtcaagcactcatatttgtaacacgtagtcgaagctggtcttttcttgaacgtcccgggactctcctgattttcgccttccttgttgcccaacttgccgctacattgattgctgtctatgccaacatcagctttgctaacatcaccggcattggatggggatgggcaggtgttatatggttatacagtttgattttttacatacctcttgatattataaagttcttcttccactacgcattgagtggagatgcttggaaccttgtatttgaccgtaagacagcatttactaataagaaagattatagaaaagatgacggagcgtccaatgtaaccatctctcagagaagtcactctgcagaagaactcagtggaagtcgttctcgcgcatcttggatcgctgagcagaccagaaggcgtgcagaaaccgccaggctcttggagggacactcggtgtcaaggcatttggaatcagtaatgaagctcaaacaaattgaccccaagatgattcgtgcagacactgtctaa

SEQ ID NO: 40 - protein, 737 aa, Thlaspi arvense
MLPLMAIPLATCRSINWSATERIPVSLLFRSILLQDDEVCSAVPLYRILDQNDGQLGPISMAEESDKPLLDPDTLNREGIDLGLLPLEEVFEYLRTSPRGLLSGDAEERLTIFGPNSLEEKRENKFLKFLGFMWNPLSWVMEAAALMAIALADSEVETISLLLYHFCSVLTGESLPVTKKKGEQVFSGSTCKQGEIEAVVIATGSSTFFGKTASLVDSTDATGHFQQVLSLCQQKNEIAQRVYAIINRFAEKGLRSLAVAYQEIPERSSNSPGGPWLFCGLLPLFDPPRHDSAETILRALNLGVCVKMITGDQLAIAKETGRRLGMGTNMYPSSSLLGHNNDDHEAIPLDELIEMADGFAGVFPEHKYEIVKILQEKKHVVGMTGDGVNDAPALKKADIGIAVADATDAARSSADIVLTEPGLSVIISAVLTSRAIFQRMKNYTVYAVSITIRIVLGFTLLALIWEYDFPPFMVLIIAILNDGTIMTISKDRVRPSPTPESWKLNQIFATGIVIGTYLALVTVLFYWIIVSTTFFEKHFHVKSIGNNSEQVSSALYLQVSIISQALIFVTRSRSWSFLERPGTLLIFAFLVAQLAATLIAVYANISFANITGIGWGWAGVIWLYSLIFYIPLDIIKFFFHYALSGDAWNLVFDRKTAFTNKKDYRKDDGASNVTISQRSHSAEELSGSRSRASWIAEQTRRRAETARLLEGHSVSRHLESVMKLKQIDPKMIRADTV

SEQ ID NO: 41 - DNA, 5382 bp, Thlaspi arvense
  source 1..5382: mol_type = genomic DNA
atgcttccattaatggcgataccacttgcgacttgcaggagcatcaactggtcagccacggaaaggtggtttcaattttcattcgtggaccctttttaaggattcctgtttcgcttctgttccggagtattcttctccaagacgacgaagtttgtagcgctgtgccactataccggatcctcgatcagaatgacgggcaacttggtcgtaacatagtggtttaagttcgtgtgtagctcgtactatgttttgatggtttgctattaccttagaattttaaataattatcttatgtacatcgacgaagttacggtttttgctattgttcactagttccaaggtgtggatcgaatctaggaatgtgggctttgaagtggtacttgtaccgtggattttgaagctataagtatggccgaggaatcagacaaaccattgctggatcctgatactctcaacagagaaggaattgacttggtaaatgatgtgtggttcctttacttttattaaaattcttggattctttagtggcaaaaatggttttgacgcttgagctggacagggtctgttgccattggaggaggtttttgaatacctaagaacatctccacgggggcttttatctggagatgctgaagaaagattgacgatatttggtcctaacagccttgaagagaaacgggtaataaaaagcttatcgtcaaagtttttccatagtgttcctgcctaagacaggagagaagcttactagttctgagatcttattcattatgaacactcttcatttctttttcacatattatccttaggccacacactgatttttctgccaagttgtggttctttacattagctcttgtataatatgttagtgttgtcctaatgacactgaaaacgaaacgatacaggagaacaagtttctgaagttcctaggttttatgtggaatcctttgtcatgggttatggaagctgcagcattgatggccatcgccctagcagatagtgaagtagagactatcagtcttttgctataccatttctgcgtacatgagagtttatgaaatcgtttctgctcttgaaactcatggtgaaaaatgtttattacagagcttaggtcctgactgggaagactttgttggaatcgtttgccttttactgatcaacgcaacaatcagcttctttgaagaaaacaatgctgggaatgctgctggagctcttatggctcgcctggctccaaaaacaagagtctgcttcttcaccttatgcattttcactcttattaccttaaatctgatcaagatccactcttgataggttcttagagatggacagtggcaagagcaagacgcgtctatcttggtacctggtgatattattagcataaagcttggggatatcattcctgcagatgctcgccttcttgaaggagaccccttgaaaattgatcaggcacgcacagattatatttagcgctaagtcacatttcttctctcttagtacattatgtaaagactgtatctgacagaatattcctgcagtcagtgctgaccggagaatcgctacctgtgaccaagaagaagggtgagcaagtcttctctggctctacttgtaagcaaggtgagatagaagctgttgtgatagccaccggttcgagcactttctttggtaaaacagcatctttggtggacagcacagatgcaactggacattttcagcaggttctttattgttccttaactcccttttctggttgaaacagtctgactggtaaaagaccaaaccttgtaaagctgtgaatacctttgcaagaacatgacatgtttctgtgacatgttttccttataggttcttacgtcaattgggaacttctgcatttgctcaattgctgttggaatggttcttgaaatcattgtcatgttccctatacaacatcgctcatacagaattgggatcaataaccttcttgtactactgattggagggatacccattgccatgcccactgtactatctgtcacgcttgccattggatctcatcgactctcacaacaggtgtcgtttctcttgactgattaactgtaacagagtgaaccaagaagaatccaacttttaagcttctgtagaactttttgttagcaatataatatgtgtttcttgttgttttaaaagggtgccatcacgaaaaggatgacggcgatagaggaaatggctgggatggatgtcctctgctgtgataaaactggaacccttaccttgaacagtctcaccgttgatagaaatcttattgaggtaccaaccaatacttcccatgtgacttgcatttcaaagtccaaagagtacagttaacaacagttgcctccatctttacaggttttcgatgactacatggacaaggacacaattttgctgcttgcaggcagagcttcacggttagaaaaccaggatgccatagatgcagcaattgttagcatgcttgctgatccgaaagaggtggagatgtttcttttacataaactctgttttatgaaaatattgtcatcttctttagaagttttacttattagaaaaggttgtttatctttaggcacgtgcaaacattaaagaaattcatttcttgccattcaatcctgtggacaaacgtacagcaataacatatattgactccgatggaaaatggtatcgcgctagcaaaggagctcccgaacaggtaacaaagaatcaccctatactacttggcactttcaaaactgactctagatgtaaaaaaatgaaagttcttttatatataggtccataaaagatatatcatcactgattcaattccaactgtactgtatacataggttctaagcttgtgccagcagaaaaatgagattgcgcaaagagtttatgccatcataaatagatttgcagaaaaaggtttgaggtctcttgctgttgcttatcaggtaaaatatttctgttatcccttttctctcttgatatggtttttattgtaagtgtactatttggggaagaagcttataatgacattagttatactcattcaatcattttaatcattgtttctaggaaattccagagagaagcagcaacagtcctggaggaccatggttgttctgtggtctgttgccactgtttgatcctccaaggcatgacagtgctgaaaccatactgagagctcttaaccttggagtttgtgttaagatgatcaccggtaattctgaaccctagagcaggcttgcttgggaccacagaggaacagacacgctctcatggtgaaaatctgataagtttggataaaaaaaatgtaggtgatcagttggcgattgcaaaggagacaggaaggcgacttgggatgggaaccaatatgtatccttcttcctctttgttaggccacaacaacgatgatcacgaagccattccattggatgagcttattgaaatggcagatggatttgctggagtgttccctggtcatactcacaacacagcgcttatatcttaccgagtttcagattttctctcttttactgatcccagtttcttttgatggctctcatgtttcagaacacaagtatgagattgtaaagatattacaagaaaagaagcatgtggttggaatgaccggagatggtgtgaatgatgctcctgctctgaaaaaggctgacattggaatagctgtcgctgatgcaacagatgccgcaagaagttctgctgacattgtactaactgagcctggcttaagtgtaattatcagtgctgtcttgaccagcagagccattttccagcgtatgaagaactatacagtaagtactaaagacaactatcatctgattccacattgctaaagataagcaaatccatgtaatataggatatgaatttcaagtagtttaatgaaattctgaagtttcttttggtttcctctgcaggtatatgcagtctcgatcaccatacgaatagtggtaaggagcttcaagatacacgatctaacttgctaagttacatctacttgagccttctcttcttatcattgtttcatgcagctcggttttacacttttagcgttgatatgggaatacgactttccacctttcatggttttgataatcgcaatactcaatgacggtaatcttttaatcctctcaatagcttcatgtttgaattgttgatgttaaaaactctaaattcttaacactttcaagtgctcttgtttatctcctacgcaacagggaccatcatgactatctctaaagatcgagtaaggccatctcctacacccgagagttggaagctcaaccagatatttgcgactggaattgtcattggaacataccttgcattggtcactgtcctattctactggatcattgtctctaccaccttcttcgaggtatcttcccgtcgctcgaaagtaatcaaattcgatttgcttaccgagaaaaacgcataaccatgaccatccctgaatcttaaatggctgcagaaacacttccatgtaaaatcaatcggcaacaacagtgaacaagtctcatccgctctgtatctccaagtaagcatcatcagtcaagcactcatatttgtaacacgtagtcgaagctggtcttttcttgaacgtcccgggactctcctgattttcgccttccttgttgcccaacttgtaagaactcttcatgtttaacctttttccatcacagaatactacatacagcatacataggtcttggttttgatttgtccgttttcatgtacacaggccgctacattgattgctgtctatgccaacatcagctttgctaacatcaccggcattggatggggatgggcaggtgttatatggttatacagtttgattttttacatacctcttgatattataaagttcttcttccactacgcattgagtggagatgcttggaaccttgtatttgaccgtaaggttagtgcatgtcttgtcctctgtctcagactttaactctgtttttttcttaaaagacaaagagtgttggttttgtcttgcagacagcatttactaataagaaagattatagaaaagatgacggagcgtccaatgtaaccatctctcagagaagtcactctgcagaagaactcagtggaagtcgttctcgcgcatcttggatcgctgagcagaccagaaggcgtgcagaaaccgccaggttctcttttgtctatacgactttttttgggtttctctttcatttcacatactaccagaaccatctctggtccacagaaaatagaaacttagtttttttgttcatttgataggctcttggagggacactcggtgtcaaggcatttggaatcagtaatgaagctcaaacaaattgaccccaagatgattcgtgcagacactgtctaa

SEQ ID NO: 42 - DNA, 1857 bp, Thlaspi arvense
  source 1..1857: mol_type = genomic DNA
atggagagtagtgtagttaatcatccattgcaagaagatgagaagggtttttctgatgaagataatgctgtaaataatgagaaagagagtttattgcaaacatctggctctgtagagaatgctcctgaaggttctggtcgttcttcggattggcgtaggggactagaccactgcataactgcacctgttggtctctatggagatatggtaatcgatgacaatgaggttaagtactctcggtccataacagagagattgtcccctgcgagtcataattcaaaattagatcgattatcggagcgggaaaagcaaaaactcattgttgagctagtcagaatacaaaatgacgggaccgtggaagttgatatagataacggtacaccggtatcggagttattggagtttcagccaaccaaagggcagccaacaatcacatatgaaaagtcatttgctgattcctttagatcaattccaagattaaaaattgtgatacttgtggttggaactcgcggtgatgtgcagcctttcttggccatggcaaagcgcctccaggagtttggtcatcgtgttaggttggcaactcatgcaaatttctgctcttttgtacgatctgctggagtagagttctatcccttgggtggtgatcctcgagaactagctggatatatggctagaaataaaggtctgattccttctgggcctggagaaatagcaaaacagagaaaacagttgagggcaattatagagtctcttcttccggcttgcacagagcctgatatgcaaactgctgcctctttcagagctcaagcaataattgcaaaccctcctgcgtatggacatgtgcatgttgctgaagctctaggagtaccaattcacatttttttcacaatgccttggacgccaactcatgaatttccccaccctttggcccgagttcctcaaagtcctgcgtattggctatcatatatagttgttgatctgatggtatggtggagcataaggacatacataaatgattttaggaagaggaagctaaaccttgcacctttcgcatatttcagcacataccatggctcaatttctcacttgcctactgcttacatgtggagtccccatgttgtgccaaaaccaagtgattggggtcctttagttgatgttgttgggtattgtttcttgagccttggatcgaagtaccaacctcgtgaagattttatccactggatagaaagaggatcaccgcccgtatatattggtttcggaagcatgcctcttgacaatccgaaaaaaacaatggatattatactggaaacactgagagatacagaacacagagggatagttgatcgaggttggggtggccttggaaaccttgctgaagttcctgaaaatgtattcctcttggaggactgtcctcatgattggttgtttcctcaatgttcagctgtgattcatcatggaggtgctggaaccacagcgactggactaaaagctgggtgtccaacaacaatcgtgccgttctttggggatcagttcttctggggtgacaggatctatgagaaaggacttgggcctgcgccaataccaatagctcagctcaatgttgagaacctctgcaattccataagattcatgcttcaaccagaggtgaaatcacgagtgatggaactagcgaaagtactggagaacgaggacggtgtagctgcagctgttgatgcattccacaggcatttgccactggctctgccactcccggagtcctcgccggagaaaagacacgaagatgatcgaccagacctgttacagtggttcttcatccagattggtaaaaagtgttgccttccatgtggtggtgtgtga

SEQ ID NO: 43 - protein, 618 aa, Thlaspi arvense
MESSVVNHPLQEDEKGFSDEDNAVNNEKESLLQTSGSVENAPEGSGRSSDWRRGLDHCITAPVGLYGDMVIDDNEVKYSRSITERLSPASHNSKLDRLSEREKQKLIVELVRIQNDGTVEVDIDNGTPVSELLEFQPTKGQPTITYEKSFADSFRSIPRLKIVILVVGTRGDVQPFLAMAKRLQEFGHRVRLATHANFCSFVRSAGVEFYPLGGDPRELAGYMARNKGLIPSGPGEIAKQRKQLRAIIESLLPACTEPDMQTAASFRAQAIIANPPAYGHVHVAEALGVPIHIFFTMPWTPTHEFPHPLARVPQSPAYWLSYIVVDLMVWWSIRTYINDFRKRKLNLAPFAYFSTYHGSISHLPTAYMWSPHVVPKPSDWGPLVDVVGYCFLSLGSKYQPREDFIHWIERGSPPVYIGFGSMPLDNPKKTMDIILETLRDTEHRGIVDRGWGGLGNLAEVPENVFLLEDCPHDWLFPQCSAVIHHGGAGTTATGLKAGCPTTIVPFFGDQFFWGDRIYEKGLGPAPIPIAQLNVENLCNSIRFMLQPEVKSRVMELAKVLENEDGVAAAVDAFHRHLPLALPLPESSPEKRHEDDRPDLLQWFFIQIGKKCCLPCGGV

SEQ ID NO: 44 - DNA, 7708 bp, Thlaspi arvense
  source 1..7708: mol_type = genomic DNA
ctaaaagagcatgagcagcaccgcgatcatcatcggaatagtccgccaccaacgcaattgaactaagaaaggctacaaaactgctttaaatccatcctgctgcttattccttttgcgtgtggccgccagaagaagatgagaaaacttactttcaccattagaaattagaataaagagtcaccgaaagtgaaagtgaataggatattcaccagaccgcctttgagaagcgccagttctgcagatcctgcatctcaatcactcaagataactcccaaatctcaaatcaactccgctaaagaaacttcctcttcgcaatcacgacgatttattaacagaaaagagaaggaattgctctttcgggagaagatccagcataactaacggaaagagtttcatcaaattcgtctggaataaagatgaaaacaagcaaaaggaaaaccgaacgttgtcggagaacttagaatcctataaacggaaaataaaatccgatcaacggaaaagaagatccgattgacggaactataagacagccaaaaagccgatcaaatcggatcaaagaaagaactctctcgctctgaaagattttaaagagagaagagagagagagcccttcaccttgtgccacccgtacttttaagagcatctccaaccatattctattttcaactccaaatgctattttgaagtaaaatcctctccaaccctagtctattttcaactttaaaatagagtaaacctaaattttactctatatttggagtaaatctaaactttactccatcttggaattagacttttttatttgcaaattaatccttgagatttattataattatattttatactatttaatatttataacaaatattataaaactgataattatgaatatttaaatactgcaattattcctacaaaattaaaaaatataataaactttaaaatacaaaataagggctaaaattaagataaacaacataaataaatttaagacaacaaatactaaaaatattaatgataatttttttttattatagtaatgtttaaataaaaattgcatatgcttttttgtgatgttatgtaatttttattatttgataaatatttaagttatagtttttcaaagaaattgtaaataataaaataagtgggatttatttataaattttaaaactaaaaaatatttataataaaatcaaataagattctatttagaatatttctttttagagtaaaaaataaagaaatacattagagtaaaacacaactctattttagagttactctatttttctattttggagtagaaaatgaagaaataattgaagatggtataacaaacgaaaagtcgcattatcttactgttaaattactataattcataatcaattggttttgtttcggtggcatccggcatcccctaatatgattgatttttatcgctgcatttattaaaatatatatatcttcatcctcgatcaccacaattcattgattcaaaagtctccaaaccccttggaagctcaacattacaatctcgccaaattcctcaaaaccagaattttccacagacccagaaacatttctcatcaaagttctctcctttcctgtctcttgaactttctctcctactcactcagttcaagaacaatctttcgatcattgatcagtgatggtagattcaattgttgggtttcatttacttgggcaatggcttttcttttcttttctcttctcttttcgattgattgattggttaattctgtgtctctgtacagtttcgaattgtgtctttcaagttaaagctggatcatttttctagttgatctcatcctcgcgcttaacgtgagagactctctgctctgtaatctttccctgcgttttcttgttttatctgaaacggttaatatagagataaaaaaacccaatttctctgacttttttttctttctttaatatatcatttcttctcccttttgctttttgtataaagtgcagctaactttccttaaggattcactggttggttcaagcactgattcgccgcgtacagcttttggcaggagccatttgcatctctgatcgacatggattagaatagtgtgcagttgtgttgtgtacaactcgctgcatagatgttactttgtggagttcaattcatatgtagtatgtttgttgtttaagaagctgatctttgtgggggcattgggtagtgagatctgttaaattgaagtcggtgatggagagtagtgtagttaatcatccattgcaagaagatgagaagggtttttctgatgaagataatgctgtaaataatgagaaagagagtttattgcaaacatctggctctgtagagaatgctcctgaaggttctggtcgttcttcggattggcgtaggggttagtcctctattggaacacgtgtatttatcttattccaagctttgccatcaaaaccataacttagagcataacaacttatcaagattgtatgttcttgaaggactagaccactgcataactgcacctgttggtctctatggagatatggtaatcgatgacaatgaggttaagtactctcggtccataacagagagattgtcccctgcgagtcataattcaaaattagatcgattatcggagcgggaaaaggtaggaatctgaaccttcattgcaatggatatgttcttcatttgattacaaatttttttggtaacttgaaaacttcttgttattccagcaaaaactcattgttgagctagtcagaatacaaaatgacgggaccgtggaagttgatatagataacggtacaccggtatcggagttattggagtttcagccaaccaaagggcagccaacaatcacatatgaaaagtcatttgctgattcctttagatcaattccaagattaaaaattgtgatacttgtggttggaactcgcggtgatgtgcagcctttcttggccatggcaaagcgcctccaggtagtttcgaaattgctttctcattctttgtatccttttgcgaaattaagattcacagagggaatcattgtgactttacatatgtatattttgtggatgcatgcatatagtacactgcaattgaacaggctcgtatcaaagtgttggcatgtgcctatatcatgtgttctcgtcattttaaatttgtttcatttcttcatcctatcgtcatatgtgttcttgacgcttttcttagcatttggtgagagatttggaactgattggcattgagagaggttaaagttagatattgtttttctctgttgcaggagtttggtcatcgtgttaggttggcaactcatgcaaatttctgctcttttgtacgatctgctggagtagagttctatcccttgggtggtgatcctcgagaactagctggatgtaagaagtcctccttgaaaagattcgtttatattttagtttctcgagttttttttgtttgatgatgaaagctgtcataaattctctctttagcttcatgtgggtatttttgaatgtagatatggctagaaataaaggtctgattccttctgggcctggagaaatagcaaaacagagaaaacagttgagggcaattatagagtctcttcttccggcttgcacagagcctgatatgcaaactgctgcctctttcagagctcaagcaataattgcaaaccctcctgcgtatggtatatccttcgttaactttaaaactctttgatccatcagaacctcattgtttgtaaacctttcttaagcactttttctatgaaactgatggtatggtttttggttctaattataggacatgtgcatgttgctgaagctctaggagtaccaattcacatttttttcacaatgccttggacgtgagttgacttctctgcttttaaacttgccttaacgtgttatttggtttcctgttctcttacatcatcttcatgtaggcaggccaactcatgaatttccccaccctttggcccgagttcctcaaagtcctgcgtattgggtaatgttattctttcttatgaccatatactctctcgaagataatgaggcttattgtttcacaacatttttttgtctcagctatcatatatagttgttgatctgatggtatggtggagcataaggacatacataaatgattttaggaagaggaagctaaaccttgcacctttcgcatatttcagcacataccatggctcaatttctcacttgcctactgcttacatgtggagtccccatgttgtgccaaaaccaagtggtgagattcccgctgcatccccagtacatatattccgctgaggtttatcatatgtattgtagcttcattctggcttctagattaatgttgttcttctggctttgtcaattggcaagaccataatgggttgtcaattttaggctgagctctttatctttccttcggtagatatctgaaagattttgctttttgtttgactaatttgcagattggggtcctttagttgatgttgttgggtattgtttcttgagccttggatcgaagtaccaacctcgtgaagattttatccactggatagaaagaggatcaccgcccgtatatattggtttcggaagcatggtaaactgatgatccttagaaaatatgctcttccttgctttggtgatattcagttataatgcatagtactgatattgttgtttcttctcttttacacaaccaagcctcttgacaatccgaaaaaaacaatggatattatactggaaacactgagagatacagaacacagagggatagttgatcgaggttggggtggccttggaaaccgtaagcagcttctgtaccttctcttttgatgctctgtgtcctgattcctgaactacttttcttctttgcttatacataattttttttatcttcaaacatcttgcagttgctgaagttcctgaaaatgtattcctcttggaggactgtcctcatgattggttgtttcctcaatgttcagctgtggtaacccctttttagattaactacatcatgtctccaaagacattatcttctaatccaggctatttcttgtgttgtggatatgaagattcatcatggaggtgctggaaccacagcgactggactaaaagctggggtaaaacattttgctttcacagaaatgtcctgtagtgaaatcagttttcttgaccatatgaaacctaatggttcctatattcttttgaattctatgatatgtctccagtgtccaacaacaatcgtgccgttctttggggatcagttcttctggggtgacaggatctatgagaaaggacttgggcctgcgccaataccaatagctcagctcaatgttgagaacctctgcaattccataagattcatgcttcaaccagaggtaaacaaacttaaattttccttctttttttccatgtatctcatgcccatgtaggatgtttgagtaaagaaccacagtagaagttaaacacaaacactctttcttgcaaaattaaacaaactaaacaaaacccaaaacctaaagctgtccttttgagttcaaaaaagtagaaagatgtaatcttgtatatacacttaaatattgcttatacagttcatacaaatagaaaatgtcatctttatagttcatagaagtagaaagatgtaatctttatatatacacttagaaattgcttatatagttcatacaaatagaaaaatgtcatctttatagttcatacaaatagaaagatttaatctttatagttcatacaaatagaaagatgtaacttttatgtatatgcgtggatatcgctttatcagttttttaatatgtgaaggccctcgcacaaaaaaaaaaatattatgtgaaagaaagctcggtgcttaatatttgaactatgaagcaactttctatatatatacacaaaactgacattgctttatagttcatacaaaaaaaaagatgtaatcgttatagttagttcatacaaaagaaagatgtgatctttctattttcgttcagtgatgttatgtgaagaaaaattcggtgtttagatttgaactatgaagcatctttctatttatatacacaaaactgatattgttttatactcatacaaatagaaagatgtatgaactataaaaatagaaagcatcatacaaatacaaatagttcatacaaacatcacacacaatgcacactagctcaaagctttttcctctgtttaaaactcataaaacttccaaaggtctctctctagctttgttgtatctcacaaacggaactggaatcggtagaatatgtaaaaccaatcatggcctatccaggtgaaatcacgagtgatggaactagcgaaagtactggagaacgaggacggtgtagctgcagctgttgatgcattccacaggcatttgccactggctctgccactcccggagtcctcgccggagaaaagacacgaagatgatcgaccagacctgttacagtggttcttcatccagattggtaaaaagtgttgccttccatgtggtggtgtgtgataacaaactcccttagatattttgatcttgtctctgcaactcgtttcatttcattcagttgtcaagaatatgattagatttttaacacagctgcaaaagatgggtttggggcatctttataagtttgttgttcagtgagaataagatgatatttgtgtgagtttgtcttaagaacaaagtaccgatctcctttattgtatacttaaaacccattcgaaacagaatctcccacgtcaaactacatttctcagcttgtaggcattggatttcatcagctcctgagcatgtgccagatgacccttcatatgatctgttaagtttcagtggctcaaaacggcacctttaagagaagcttgaagtatcaaaacgtgagattaaaacggaagttgcagaaacttgcagaaagcagagtatgagagattgagagaagaagaagggagaagggaacacatacaaatatttaacgagttcacgcccataacacgctatatctcaccaagactaagctggaaatccactagatatcagagagaatacatatttagagacgaacaagtgaccgttttaatatctttcactagcacttaaaattcactatgttaaatcaattaaaaccagaaaaaaaacaatagctaaacttatgctaagatgtgtttttctctccactctcactagcgactacattgtgtctatctcgttctcttcctcatgtgtactacaatctctctcacgtcttcagcttcactttatagtgacaacaacattagacctaaagcaccttgccaaacgccaaaacagcttcctatagatgcgtttgcttttttatcatatcttagaggcaaatactccttagcttttaaattcacaacaagacaaaagcaagaggctctccttgtggtcccaaaagtaactcaatccaatatgaactcaccaaagtaatcatagttatgccttgagttacttgacttgaatttgcttgagcgacaagttctcgtcttcacaaatctccacctgagaacatgatgttcaagccaaacgacatacactcttgagaaagatatagaacgccttcttctagcttatggattctaatcttggtttctgagtatgattacccagtcttgatactaagcagatttaaataccataaaactgatagatccttgctggatttaa

SEQ ID NO: 45 - DNA, 732 bp, Thlaspi arvense
  source 1..732: mol_type = genomic DNA
atgggaagagggaagatagagataaagaagatagagaatcagacagcgaggcaagtgaccttctgcaagaggagaactggtcttatcaagaagactaatgagctctctgttctctgcgatgctcacattggtctcatcgtcttctcctccaccggaaagctctcgcagtactgttccgaacccctcaggatgcctcagctcattgaccgatacttgaagaccagtggaatgcgacttcctgatcctaatgacggccgggaggaattgtaccaagagatggaagtactaaaaagagagacatgtaagcttgagcttcgtctgcgtccataccatggacatgacttaacctcccttcctccacacgagctcgatggtctcgagcaacagctcgaacattctgtccttaaagtccgcgagcgtaagaatgagttgatgcagcaacagttggagaatctaagcagaaagaggcggatgctagaagaagataacaacaatatgtaccgtttgcttcatgagcatcgtaacgcggttgaatttcagcaagctgggatagagacgaaaccaggggagtatcaacagtttctagagcagcttcagtactataatgatcatcagcaacaaccaaacagtgttcttcagcttgctacgcttccttctgagattgatcctaattaccatctccagcttgctcagcctaatcttcaaaacgatccaacggccaagattgattag

SEQ ID NO: 46 - protein, 243 aa, Thlaspi arvense
MGRGKIEIKKIENQTARQVTFCKRRTGLIKKTNELSVLCDAHIGLIVFSSTGKLSQYCSEPLRMPQLIDRYLKTSGMRLPDPNDGREELYQEMEVLKRETCKLELRLRPYHGHDLTSLPPHELDGLEQQLEHSVLKVRERKNELMQQQLENLSRKRRMLEEDNNNMYRLLHEHRNAVEFQQAGIETKPGEYQQFLEQLQYYNDHQQQPNSVLQLATLPSEIDPNYHLQLAQPNLQNDPTAKID

SEQ ID NO: 47 - DNA, 5332 bp, Thlaspi arvense
  source 1..5332: mol_type = genomic DNA
ttacatttcaaacagatttgacataacatgtaatattgcttaggacatttgtgttcatcacgtccgtctttcaatgaccaattaatgctgttttgtttgtttgttttaaccacgttttagcaaaagtacatttatacaggacttttttaatccgctcgttacttaatcgacaaatatataatgtccctcaatactatttggtatatattttttacaaaacatgccactgaaaatgattcagttgtatatgttcatatcatcacataattgtgttcaacctgatgtagcggctgattactctatgcacaatgttagaaaatctgaatattaactcgatgatctaaataccaatttcttttccagaattttaatacgttcgagtttcagcataaaagcttttactcacaccacttgtttccccttgcgttgtgtggcaatgtcgctgtataaatgtacatattccttttgttttcctccaaaaagtttattaatgaaattcagattctacgatataataataaataatgttacttcaaaaataaaagagtgtgccaatatggactgtgcttttaccatgagggatttaaactcgattttttttttgttacactgaagttgatgaatatagacattaaattaactcgaatagacaggaaaaatccgatgtccaaaaaaaaagacaggaaagagatatcacaaataatctaacaaaactattatggctaagtatcacatttttagatatatcaacggttcaaagcccgatccatactgtcatacgtaaaaaaatattgttttggcaaaactacatataaggtgatgttactatagaaataaaaccaaattaagaggaaggtgggtaagaaaaaataagagagagtcactttcaagtattgttacattttgtcttttcccatttggctatctctctcttttgccttttcccttttgtggtaactttattttccctataacattttttcattaagtttccatttatggattattttctagttgcgtcaagcatggaatatcaaagaaattagttcgagatttgactaaaaagggtcatttctgctaaataaattactaacactgccattaggccaatcacatgtctagtaacaatttgacatttcacttactattgtaacttttgatagatattgagactttacaaaaaaatattttaagaaaattttgttgaagaaaaaaacttgaatatcattcactataaaaaaaaggaaaaaatgtataggtttttgttacaatactgaacgtaaattgtgaccatgattccataaagcagtaacatttcaaactctaaaaattgttgatattaaaaacttgaatatcatccactataaaaaaaaggaaaaaatgtataggtttttgttacaatactgaacgtaaattgtgaccatgattccataaagcagtaacatttcaaactctaaaaattgttgatattaatctagtttacagaaaaagtagaagaaagaaaaaatgaaattagaaaatgttgagctgacatcacacgccacacacagaattgtatagaaaagaggaagtgtaattaagcatattcttccgcattaacctctctcctctctgtgtcatccaacctcatatactactgatctccatttccagatccccaagaacacaactcatcaacaagtactttaaatactctatctctctctttagagatctctctttctctctctctctctctctctctctctctctctctttctctctgtttctcactaattactctctacctctcttacatacacaaacacaatctctctcgcttctctctaattcacacatcaagataaaagaatcaaaccatccttgaaatgaatcctgtaattaattataggatttcagtttttgagttttttgttcttaatttggttttgtagttaattagggtttctttatagtttatctaccttgcatatatatatgtatgacatagacttacatgacactgaatcaggaggaagaggaaggaacgaataagaggaagaggagagagatgggaagagggaagatagagataaagaagatagagaatcagacagcgaggcaagtgaccttctgcaagaggagaactggtcttatcaagaagactaatgagctctctgttctctgcgatgctcacattggtctcatcgtcttctcctccaccggaaagctctcgcagtactgttccgaacccctcaggttaacttcttcttcctctccctctctcgaattagggtttcagagatctatacatacatgtgtatgtattatctaattaaatgagtaaataagagatatatagctcgtgcttgtgaacagacaattctcatcattagtttctgaagttagcaggtatcatgaatactatgtttcagattttaatttgacccagaagttaagaatctctctgatgctatatatatatatatatatatatatatatatttacttttgcagtatttgatttgtatatatctgtagatgcatgtatataatcattgtttattgatatcggtcaaattctgtaatttctacacctgccaagcaaagagatgttttttcaaagatttttcatttcttgatcaaccttttttcccatataatgtttacgacatatatatatatatacctagtatatatataagtcttatttgaatcattcgatataagtaccattaattataaaggatttttattaaaagttgtaaatctaagtcatgaaccagaagacgaatttattcacatctagcctaacaatataagaagaaaggtatgctgatatagtttgaattttgctatcataaaacaaaaatggggaaaatgtatactctctccgtttttttatataaaatattttatgaattgttttttgttcaaaaatagttgatgttctcacatatctatgcagaaattaattgcattttattgattttaagtgtttaaattttgtatattgtttttttattggttgaattgttttggaattaatgactaaaacatgttttgttttggaaatatacaaaattaaatgatttcttaatctatataaaaaaaccttaaacatcgtatataaaaaaacggagggagtatgttatttgatttttgtgttcctccaatgatgattaaagtcttgacgatgtacgattaaagatactatttgataacaatttattattaccaatgttgaataggatgcctcagctcattgaccgatacttgaagaccagtggaatgcgacttcctgatcctaatgacggccgggtaatatatatatacacgcatacttattcctttgtcttagccaattcaaaacaaagaatataacattctcacaaaaatcaataatcatttggagttttttgtgtgtatatataggaggaattgtaccaagagatggaagtactaaaaagagagacatgtaagcttgagcttcgtctgcgtccataccatggacatgacttaacctcccttcctccacacgagctcgatggtctcgagcaacagctcgaacattctgtccttaaagtccgcgagcgtaaggtaacgtaatatatgttcacatcaatatacatattctcttaaaactatagtgattattacacttttgtttgatctctcttttctatctctactgttaataaattgaagaatgagttgatgcagcaacagttggagaatctaagcagaaaggtcgctctctaattaatcattcttttgataagtactcttaattttattttcctctaattagtcatcttttataccgcggatttaatgatcattaccgtgtctatataaatatatgatcagaggcggatgctagaagaagataacaacaatatgtaccgtttggtaagtttggttactagagaacgtttaatttggttgcacattttcggtttgtgtgacataattattcaacgtattaattgcagcttcatgagcatcgtaacgcggttgaatttcagcaagctgggatagagacgaaaccaggggagtatcaacagtttctagagcagcttcagtactataatgatcatcagcaacaaccaaacagtgttcttcagcttgctacgcttccttctgagattgatcctaattaccatctccagcttgctcagcctaatcttcaaaacgatccaacggccaagattgattagtcctcaaaagtgaatcatttatgtcttttattactacctattttgattatagccaatgccttcttctgtgtctgttttgtgtggttatggaaacctaatattgtttgaagtacaattcacttgaaaagcgtttatggtctttctttgattaaataatttaatcttcattatcacacatttgccaactagagatcaactgtttatgtaatcgcagattttgagatacatttttggccatagaaagagtgggagacacatgagtaaactattcttaggttgttgacaaaaggaagaaccaacacctcagaatcttaataatgtgtgatcgacaagcttatcaaggaccttcactgaaaatagattttatatatttcagacagattagatcgcaatcccaaaaatagctcttataaactcagccaagattgcaaaaattcaatcaacttctgtagggtattgcaagggacgatcaagaagcaggaaaagagttctggaagaagaatcttcctttcctcttttcctcttcaatctgttttcatcataaagacgagaaaaaattgaatacttggcttcttgatcgctcgaataaccatcatcatcatctagatcccccaaacgctgcgttttttgttggctgaaccgtctacatccatgacaggaggatattttatatccgagagtggccatggaaaagagtaccaagaaggccgtgttactgcttttgtcgtgatctcctgcattgtggctgcaatgggaggtctcctcttcggttacgatatcggtatctcaggtttgtttcttcccaaccaagaaaccccaagatttctccttctctcttcttctttctcttctcatcatggcgggatatatttttttcaggaggagtgacatcaatggatgagtttctaacgagattcttccccgacgtgcaacgccaaaggcaacacaatacagggcatgaaacagagtattgcaaattcgacaatgagcttctcactctcttcacctcctctctctacctcgcggctctattcgcttccttcctcgcttcaacgatcacaaggctttttggccggaaaatctcaatgaagatcggaggtctcgcttttctctccggagctcttctcaacggt

SEQ ID NO: 48 - DNA, 1071 bp, Thlaspi arvense
  source 1..1071: mol_type = genomic DNA
atggctgcagtagaaagagtggagagtttagccaaaagcggaatcaaatctatcccaaaagattacgttcgtccgaaagaagagctcgagagcatcaacgacgttttccaagaagagaagaaagaagaaggtcctcaagtccccaccatcgatctacaagacatcgagtcagaagacgaaacgatccgcgagaagtgcacagaggagctgaggaaggcggctatggattggggagtgatgcatttgatcaaccatggtataccggtcgatctaatggagcgtgtgaagaaaaccggagaagagtttttcggttctcctgtggaagtgaaggagaagtatgccaacgatcaagccacagggaagattcaagggtatggaagtaagttggctaacaacgcgagcggacagttggagtggcaagattacttcttccatcttgtttatcctgaagataagagagatctaacactttggcccaagacaccaagtgattacattgaagcaacgagtgagtacgcgaagtgtcttcgtttgctagcgacaaaagtcttcaaggctctttctatcggtctagggttagagcctgaccgtttagagagagaagtgggtggtttagaagagcttcttctacagatgaagatcaattattacccaaaatgccctcagcctgagctagcacttggcgtggaagctcacaccgacgttagcgccttaaccttcattctacacaacatggttccaggtttgcagctattctacgagggcaaatgggtcattgcaaaatgtgtccccgactcgattgtgatgcacattggagacactctagagattcttagtaatggcaagtataagagtatacttcatcgtgggttggtgaacaaggagaaggttagggtttcttgggctgtgttttgtgagccaccaaaggaaaagattgttcttaaaccgttgccggagttggtgactgttgagtctccggctaagtttcctccaaggacatttgcacaacatgtcgagcataagttgtttaggaaggaacaagaggaattggtgtctgagaaaaaaagttga

SEQ ID NO: 49 - protein, 356 aa, Thlaspi arvense
MAAVERVESLAKSGIKSIPKDYVRPKEELESINDVFQEEKKEEGPQVPTIDLQDIESEDETIREKCTEELRKAAMDWGVMHLINHGIPVDLMERVKKTGEEFFGSPVEVKEKYANDQATGKIQGYGSKLANNASGQLEWQDYFFHLVYPEDKRDLTLWPKTPSDYIEATSEYAKCLRLLATKVFKALSIGLGLEPDRLEREVGGLEELLLQMKINYYPKCPQPELALGVEAHTDVSALTFILHNMVPGLQLFYEGKWVIAKCVPDSIVMHIGDTLEILSNGKYKSILHRGLVNKEKVRVSWAVFCEPPKEKIVLKPLPELVTVESPAKFPPRTFAQHVEHKLFRKEQEELVSEKKS

SEQ ID NO: 50 - DNA, 4065 bp, Thlaspi arvense
  source 1..4065: mol_type = genomic DNA
gtaaagtagacagctttttggaatttgcagagattatggtacaggaataaacactgaatcgagattatagccactgaatcaatcaaagccttgagcaaatgcgattgagagctgaaatttaacagggtttcgagccacaatactataaactagtgactgaaaaacttcgatcactaaagcttcagctagatttggtggaagaagaaatctaccactaaattctaaaaggctccatctttgtagaatcaattgtgaacggattctaggtaaaggatgaaaattgaagacctgattgctctgtggtggcttttgcaggcaataagaaggatcttcttctcaaggaaatcgatcttccagggctgagctgatcccattgagtcctcggcgacgacgacgacggaggagaaggagaagagaaagaagtggtgggtttgcgagaaacggcggagaaaagtcgagggcttgtcaaattgaacaacatcttgctacccattttccccgggagaagcaaggagagatccttttcctctgttttccgggaaggaacctgtggttgaagatgagttactgtgagcttcaggttccttctgatttttttattttattttaattcaatattttattatttatttatttaatttagtagagaggatttggagggaagaaattaggaaagataagagaggtgtggagatgaattcctaggcagatctctgatattttttccccctctcatcttaaagaaaaagaatatccacgtcagcaataaatatcccacctttctgtattttttgtaactttataagaattttatttactttaatattttttaaatgatacagtgatttctctttgtatataaaaaactttgaagtactaatttactttagacaaacaaatgtgatattattatttcctttgttcagttgatcttttatacaattagtctttttgaaactgaaaccagtcaaatactcaaaatgtttttagtgatgctaaaaattacagaaattatgataaaatgtggacattaaaaataaaataaaaaggtggataatgtacagacacctatattgattgaactatttaaaggtggacacgtggagatacgatttaggcacgtgcacacggacgcagaagaaacgccaccaaaagtctgacgttgatagtcatcggtttaactctattgggcttcttttttatgggcttttaaatccaaagcccttaccaagctaggacatactgtagtaacttctcttccagaattaaaagaaaacttttttgaccgttggatctcaaggatggtgaaaccatcagtcaacctctctcgttgacctgaccgtgaagtgtgttgtcactcactcacctgttcttcctccctacccaaccggtagctccacacttcctaattccttgattaattcttaattctaatggaaaattaatataattatctttgttatataagaaagcctctgcatatatttcatttgacatgcaacaaaatcggcaagaagaatagtagaacaaagtatttattcgttagttatcttacacactgttttttcctctgctttattaagtttctttacttacttactctgtttctttgctctgttttagcttttaaaagaagacaataaagatggctgcagtagaaagagtggagagtttagccaaaagcggaatcaaatctatcccaaaagattacgttcgtccgaaagaagagctcgagagcatcaacgacgttttccaagaagagaagaaagaagaaggtcctcaagtccccaccatcgatctacaagacatcgagtcagaagacgaaacgatccgcgagaagtgcacagaggagctgaggaaggcggctatggattggggagtgatgcatttgatcaaccatggtataccggtcgatctaatggagcgtgtgaagaaaaccggagaagagtttttcggttctcctgtggaagtgaaggagaagtatgccaacgatcaagccacagggaagattcaagggtatggaagtaagttggctaacaacgcgagcggacagttggagtggcaagattacttcttccatcttgtttatcctgaagataagagagatctaacactttggcccaagacaccaagtgattacatgtaagcttttgattcgtttgttttatcatttataaaacaaagttcttgctttggttatagtaacgttttttggatgattgtagtgaagcaacgagtgagtacgcgaagtgtcttcgtttgctagcgacaaaagtcttcaaggctctttctatcggtctagggttagagcctgaccgtttagagagagaagtgggtggtttagaagagcttcttctacagatgaagatcaattattacccaaaatgccctcagcctgagctagcacttggcgtggaagctcacaccgacgttagcgccttaaccttcattctacacaacatggttccaggtttgcagctattctacgagggcaaatgggtcattgcaaaatgtgtccccgactcgattgtgatgcacattggagacactctagagattcttagtaatggcaagtataagagtatacttcatcgtgggttggtgaacaaggagaaggttagggtttcttgggctgtgttttgtgagccaccaaaggaaaagattgttcttaaaccgttgccggagttggtgactgttgagtctccggctaagtttcctccaaggacatttgcacaacatgtcgagcataagttgtttaggaaggaacaagaggaattggtgtctgagaaaaaaagttgaagtttgagtctaatatgagtaaaaactccatgttaaagtttctcgtttcttttgtgtttatgtctttatcctgtatttgatgcgtcttgttgaagatgacacgaaatatttctaccgattgtgaaattgttaaaaatttcactctattataagttggttttatgtatcgccgaagtgacagatgttggaagaaatctagtcattggaaactgtattacattcgtgctaattgcatttcttttcaagtttaaaccaaattagtggaacgtctccggttctagtctgtgtgacttagttatagcttggtgaatagtaaaggaagctcagtaaatacgagtaacatagaaagatatttatgaaccacattgttgtattaaggacttgagaatattctgatttacatttccagtttgttcttttttgttatgctactaactgtaaaaaaactactcttctccctcctttaatgataattggcattccctgcttggaaagagttcactctattttccttgaagaccttaaaaccaagaatagattgcacaactcaatgattagtgttggaaaatagaaaaaaaaaaatcaaaaattgagtttcaagtttcacaaggctgcacctttggttgacgaaatcttgtgagagatccattgttttgctggagctccgatttgatagagagctgcacaggtttaatacgggtcaagagagcgtatacaaataaactagagtgtgtttaagaaagatccattacatcaaaccatgagctgtaccttgctgaaaagatctgttggtaaatctggctgaactctcttttccgaatgaaaactaaactcctgtgtttgcattcccaaaacagtttgcatcctcatcaggtaactcaattttacagacatacagaagatcaagcgcaaaaagaataattggtaaagactaccataccaaaggtactgttgtttctcgcttgctgtttttgaaaatgcccatgtctcctctgcttgagagtatctgttttgcaggagtgtgaaactatgtaaggtgtgacacgtataattaacgcaagcgaaatgagactcgagaaaggataataccttcttgttcagaggacgagctttaaaaacgtgttttccctccgagacatcatgcttaggtatatccataacgcttggtctgcaagaaaaaaatacacttttatgtttacattcacttcaaccacaagttgaagtgagaaatcaagcaatccttcttaatgttccatgtgactttat

SEQ ID NO: 51 - DNA, 645 bp, Thlaspi arvense
  source 1..645: mol_type = genomic DNA
atggttgtgaagctatacgggcaggtaacggcagcttgtccacagagggtcttgctttgttttctggagaaagaaattgaatttgagattgttcatatcgatcttgatacatttgagcagaaaaaaccagaacatcttcttcgtcagccatttggtcaagttccagccatagaagatggagatttcaagctttttgaatcaagagccattgcgagatactacgcgaccaagtactcggaccaaggcacgaaccttttgggcaagtctctagagcaccgagccatcgtggatcagtgggccgatgttgagacccattacttcaacgttctggcccaccccattgtgattaacctagtcatcaagcctaggttaggcgaagaatgtgacgtcgttttggtcaaggacctcaaggtgaagcttgaggaagttttggacatatacgagaaccggcttgcttcgaaccggtttttggctggtgatgaattcaccatggctgatttgacgcacatgccagctatgcggtatttgatgggtataatcgatataaaccggatggtcaaggctcgggtgaatatgaaccggtggtgggaagagattacggctagaccggcttggaagaagcttatggtgatggctggttcttga

SEQ ID NO: 52 - protein, 214 aa, Thlaspi arvense
MVVKLYGQVTAACPQRVLLCFLEKEIEFEIVHIDLDTFEQKKPEHLLRQPFGQVPAIEDGDFKLFESRAIARYYATKYSDQGTNLLGKSLEHRAIVDQWADVETHYFNVLAHPIVINLVIKPRLGEECDVVLVKDLKVKLEEVLDIYENRLASNRFLAGDEFTMADLTHMPAMRYLMGIIDINRMVKARVNMNRWWEEITARPAWKKLMVMAGS

SEQ ID NO: 53 - DNA, 3574 bp, Thlaspi arvense
  source 1..3574: mol_type = genomic DNA
ttaaatgtcggattccaattgagttaagtaagtctcatctatatgtaaatgggtttttggaatagtatcatcttataatcattttacacagaatcaaagttattagccttgctaaatctaggttgtattgatcgaatgatcttcgaaaaaactcagttagctgtaaatgctttgcagggtatgttaatgaatgaaaatctacataatcacatccctcttgaactaatatgtctgtttatagatcaaaaacaagttaggcatgtgaaagttttgtaatcttgttctggttatggtatttgtgcaagctttcgaactcgtgtataaaggttttcttctcattttcatgttacaacaacgcagagatctcaggtcaaggttaccaccacttcagatccactcacacgatgctcttatccaagaagtccagctttggatctcggcttcacttcagctatttttctaatgatggctcagataatcgtcagcgtcggaagcggctgtctctgttgtagaaaaggtcctgctccttccagatctaattggattatcgccttaacctgcttcgttgtttcctggtaatcctcatcatttctttctcctatcgagctttcctacttcacaattttgctggacagacacaagtgacgaatctttgtgtttactcttaggttcacttttgtgatagctttcctcttgctgctaaccggagctgcactcaacgatgaacacactgaggagtcaatgtatgccggttactactcctgctacattgtgaaaccgggagttttctctaccggttctttgctttcgcttctcactgttgccctcgggattgtctactatttgtgtttgacttcgagtaaacaaaacgttgctgacacagcgacgacggcgaaccgaggaggaggtatagcaatgggacagcctcagattccggagagagtggaagatcctgtctttgttcatgaagatacttacatgagaagacagttcacttaaaaacagaacatggctttctttggatacacgttaggttttaggatgataattagataaacaagagatgctgttttttttttttggggattttatctttgtgattttagtatgtgaaactaagaccatgaaacttggtagctggtaatgatgattaatgatgattgataacgtacagaaaaccaaacaaacgtctccttttacttatctatgtgaatttaaagtggttgttgggaagtgttcacaaaaatcagaaattgaaataaataattaacctatccaccggtccgaccaaacacgggacatctaatagaaatcaattttgtttatctcgaatcataagacgaaaactactgaaatttttatgttaataagtttgtagagcaactttggatgatatccaccgttagtggaacaaccaccacatgctctacccaaccagcacaacaaccagctccttttaaatgtttttgagtctaataaaatgccaacacatctactactctcacttcttcacatttataaataagatccagagaagcaattttaaaataacacattgaaattgtttcaaagcttatttttttttaaatttatatcaataaaaatttggttgcaaagttattgtaacgcttatagaatggttgtgaagctatacgggcaggtaacggcagcttgtccacagagggtcttgctttgttttctggagaaagaaattgaatttgagattgttcatatcgatcttgatacatttgagcagaaaaaaccagaacatcttcttcgtcaggtcaaacattttatattttctaattttgaaaagtcatgaattgtatttaatatcattaatgttttcttttagccatttggtcaagttccagccatagaagatggagatttcaagctttttggtaagatcctaataacagcattgtaaaacattctttaatacttgttttatatcattaatatgtagattgtgttttgaaatttggcgtgtgtgctcagaatcaagagccattgcgagatactacgcgaccaagtactcggaccaaggcacgaaccttttgggcaagtctctagagcaccgagccatcgtggatcagtgggccgatgttgagacccattacttcaacgttctggcccaccccattgtgattaacctagtcatcaagcctaggttaggcgaagaatgtgacgtcgttttggtcaaggacctcaaggtgaagcttgaggaagttttggacatatacgagaaccggcttgcttcgaaccggtttttggctggtgatgaattcaccatggctgatttgacgcacatgccagctatgcggtatttgatgggtataatcgatataaaccggatggtcaaggctcgggtgaatatgaaccggtggtgggaagagattacggctagaccggcttggaagaagcttatggtgatggctggttcttgaattatttcgaatttatggtgatctgaaccaaataagcttcatattttctgtactattttctttatctatgttttgtaagtttcaattgataaaataaatttacttttaaagatttaaattcgttaatattttctttatctatgttttgtaagtttcaattgataaaataaatttacttttaaagatttaaattcgttaattctcaattttaaaggggactatagatttacagttatcattttgttacgtattactacaaattgctgaattgttatagaactttttttccaattgtttgtgaacctaatactttaaatctgaatagacatcatgtatttaattatttcccatgttatattttcatctagtatatatagttcattcatttgtagccatgcagatgcacaatgaatctcgattttttttttttttgtgcgagcacaatgaatctcgatctcttagtgtttttgaattcagtaacttctttgcatctttcactaaactatctctccaaccaaaaaaattcatatttcttcagcagccaagagagaaataaaaaatttcaaaaattactttttacatagagaaaaagaagggaacatccgcccatttatatgtatattagttaacatgataaaaaatccgaccctaagttttttcattaatcacaaaagccctctctttggttgtttttcttggcttctctcattcatgtcgacttagaagaagaagctaatcttgagcttgaagtctttagcactgattttgcataagccaatggcaaagctgcaattttcttggctttccctacataagctctctttgtaaaattgttgtaatcattcgcttcaatctcgtccagtatcctcctgtataatagcagcgatgcccatacctgaaaaaattgcacagccaagtgtaagggggttaaatcgatccaaacaaaaaagctttggggcgaaaacaaaacaaaaatgtaaacggtaaagtgtaaacgtgggggcttaagagtgttgttagagacacttaccggccatctgcttgcggcgtcgagctccgtgacgcctttctcagcttcgtcgaagaacattcttgctcgtttaagctgcattttcatgaagttt

SEQ ID NO: 54 - DNA, 1155 bp, Thlaspi arvense
  source 1..1155: mol_type = genomic DNA
atggttagtcacaaagagaccgtgtgtgtaacaggcgcatcgggattcatcggttcatggcttgttatgcgattattggagcgtggttactttgtccgtgccactgttcgcgaccccggaaatttgaagaaagtgcaacatcttgttgatttacctaacgccaagacgcaactcactctatggaaagccgatttatctgatgaaggaagctacgatgacgccataaacggatgcgacggcgttttccatgtagcaactcccatggattttgaatcaacggatccggaaaacgaagtgataaaaccaacagtgaatggagtgttggggataatgaaagcttgtgataaggcaaaaacagtacgaagaatcatctttacttcttctgccggaactgttaatgtcgaggaacatcagaaaaatgtctacgttgaaaatgattggagtgatcttgactttatcatgtccaaaaagatgaccggatggatgtatttcttgtcgaaaactttagccgagaaagcagcgtgggattatgcgaaggaaaatggattagacttcattagtataattcctacattggtgatcggtccattcataacaacatctatgccgcctagcctcatcaccgcgctctctcctatcactcggaacgaggcacattactcgatcataagacaagggcagtacgtgcatttggacgacttatgcaatgctcatatattcttgtacgaacaagctgatgccaagggacgttatgtttgttcctctcacgatgccacgattcttagtatctctgaatttattaggaaaaagtaccccgagtataacgtgccttcaacgtttgaaggtgtggatgaaaatctagagagcattgtgttcagttcgaagaagctgattgatatgggatttagctttaagtatagtctcgaggagatgttggtcgaatcgattgagacatgtcgtcaaaagggttttctcccggtttctttaccagatccatcaatatttgaggacaaagttccgactagtgatgacaagattgagcacaaaaccggagctggtttacccgatgatgtggtgccctgtaagaagacagaaccggtggtaatccgcgagaaaaccgatgcttgcatgccggcagagcagatgtgtgcttag

SEQ ID NO: 55 - protein, 384 aa, Thlaspi arvense
MVSHKETVCVTGASGFIGSWLVMRLLERGYFVRATVRDPGNLKKVQHLVDLPNAKTQLTLWKADLSDEGSYDDAINGCDGVFHVATPMDFESTDPENEVIKPTVNGVLGIMKACDKAKTVRRIIFTSSAGTVNVEEHQKNVYVENDWSDLDFIMSKKMTGWMYFLSKTLAEKAAWDYAKENGLDFISIIPTLVIGPFITTSMPPSLITALSPITRNEAHYSIIRQGQYVHLDDLCNAHIFLYEQADAKGRYVCSSHDATILSISEFIRKKYPEYNVPSTFEGVDENLESIVFSSKKLIDMGFSFKYSLEEMLVESIETCRQKGFLPVSLPDPSIFEDKVPTSDDKIEHKTGAGLPDDVVPCKKTEPVVIREKTDACMPAEQMCA

SEQ ID NO: 56 - DNA, 4705 bp, Thlaspi arvense
  source 1..4705: mol_type = genomic DNA
cctccgttattatggagccttgtaattagtgttcagaaaatcaatcccaacaatattatctaatattttctatagaaaaaatatagtttaacaagtgtttagatgcctcatatcaaactctctaagattgtgtttctaatctagtactaagtacaagtccaatgactccaaccacaatcatatttattctacacatgaggggtgaaattttaattgttaaataaaaaaatatagatacaattattctattctatagtttatatgttgacaaagaaatctttggtaaattttatttttaaaagtcaaaaagatatttgttaaaagtattgttgtttgaggaaatgctccaaaataatccagaataagaaaataatatgaaacggtactccattgagaaaaaactgtgaaaataacccatattttatgattttttttaaataccagtgctacatttctaactatttttacttgtagtctaatggcatactgaatgcagagtactggcagactgattggtcttacaattgaatatagactattggcagactaaatgcaaattactgacagactaacgacagattaaaagcatattagtggatcctgcttatggatcatttgaaaaaggttcatgtttggtaacaaaattaccaaatgaataatttcaaaaattatcccatttgagtaaagtatatttttatttttggctaatttcttctttgaattaacccaaattatggtgtttgtaaattgtaactgtgatttttttttacatcaagaagtgaatggttgacaaaaaaaaaagaatgattaaggtttaaatactaaactcgtatatttaaattataaacttttcgaattcattttttttaattttatcaatctttagaaaaatattatatgtttctagaaagtttctataaattctaatgtgtttaccattaaaaatatattatttattttcctcatttgaaatatcaatttatttatatataactcataattttaaaaatttatatatcaactcatgtcaaaattttaatttcatatcagtttatttcaataatgtttttacacgtattaaaatctaatcaattacactcttttattattaacaaatttgacgtattaaattttatatgataaattttgttaacgtattaaaataatataaactcttcttgtagtttagtaacaaaataaaaaaattacaaaaaacaataagtaagaaaaggaaacttcaactcttgtcgctatctcactaaacaaacaagcaaacccattcggcgtagttcacccaccagtacaactaaaactatatcctaaaaaataataaaatcaaacttaccagtttgtcacgtaccacacaacccagtccttcgccaaccaacgttcctcacgtgcttcgccggttggtactcacgtgaccggcagctttctaatagcaattctcaatacatatataatctcttcataccaaagctacatatatagtctcttcataccaaagctacatagcaaagtctatcccaaagcacagtccatctttataatacacaaaaatggttagtcacaaagagaccgtgtgtgtaacaggcgcatcgggattcatcggttcatggcttgttatgcgattattggagcgtggttactttgtccgtgccactgttcgcgaccccggtacgtatatatgttcttacaaactaactcgttaattaatttttcctaagattaattaaatctatgtgtggctaatttacgggttttattgggttatttatataggaaatttgaagaaagtgcaacatcttgttgatttacctaacgccaagacgcaactcactctatggaaagccgatttatctgatgaaggaagctacgatgacgccataaacggatgcgacggcgttttccatgtagcaactcccatggattttgaatcaacggatccggaagtaagttataatatgaacctcttttaggtttcatatcaaccctaaaagatcttgttggttaaatttttttttttttttaatcagaacgaagtgataaaaccaacagtgaatggagtgttggggataatgaaagcttgtgataaggcaaaaacagtacgaagaatcatctttacttcttctgccggaactgttaatgtcgaggaacatcagaaaaatgtctacgttgaaaatgattggagtgatcttgactttatcatgtccaaaaagatgaccggatgggtaaatatatataaattatatatcaaacatcacactaaccctaagtttattcttcttcatagttatttatgttttaataaaatgtttttggcagatgtatttcttgtcgaaaactttagccgagaaagcagcgtgggattatgcgaaggaaaatggattagacttcattagtataattcctacattggtgatcggtccattcataacaacatctatgccgcctagcctcatcaccgcgctctctcctatcactcgtgagctctcacttaatttctactctttttgtaaactaagagatttcttgaccttaggccaggtcttgctcataaaatcaatgtccactgagcaatattctattttataagcctaataatcataaatggcaaggaaaacaacctatttttttttaaagaaccttaacgtctaaccaaaaattacaaaaagttatatggacccaatgcaaatacatctttggcacatgccaagaaccggatcgtaggaagatctttctggctagcgactagagcatgtcaactagtctcttctatcttccatcttcttttgttggaatttgaaatgttaattttttttaaaaacggtgaaaatgtttcagggaacgaggcacattactcgatcataagacaagggcagtacgtgcatttggacgacttatgcaatgctcatatattcttgtacgaacaagctgatgccaagggacgttatgtttgttcctctcacgatgccacgattcttagtatctctgaatttattaggaaaaagtaccccgagtataacgtgccttcaacgtaagatttatagatttaaattacggagtttaagccttgttttgcttaagcaatcaagatcgtttgctgaaaaactatttggaacaggtttgaaggtgtggatgaaaatctagagagcattgtgttcagttcgaagaagctgattgatatgggatttagctttaagtatagtctcgaggagatgttggtcgaatcgattgagacatgtcgtcaaaagggttttctcccggtttctttaccagatccatcaatatttgaggacaaagttccgactagtgatgacaagattgagcacaaaaccggagctggtttacccgatgatgtggtgccctgtaagaagacagaaccggtggtaatccgcgagaaaaccgatgcttgcatgccggcagagcagatgtgtgcttagagatttaaccggtatctattatatatcaattgtcgtatcgttttagtattggatgtgtgttttgaatttgttactttctgaatcgtagattattattgaagggtggtatggactgattctgatagtaacatgattgagtgtaacattatgaacttttacatatttaagaagcagagtgatatttaaacaatgttgccttgagtcaaattagtcgtgtaatgtcgcttgaatgcttccataatctataattagacaatttcaacatctcaatcgcaggcatgcatttcttaacaaagagttatatattcggtggtcactggaaatattttatacatatataatataatttaactactatgcagattacagagtataagaaaacaattcttggctaaatagattttcagttacaaaataaagaaaaaccaaatgactgaactttttttatttgaagaacaaaaaatgaaaatttagtcgttcgagttaatttacggatcctatcagctaaaaataatttagttctgttgagatgtccatgctttttttggaatttggattttcctttttccatatgtgaaacctttccatataccaaaaactctatcatcaaactacccagcctttgtatctctatgccaaaaaaaaaaaattaaacaaaatctttaatttgaagtcggactgtcggagtatatggtttttcttgtacgtacgttttccaaatgcttcagtaaattgctcttcgaagtctttcatcgtcagctggaaagaaatacaaaagtgattcctcttgtgcctcaaatgttcttttgtagcttaggagaggatattgggagatgggttttaaagaatttttcaaattttgagattctattgtttataaatttttaaaatcttaatagttaatattgttattggttgaaagattttaaaattctatccaaaatcctttgttatttaaaaaatttagcttttaaagattttatgatttattaaaatcttgtgttattggaacatgaattttaaacaatctaacttataaaataattctttcaaaatactacctatatagcttagattctcaaagtcattataataaaatattttgatagattttttaaatataaaaactctttccaaatttaacaaatattttttacaagtttaacaaatctcccaactcttgaaatcaatcaaaactttataatttcatctcttaatgaccctcccttactctttgctttttgttgtttgtttctcaattc

SEQ ID NO: 57 - DNA, 1188 bp, Thlaspi arvense
  source 1..1188: mol_type = genomic DNA
atggtgatgggtacacaaccgtcgttggaagagatcagaaaggcacagagagcggatggccccgcaggcatcttggggataggcacggccaaccctgcgaaccatgtgatccaggcagagtatccggactactacttccgcatcaccaacagtgagcacatgactgacctcaaggagaagttcaagcgcatgtgcgacaagtcgatgatacggaaacggcacatgcacctgacggaggagttcctgaaggagaatccggacatgtgcgcctacatggctccttctcttgatgtgaggcaggacatcgtggtggtcgaggtccctaagctagggaaagaggcggcagtgaaggccatcaaggagtggggtcagcccaagtccaagatcacccacgtcgtcttctgcactacatccggagttgacatgcctggtgctgactaccagctcaccaagctcctcggtcttcgcccttccgtcaagcgtctcatgatgtaccagcaaggttgctacgccggcggcactgtcctccgactcgccaaggacctcgctgagaataaccgtggtgctcgtgtccttgtcgtctgctccgagatcacagccgtcaccttccgtggcccctctgacacacacctcgactccctcgttggtcaggctctcttcagtgacggtgctgccgcgctcattgttggtgcggaccctgatgcctccgtgggagagaagcctatcttcgagatggtgtctgctgcacagaccatcctcccagactcggacggagccatagatggacacttgagggaagttgggctcaccttccatctcctcaaggacgtccctgggctcatctcgaagaacatagagaagagtctagaagaagcgtttaaaccgctcgggataagtgactggaactctctcttttggatagctcaccctggaggtcctgcgatcctggaccaggttgagttaaagctaggactcaaggaagagaagatgagggccacgcgtcacgtgctgagcgagtacggaaacatgtcgagcgcgtgcgttctcttcattatggacgagatgaggaggaagtccaaggaggatggtgtggccacgacaggagaagggttggagtggggtgtcttgtttggtttcggaccaggtctcaccgtagagacagtcgtcttgcacagcgtccctgtttga

SEQ ID NO: 58 - protein, 395 aa, Thlaspi arvense
MVMGTQPSLEEIRKAQRADGPAGILGIGTANPANHVIQAEYPDYYFRITNSEHMTDLKEKFKRMCDKSMIRKRHMHLTEEFLKENPDMCAYMAPSLDVRQDIVVVEVPKLGKEAAVKAIKEWGQPKSKITHVVFCTTSGVDMPGADYQLTKLLGLRPSVKRLMMYQQGCYAGGTVLRLAKDLAENNRGARVLVVCSEITAVTFRGPSDTHLDSLVGQALFSDGAAALIVGADPDASVGEKPIFEMVSAAQTILPDSDGAIDGHLREVGLTFHLLKDVPGLISKNIEKSLEEAFKPLGISDWNSLFWIAHPGGPAILDQVELKLGLKEEKMRATRHVLSEYGNMSSACVLFIMDEMRRKSKEDGVATTGEGLEWGVLFGFGPGLTVETVVLHSVPV

SEQ ID NO: 59 - DNA, 4151 bp, Thlaspi arvense
  source 1..4151: mol_type = genomic DNA
cgaatccgcatagagaagctgaagctcaggtttctcaattatttcttctttctttttcaaaaggcttatcattgacttgtctctgtttttttttttttttttttgtgatcattgattcagtctctccaacttgatgaaaaagataaccatccaagagcccacctttgagagaattattgtagtatacaggtacaagagtagagtaatggtgaaatctttggagggatgtgagtttgattctcattttggctttggattcacatacacgcaggagagtttcagggaagagagaatcagaaaggaacatatatgtgaaacacttcaagagcattcctatggctgatatggaaattgtgcttgtaagcatccattaaccaacactttgtgctcaaatgtatcttactaattataaattaactgattgctcataacggcacagccagagaagaaaaatccaggtcttacgccactggactgggtcaagtttcttgtctctgctgccattggactggtcagttctgctacactattctaaagactttaataatcagactctgaaaattttgatttctcaaactctgcttttacaggtcacagtggttagttcagtgagcctaaaaaaaactgacatcagagtcattgctgccatacttagcacggtcgtggcctactgcgttaaaacatatttcacgtaatctacactcaataatcagacaacaccctcgcttatagctaaattaatccttaattagctctgtctcttcctctgtatgaaatgtaggttccagagaaacttggtagattatcagagccttatcacaagatctgtgtatgataagcagctagacagtggaagaggcactctgcttcacctctgcgatgaagtcatccaacaagaggtgctaataaatagtccttttttcttatttttcttatgtcatattcttgtgttttgctaattaaaaaactgatcaaacgcaggttaaagaggtcattatttccttcttcatgctgataaagcaagggcgtcccacgagcaaggaggagcttgacatgcaaagtgaagcattcatcaaagaagaattcaacgaaagttgcaatttcgatgtggatgatgctgtcaagaagcttgagaagcttggacttgtctctcgtgtgagtctccttcatctctcttctataatcttttctttttctccacgcatatgcttgtatatactcagtaagtaaactaatattgtgaaaatggaaagggttggtcaggattcagaagacaagtatagatgcgtgaatatgaaggaagcaaacgagataatgggaacaacgacggaagagatggtactcaaggcaagaagaggtggtgaatatgaagatgaagaggcagcagagactgagccacggatgaatcctcaagatgaactcacagcaaaggaagaacgttatcaatctcaattagacgagttcgagaccttgtggttgtaaaaagatttgacgtcattcaagacaaataaatttgatattgtattaaagaaaaagaaaaaaagacataaaggcagacaaattaattgaaaggacccatcaacaatgagtccaccacatttcacatttcacactaaatgaattctcccttaattatttcatacataatttatattaaaatgaaaaacataagactcgatgatggaaagtatgtgtaggtaggtaggcatataagaggagagcgctctgtatggtgatgggtacacaaccgtcgttggaagagatcagaaaggcacagagagcggatggccccgcaggcatcttggggataggcacggccaaccctgcgaaccatgtgatccaggcagagtatccggactactacttccgcatcaccaacagtgagcacatgactgacctcaaggagaagttcaagcgcatgtgtgagtatccctcttaatactccctattttgtatctcttcatttcttttggcatatggttcccatagttaacaattactgtaacaggcgacaagtcgatgatacggaaacggcacatgcacctgacggaggagttcctgaaggagaatccggacatgtgcgcctacatggctccttctcttgatgtgaggcaggacatcgtggtggtcgaggtccctaagctagggaaagaggcggcagtgaaggccatcaaggagtggggtcagcccaagtccaagatcacccacgtcgtcttctgcactacatccggagttgacatgcctggtgctgactaccagctcaccaagctcctcggtcttcgcccttccgtcaagcgtctcatgatgtaccagcaaggttgctacgccggcggcactgtcctccgactcgccaaggacctcgctgagaataaccgtggtgctcgtgtccttgtcgtctgctccgagatcacagccgtcaccttccgtggcccctctgacacacacctcgactccctcgttggtcaggctctcttcagtgacggtgctgccgcgctcattgttggtgcggaccctgatgcctccgtgggagagaagcctatcttcgagatggtgtctgctgcacagaccatcctcccagactcggacggagccatagatggacacttgagggaagttgggctcaccttccatctcctcaaggacgtccctgggctcatctcgaagaacatagagaagagtctagaagaagcgtttaaaccgctcgggataagtgactggaactctctcttttggatagctcaccctggaggtcctgcgatcctggaccaggttgagttaaagctaggactcaaggaagagaagatgagggccacgcgtcacgtgctgagcgagtacggaaacatgtcgagcgcgtgcgttctcttcattatggacgagatgaggaggaagtccaaggaggatggtgtggccacgacaggagaagggttggagtggggtgtcttgtttggtttcggaccaggtctcaccgtagagacagtcgtcttgcacagcgtccctgtttgacttttacaattcaagtcttttagagtttgatcgatgagtgagattgtgtactatgcaagttatgtatatttgatggcaagcggagagtgggagtttatattaacgagagaaagggagaagagagagtgtggtaggttagatggacgggcagatgaagcactacgtgtctatgtgttagctcccttttatgtggttctccttcttgcttcccttttatttcgttacttaccaagaatattgaatggtggtttttttattgattttacgtatattcgcaacaacaagtcatattaatttctatgtgcgtaattattatatattcgatgtatatacatgtaaagatacgtacggcctactggagaggttagttgtatagattttcccacacgtgcatacttaactccatcattagctcctactaatctaattttccttcttaatatacctgataatcctataactaactaaaaattacattaggtacgtctcgttttctctctcccaccccatcatgatgatagtttgaatctgctcctttttcggcttccaacaacgcaagatgcatttcgcatcccatcataatgatatttgtgttgttgctgttctttatgttagactaataaaagatgcatttgtaagcttttgcctcattaattttattcggaaaaaaattgaatatgcgtaatatcacatcgttttttgttagtattcatttattcaattcatgtcttaataatgttgtagatatttttattggaaaaataatatcaatgggcccataaaagaaggtgggccctagagctggttatgcttagccatgtgggcagaatataaccagagtgtgttgtcattgtggtaagtaggggcactccaatgcgggtgtttttatcaacccggcccggcccaaagcctatccgtcgaaaacccgtgtccatcaaaaactcgccaaactagcggttatatttagggttgggcaaattatccgttatatttgattcgatctgctatttgtttcgattcgattcgaaaaatccagatatccatagctttacggagcaaagcaaatactaaattttgatattcgttaaaaacggagcaaatcacaaatattaaaattttaagagtcggatatccgatctgctctaata

SEQ ID NO: 60 - DNA, 759 bp, Thlaspi arvense
  source 1..759: mol_type = genomic DNA
atgtcttcttccgcatgtccgtcaccgttaccctcagtcagcaaacttcatgtcgattccgttacttttccaccgtccgtcaattcaccggcttcctccaatcccctattcctcggtggcgcaggggtgcgagggttagatattcaaggaaagtttgtgattttcaccgtcatcggagtttacctagacgctgtcgccgtcccgtcactctctgttaagtgggagggcaaaactacagaagagttaacggaatccgtcccttttttccgtgaaatcgtcacaggtgcgtttgagaaattcataaaggtgacgatgaaactgccgttaacgggacagcagtattcggagaaagtaacggagaattgtgtggcgatttggaaatcgttagggatttacacagagagtgaagctaaagctgtggagagatttttggaagtcttcaaggaccaaactttcgctcctggtgcatccatcctcttcgctctctcccctaacggctccctcacggttgcgttttcgaaagacgatagcattcctgaaaccggaaaagcggtgatcgagaataaattgttggcagaggcagttcttgaatcaataattggaaagaagggtgtgtctcctgggactaggctgagtatagcagagaggttagctaagctgatgaagaagaagaaggtcgaagaagatgcatcatcactgactgatcaagaggaagctacagatctctccctcggagataaattggccaaagagaactga

SEQ ID NO: 61 - protein, 252 aa, Thlaspi arvense
MSSSACPSPLPSVSKLHVDSVTFPPSVNSPASSNPLFLGGAGVRGLDIQGKFVIFTVIGVYLDAVAVPSLSVKWEGKTTEELTESVPFFREIVTGAFEKFIKVTMKLPLTGQQYSEKVTENCVAIWKSLGIYTESEAKAVERFLEVFKDQTFAPGASILFALSPNGSLTVAFSKDDSIPETGKAVIENKLLAEAVLESIIGKKGVSPGTRLSIAERLAKLMKKKKVEEDASSLTDQEEATDLSLGDKLAKEN

SEQ ID NO: 62 - DNA, 3963 bp, Thlaspi arvense
  source 1..3963: mol_type = genomic DNA
ttccccacaaacaatgtgtgtgtcagctatatatttatgggaaggctgaaacgtttaaatactagcctctgcatcgtttgttgtaattttgtgataaaattgtaacatatttaatcgttttatctctaggtacttttatggaatattgtaaaagaattctatttttgtgtaaaatccaaattagcgtattatacttgaatacacaagcatttcggcacatagttcaattggggttaattcatgaatcatttttaattcaaaaatttcgacccatcaaaattagacaatgaccaaagaaattataaaaaatgagatgatgtttgaaaatgtttagtgaggaagtgaaggtatagattatagacgtttattgtcaaatttcaacggatgttatatacgttgttaaatttaaaacaactttagaatatatatggcgcgtgattttgttaaatcgactttggtaggaaaatgttattttgcgattttgtcaagaaaacgtaattttgtgattttgaccaaaaaaaatgttatttacagtaatagaaaaaaataattttataatttaaaaaacatgatttttgattttattgaaattttttttttttaattttagtgagaagtgattgcacatttttaatgggaaaattttctacaattttaattaaaaaaatgatttcagcataattttagtaaaaaaattgtgattttacagttttataaaaaaatatacgtttttatttttttgaaataattgaaaaatctcatttatattatgttgatttattcatctgaaatttgcatttaaatattctatctaaataaaagtattttaattatttgttattttaaaatagattatttccattttttatcaataaacaaccccatatgttgttaaacaaagaaagaaaggacaatctatatttcttagttatttttattattatattttctttattctttattctctttatttatattatttttataatgttcattaattaaaacctacctcttacagaacttttttgggttgttttaaaaggatcagacatagtgtaaactaaaaaattaaactgaaccaaaccaaactatgtcttatacttatcaagtaatatacactacatgataagcttaaactagattctatctttatcataaattaattttatctttatcacattgtggtctacataaatcctcattacataagtatcttaattaaatcttctagattcttccttaacagtaaaccaagattaaaaatgatctgatttgaactgataaaacctaaactaaaaagaaattggcatacctaccgactctaaagcggtcagagttcccgcaaccaccaaaattataaaccactgaatcttccaatcaacaaaaccacgtgtttacccatccaacactcgtaagctattgctagttgctacctacctccatcttctcttataattaccaatctccatcaaattcttttttacttttttttacattataatcaaacctaacaccctcaaagtcaaaaccatgtcttcttccgcatgtccgtcaccgttaccctcagtcagcaaacttcatgtcgattccgttacttttccaccgtccgtcaattcaccggcttcctccaatcccctattcctcggtggcgcaggttcgagtgctgtttaattcatttgctcttgcagcattcttttttttttttttaaatgcgttttttgttgcctctttgagtttctctgtttcttgctaacttacgcaggggtgcgagggttagatattcaaggaaagtttgtgattttcaccgtcatcggagtttacctagacgctgtcgccgtcccgtcactctctgttaagtgggagggcaaaactacagaagagttaacggaatccgtcccttttttccgtgaaatcgtcacaggtgcttgtcatgcaacaaactgttttaagtcaaaaagggattttaatttgtttagaccaaactatttgaccgtcattaatttattattaccatttttagttaattatgatttttttttgtttttgggcacgattaggtgcgtttgagaaattcataaaggtgacgatgaaactgccgttaacgggacagcagtattcggagaaagtaacggagaattgtgtggcgatttggaaatcgttagggatttacacagagagtgaagctaaagctgtggagagatttttggaagtcttcaaggaccaaactttcgctcctggtgcatccatcctcttcgctctctcccctaacggctccctcacggtacttcttgtcctatcacacagctttgcgtttgcgtttacattactattacgaactgtttacaatgaaacgcaaattaatgtagaaatagcgtttaaaacgaactgtgcccaaaacatgtatttttaagtaagatttggttttaaaaattgttttacgtgtattaaacgcaattgacgttagaaaaacagtcgtaggttgtaaaatgtctttgttgtgaaaaaaaaatcgttttgcaggttgcgttttcgaaagacgatagcattcctgaaaccggaaaagcggtgatcgagaataaattgttggcagaggcagttcttgaatcaataattggaaagaagggtgtgtctcctgggactaggctgagtatagcagagaggttagctaagctgatgaagaagaagaaggtcgaagaagatgcatcatcactgactgatcaagaggaagctacagatctctccctcggagataaattggccaaagagaactgatcctatgccgagatttttcttggtttctgagggattttatgtttctgttatatttaatcctattccatcgcatgtttaatttatctctgttcgtaagaataattaaataacctcatggtattggatcaaagtttccaaacaaaacaaaattgtattgtcaaatatttaatacaagccaaacttgataaacatttagttctaggcttgatcacacggatccgcaaggttaagtaattggttaaccgaagggaagggaaaacacctacacgaatcccggaccgggcctaaattcttttaactagaacacggcccaatctgtagaccagttcggcccattgtagtatcaaaaatattccataaacgaaatcatttgatttcaaatggaggaaatcattaaattagcagacgcggaaaaccactatctcatcaaaataaattgcgaaatttattgaataaactaatgaatcaataaataaatgaaatggtccaaccatctaaaaattgattttctaagatgtcactgtatcaatatcacccattcatgttcatataaaccctacccgccagcataatgcatattgagtattgacgacttataaacaaaagaagaaatcacttacagaaaaatgatttcttgaggaaatagatcacaggatgtgtatgttgattaatgacctagggtacaataatgcagtttgattcactactagacccataattttttttttttaaaaataaaaagggctttcatgacccataaaaattattattatctttttttcccataaaaattatttgataattgtttacgtgtttgtttgttttctcgttcatattgatatttctctcatccgaattcttgcattttcaacgtgtcaattatctttttaaaactacgtaaatactattatgataccttgtaggcttgtactgaatatttgtgagtattttaggctttataaattcttatgccgactttgttatcaagccttcgacacattttctgtggcgttcttgagactataaaatttgggagacgattgaaccattttgtttttatctataatggtttttcttttgtatcctttatttgattttttttcttctttttttggctggttctagagaccttcttagccacagtgattcttctgtcatcagttcatcaaatcctgaaaagagcatcatagc

SEQ ID NO: 63 - DNA, 1239 bp, Thlaspi arvense
  source 1..1239: mol_type = genomic DNA
atggctccagggactctcaccgagctcgccggagaggctaagctcaactctaaattcgtccgggacgaggacgaacgtcccaaggtggcatacaacaagtttagcgacgatatcccggtgatatctctcgccggactcgacgatgttggtgggaaaagaggagagatctgccgtaagatcgttgaggcttgcgagaattggggcgtgttccaggtggtcgatcatggtgtcgataccaatttggtagaggatatgactcgcctcgctcgcgacttctttgctttaccacccgaagagaaacttagtttcgacatgtctggtggtaagaaaggcggcttcatcgtctctagtcaccttcagggagagactgtgcaagattggagagagatcgtgacgtacttctcgtacccggtgagaaacagagactactcacggtggccagataagccggaagggtgggtgaaagtgacggaggagtacagcgacaaactgatgggtttagcttgtaagcttcttgaggttttgtctgaagctatggggctcgagaaagaagcacttaccaatgcttgcgtcgatatggaccaaaagatagttgttaattattaccctaaatgccctcagcctgatctcaccctcggactcaagcgtcacactgatcctggaaccatcactttgctgctccaagaccaggtcggtggattacaagccacacgcgacgatggcaaaacatggataacggttcagccaattgagggagcttttgtcgtgaatctcggcgaccatggtcactatttgagcaacgggaggttcaagaacgcggatcatcaggcggtggtgaattccaactcgagcaggctatctatagccacgtttcagaatccggcgcaggatgcaaccgtgtatccgcttaaagttagagaaggagagaagccgatcttggaggagccaatcacttttgcagagatgtataagagaaagatgggaaaagatctggagctggctcgcctcaagaagcttgcgaaagaagaaaatgaccagaagctggccaaagaagaacatgacaagaacctggccaaagaagaaaatgatcagaagctggctaaagaagaacatgaccagaagctggccaaagaagaacatgacaagagccttgccaaagaagaaaattaccaaaagctggccaaagatgaacatagccacacggaagctgttaagcgtctcggccaaatcctcgcttag

SEQ ID NO: 64 - protein, 412 aa, Thlaspi arvense
MAPGTLTELAGEAKLNSKFVRDEDERPKVAYNKFSDDIPVISLAGLDDVGGKRGEICRKIVEACENWGVFQVVDHGVDTNLVEDMTRLARDFFALPPEEKLSFDMSGGKKGGFIVSSHLQGETVQDWREIVTYFSYPVRNRDYSRWPDKPEGWVKVTEEYSDKLMGLACKLLEVLSEAMGLEKEALTNACVDMDQKIVVNYYPKCPQPDLTLGLKRHTDPGTITLLLQDQVGGLQATRDDGKTWITVQPIEGAFVVNLGDHGHYLSNGRFKNADHQAVVNSNSSRLSIATFQNPAQDATVYPLKVREGEKPILEEPITFAEMYKRKMGKDLELARLKKLAKEENDQKLAKEEHDKNLAKEENDQKLAKEEHDQKLAKEEHDKSLAKEENYQKLAKDEHSHTEAVKRLGQILA

SEQ ID NO: 65 - DNA, 4251 bp, Thlaspi arvense
  source 1..4251: mol_type = genomic DNA
aaaatgtagggaaaaaaaaacagagagaatcagatacagagagacgaaatccgaatcggagtctatctatctacttgaaattggtgacagaagaggaacagagaattgctaataaaattctaagataggcacaaaaaaacaagaacaagtttcatttggaacaagaagatgactgaaagaaactgcaagaagagaatcatggaaagacaaaacatacctaatgaaaagggaattttagagtcagaatcagagttactgattgaaaagatagcctgcgatctagtgactcttcttgctgatgatggagtctccattaggatcttgatcgtcttgcaagcaacaacggctaaagatcaagtgaaagaatttctgggaaaatcaatctgaatttcaatatctgagatatcaataaagaagagaaaggggataaacaattgcgaatctcttaggtagcagtattataggtatggggaaaaggttaccgtttgaaataaactagccgttgcgtctaaacggaaggaaggagccgtaatactagccgttgggtccattctctcatggaaagaaatcaagaatggttaaagacttaaagtagctgtttgggttgaaatctcagtacgttaggggcttttacggattaagaaggctgcgattaatagctcggcccatttatctctctgttgcgagttgcaagctgctaaaagttttataagcttttttattcggttatttgccaatcgtttaaattactcctatgatttataaatcttggaatgtgtgtatgagacgaagtattcattgttttcaatctatggtgtctgttttatgttttgcttttttttttttaattaaatatacaactcacaattcacaagccatgacaaaacatatcaaaatgttgataagagtaatgaaaactaaatacactagaaaagtcagatccaacatttcgtcttctcggattaaggacgatataaatataatatatatagtgaaaaaaaggtttttcttctctgatctcaatgggattatgtacaaataaccatgcaaaattgatgatcatttggatttttgtagtgcaaacccgtaacgtaacaatttttgccatgtgaattcaccgagaacgaccattttttttctttttttaataaaaaaatatgcaacccattttttaagacgtggcattaaattcaccgtaaaaaacatttaactatattgggcatggtgttaaagcaactttactagaaacgatttatgtacacaatggaatgtagcgcagaaaatatgatttattagaaagaaattaaagacagaagcacgtataggaagttgagtaactgcgtgtttctagaaacagaacggacggtatagtactgtggtaggtagcgagcgacctcttcgttcgtcagtcttcacaacctttgaaaggtttttcagctaccagtcatctccttatatattggttacctcttttatctctctttataaacttaattagtcttttgcctttgtaaccaaacactcacaaaaaaatataaaaatggctccagggactctcaccgagctcgccggagaggctaagctcaactctaaattcgtccgggacgaggacgaacgtcccaaggtggcatacaacaagtttagcgacgatatcccggtgatatctctcgccggactcgacgatgttggtgggaaaagaggagagatctgccgtaagatcgttgaggcttgcgagaattggggcgtgttccaggtggtcgatcatggtgtcgataccaatttggtagaggatatgactcgcctcgctcgcgacttctttgctttaccacccgaagagaaacttagtttcgacatgtctggtggtaagaaaggcggcttcatcgtctctagtcaccttcaggtaaagccacacaatttacgttacgttttcatgcgactcaaacttttatactgtagacttaattagacctataatcacatttcgggccgcaaaaatacgaattatgttttgatcttgtagttaaatacgaaataatgatgataaacgcagggagagactgtgcaagattggagagagatcgtgacgtacttctcgtacccggtgagaaacagagactactcacggtggccagataagccggaagggtgggtgaaagtgacggaggagtacagcgacaaactgatgggtttagcttgtaagcttcttgaggttttgtctgaagctatggggctcgagaaagaagcacttaccaatgcttgcgtcgatatggaccaaaagatagttgttaattattaccctaaatgccctcagcctgatctcaccctcggactcaagcgtcacactgatcctggaaccatcactttgctgctccaagaccaggtcggtggattacaagccacacgcgacgatggcaaaacatggataacggttcagccaattgagggagcttttgtcgtgaatctcggcgaccatggtcacgtacgtcagtatactctctcaatctctctgcttatatttttctccgtttgtgattttgacttggccaaacttgcttgtctattgaagtatttgagcaacgggaggttcaagaacgcggatcatcaggcggtggtgaattccaactcgagcaggctatctatagccacgtttcagaatccggcgcaggatgcaaccgtgtatccgcttaaagttagagaaggagagaagccgatcttggaggagccaatcacttttgcagagatgtataagagaaagatgggaaaagatctggagctggctcgcctcaagaagcttgcgaaagaagaaaatgaccagaagctggccaaagaagaacatgacaagaacctggccaaagaagaaaatgatcagaagctggctaaagaagaacatgaccagaagctggccaaagaagaacatgacaagagccttgccaaagaagaaaattaccaaaagctggccaaagatgaacatagccacacggaagctgttaagcgtctcggccaaatcctcgcttagagtccttgttgctccgctgtgttggtttcggtttggttttgtaatttgttttatcgtcaagttaacgaacgtggtgtgtttgagtttcttctcaatatagaaataatgacaagtaatgcaattgtacttgctctgtttgtttggttactttttatgtcagaagcacatacattactttctcgctattcgtgtggatgtaaatttaataagataatgacggttaaaccggtagtaggaccattttatttctttatggtttctttacgccacgtgtccgtctaaaagaaacttaccttgaaaaagtgaataattcatccctctttttcaagtttcgatttttttttctcttttcgagtcattttgttctaatcaacgacgacgcgacggagatgaatccttttcacggtgccgacgggaagcaaagaccggcgatgtatccggacatcgatcaatcaattccggataatcctttcgctcagacaaacccttatgccgcttctgcccctaatctctacccttctccctcctcgaacaatctcgggcctaatcttttcccggaccacggagatgcatccaacaaccagtcaccgtctgctccgcctcaagcgacggaggaggttctcatccgtgtcccgggctccattctcaaccttatcgacaaatcctacagcgtcgaactcgcgtgcggtgacttcaccatcgttcgaatcgtccagggccagaacatcgtcgccgttctcgccaacgtcgccgatgagattcaatggccgttgaacaagagcgaggtagcagcgaaggtcgatgggtcgcactatttcttttcaatccacccgccaaaggaagagggtcacggatctgggtccgactccgatggtgagaagcaggggaaaaagagcaatccgaaaccggaggacgaaattctcaattacggattgaccattgcttcgaaagggcaagagaacgtattaaacgagctcgaccgggttctccgggactattgctgcttcacggagcagaggatgtctgaaaaggcgaaggagacgggagaggaggtgctgggaaactccatggctgcggcgacttcgccggaggagctgaagggtgagaggaaagacatcgtggagggtcaatgcgcggcgtattggaccacactcgcgcctaacatcgaggattacagtagcaagactgctaagttgatagcttccgggtcaggtcagctgatcagagggatactt

SEQ ID NO: 66 - DNA, 1545 bp, Thlaspi arvense
  source 1..1545: mol_type = genomic DNA
atggccactctcttactcacaatcctcctccccactttcctcttcctcctcgtcctcctcctttctctccgccgcaaccacaaccgcagtagccgtctcccaccaggcccaaaaccatggcccatcctcggaaacctccctcacatgggtcctaaaccccatcgaaccctagccgccatggtaaccacctacggtccaatcctccacctccgattagggttctccaacgtcgtggttgctgcgtctaaatccgtggccgaacagttcttcaaaatccatgatgccaatttcgctagccgaccaccaaattcaggagccaaacacatggcatataactatcaagatcttgtctttgcgccttacggacaacgatggagaatgttgaggaagattagttctgttcatttattttcagctaaagctcttgaagattacaagcatgttcggcaggaagaggtaggaacgctcacgcgcgagctagtggatgcaggcacgaaacccgtaaacttaggccagttggtgaacatgtgtgtggtcaacgcgcttggaagagagatgatcggacggcgtctgttcggcgccggagctgatcacaaagcggaggagtttcgatcgatggtgacggaaatgatggctctcgccggagtattcaacctcggagatttcgtgccggctctggattggttagatttacaaggcgttgctggtaaaatgaaacggctacacaaaagattcgacacttttctatcgtcgattttgaaggagcacgagatgatgcacggtcaagatcaaaagcataaagatatgctcagcactttaatctcgctcaagggaactgattttgacggtgacggcggaagcctaacggatactgagatcaaagccttgctcttgaacatgtttacggctggaactgacacgtcagcaagtacggtggactgggccatagctgaactgatacgacacccggatgcaatgaccagagcccaagaagaacttgattcagttgtaggccgcgataggcccattaacgagtcagacctttctcggcttccttatcttcaggcggttatcaaagagaatttcaggcttcatccgccgacaccactctcgttaccacacatcgcatcagagagctgtgagatcaacggctaccatatcccgaaaggatcgactcttttaacaaacatatgggccatagcccgtgacccggaacaatggtccgacccgttatcgtttcgacccgagagatttttacagggtggagaaaaatccggcgtcgatgtgaaaggaagcgatttcgagcttataccgttcggagccgggaggagaatctgcgctgggctcagtttagggctacggatgattcagttactgacggcgacgctggttcacggatttgattgggaattggccggaggaattccgccggagaagctgaatatggaggagacttatgggattactctgcaaagagcagttcctttggtggtgcatcctaagccaaggttggctcccagtgtttacgaactcgggtcgcgctaa

SEQ ID NO: 67 - protein, 514 aa, Thlaspi arvense
MATLLLTILLPTFLFLLVLLLSLRRNHNRSSRLPPGPKPWPILGNLPHMGPKPHRTLAAMVTTYGPILHLRLGFSNVVVAASKSVAEQFFKIHDANFASRPPNSGAKHMAYNYQDLVFAPYGQRWRMLRKISSVHLFSAKALEDYKHVRQEEVGTLTRELVDAGTKPVNLGQLVNMCVVNALGREMIGRRLFGAGADHKAEEFRSMVTEMMALAGVFNLGDFVPALDWLDLQGVAGKMKRLHKRFDTFLSSILKEHEMMHGQDQKHKDMLSTLISLKGTDFDGDGGSLTDTEIKALLLNMFTAGTDTSASTVDWAIAELIRHPDAMTRAQEELDSVVGRDRPINESDLSRLPYLQAVIKENFRLHPPTPLSLPHIASESCEINGYHIPKGSTLLTNIWAIARDPEQWSDPLSFRPERFLQGGEKSGVDVKGSDFELIPFGAGRRICAGLSLGLRMIQLLTATLVHGFDWELAGGIPPEKLNMEETYGITLQRAVPLVVHPKPRLAPSVYELGSR

SEQ ID NO: 68 - DNA, 7303 bp, Thlaspi arvense
  source 1..7303: mol_type = genomic DNA
ctctttttcgtttaaaaataaatataaaatcagttctttatgaaattcaaatgcggtctatgatccataaacgtggccttggcatttacccgaatcccatccgtctccatcatttgcctttcgatcgtatatggaagttggaacgacgtcgacgccaattctgcggtaatcgtaatagtattaaaattgtggagccaacggaaacacaatgcatttttgcatttccatgcacacctttgtttaattttctaaagagaaattagtatatatataatttgtattgatctaatttttttaaatccagttcgactttgaaggaatttaagttcgacttgtatataattcttgtcaagacctcgatcccgcatccagtaataaaataataatgattgcaaaggacaacactaataaagaaaatgctataacgagaaagaattctgaaagacgtactaacgaaaattgtgtcctagtgaccataacaggagtatcatccaacactatgaaccacatatcttacctacccttcacgagtactctccagctaccacgattaacgataaatatatggtccagagaatttccgaatgatccgctaatattcgaataactgaaattatcaagcagtgagtttaattttttgtaaccgtatatctttttagccttttaaaatgataaactcatttatgaattaattgaatatatattacgtttaacccatccattcatattgtcacatatttttgaattttgttaacttcctttttatttcttttcgtattttctcaaaatcatatgtagtcattatatatacacacaaaaaaaagagatagatattgactaaaaggtagtgaaaaattacaaatctatagacattacaccataaaaaccatttttcaatagacaaatatatggttttactatgtgttgcgggaatcaacggaatcaacttgatgttagtatttacaaatagacgataaatcttctaaaattaattataaacacgtgaaagcttaaagacattgtgtaatatcttctgataatttttaatgttcaagacagtattttgattatgaagcttggtcgaaaccaaaaacaaaaatcaaaagacaagaagaattgaagaagtgaagaacacaacgtttcgttttaaaccatacaaaaaagtggttcacgatgtgatcttaaatgtatatctctttaataatcacttttccgttgaaaaggagaggaaaaaatcactgtagacttagtttttggaaagtatttctcatccccttttcgcaaggggattgtgtttttttttatcgaaaatacatggtccatggttaaatgggtcaagagtataatttagtactataattttataatcaaaatgttctaagttctatcttatatttatataaaaatagaagaagatgttctaacttatatcatattaattagtggctttacttaaccaaaagattgttacttatatttttcaacctacgttctactaacgactaagtattcagtaagatatatagctacctttcatagtattaatcaaatcttacgaaccagaaagctcacctaccattcgactctctttcgcttccaacttattctacataagataagattttctcatctacatattcgtatgtttgacgtcgtggtgggatccatgatttatccactttaacaatagaaaacaaaaacctaatcagagaaatataagctcttatgttttaaaaggaaaaaagcaatggaaattgaagctttttaaccaacaaaaaaaccagagaaatatacattattataattttatatattctgtcaagacgataattatcaatgtatatgtgtactaataatgttattactcgattacacccgtagttaataagccacttaaaattaactatttacaaaaaaaaaaaatcttttggtgactttgatttgatggtgtgaaaatctacatctcattcttcagtattcagatgattataacaaaatgttttttttctaaacttgcatttccgataatctaatactaaaaaatgtatcggcagaaagaatactagcttgaaaaacctttttataaaaaagaaaagtaaatatactagctaatggtgcccaacaggagatcgaagtggtgatgtttaattagttaaaggtaaaagaaaacctatttctaacttttgatgaaacaataaaaacaaaaaaatctttctaacctaaccgagacatgcatagatagattattggatttcgcaaaaaaaaaaaaatcaccctttatttatagcttatgtttgttattcctcatttctcacttaccattcaaacccaacactatggccactctcttactcacaatcctcctccccactttcctcttcctcctcgtcctcctcctttctctccgccgcaaccacaaccgcagtagccgtctcccaccaggcccaaaaccatggcccatcctcggaaacctccctcacatgggtcctaaaccccatcgaaccctagccgccatggtaaccacctacggtccaatcctccacctccgattagggttctccaacgtcgtggttgctgcgtctaaatccgtggccgaacagttcttcaaaatccatgatgccaatttcgctagccgaccaccaaattcaggagccaaacacatggcatataactatcaagatcttgtctttgcgccttacggacaacgatggagaatgttgaggaagattagttctgttcatttattttcagctaaagctcttgaagattacaagcatgttcggcaggtaacagtattagttataaacgttattctcatttcctaacgctataactcacctatatatagagatagtcatgccgttaatttttatatttatgattagtttattttatatcattatttaactgtggtacgaactctagaatttaataaaataaatttagagaagtacgtatagagatttttaatggtggagtggcgtttttttgtttagtattatgttttcagtgttgataaaataaccaaaaaataataatgttagtaatcgttaaaacacaaaacaaattttgttgataaaacgagatttatgactttttatcaacagattttgttttgaaaaacgagaactagaacataatactataacaagttccaatcctgtagaactagtcaccaagtcataaaactggaacttttttatcaaaaaatattgtttcgtttttcacgatttgcaacctttttcgttattttaacaatattggaaacattgcaacctataaaccccaatttaaacgagtaaaacaaaaaaaaaatactaaagtaggcctaagtgcaactgtgcaatactgtcgttgccatagacgtacagtacgatgtatccttgataataccatggaaaaatggaaagttcatcagaaacccaaggacaaaatacttcaatatgtatggaccacatttttccttaaataagatgtatcaacgagtcaccacgagacacgagtgattcactttcagtagtataagattgtttttgcattttcgtttaaaaaaatgtagatgaacatacaagatgatcctgacaatttatttataggtagattacttttcattgatggaacaattgaatatacaaaaatgtaaactagacttcagaattttaagtttaggggttaaagtaacttaactagggtctaaagtgtatataggctttatatttgagcttaggatttagatattggttaattagtggattttaatcgatttaaatttagaagcaagattatttgttattaagttattactttttaaaatcatagttaaagtcatgtgttaggggtttaataaattctaaatattagttgaaatttaatgttattaatttttataagaaatcgattttacattcgattttagtttttaacatctttgtaagtgcttttgagttactaaagcaactttcttatttaaaaacaggcttattgaattttttttaaaaattacgttttgaaagcatttgagtaattgtaaatcttaagtaaatttataaatcaattataaccctaactaaaatttattaatcaactaaacttcatacaccaataatcctacttagagttttataaggtttttgctgttaatcaaacatcaagatttaaaaatgagtaaaataaattccaaaatcaactaatatacccaaaattttccatttatagtttaagtagagcaaaattgaattttacaggaagaggtaggaacgctcacgcgcgagctagtggatgcaggcacgaaacccgtaaacttaggccagttggtgaacatgtgtgtggtcaacgcgcttggaagagagatgatcggacggcgtctgttcggcgccggagctgatcacaaagcggaggagtttcgatcgatggtgacggaaatgatggctctcgccggagtattcaacctcggagatttcgtgccggctctggattggttagatttacaaggcgttgctggtaaaatgaaacggctacacaaaagattcgacacttttctatcgtcgattttgaaggagcacgagatgatgcacggtcaagatcaaaagcataaagatatgctcagcactttaatctcgctcaagggaactgattttgacggtgacggcggaagcctaacggatactgagatcaaagccttgctcttggttcgtataacgatcaattaattgtagtattaatttcatactttatgaaaagttattgattccttcttaaatacagaacatgtttacggctggaactgacacgtcagcaagtacggtggactgggccatagctgaactgatacgacacccggatgcaatgaccagagcccaagaagaacttgattcagttgtaggccgcgataggcccattaacgagtcagacctttctcggcttccttatcttcaggtatacccttaaccgggaattaggaattgtcttggttaacgagctctattgttaatccggtttaaacggttttgttttaaaaacaggcggttatcaaagagaatttcaggcttcatccgccgacaccactctcgttaccacacatcgcatcagagagctgtgagatcaacggctaccatatcccgaaaggatcgactcttttaacaaacatatgggccatagcccgtgacccggaacaatggtccgacccgttatcgtttcgacccgagagatttttacagggtggagaaaaatccggcgtcgatgtgaaaggaagcgatttcgagcttataccgttcggagccgggaggagaatctgcgctgggctcagtttagggctacggatgattcagttactgacggcgacgctggttcacggatttgattgggaattggccggaggaattccgccggagaagctgaatatggaggagacttatgggattactctgcaaagagcagttcctttggtggtgcatcctaagccaaggttggctcccagtgtttacgaactcgggtcgcgctaaacctaagtttgcttgccagcaaaggtaggtcttgcacggaggttaaaaagcttgtttctggaatttcctcaaataaagtttaaaatagcgtaatgtggtccggagtataaggctatgttatctgcgtcgtcgatccgatgccgaggaagaagacgatggctctcgaaagcaaacctgatccgcggacccgacccgttttatacgactaagattttcattcataatagtatatgataattttaaattttaaatttttatcgtgtaatttttacagtactgaattttgtattaaagcatcttccttactgtaccagatgcatttacaactctagatttttttctgccagcaaaatcagatttttccatcttttttatatatattaaagtggaacaccactacaacctgcaattctgcaacacatgttacattacacattcatttggcgttttccaataagttataaacagtgaatgtttgtagacaatagacaattataaacagtaaatcatgtttcccctctgttttcaaaagtgaaaaagtttctaaatatgtatatgtattatactttaataactcgtcggaccatactattcccaccttctaattaaccttgtatacactggtttaccaacaccatgtctcttccttgttcaaaaaaactccatgtctctttaattactttttttcttcttctcgaaatttgttgataatagaaactagtcgaagtttctctttaattctgttttcatttaatataagcgtcgttgttataatttcaaatttcaaaaaggtacttacaagttcacacggagagctacaataatggaaaagctgactcaagaagggctagaaaagaaacaagagtcacggaacaagtagtcactttcaatctcagtatacagtacactagtgtctgcttgcatctctctctctctctctctctctctcttggtctaaaaatatcagaagagaaaatctctctatctatttatctctctttccctttctctttcaacaagacaatagtgacgacatttgtgtttccatggctgttctggttctctatctcctcggtttcctcatggctggccactgtagtaagaaactctcgctcttcactacattacttatctctctcttttttttaaatgtgtctattgatgggaactaaatttcaataaaattctttcggctcttagctagttatcactacatctcttaagttcacgaccctcaaaaagaaggtttcctcaaatcttcttggcttcacacttacaatttgtttcatttggtaggcgcgacgtggtgtgtgtgcaaaacaggactgagtgactcagtgctacaaaagacattagactatgcttgcgggaatggagctgactgtaacccaactcacccaaaagggtcttgcttcaaccccgacaatgttagggctcattgtaactatgctgtcaatagcttctatcaaaagaaaggtcaagcttctgaatcttgtaatttcactggaactgccactcctaccacaactgatcccagtaagtttccctcagaaatctgatgcttttactgtgattgtttacaactctaaatagtttttggagtcacagaaagtccaccgtcattttggttacttaaaatgatttggtattagagattagtgaatattaaagtttaaagtaacaatttggttttagtaatgtatgaggaggatctgatttggttacagtacactaaatgtttttttttttgtgtgtgtgcttttttgctttgatgggtgaacattttgtcacaggctactcag

SEQ ID NO: 69 - DNA, 1581 bp, Thlaspi arvense
  source 1..1581: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaagacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 70 - protein, 526 aa, Thlaspi arvense
MDESSIFTAEKVIGAEKRELQGLLKAAVQSVEWTYSLFWQLCPQQRVLLWENGYYNGAIKTRKTTQPAEVTAEEAALERSQQLRELYEALLAGESSSEARACTALSPEDLTETEWFYLMCVSFSFPPPSGMPGKAYARRKHVWLCGANEVDSKIFSRAILAKSAKIQQTVVCIPMLDGVVELGTTNKVKEDIAFVELIKSFFHNHPKSNPKAALSEHSINEEHEEDEEQEEEEEEEVEEEMTMSEEIRLGSPDDDDVSNQNLLSDFHVESTHTLDTHMDMMNLMEEGGNYSQTVSTLLMSQPTSLFSDSVSTSSYIQSSFATWKADNFKEHQRVETKSTSSSQWMLKHIILRVPLLHDHTKEKRLPREELNHVVAERRRREKLNERFITLRSLVPFVTKMDKVSILGDTINYVNHLRNRVQELETNHHEQKHKRMRSCKGKTWEEVVEVSIIESDVLLEMRCEYRDGLLLDILQVLKEHGIETTAVHTAVNERDFEAEIRAMVRGKKPSIAEVKRAIHQTISNIKL

SEQ ID NO: 71 - DNA, 5726 bp, Thlaspi arvense
  source 1..5726: mol_type = genomic DNA
atctaatcaagagaagctagctaacaatatcgaagggaagacatgaacacatcctcacttttgcctaatcgaatgaaagctaaaaatggatctagggtttaagggatctaacgatggaaagcaaaggactcaagagaacggcggctaggaacggcggaacgacggcggctctcctcagcggcggcggtcggcggcgaaaacgaacggcggcggcggcttgctcggttagggttcctaggttatatctctgagtgctttgggagttatgatggaaaacctaattctctaactctgtttatatagggaagactcaggaaaaatcctaatatccttttctcttgggcttctgacacatgaacctttccttttcctattgagcctagatccaggacgttacaactagaatcagaatcgaacaacaagcgtatgcgcttaagaaatgatttttactcacactatatgggcgcgcatggcattgtcgtaagaattaaataagcttcgtcccactccttaatggatttacgcacacagtcaatccacgtggttgtcataactaaatataattggtgaaaaaactaatttaaagacaaagtaagaagcggaaaccgaaggaaaagccaaacaacattatgtttatcgacaaaaaagccaaaacaacatttaatgttaatttttgtccaaaccaggaatacatcactcatacggtcatatataccataccccaaattgtatgtactacatgtgtacgtaactgatttatgtatatctatctatttctcttttattgtaggggtcaaaatctacctatcgatatttataaataggattttataagacattgttaaaatggaaaacatggtagctacttggccagcgattaacatcaataattaaaaatacgaattgtgaaacatctcactctcttccttttatcacaccaaaccacttctctttctttacttaccggtcaggtcaacaattctaccattccctttatttagtttttttttttcatctatctctcttttcatttttaacttttaaaaaatattagtccgaaaatcgcactcttttatattcgttgatgggcattaaaatattgtaaaaattaaaaaaaatgtgtaaattaaaacgcaatttgtttgcacctgcagcagtagtaacacacaagtcaaagcaatgtctgttttctcttgtatgtcggttagctattttatgtactgaaatatgttattctttctgaccaaaacatattaagaaatttgcattctgtaatttgatttatccaatcagtagaggacacgtaaccaagataattagatatttttattctatctgtatactactaattcatccaacgtctgttggacgaccaaccagtcaaaactcataaaagtcaacttttactacatcgtgaattttttggtcatgcatttatatacacatacatggaagaaaaaacaatactttacctatgtcaaaatgctgcaaaagcattaagatggtatgtatacatacatattgatataacagtggtcccatatatcattttaaatcatcataaagagtatccatcttctttcatattaacatccccttcaaagttataagatttttctctcttcattagagagagagagagagagaattaacacaagttttaatctccgggaaagatggatgaatcaagtatttttacggcagagaaagtgatcggagctgagaaaagagagcttcaagggctgcttaaggcggcggtgcaatctgtggagtggacttatagtctcttctggcaactttgtcctcaacaaaggttcttttttttttttaataaatttcatcgatctctcacaataaaaaccctaaattttatatcatttattattatatgtttaactacataattatcagtattttaaccgtccatgtgctttatttggttccatttctgtctcatattttacttgaggttcagactgccgagcacatctctcgtttgtctcgaatctgtgagactttttcgtttattggcacttctgtgtcaattgagttatactgaagtaattatatgtttaaatgaattagggttttgctgtgggagaatggatactacaacggtgcaataaagacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggtacccaactctctctctctctctctctctctctctctctctctctctctctctctctctcttttgtctatactgaagtttcttaatttatctttttatcatctcctactgaagacaaaaatagtattgtgtgttaaatgcgaatcacgaatattgtggaagcattaaaaacaaactgaggaggttgagttactgaaagaagaaatgtattggagttgatgaaacgtacactccattttagtgaacataattggaccgttgagattcttatttttttgctgattgattatctaaagtagaagcataaatagataatacataaatgcataacaaattgtgttagttatgggtatagttaatgctttttctctatgagaggaaaaaaaaaaaaatatataaatgtggaagtaataattttgtaggatgccaggaaaggcgtatgcgaggaggaaacacgtatggctatgtggtgcaaatgaggttgacagtaaaatcttttctagggctattctcgcaaaggtctatttcctttttcatttaccactactctatgcatctacttctctacctatttatatatctcatctttcaaattaattaattttctgtctttattttcttggatgctccttctacatcggtcggttccttaatggttagagtgccaaaatccaggtaaacgttgtctttattgattaattctaattttgagtaatattttacatttattttacatgtttgaaaattgttttgtgataaaaaaaaagcagacagtggtttgcattcccatgcttgatggcgttgtggaactaggcacaacgaacaaggtacggcgtagttatctttttatatatgcataaccaaatggttaagaaaaaggttagaagagaaatagatcatgcttaagttttatcagttaaattaaaaatgtaaaaataagatattatgttcattaataatgtatagtccctgttagttaaaaaaaagaataaaatatttaaccatttgaagtcataatataaaaatattgtttttggagatagtacataattctcacaataaaaaaaataacaaagggatgattaagggaaggagttggatacatgttgtttgtctgtgtgtgtgaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttaggtatacacttatacattaaattagttaacgatatcattacacgtatctatttattttgttaacaagaaatttaaaaatattcgccatttctttgttatgtctaaagaaaatctataaaattttatgaatagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatagtttaagactctaacaatattgatgccaactctagaaaaggacaactgaacttattttgttttaagccttggctatattacattatcagttttacacttctacgtatacacaactttaatttttctatattactacatgcatactagatgttagaaatatcttatataagcatacatattaaattagttatgcgatatttaaaattaatgtatataaatattcagtttcaaagcttacgaatacgtaaaattaatcaatacaaaccgaaaccaaaaagccacttctcatctcaaaatgttaattacaaatttacaataagccgaatcactatctaaaaagcttttgggatcccgagacccaaaacatttcttcgtataagtgcaacaattatcgagtgtgatgagacacgtaaaaagcaaggatcgggacaacgattttcggctatatagtagtttctcatatctgactttattagcaaattactacataaaattgcttttaagtatgattaacggaacaagtataatgcgtttatttaattacagatctggctaaatgtaatatggtatcaaagctggaaagacaaatagtctaaggtttggaatttttcatctgtctataaaattcttaagacactgatagatcattaacttattttggagtcaccaatggacaaatcaataactattgatgtccaaaacccacaagacaagcctattttttttgcacgtgcaagtacaccggtggaagaattcatttgtatactgaatctgaaattataaaagattagaattggttgaagaatttacttcaaaacaagctagaagatggtgttatttaaataaacaatgttaaattatatcaatgactttcaaattttatcaaaacattttttgaaagagaatcacgcacaaagtatttcgaattacaaaacaattttttgcaataaattttatagttaaagcaaattttgtcaattattgaaaaaaattacttagataaaatatctaatttttttttttaaacaatgtctctataaaagatggattagccaagaaaatctgatggatgctacaacgcagggttggagatatggtgctgacactacat

SEQ ID NO: 72 - DNA, 2583 bp, Thlaspi arvense
  source 1..2583: mol_type = genomic DNA
atggaagggaactctttaagttcatcttccttagtttcagccaatcgggtgggtcaatgtgacaatcttgagcatagtagaatgacttgttctcgaaatttcattgagatgcatttgactgaccagctgagaaaaactcagatagtgaacgagaccaataaggatcttgttgtcgaggcactgagatcaatcgcggagatattgacttatggcgatcagcacgaccctttattctttgaattttttatggagaagcaagtaatgggagagtttgtacgtattttgagggttagcaagacagtaacagtttccgttcagttgttgcaaaccatgagtattatgatccagaacctaaaaagtgaacaagccatctactacttgttcagtaacgaatatgtaaattatttgataacatatacatttgacttccaacatgaagagcttctatcttactacatatccttcctaagagctgttagtgggaaactaaaccagcatacaatatcgttgcttttgaagaccgagaacgatgtagtagtttcttttcccctttacgtcgagggcataaaatttgcatttcatgaagagaacatgatacgcactgcagttcgtgccctgactcttaatgtgtatcatgttggcgatgaatctgtgaatgattatgtagttagtccaccgcacactgagtacttttcaaaattagttacatttttccaaaagcaatgcatggatctaagtgcaatggtgttgaacactctaaagagcccatcccgagactcaggtggaaaattgtttgctgccgttgatgggatcgaggacacgctgtactactttagtgatgttatctctgctggcatacctgatatcgggaggctgataacagatcacattctacagcatctaactctcccacttcttctcccgtctttgtgctctgaggctgataaatcagttgatcctgtgacttctctctatctgctctgttgcatcgtgcggatagttaaaatcaaagatttggcgaatttgactgctgctacttttttctgccctgtagaagctttcatttcaagttccctagtgaaacctaatagtagcttggctcctgaacgtcttacatatggaaatgggcatccagacaatggtgttgctgaggaaacagttcaacagtgtccaagcactgcagtctcgagtgaatatggaaattcccacgtttgcagtgaaaatactacgaaaagcatcttcaacaattcacgtattacgtttagggatactttacttcaatatatttctgagggagatgatgtgcaagctcagggttccttgtttgtgctagccacgttgttgcagacaaaagaacttgaagagtcaatgctagatgcttttggcattcttcctcagcgtaagcagcacaaaaagcttttgctgcaatctttggttggggaagacactggtgaagaacaactgttttcaccacaaaatggttccatgagagatggcttaagcagcgaacttgattggtatctacggaggttggaggagcagtttggagtatgctgttcactgcctggggctgcaatgtgcccgcgtgtacatagacatcaggtggtggatgcattggtcattcttctctgccgtgaaaacatatctgcagaaacattatgggatggagggtggcttttacggcagttgcttccttatagcgaggcagagtttaatcgtaaacatctcaaaatgctgaatgattcatatgagaagtgcacaagtgcactaatccgggaggttaaaggtacctggcctgatctactcatcacggtgctgcttgatgagtggaaaaagttgatcgaagctccatcccctcaaaaagagcctaaatctgttcttctccagctggataaatcctcttctagtggtaacactgttagcgaatcatcattcacagccggtgaaaaactgtgcgaggtggtgaaggtttttgtgcttcttcaccaactccagatcttctcgcttggtaggcccttgccagagcagcctcctatccatcctcctgcagacagatctgaaacgtctcgcgccacaagtgctggtttggatgtttcagtccccaaaactggcaccgaagtgaagctagttgatgctgtacactgtaggattgcctttgaaagaggcaaggagcggaatttctcgtttctagcattatcatctggtgtgtctgggtggattgtccttgctgaagaatcgctttcgaagccagatcacggaaccgtccgtgttaccgcacctttagccggctgcaaaccccggatagatgaaaagcacccaagatggctacacttgaagatccggccatcgacgttaccattcttggatccaacaaagcgaggagtctatgagaagctcaagtccaaaggtctagtagacgggagatggacattagcattcagagacgaagagtcttgttactctgcttactcaatggttgtacatgagatcgatctacaatgcagcgaggttgaaaggaggttaaagccattgttcgaccttgagagaaaccagcaagatcaatcaaacgttgcttctctatag

SEQ ID NO: 73 - protein, 860 aa, Thlaspi arvense
MEGNSLSSSSLVSANRVGQCDNLEHSRMTCSRNFIEMHLTDQLRKTQIVNETNKDLVVEALRSIAEILTYGDQHDPLFFEFFMEKQVMGEFVRILRVSKTVTVSVQLLQTMSIMIQNLKSEQAIYYLFSNEYVNYLITYTFDFQHEELLSYYISFLRAVSGKLNQHTISLLLKTENDVVVSFPLYVEGIKFAFHEENMIRTAVRALTLNVYHVGDESVNDYVVSPPHTEYFSKLVTFFQKQCMDLSAMVLNTLKSPSRDSGGKLFAAVDGIEDTLYYFSDVISAGIPDIGRLITDHILQHLTLPLLLPSLCSEADKSVDPVTSLYLLCCIVRIVKIKDLANLTAATFFCPVEAFISSSLVKPNSSLAPERLTYGNGHPDNGVAEETVQQCPSTAVSSEYGNSHVCSENTTKSIFNNSRITFRDTLLQYISEGDDVQAQGSLFVLATLLQTKELEESMLDAFGILPQRKQHKKLLLQSLVGEDTGEEQLFSPQNGSMRDGLSSELDWYLRRLEEQFGVCCSLPGAAMCPRVHRHQVVDALVILLCRENISAETLWDGGWLLRQLLPYSEAEFNRKHLKMLNDSYEKCTSALIREVKGTWPDLLITVLLDEWKKLIEAPSPQKEPKSVLLQLDKSSSSGNTVSESSFTAGEKLCEVVKVFVLLHQLQIFSLGRPLPEQPPIHPPADRSETSRATSAGLDVSVPKTGTEVKLVDAVHCRIAFERGKERNFSFLALSSGVSGWIVLAEESLSKPDHGTVRVTAPLAGCKPRIDEKHPRWLHLKIRPSTLPFLDPTKRGVYEKLKSKGLVDGRWTLAFRDEESCYSAYSMVVHEIDLQCSEVERRLKPLFDLERNQQDQSNVASL

SEQ ID NO: 74 - DNA, 7185 bp, Thlaspi arvense
  source 1..7185: mol_type = genomic DNA
cgtggatggaggaagggtttctcgtcgtgttcgtcgcgaaagatggttcctcttctgcgccgccaccgccggagatcgaggagaatcgcaacgtgagagtcgtggaaatcaccggtgatgaatgaatgaataattatgtctttaaaacttcaaaatctgtgtattatatgaacagagtaatcatgttgtgatcgtgctgctgttttcgtatgtaatagtgaaagttgcataaaagcccttaaactaatcaaagttttcaaaataacccatcatttaagaaaactataaaacgatcaaaattaatgtaaaatctacatttgagcttccgactacaacatttattcgattcaatttcccttttaataatctaacaaattttccaaatttcaacaaatctcgaaccgaatgattatttgattcaaagacaaactattatgttttcgctacaacccaaaacgatgaatttaagtgtacagtcggaattgtattactgtatagattttaaattagatgggacatttgtttaaacaatttaattaagtgagggttattttacaactttgctatattttgaggggttttgttataattttgtgacttaatagcaagagtcgtctttgaaggaaccgttgtgacgtggcggtcttcaacactcgaatggtcgatcgtaaaaataaatgttttccctaattaaaatgccatagacggatccccccattctaaagattctttctttgaattcgatattgaacccgacccggatccgaatctggatacttgacccaacgccagctcaacgccccacttcggtgcttgcttctattcgtctctccgtcaacaattctgttgtttaccggaatacctagagccttcgagaaattcaaacacgacgcagatatcattctttcgcttacgctattgatttgtatgattttgtcgtcggtgaagcaatccgaaccggaactggaatcggatttcatcaggacttgtggggttccctttaatcgaggagcaaccatggaattggggttttcgcgggagtgaaggatgatccagagaagactggattcagaggattgatccgataatcatgtggttctccttcttaagacccagagatcgattctccttggtcgaactcaggttaaatttttttttgtttgatcgggtcgatcccgcaacaagttccatttgcgatatatatctctgtttcgtttgatccctattgtgactaagattagcggatttttgtgaattctggccttaaaatgtgtcttttaagggtggttgagaggtttttcgtcgtaatctctttggtgtgaatgacttgttctcgaaatttcattgagattttagtgtacgacaagtatttgttcggtgattgtaagcgttgaaattttgagttttttcgtcgtagtctcttcggaattagaatgggcagtttgcagttatctgttagggttttcgagaatttgaacatttgatcgcttgaatggtgtcatggaagggaactctttaagttcatcttccttagtttcagccaatcgggtgggtcaatgtgacaatcttgagcatagtagaatgacttgttctcgaaatttcattgagatgttagtgtacgacaagtatttgttcggtgattataccagatgatgaagcataaacgttgaaactttgggttttgtttttgtaggcatttgactgaccagctgagaaaaactcagatagtgaacgagaccaataaggtcagtgggataaaagtctttttcactttagcaactttgtcttggcattactgttctgttttttccttgtggattcgatctttgtttaccgttttgaaggatcttgttgtcgaggcactgagatcaatcgcggagatattgacttatggcgatcagcacgaccctttattctttgagtgagagtctttatgtcaattatcagtcaatgcccttcattttcttattggcatatatgctaataatactttgcgctcgttgcagattttttatggagaagcaagtaatgggagagtttgtacgtattttgagggttagcaagacagtaacagtttccgttcagttgttgcaaaccatgagtattatgatccagaacctaaaaagtgaacaagccatctgtgagttcatcagaagcagtgtatattttgtctttgataattcgtaatgattatagcctattgatgatcttcttttgaaactacttgcagactacttgttcagtaacgaatatgtaaattatttgataacatatacatttgacttccaacatgaagagcttctatcttactacatatccttcctaaggtacattcttctgttgttgacctcggcttcaggaagtacatgttgactgtatctggtaactgttttgttttctttttgtagagctgttagtgggaaactaaaccagcatacaatatcgttgcttttgaagaccgagaacgtgagtattctttaagttgcatcttccttaaacttctataatagtcctttatggattttttttttctgccatattttaattctgcgtttctgcgtctaggatgtagtagtttcttttcccctttacgtcgagggcataaaatttgcatttcatgaagagaacatgatacgcactgcagttcgtgccctgactcttaatgtgtatcatggtaagtcactcagtcctttcgatttgtcactttaatgactcagaagcttttagcaatgtttctgatgcctttagttttttttttcttttcgcagttggcgatgaatctgtgaatgattatgtagttagtccaccgcacactgagtacttttcaaaattagttacatttttccaaaagcaatgcatggatctaagtgcaatggtgttgaacactctaaagtaagattcactgcatcttcatttcccagggcaataatatcgtgttaaatacgtatcatatccccttttcatctattgactcattagttatattcttctgaaagatttccatttactttgtctaaaggagcccatcccgagactcaggtggaaaattgtttgctgccgttgatgggatcgaggacacgctgtactactttagtgatgttatctctgctggcatacctgatatcgggaggctgataacagatcacattctacagcatctaactctcccacttcttctcccgtctttgtgctctgaggctgtaaatgtataacatatctactttttcgatttattttgtccttttgtgctaattttgtaagccttgttagttttttctatgcaagagtacaatttctaatgtcagtatactgtttgttctgtggttatttcaggataaatcagttgatcctgtgacttctctctatctgctctgttgcatcgtgcggatagttaaaatcaaagatttggcgaatttgactgctgctacttttttctgccctgtagaagctttcatttcaagttccctagtgaaacctaatagtagcttggctcctgaacgtcttacatatggaaatgggcatccagacaatggtgttgctgaggaaacagttcaacagtgtccaagcactgcagtctcgagtgaatatggaaattcccacgtttgcagtgaaaatactacgaaaagcatcttcaacaattcacgtattacgtttaggtaacatgttatctaacaacctgagtgaatttgagatagttgctttttacactccttaagctgtcttaatcataatatattcctatatctctccgaagctgatgttccttttgtaaatagggatactttacttcaatatatttctgagggagatgatgtgcaagctcagggttccttgtttgtgctagccacgttgttgcagacaaaaggtattggtagtggctttgtgcatctccttattttatttctagttttgacagggaaattcgcatctcacgcttacattctctttgtggtagaacttgaagagtcaatgctagatgcttttggcattcttcctcagcgtaagcagcacaaaaagcttttgctggtgagtatctgtcattattctttatcttctgctcttttccagcaaattccaaggcagattgccagggaaattcgcatctcatggtttatggaccgtctactatagtctggaccaaaaatataagctttacttcttggatagctaaacttaatttggtggtcatctctgacagcaatctttggttggggaagacactggtgaagaacaactgttttcaccacaaaatggttccatgagagatggcttaagcagcgaacttgattggtatctacggaggttggaggtacttcagtcactgcatatcttagaattttgcaaacacgttctacttctccatctctttggtgttgtgttaaattgtgattgacttactcattcaagtaccctcaacaggagcagtttggagtatgctgttcactgcctggggctgcaatgtgcccgcgtgtacatagacatcaggtaaattacgaatgtttgtaaacctaaattattagcctcgcttcttctgaaaagtgaaaattccatctgcttgagttaggttctttcttcatcacagagcaagttcttgtcttgaatcaatgaaaaaattgtggtataggtggtggatgcattggtcattcttctctgccgtgaaaacatatctgcagaaacattatgggatggagggtggcttttacggcagttgcttccttatagcgaggcagagtttaatcgtaaacatctcaaaatgctgaatgtatgtgtgttgcttttaagatgttgacactgttacactctccctttcgtcattatttaccagtttcctgctgatgtcaggattcatatgagaagtgcacaagtgcactaatccgggaggttaaaggtacctggcctgatctactcatcacggtgctgcttgatgagtggaaaaagtgtaaaagaggcaagtaaagaagcaaaatgtttggattttgattctaaatttacatgttcagtgaatctaataccaaatctcttgtgacagtgatcgaagctccatcccctcaaaaagagcctaaatctgttcttctccagctggataaatcctcttctagtggtagaatttttgttttacaatcaatgtgaatgatatagacactcccttgttcagttcctgctaatgcaaaccgttaacaactgaacttttacaggtaacactgttagcgaatcatcattcacagccggtgaaaaactgtgcgaggtggtgaaggtaagtccattgtgttccctctgtattttcaatcagcgcttgtcgttgatgtgtaaatgctacagaaatctgagaatggagcattttactccttaggtttttgtgcttcttcaccaactccagatcttctcgcttggtaggcccttgccagagcagcctcctatccatcctcctgcagacagatctgaaacgtctcgcgccacaagtgctggtttggatgtttcagtccccaaaactggcaccgaagtgaagctaggttagcacatgatgctctttatgcttgcttagttcaacatctaccaaaccgacaccatgtccgtagttaaactgatgtttgaaatttcccggtgcagttgatgctgtacactgtaggattgcctttgaaagaggcaaggagcggaatttctcgtttctagcattatcatctggtgtgtctgggtggattgtccttgctgaagaatcgctttcgaagccagatcacggaaccgtccgtgttaccgcacctttagccggctgcaaagtacgtgctcacctacattgtctttttttttgtaacgatattgcttgtgaacaccacttacaaggacagatataatcatgatgttggcatagagaaatgagctatcttttgtttggtattcagccccggatagatgaaaagcacccaagatggctacacttgaagatccggccatcgacgttaccattcttggatccaacaaagcgaggagtctatgagaagctcaagtccaaaggtctagtagacgggagatggacattagcattcagagacgaagagtcttgttactctgcttactcaatggttgtacatgagatcgatctacaatgcagcgaggttgaaaggaggttaaagccattgttcgaccttgagagaaaccagcaagatcaatcaaacgttgcttctctatagctcttgggtttgtttttgttttattcattaaattttttgtttcttctttgtaatgttgctataatgtgataatatcgttgtatatttgaaagtgatagtgaatttcagtttttatcacacaaaaatatgactagtgtgagtgtcgagaactctgcttactcttataattctgtgattctgcgtctcttcttgttgaatatgaaaccctaaaacaaatgaacccttactcttctttatgcatcctgtagctgtcaacgagagcagttttgtatgagttacggacactctgtttccagatggagagagaagcaatcaccgtccgtattttgagcaatcctctttttaacgcagctaatggatgcaacattggcttttagacttgtaactatgtctctctctctctctctcttccttcagccgcagtttaaaagacccacatacagtaccacatcatacctcagatgtgaatggtttgtcttcactaaaaggtttgctggaaccaaaacctaaacaggaaacataactccttgacccacagtaaattaaactaccagaatcactctgaaagaagttcctcgaccagaaatatccaaaagccacataaacagggtctggtcagacataacagtggcaacatcgtaataagaaagatgccaagaagttgcagaggctaatgtttgaaaaaaaaagaagccatgagagaccaaaattaagagaatttgtaattaatacagtactcaggaaaagagtttcaaccacttgcgccaacaaactagatagacattgttatccaacacacaaactcatgtccaaccaaaagatagagagataaagagacattatccaacacaacacatgtccaaataaaagagataacagataaaagatagaagagtgggttctccttttcttactacaaacacccatgaattcgattccctactttcggccaagtaccctgaaacacaacacaaaactgaattgttatacaaaactatgt

SEQ ID NO: 75 - DNA, 1026 bp, Thlaspi arvense
  source 1..1026: mol_type = genomic DNA
atggataactcagctccggattctttgtccagatcggaaaccgccgtcacctacgattctccttacccactctacgccatggccttctcttccatcggtactccctccggccaccgaatcgccgtcggaagcttcctcgaggattacaacaaccgcatcgacattctctccttcgattccgactccatgacggtgaagcctctcccgaatctctccttcgatcatccttatcctcccacaaagctgatgttcagccctccctccctccgtcgtccctccgccggcgatctcctcgcttcctccggcgatttcctccgtttatgggagatcagtgaagattcctccaccgtcgagcctgtctcggtgctcaacaacagcaagacgagcgagttctgcgcgccgttgacctccttcgactggaacgacgtcgagccgaaacgcctgggaacctgcagcatcgacacgacctgcacgatttgggacatcgagaagtgcgtggtggagacgcagctcatagcgcacgataaggaggtccacgacatcgcttggggagaagctagggttttcgcgtcggtctccgccgacggatccgtcaggatcttcgatctacgcgacaaggagcattccaccatcatctacgagagtcctcagcccgatacgcctctcctaagactcgcctggaacaaacaagacctgaggtacatggcgacgatcctgatggattcgaataaggtcgtgattctcgacattcgctcgccgacgatgcctgtcgccgagctcgaacggcaccaggctagcgtcaacgccatagcttgggctccacagagctgcaaacacatctgctctgctggtgacgacacgcaggctctcatctgggagctcccgactgtagctggacccaacggcattgatccgatgtcggtttactcagccggctcggagataaaccagttgcagtggtcctcttcacagcctgattggattggcatcgctttcgctaacaaaatgcagctccttagagtttga

SEQ ID NO: 76 - protein, 341 aa, Thlaspi arvense
MDNSAPDSLSRSETAVTYDSPYPLYAMAFSSIGTPSGHRIAVGSFLEDYNNRIDILSFDSDSMTVKPLPNLSFDHPYPPTKLMFSPPSLRRPSAGDLLASSGDFLRLWEISEDSSTVEPVSVLNNSKTSEFCAPLTSFDWNDVEPKRLGTCSIDTTCTIWDIEKCVVETQLIAHDKEVHDIAWGEARVFASVSADGSVRIFDLRDKEHSTIIYESPQPDTPLLRLAWNKQDLRYMATILMDSNKVVILDIRSPTMPVAELERHQASVNAIAWAPQSCKHICSAGDDTQALIWELPTVAGPNGIDPMSVYSAGSEINQLQWSSSQPDWIGIAFANKMQLLRV

SEQ ID NO: 77 - DNA, 4435 bp, Thlaspi arvense
  source 1..4435: mol_type = genomic DNA
acacatatctaatactataaaaaaaggtgtatagcgtccgcgaaactcatgtcttccataatcattaatcaaaaaccgatatctctaatcaagatgcatttgacaagaaaaaagaatcaaaatgcattgccaagaaaaaaagaatcaagatgcatgattgtttggtgagtaaacgagacatcttaattttaatataacaattcgttccttgtttttttttttcctgtatcgtacgtgattcatgtgttcaactgttttgttgtcatctcatgtgttagttaaattctatcttattgattttaatacatagatatttcggtgcagcgcaaaaacattattatatttcggtgtagccgcatttagcacaatcatacctaaatattaccaaattgtgctccactatcttacgtgattcatgtgttaaactgttttttctttcctgtggtctaccaaaattaagtttttaattgcaaagacgattgccaattttaattgacgatctaatttgtcacagataacataattatctgatggcgtataagttataagctcttttattttttgaacaaaatatttttgttcttagctgagctgggaaatgtcgtatttaaagtattaaacctataatctcttcggtaatatgttaaataaatctgttttttcaggtcgttagtaaattatttcggctacagacgccatatcaaataatatatcaggttgctgtgaatgagtatctcaatttgcatgctaagaactatataaactgtattaacttttatcttaataccatgagtaagtcgaattatcgatctatctattatttttatttttaaacgtgtggcctgattgataaaataaacttgtggtcatatcaaatctgaaactggagtaaacgtgggattggggatgaagaaattagggcatgtcaacgtcttgggacttaccgtcatgtcccttcactgtaatattggacctatcattttcttccttatcatcttagttttttttgtcgtctcatcctctttaaaataaagtaaaagtaattaacactattatattttctcacaaaattagatattatatgctcttttcaaaaaaatgttaggcatctaacatgatatttttgagatagaaaatgacacaaatactcttcaccccaacctcacgcatatctctatatttattacatcgctattacacacattacacatagtattcatgtacatgtctatttacgcaaaggatcacacgttccacaaaccatttgctaaatcctaaatgagtagtaacaagatattgtccgtctcaaaacaaaatatatatttgtagagaatatcataattttagtatcgtcttcaaaatggtttgaaaatgttattgactttttgtagacaacattttaatctgtgtgtttgggaaagaaatgtggaaagctcgcttagcgttgaaaaggcctactatgtaaagacacaaggcccaaaaatgcgttgaaaggcccactatcaatagatattgattgacacagagggtttagcatgcagtcaaaatatcgcactggacactgaccatagaactttctgtcattttcctctgaaaaacggagatgtcgccgtgaagaatccaaaaatcgaatcgaatcagatcaaaccattccacattttcgtgcagagataatggataactcagctccggattctttgtccagatcggaaaccgccgtcacctacgattctccttacccactctacgccatggccttctcttccatcggtactccctccggccaccgaatcgccgtcggaagcttcctcgaggattacaacaaccgcatcgacattctctccttcgattccgactccatgacggtgaagcctctcccgaatctctccttcgatcatccttatcctcccacaaagctgatgttcagccctccctccctccgtcgtccctccgccggcgatctcctcgcttcctccggcgatttcctccgtttatgggagatcagtgaagattcctccaccgtcgagcctgtctcggtgctcaacaacagcaagacgagcgagttctgcgcgccgttgacctccttcgactggaacgacgtcgagccgaaacgcctgggaacctgcagcatcgacacgacctgcacgatttgggacatcgagaagtgcgtggtggagacgcagctcatagcgcacgataaggaggtccacgacatcgcttggggagaagctagggttttcgcgtcggtctccgccgacggatccgtcaggatcttcgatctacgcgacaaggagcattccaccatcatctacgagagtcctcagcccgatacgcctctcctaagactcgcctggaacaaacaagacctgaggtacatggcgacgatcctgatggattcgaataaggtcgtgattctcgacattcgctcgccgacgatgcctgtcgccgagctcgaacggcaccaggctagcgtcaacgccatagcttgggctccacagagctgcaaacacatctgctctgctggtgacgacacgcaggctctcatctgggagctcccgactgtagctggacccaacggcattgatccgatgtcggtttactcagccggctcggagataaaccagttgcagtggtcctcttcacagcctgattggattggcatcgctttcgctaacaaaatgcagctccttagagtttgaggttaggttttatatttaactatgaagtcgatgatatgaatgatatcacctggttaggcctaatgaattcgcaaagaaagttgaactgaaaactcacttgaatgctttcatggctggctattacagattgtcttgtagctataagagaactttgttagtaaatgatctcacagttgctgtaatggctgataaaatgatattcatgtagacgatgattgggtacaaaacacctttgtactttgtgcttcaggtttttgcaactcatatagagaatctagattgaacaataacattgcacatgtagaaaatcaaataacatttccttattatatacatctcgcttgatgtcagatgcagatggtgaagtggtcaaaatcagatttagcatagacccgatgtgacaagtaggttcggtgcacgcgcactctcgcgtcttgggtcagcaagcaatgcctctgtatctacctgattgtaactaaaactgatttcatttgatgaaccaaatgccactgttatctttctgttttgtgtataaaccccaaccagaatttattgcggtttctggtgtttagatttgaagtggaagcttattacagtataatgcaccctgtctttcctttgttgggtattcttcggataaaacagctacttgtcatcggttatttagcatctaaacaaagcaattataaaatatgtgttacaggttccaacttcgaacacctgtttttagtcagagttttttttttgtattttgcaaattaacttcaagtgacaagcaaattctcaatttattataccttcttcgtttgagaaaacctctgaacgtccgactgttatatttctctatctcattctttacaaagagataaatttatgttaaacttctctatttaccatttttaaaatttcatataaccaatttacataactttggagaacgttgatattcatattttccatttaataaaatgaataaaaatagaaaatttagcttaagggaaaggaaaatatgtgtcagattcttgatttcacaaaaccctacaagcaaccccctctacatataaatagcaacgtccttgtaacttaaaaggcaagtgataataatagtgcaaaaagatttgaaaaaaaaggttagagagaatgtccaccagcgagcttgcctgcacatatgccgctctaatcctccacgatgatgggattgacatcaccgttagttttctcttctctctaattctcaattttaacctctttttgatctagtttctactctttttctttgctcttcatgcatgtacttatatatgttgtagttgcgaatttcgtttatatagtttcatgtcggtaactttcttgtttattgtggatttactaggccgagaagatatcaaagctggtgaaagcagccaacgtgaatgttgaatcatactggcctagcctctttgctaagctatgtgcgaagaagaatattgacgatctcatcatgaatgttggagccagtggtggtggtgaggcagctccggttgcaaccaacgttcctccaactgctcaagcagctccagcagttgaggagacaaagaagaagaaggtatatcctattgtctttctaacagtcttagcgttattgcaatccaaatcatcaactatataatttaacaagcttatgattaaaactaaccaatgggaatattgttttaaaaattctcaggaggaggtgaaggaagagagtgaagatgaca

SEQ ID NO: 78 - DNA, 1275 bp, Thlaspi arvense
  source 1..1275: mol_type = genomic DNA
atggaggtgaaagagagtgaaagagtggtaatagccaaaccagtggcttcaaggccttcatgctctagcttcaggactttcactgagcttctgactgattcagctactgtgtctcctcaatcgaattgccacgagattgtagacgctgccattagaccaaagactctcaggtttaaccagccagctgcagctgcggtctcatgtccacgggctaaagaaaatggcaatggaaagtcttgtgatgacacagatggcggaaactacgtcgtttacaaacctaaagcaaagcttgtctccaaagcaaccgtctctgtgttggctaacatggggaatcgtcaacagacttggagacaacccgaagcagtagcgtatgggaagagtgtgagtcaaggtactggtcctaatctagtccagagagtttcatcctttacagaaacagagacatcggtcggggacagatcatctgtggacggatacaactggaggaaatacggacagaagcaagttaaaggaagtgagtgtccaagaagctattacaaatgcacacacccgaaatgtccggtgaagaagaaagtagagaggtcattgggaggtcaggtctcagagattgtgtatcaaggtgagcataatcactcgaagccgtcttgtccacttccacggcgcgcttcgtcatcatcctcttcagggtttcagaaaccaccaaaagggcttgtctctgaaggatcaatgggacaagaccctaatagtaatgccttttatcatcatcctctttggagcaatcaaagcaatgactcgtctaagatgtatgagggttgtgttgtaactccattcgagttcgctgttccaagatcggcgaattcaaccggcggaacttcggattccggttgtcgaagtagccagtgtgatgaaggcagcaatggaggagagcttgatgatccaagcagaagcaaaagaagcaggaagaacgagaagcaatcaagtgaagcaggagtatcgcaaggttcggtggaatcagacagtcttgaagatggatttaggtggagaaaatacggacagaaagttgttggaggcaatgcgtatccaagaagttattacagatgcacgagcgcgaattgcagagcaaggaaacacgtcgagagagcgagtgatgatccaagagcttttattacaacctacgaaggtaaacacaatcaccatttgctcttgagacctccaccttcgtctacggttcttcttccctttaactccacacaacattctaatcaagccatttga

SEQ ID NO: 79 - protein, 424 aa, Thlaspi arvense
MEVKESERVVIAKPVASRPSCSSFRTFTELLTDSATVSPQSNCHEIVDAAIRPKTLRFNQPAAAAVSCPRAKENGNGKSCDDTDGGNYVVYKPKAKLVSKATVSVLANMGNRQQTWRQPEAVAYGKSVSQGTGPNLVQRVSSFTETETSVGDRSSVDGYNWRKYGQKQVKGSECPRSYYKCTHPKCPVKKKVERSLGGQVSEIVYQGEHNHSKPSCPLPRRASSSSSSGFQKPPKGLVSEGSMGQDPNSNAFYHHPLWSNQSNDSSKMYEGCVVTPFEFAVPRSANSTGGTSDSGCRSSQCDEGSNGGELDDPSRSKRSRKNEKQSSEAGVSQGSVESDSLEDGFRWRKYGQKVVGGNAYPRSYYRCTSANCRARKHVERASDDPRAFITTYEGKHNHHLLLRPPPSSTVLLPFNSTQHSNQAI

SEQ ID NO: 80 - DNA, 5443 bp, Thlaspi arvense
  source 1..5443: mol_type = genomic DNA
ttgaatgtgatatatgatattgcggctggccgagctggctggtttaagtactcaaccagacagtaaagtaaaacagccgcatagattgaaatctcgtggaacgtataaccagctcctgtctgtcctagaattggcggttcgggatagaactgttccttacatctcttggaatctccggttgagttgatgctaattgtatcactacttgtgatgtgatctagaaacaaacatagcttatgttttgcgtttgcggtctctgagaaatcgttggggttctatgattcttctggcctgacaattaaaatcattttatggacccaattgaaagactaaaccggacgcagcctattgcaatttaaagaacaataacgagcatgtaggttagaaaccggtttgtagttccacttgagtcgaaactgtactacttgaaaaatttgggttttagacttaaactggtttcaagctccttttatctagggtggagagaacttgttaggaaaaaaaacaaattgttgtaatgcgcggaacggatcgacttatgctctataaaagtctgcggacgcagccaatattaaaggcatgttattataagaaaatgtcttgcggatgcaaaattaaggtaaaataaaacttctgttttaaactgtaactgtgtgtacattgattatgctcaaacaaaccaccttcttaacgaatctttaaggaggtgttattggttcttatattttaatggatttgagaatctaaaccaaattcagtattattggtttttgtattttaaaatttatattgaaatatagtgttattagttttatgattttaaaattcataatgaaatacagtgttattggttctattattttagaatctaaatattataaggattnaagaaaatgtcttgcggatgcaaaattaaggtaaaataaaacttctgttttaaactgtaactgtgtgtacattgattatgctcaaacaaaccaccttcttaacgaatctttaaggaggtgttattggtttttatattttaatggatttgagaatctaaaccaaattcagtattattattttaaaatttatattgaaatatagtgttattagttttatgattttaaaatccataatgaaatacagtgttattgtatctatcattttagaatctaaatattataaggatttgagttttaattgaatttgaatggatttggaaggattttcatgactaaaatataaagatccaaatacaaagtctaattttggtatttaaatagatttgattttttttttattttattgattttgaaatcaataaatttatcaaatccaaaaaagatttataaatctaccaaaatacaagaaccaataacccctagtaagtaatccagcaaactgtttgtataatgtcacctctaatagtagttgaaaattacaaactaataattgtttaactttcataaatgtttctgttaatggataattcataatattttttgtattgtgatactttattatattcatgcgaataataatattttagtttcggttcaatataaacttctgaaattgggacaacagcaacgtgataatattatatatcttcttatttctgacaatggagaggggtgaaatcgagaatgaaccggtttaaatttcggtacggttcagttcactttgtctaaacgagttggtatggttcacgcgtgctttacttctaacaagacaagctcgcgcgcgtgtgtcaacaaccaaccaaagtcactttggccgctggagcctcaaccaaaattcgaaacgacgccgtttctttggccgcgtttccaactaactttcaaaggcctataaaactcaactaacgttcaaccaaaactatctgtctcttctctgtctttcattttcatcgccttctttcatcactatagactagctcatctaaagtacggaccttgatccatccaacgtaccctaccttgacacgtgtcccttgacagtgtgtcggcaagactcaacgaactgaaccgatacgacggatacatcaacgcccaccttaatgaaaaacccgaattcggatcttcctaaagggttttgcctcaaactccttttttgaacttcagattctgcatcatcatctctctcaccgtgagtaacttacttatgtcctctcttttgcgtcacttatggttttacatttgtattcatcttttgggtttttgatttggaatgattcagaacctaatcacaactgtatcggcgcatgtgggtatcatcaagtttcttttttttttttttccggcatcatcaagtttctttctttttgctatagtttgcatcaaactttccaaggctctatatatagatcgagattatgtattgttggatttgatgcttctcaccgtccaaatccaatcttttgttcctctccttttaaaaccttctcgatacttgacttaagactctagaaaatgagctccgattcagaatctgaatctcttataaatttgattgaggctatgtgaagctaatgtgttgatttttaatctgtggaacatttttcaggttgaatcaaagctttttgactggaaacaattttgggtggtaaggttggaacaatggaggtgaaagagagtgaaagagtggtaatagccaaaccagtggcttcaaggccttcatgctctagcttcaggactttcactgagcttctgactgattcagctactgtgtctcctcaatcgaattgccacgagattgtagacgctgccattagaccaaagactctcaggtttaaccagccagctgcagctgcggtctcatgtccacgggtgattcatcttattaatctcttggcaatataatatatcttcttatttctgaatcttctgcattttagcactgactcgtataagtttttctcataatcttaggctaaagaaaatggcaatggaaagtcttgtgatgacacagatggcggaaactacgtcgtttacaaacctaaagcaaagcttgtctccaaagcaaccgtctctgtgttggctaacatggtaagttgtttctagtgctacaaagatcaagaatttctcggaattagtttcttaactctgtttttgttttagcttcaggggaatcgtcaacagacttggagacaacccgaagcagtagcgtatgggaagagtgtgagtcaaggtactggtcctaatctagtccagagagtttcatcctttacagaaacagagacatcggtcggggacagatcatctgtggacggatacaactggaggaaatacggacagaagcaagttaaaggaagtgagtgtccaagaagctattacaaatgcacacacccgaaatgtccggtgaagaagaaagtagagaggtcattgggaggtcaggtctcagagattgtgtatcaaggtgagcataatcactcgaagccgtcttgtccacttccacggcgcgcttcgtcatcatcctcttcagggtttcagaaaccaccaaaagggcttgtctctgaaggatcaatgggacaagaccctaatagtaatgccttttatcatcatcctctttggagcaatcaaagcaatgactcgtctaagatgtatgagggttgtgttgtaactccattcgagttcgctgttccaagatcggcgaattcaaccggcggaacttcggattccggttgtcgaagtagccagtgtgatgaaggcagcaatggaggagagcttgatgatccaagcagaagcaaaagaaggtataatattagagagctttgaatagtttcaaaacaccaatcttcttcgtttactgttacattgacttttatgaataataatatacagcaggaagaacgagaagcaatcaagtgaagcaggagtatcgcaaggttcggtggaatcagacagtcttgaagatggatttaggtggagaaaatacggacagaaagttgttggaggcaatgcgtatccaagaagttattacagatgcacgagcgcgaattgcagagcaaggaaacacgtcgagagagcgagtgatgatccaagagcttttattacaacctacgaaggtaaacacaatcaccatttgctcttgagacctccaccttcgtctacggttcttcttccctttaactccacacaacattctaatcaagccatttgatgaatgatggtatattgatacattacttcttcaatcttcatagcaacattagttcatcattttcttgttgttgcactgtgaatttatttattttacatcaaaattgtaaggagaagcaaaatagttgttatttaacaaacgaaaaaaaaagcaaataaaaagagtgttatattttaggtttaacaacttatgttcccatgttgtagctttcttctgttagtatattgggtttgatctacagtagtggtaaaaagaaactacttttactaatcacaaaaacagtaaatgtaaaacgcaacacacacttaccgcttaataaatcatttgaagcttaaccaccaattagttgaaatacaaacatgaaaggtttggttacaattgatttcatggcaataaacaatttttatttaggaaaatagcagaaaatcggaatatttaatttggagttaagaaccattgaagatggaaaccatatcactttaatgtatcagctttctaattagaaatataaagatgaaatattcttaaatatgaaaccgtttgatcaaagatcccaagtcgataaagcgtcaaaacttgattaaaggtagagaaattttctgaaacatatccaaagctaacgaaaacagcacaaaaaaaatgtctttggagatataatgcaaactcagggcaaaggaagatcccttaacgattggccttggcaacacgctcaatctcatctttcttcttgatagcatagctgcaaagacaagacaaagatgcatctcatcagctactaaagtaacacaaatgtgaagccatttaatagacaagacgatgtagagaatttgttttataaaacctgttggaagatcccttggcagcattgatgagttcatcagcaaggcactcagcgattgtcttgacgtttctaaacgcagcctcacgtgcaccagtggtgatcaagaagattgcttggttcacacgtcttagaggagagatatcaacggcctgcctcctaaccacaccagcagatccaatcctggtagcatcttctctcggaccactgtctcaccaaaaataaacgaacatcatgagaaaagaggttcataacaacacaacaagaaagtgagagggtttcagtatcaccacatgtttgatttacttacctgttaacaatggcatcaatgataacctgaatcgggttctggtcagacaagagatggatgatctccatggcgtgcttgatgatcctgacggccatcagcttcttaccgttgttccttccgtgcatcatgagagagttcgtgagcctctc

SEQ ID NO: 81 - DNA, 404 bp, Thlaspi arvense
  source 1..404: mol_type = genomic DNA
caaacacacgctcggacgcatattacacatgttcatacacttaatactcgctgttttgaattgatgttttaggaatatatatgtagctatctgatcggtcagtattttcacaggtcgtgatatgattcaattagcttccgactcattcatccaaataccgagtcgccaaaattcaaactagactcgttaaatgaatgaatgatgcggtagacaaattggatcattgattctctttgataatactcaccgatcagacagctctcttttgtattccaattttcttgattaatctttcctgcacaaaaacatgcttgatccactaagtgacatatatgctgccttcgtatatatagttctggtaaaattaacattttgggtttatctttatttaaggcatcgccatg

SEQ ID NO: 82 - DNA, 404 bp, Thlaspi arvense
  source 1..404: mol_type = genomic DNA
caaacacacgctcggacgcatattacacatgttcatacacttaatactcgctgttttgaattgatgttttaggaatatatatgtaggaacgtattacctacacattttcacaggtcgtgatatgattcaattagcttccgactcattcatccaaataccgagtcgccaaaattcaaactagactcgttaaatgaatgaatgatgcggtagacaaattggatcattgattctctttgataatgtgaaggtaatacgctcctctcttttgtattccaattttcttgattaatctttcctgcacaaaaacatgcttgatccactaagtgacatatatgctgccttcgtatatatagttctggtaaaattaacattttgggtttatctttatttaaggcatcgccatg

SEQ ID NO: 83 - DNA, 404 bp, Thlaspi arvense
  source 1..404: mol_type = genomic DNA
caaacacacgctcggacgcatattacacatgttcatacacttaatactcgctgttttgaattgatgttttaggaatatatatgtagtcagctccgagttatcaattttcacaggtcgtgatatgattcaattagcttccgactcattcatccaaataccgagtcgccaaaattcaaactagactcgttaaatgaatgaatgatgcggtagacaaattggatcattgattctctttgataattgaaaactcggagccgactctcttttgtattccaattttcttgattaatctttcctgcacaaaaacatgcttgatccactaagtgacatatatgctgccttcgtatatatagttctggtaaaattaacattttgggtttatctttatttaaggcatcgccatg

SEQ ID NO: 84 - DNA, 404 bp, Thlaspi arvense
  source 1..404: mol_type = genomic DNA
caaacacacgctcggacgcatattacacatgttcatacacttaatactcgctgttttgaattgatgttttaggaatatatatgtaggaacttgacagtataatcttttcacaggtcgtgatatgattcaattagcttccgactcattcatccaaataccgagtcgccaaaattcaaactagactcgttaaatgaatgaatgatgcggtagacaaattggatcattgattctctttgataagattttactgtcaagctcctctcttttgtattccaattttcttgattaatctttcctgcacaaaaacatgcttgatccactaagtgacatatatgctgccttcgtatatatagttctggtaaaattaacattttgggtttatctttatttaaggcatcgccatg

SEQ ID NO: 85 - DNA, 1998 bp, Thlaspi arvense
  source 1..1998: mol_type = genomic DNA
ctataaaatataatatttagcaccttgacaggattgtcagttattatattgttagagtgataaagatcatcatcacgtacatataaagagattataagataaaaataattttcggtactgtaggttttctgcaatttaaattatttaaaatatgtaattgagaaaatattcggtcgcctggtttatatagtcaagtgttacgaaaacatgtttgttttgtatgcaatttttgttttacttatttgagacataaatgagttattttataatgggctgttacagatatgtttaattaatataaattattggtttttaacttgttgattttttatttttatttttcaagctttagaaactgatgccacgtggcattgtgggagagagttttttttgcttaggtggatagcctaagaagccccaaataatcctttttatttagtatagattagtcacggcttccacgaaacaagttcaattaaatttaacggaaataaatggtaattaatgaaagtttgaattgtattaattactgcttccacgaaacaaccaaaaaaatcatttgacaaagtaatttgtgtacggtattagccacatggggccaatcctctatgtatatcggagttgttttgccaaatgcccaattcttactctagtaaatattaaaccattttgtagatcctaatcttgagtcaactcaaagtcctatgtttggaaactaaaaataagtttaaatcctctagactacgactaagttgtaaaaagaaatgttcaaaattccttagacattgaaattgaatacttcaattaaagaaaattttaccagcatgttcatagtagtagaccaccacagaacaaaattaatagtttttacattgcattcttatataataattttggagtataattgtaattataaacaaaataactaaaagaaaaggaaagtattcttgacatatttataggtactaatttgctagtacgactattaattatggagattaaatctagtatttgactaaccagaaacattttaaaggtaagtgataagttgataacctcgcaattatttttgaaagctattaatcactttagcagaatgtatacaagttctatattaacaagtttatctcaaaatcttgagatcctactaataaatcataacttttctcttattggtatgtcgaatctagtgtttaccaaactagaggtgttgaccgttagagacaattaaacaacttacatacatacaaaagtacaagccgaagaataatagtaataacagtgcattttctacaataattaaaacaaaaaagtatattaaaaattagcatttattccaattaataccaatattcgaaaattaatatggtagaaaagcaaaaagcttacgtaaattccacagattgtcaaaaccctgcaatgaaaaagttgccaaaaattgacgaggttggtccacaaaatttaaggttctttgcttcacactctatttataggcgaagagatgaaacagaggaaattaattactccttaacaaaggttgttttcactcaaccacatgccttctcaagtgtctgctgctcacattccccgagattctcatttacttactcctctatttggtacgtcctttatattacaattctagtattttttttttcattattcgttttgttcatacttcgtttcataaacatatatagttaatcttatattatacagtaactttgataaatatgtatcttaatttactcacgcagtttagagagacgattgatacgtggggactacttacgtacctgcatgattatataagttataaaagttattgcagaacattaaattactttgatagctcgagagcaatcattatataaagctatatttttaatcacaaatatacatctactcgaaagttttttttttaaaaaaaagatgaaatccctaaacaaacgttcccaaatgttctcatcactctccaaccacgattcaccacattca

SEQ ID NO: 86 - DNA, 1978 bp, Thlaspi arvense
  source 1..1978: mol_type = genomic DNA
agtaactgagaggaatgtaactgagagaaaaaagaaaagtaataattaaaataattgaaaataataagtaagagcatctagatagtccttaccaaaaaaaagatcatctagatagtctttccatagtatctgatttatataatactaatataatgtatttatataattaaattataaattaataaagagatgaaccaattcttaacagacatttgcctagagacatatctttattcttaacaaacatttggctagaaacatatctaatagtttcttgtttcaaaaatgtcttcgctttttttctgtttttctcccttcctttttccttattttttttatttttcttattcactaaatatactcccataatatctgctatataatattaatataatatatttatataattaaattaaaaattcataaagagatgaaccaattcttaatagatatttggctaaatacatatttaatatttttttgtttgaaagatatctttgctattttttttctgtttctttctcttcttttcttcttatatttcttattttttttattcaataaatatattttaaaaaacaccaattttcttataagaatatctgaaaataattgagaataattaaattaaatattttcttttagtaaaacttataattatatatatgctaaaaagatatgtgatatttattattctatctttaaagatttttttatcgtgaatcactattttcagttatccaacctaaaatgaatactcagagagcaaccacatcgaaagtgttagagattgaatgaatacagttcttaaaaggtttatttcacattaatgaagtctatcaaaacaaaatttatctaattacatctctataacaaaaatgataattctaaagttaaatgaaaaaaaaaaacaaaaaaatagagagaatttttctttaaaaaaaagataagaaagatcaataaaaccgacttcctcacattataaatagaaaagtcttcataatttgaagattcaatccgaaaatccctaaaattctagaaagtaagaccaaatgttcaaagttttctagtttaattttttcaagtgttcttacacacagtttagcaaaacgtgattgttaagctatctttgtaatcactctgagcttttgagtaataatgagatttaattttttaaacacacaaccttatattgctttttttctttgagtgatatcttggttaacaattgacgtcgtctgtagaaaataaagaataaattcttaagcaaaacaaggtttagagatgagctctccgaataagagcaatcaacacactcaaatccctacctttgatgagcacagcaacccccattgtcattggaaatcaaacaccacctccaccactagccacatgaacattgacccttgtctctaccggtgatcttgctgctgcaattcaacccgccctaaaaatcgatcacctaagaccttagttccaaattcgataacttgcaagctcagcaagcaagggatcaggaacaactcaacctcatggcaacctcggccactaaacaagttgcaccacttggacgaagacttgtctttgagacacccaccgtccacatcgtagcaagagccacaacagacacccaagcaaacaagacgaatgtcccagccctaactaatgaagcgttacatgccaaggtgggttatgaagtctcacctgccaaggtgggttagacgaatgtctctctgttagacacctccacttgaccacttgtttgaggatggtaaggagtagcaaccttatgcttaacaccatgcttccttagcagattctcaaaaactttgttgatgaagtggcttcctccatcacttataacacaccttggaacaccaaaccttgggaagatgattatcttgaaaagtttcagcacaaccctagcatcatttgttggactggctatggcttcaacccactttgagacatagtctac

SEQ ID NO: 87 - DNA, 1424 bp, Thlaspi arvense
  source 1..1424: mol_type = genomic DNA
tgctttgggagttatgatggaaaacctaattctctaactctgtttatatagggaagactcaggaaaaatcctaatatccttttctcttgggcttctgacacatgaacctttccttttcctattgagcctagatccagaacgttacaactagaatcagaatcgaacaacaagcgtatgcgcttaagaaatgatttttactcacactatatgggcgcgcatggcattgtcgtaagaattaaataagcttcgtcccactccttattggatttacgcacacagtcaatccacgtggttgtcataactaaatataattggtgaaaaaactaatttaaagacaaagtaagaagcggaaaccgaaggaaaagccaaacaacattatgtttatcgacaaaaaagccaaaacaacatttaatgttaatttttgtccaaaccaggaatacatcactcatacggtcatatataccataccccaaattgtatgtactacatgtgtacgtaactgatttatgtatatctatctatttctcttttattgtaggggtcaaaatctacctatcgatatttataaataggattttataagacattgttaaaatggaaaacatggtagctacttggccagcgattaacatcaataattaaaaatacgaattgtgaaacatctcactctcttccttttatcacaccaaaccacttctctttctttacttaccggtcaggtcaacaattctaccattccctttatttagtttttttttttcatctatctctcttttcatttttaacttttaaaaaatattagtccgaaaatcgcactcttttatattcgttgatgggcattaaaatattgtaaaaattaaaaaaaatgtgtaaattaaaacgcaatttgtttgcacctgcagcagtagtaacacacaagtcaaagcaatgtctgttttctcttgtatgtcggttagctattttatgtactgaaatatgttattctttctgaccaaaacatattaagaaatttgcattctgtaatttgatttatccaatcagtagaggacacgtaaccaagataattagatatttttattctatctgtatactactaattcatccaacgtctgttggacgaccaaccagtcaaaactcataaaagtcaacttttactacatcgtgaattttttggtcatgcatttatatacacatacatggaagaaaaaacaatactttacctatgtcaaaatgctgcaaaagcattaagatggtatgtatacatacatattgatataacagtggtcccatatatcattttaaatcatcataaagagtatccatcttctttcatattaacatccccttcaaagttataagatttttctctcttcattagagagagagagagagagaattaacacaagttttaatctccgggaaag

SEQ ID NO: 88 - DNA, 1982 bp, Thlaspi arvense
  source 1..1982: mol_type = genomic DNA
tttaagactctaacaatattgatgccaactctagaaaaggacaactgaacttattttgttttaagccttggctatattacattatcagttttacacttctacgtatacacaactttaatttttctatattactacatgcatactagatgttagaaatatcttatataagcatacatattaaattagttatgcgatatttaaaattaatgtatataaatattcagtttcaaagcttacgaatacgtaaaattaatcaatacaaaccgaaaccaaaaagccacttctcatctcaaaatgttaattacaaatttacaataagccgaatcactatctaaaaagcttttgggatcccgagacccaaaacatttcttcgtataagtgcaacaattatcgagtgtgatgagacacgtaaaaagcaaggatcgggacaacgattttcggctatatagtagtttctcatatctgactttattagcaaattactacataaaattgcttttaagtatgattaacggaacaagtataatgcgtttatttaattacagatctggctaaatgtaatatggtatcaaagctggaaagacaaatagtctaaggtttggaatttttcatctgtctataaaattcttaagacactgatagatcattaacttattttggagtcaccaatggacaaatcaataactattgatgtccaaaacccacaagacaagcctattttttttgcacgtgcaagtacaccggtggaagaattcatttgtatactgaatctgaaattataaaagattagaattggttgaagaatttacttcaaaacaagctagaagatggtgttatttaaataaacaatgttaaattatatcaatgactttcaaattttatcaaaacattttttgaaagagaatcacgcacaaagtatttcgaattacaaaacaattttttgcaataaattttatagttaaagcaaattttgtcaattattgaaaaaaattacttagataaaatatctaatttttttttttaaacaatgtctctataaaagatggattagccaagaaaatctgatggatgctacaacgcagggttggagatatggtgctgacactacatactattaattggttgatagaatcggtaaatatagatatgaatatcgaaatggagacaacaggaaactcaaccgtatggaaggaataaaatagaggaactttaaagagaacttgaggatatacaaacagatgactcacgtatgcaagagaagcttaatgagataacaaataactaaaggatgcgtatcgggatgagaaacaattctggcaacagaagagcagaaacttatggaacaaatgtggagattttaatacataattttataatacttcaacaaagcaacggaggactataaacagaatagtgggactacatgatagtcagggtgtatggataacttaggttgcatggaaactccctccgaggtacgtttctcgcttccgaaacgtttcggaaaccgaaactctcggaagctcgtcggaaacacaaaaatcacgtttcctaaaaattctaatttgtgaatactttggaaacacgtttccattttaaaaacacatgtttccattttggaaagaagataaaaactaattttttttgttttatgaaataaatagataattataaaaattagatttaaatgatcaatttaattatttatagtaaataataattgatttggtaactaaaattaattttgatattattaagtttgaaaattattctttccatgaattagaattagagatagtttggttaatgaaactaataaaattgataataaagaaaatcttctaaatttttgactttacatattttaatttttatagtttaataattatgtaaaaattatatattagattttgttgatttaaatattctacttataacataattagatctttaataattaatatatatatatacacatttcc

SEQ ID NO: 89 - DNA, 24 bp, Thlaspi arvense
  source 1..24: mol_type = genomic DNA
attgtatatcatgatccatggcga

SEQ ID NO: 90 - DNA, 24 bp, Thlaspi arvense
  source 1..24: mol_type = genomic DNA
aaactcgccatggatcatgatata

SEQ ID NO: 91 - DNA, 24 bp, Thlaspi arvense
  source 1..24: mol_type = genomic DNA
attgatctgatggatttggaaggt

SEQ ID NO: 92 - DNA, 24 bp, Thlaspi arvense
  source 1..24: mol_type = genomic DNA
aaacaccttccaaatccatcagat

SEQ ID NO: 93 - DNA, 24 bp, Thlaspi arvense
  source 1..24: mol_type = genomic DNA
attgttcgtgctgatgaatcttct

SEQ ID NO: 94 - DNA, 24 bp, Thlaspi arvense
  source 1..24: mol_type = genomic DNA
aaacagaagattcatcagcacgaa

SEQ ID NO: 95 - DNA, 24 bp, Thlaspi arvense
  source 1..24: mol_type = genomic DNA
attgacaacggtgcaataaagacg

SEQ ID NO: 96 - DNA, 24 bp, Thlaspi arvense
  source 1..24: mol_type = genomic DNA
aaaccgtctttattgcaccgttgt

SEQ ID NO: 97 - DNA, 24 bp, Thlaspi arvense
  source 1..24: mol_type = genomic DNA
attggcatgccctagcttccgatg

SEQ ID NO: 98 - DNA, 24 bp, Thlaspi arvense
  source 1..24: mol_type = genomic DNA
aaaccatcggaagctagggcatgc

SEQ ID NO: 99 - DNA, 24 bp, Thlaspi arvense
  source 1..24: mol_type = genomic DNA
attgatgcacggcattatcgccgg

SEQ ID NO: 100 - DNA, 24 bp, Thlaspi arvense
  source 1..24: mol_type = genomic DNA
aaacccggcgataatgccgtgcat

SEQ ID NO: 101 - DNA, 24 bp, Thlaspi arvense
  source 1..24: mol_type = genomic DNA
attgagtttcctggaccagtgtta

SEQ ID NO: 102 - DNA, 24 bp, Thlaspi arvense
  source 1..24: mol_type = genomic DNA
aaactaacactggtccaggaaact

SEQ ID NO: 103 - DNA, 24 bp, Thlaspi arvense
  source 1..24: mol_type = genomic DNA
attgccatcgaagacgcgactgtt

SEQ ID NO: 104 - DNA, 24 bp, Thlaspi arvense
  source 1..24: mol_type = genomic DNA
aaacaacagtcgcgtcttcgatgg

SEQ ID NO: 105 - DNA, 24 bp, Thlaspi arvense
  source 1..24: mol_type = genomic DNA
attggttctctgcgatgctcacat

SEQ ID NO: 106 - DNA, 24 bp, Thlaspi arvense
  source 1..24: mol_type = genomic DNA
aaacatgtgagcatcgcagagaac

SEQ ID NO: 107 - DNA, 24 bp, Thlaspi arvense
  source 1..24: mol_type = genomic DNA
attggaccgatacttgaagaccag

SEQ ID NO: 108 - DNA, 24 bp, Thlaspi arvense
  source 1..24: mol_type = genomic DNA
aaacctggtcttcaagtatcggtc

SEQ ID NO: 109 - DNA, 20 bp, Thlaspi arvense
  source 1..20: mol_type = genomic DNA
cggaagaggctgcgttagag

SEQ ID NO: 110 - DNA, 20 bp, Thlaspi arvense
  source 1..20: mol_type = genomic DNA
gcagctaagggaactttacg

SEQ ID NO: 111 - DNA, 20 bp, Thlaspi arvense
  source 1..20: mol_type = genomic DNA
cggaagaggctgcgttagag

SEQ ID NO: 112 - DNA, 20 bp, Thlaspi arvense
  source 1..20: mol_type = genomic DNA
caggaaaggcgtatgcgagg

SEQ ID NO: 113 - DNA, 20 bp, Thlaspi arvense
  source 1..20: mol_type = genomic DNA
cgccgtcggaagcttcctcg

SEQ ID NO: 114 - DNA, 20 bp, Thlaspi arvense
  source 1..20: mol_type = genomic DNA
caaacaagacctgaggtaca

SEQ ID NO: 115 - DNA, 20 bp, Thlaspi arvense
  source 1..20: mol_type = genomic DNA
agcattccaccatcatctac

SEQ ID NO: 116 - DNA, 20 bp, Thlaspi arvense
  source 1..20: mol_type = genomic DNA
tcgactggaacgacgtcgag

SEQ ID NO: 117 - DNA, 1188 bp, Thlaspi arvense
  source 1..1188: mol_type = genomic DNA
atggtgatgggtacacaaccgtcgttggaagagatcagaaaggcacagagagcggatggccccgcaggcatcttggggataggcacggccaaccctgcgaaccatgtgatccaggcagagtatccggactactacttccgcatcaccaacagtgagcacatgactgacctcaaggagaagttcaagcgcatgtgcgacaagtcgatgatacggaaacggcacatgcacctgacggaggagttcctgaaggagaatccggacatgtgcgcctacatggctccttctcttgatgtgaggcaggacatcgtggtggtcgaggtccctaagctagggaaagaggcggcagtgaaggccatcaaggagtggggtcagcccaagtccaagatcacccacgtcgtcttctgcactacatccggagttgacatgcctggtgctgactaccagctcaccaagctcctcggtcttcgcccttccgtcaagcgtctcatgatgtaccagcaaggttgctacgccggcggcactgtcctccgactcgccaaggacctcgctgagaataaccgtggtgctcgtgtccttgtcgtctgctccaagatcacagccgtcaccttccgtggcccctctgacacacacctcgactccctcgttggtcaggctctcttcagtgacggtgctgccgcgctcattgttggtgcggaccctgatgcctccgtgggagagaagcctatcttcgagatggtgtctgctgcacagaccatcctcccagactcggacggagccatagatggacacttgagggaagttgggctcaccttccatctcctcaaggacgtccctgggctcatctcgaagaacatagagaagagtctagaagaagcgtttaaaccgctcgggataagtgactggaactctctcttttggatagctcaccctggaggtcctgcgatcctggaccaggttgagttaaagctaggactcaaggaagagaagatgagggccacgcgtcacgtgctgagcgagtacggaaacatgtcgagcgcgtgcgttctcttcattatggacgagatgaggaggaagtccaaggaggatggtgtggccacgacaggagaagggttggagtggggtgtcttgtttggtttcggaccaggtctcaccgtagagacagtcgtcttgcacagcgtccctgtttga

SEQ ID NO: 118 - protein, 395 aa, Thlaspi arvense
MVMGTQPSLEEIRKAQRADGPAGILGIGTANPANHVIQAEYPDYYFRITNSEHMTDLKEKFKRMCDKSMIRKRHMHLTEEFLKENPDMCAYMAPSLDVRQDIVVVEVPKLGKEAAVKAIKEWGQPKSKITHVVFCTTSGVDMPGADYQLTKLLGLRPSVKRLMMYQQGCYAGGTVLRLAKDLAENNRGARVLVVCSKITAVTFRGPSDTHLDSLVGQALFSDGAAALIVGADPDASVGEKPIFEMVSAAQTILPDSDGAIDGHLREVGLTFHLLKDVPGLISKNIEKSLEEAFKPLGISDWNSLFWIAHPGGPAILDQVELKLGLKEEKMRATRHVLSEYGNMSSACVLFIMDEMRRKSKEDGVATTGEGLEWGVLFGFGPGLTVETVVLHSVPV

SEQ ID NO: 119 - DNA, 1188 bp, Thlaspi arvense
  source 1..1188: mol_type = genomic DNA
atggtgatgggtacacaaccgtcgttggaagagatcagaaaggcacagagagcggatggccccgcaggcatcttggggataggcacggccaaccctgcgaaccatgtgatccaggcagagtatccggactactacttccgcatcaccaacagtgagcacatgactgacctcaaggagaagttcaagcgcatgtgcgacaagtcgatgatacggaaacggcacatgcacctgacggaggagttcctgaaggagaatccggacatgtgcgcctacatggctccttctcttgatgtgaggcaggacatcgtggtggtcgaggtccctaagctagggaaagaggcggcagtgaaggccatcaaggagtggggtcagcccaagtccaagatcacccacgtcgtcttctgcactacatccggagttgacatgcctggtgctgactaccagctcaccaagctcctcggtcttcgcccttccgtcaagcgtctcatgatgtaccagcaaggttgctacgccggcggcactgtcctccgactcgccaaggacctcgctgagaataaccgtggtgctcgtgtccttgtcgtctgctccgagatcacagccgtcaccttccgtggcccctctgacacacacctcgactccctcgttggtcaggctctcttcagtgacggtgctgccgcgctcattgttggtgcggaccctgatgcctccgtgggagagaagcctatcttcgagatggtgtctgctgcacagaccatcctcccagactcggacggagccatagatggacacttgagggaagttgggctcaccttccatctcctcaaggacgtccctgggctcatctcgaagaacatagagaagagtctagaagaagcgtttaaaccgctcgggataagtgactgaaactctctcttttggatagctcaccctggaggtcctgcgatcctggaccaggttgagttaaagctaggactcaaggaagagaagatgagggccacgcgtcacgtgctgagcgagtacggaaacatgtcgagcgcgtgcgttctcttcattatggacgagatgaggaggaagtccaaggaggatggtgtggccacgacaggagaagggttggagtggggtgtcttgtttggtttcggaccaggtctcaccgtagagacagtcgtcttgcacagcgtccctgtttga

SEQ ID NO: 120 - protein, 300 aa, Thlaspi arvense
MVMGTQPSLEEIRKAQRADGPAGILGIGTANPANHVIQAEYPDYYFRITNSEHMTDLKEKFKRMCDKSMIRKRHMHLTEEFLKENPDMCAYMAPSLDVRQDIVVVEVPKLGKEAAVKAIKEWGQPKSKITHVVFCTTSGVDMPGADYQLTKLLGLRPSVKRLMMYQQGCYAGGTVLRLAKDLAENNRGARVLVVCSEITAVTFRGPSDTHLDSLVGQALFSDGAAALIVGADPDASVGEKPIFEMVSAAQTILPDSDGAIDGHLREVGLTFHLLKDVPGLISKNIEKSLEEAFKPLGISD

SEQ ID NO: 121 - DNA, 1239 bp, Thlaspi arvense
  source 1..1239: mol_type = genomic DNA
atggctccagggactctcaccgagctcgccggagaggctaagctcaactctaaattcgtccgggacgaggacgaacgtcccaaggtggcatacaacaagtttagcgacgatatcccggtgatatctctcgccggactcgacgatgttggtgggaaaagaggagagatctgccgtaagatcgttgaggcttgcgagaattggggcgtgttccaggtggtcgatcatggtgtcgataccaatttggtagaggatatgactcgcctcgctcgcgacttctttgctttaccacccgaagagaaacttagtttcgacatgtctggtggtaagaaaggcggcttcatcgtctctagtcaccttcagggagagactgtgtaagattggagagagatcgtgacgtacttctcgtacccggtgagaaacagagactactcacggtggccagataagccggaagggtgggtgaaagtgacggaggagtacagcgacaaactgatgggtttagcttgtaagcttcttgaggttttgtctgaagctatggggctcgagaaagaagcacttaccaatgcttgcgtcgatatggaccaaaagatagttgttaattattaccctaaatgccctcagcctgatctcaccctcggactcaagcgtcacactgatcctggaaccatcactttgctgctccaagaccaggtcggtggattacaagccacacgcgacgatggcaaaacatggataacggttcagccaattgagggagcttttgtcgtgaatctcggcgaccatggtcactatttgagcaacgggaggttcaagaacgcggatcatcaggcggtggtgaattccaactcgagcaggctatctatagccacgtttcagaatccggcgcaggatgcaaccgtgtatccgcttaaagttagagaaggagagaagccgatcttggaggagccaatcacttttgcagagatgtataagagaaagatgggaaaagatctggagctggctcgcctcaagaagcttgcgaaagaagaaaatgaccagaagctggccaaagaagaacatgacaagaacctggccaaagaagaaaatgatcagaagctggctaaagaagaacatgaccagaagctggccaaagaagaacatgacaagagccttgccaaagaagaaaattaccaaaagctggccaaagatgaacatagccacacggaagctgttaagcgtctcggccaaatcctcgcttag

SEQ ID NO: 122 - protein, 124 aa, Thlaspi arvense
MAPGTLTELAGEAKLNSKFVRDEDERPKVAYNKFSDDIPVISLAGLDDVGGKRGEICRKIVEACENWGVFQVVDHGVDTNLVEDMTRLARDFFALPPEEKLSFDMSGGKKGGFIVSSHLQGETV

SEQ ID NO: 123 - DNA, 1239 bp, Thlaspi arvense
  source 1..1239: mol_type = genomic DNA
atggctccagggactctcaccgagctcgccggagaggctaagctcaactctaaattcgtccgggacgaggacgaacgtcccaaggtggcatacaacaagtttagcgacgatatcccggtgatatctctcgccggactcgacgatgttggtgggaaaagaggagagatctgccgtaagatcgttgaggcttgcgagaattggggcgtgttccaggtggtcgatcatggtgtcgataccaatttggtagaggatatgactcgcctcgctcgcgacttctttgctttaccacccgaagagaaacttagtttcgacatgtctggtggtaagaaaggcggcttcatcgtctctagtcaccttcagggagagactgtgcaagattggagagagatcgtgacgtacttctcgtacccggtgagaaacagagactactcacggtggccagataagccggaagggtgggtgaaagtgacggaggagtacagcgacaaactgatgggtttagcttgtaagcttcttgaggttttgtctgaagctatggggctcgagaaagaagcacttaccaatgcttgcgtcgatatggaccaaaagatagttgttaattattaccctaaatgccctcagcctgatctcaccctcggactcaagcgtcacactgatcctggaaccatcactttgctgctccaagaccaggtcggtggattacaagccacacgcgacgatggcaaaacatggataacggttcagccaattgagggagcttttgtcgtgaatctcggcgaccatggtcactatttgagcaacgggaggttcaagaacgcggatcatcaggcggtggtgaattccaactcgagcaggctatctatagccacgtttcagaatctggcgcaggatgcaaccgtgtatccgcttaaagttagagaaggagagaagccgatcttggaggagccaatcacttttgcagagatgtataagagaaagatgggaaaagatctggagctggctcgcctcaagaagcttgcgaaagaagaaaatgaccagaagctggccaaagaagaacatgacaagaacctggccaaagaagaaaatgatcagaagctggctaaagaagaacatgaccagaagctggccaaagaagaacatgacaagagccttgccaaagaagaaaattaccaaaagctggccaaagatgaacatagccacacggaagctgttaagcgtctcggccaaatcctcgcttag

SEQ ID NO: 124 - protein, 412 aa, Thlaspi arvense
MAPGTLTELAGEAKLNSKFVRDEDERPKVAYNKFSDDIPVISLAGLDDVGGKRGEICRKIVEACENWGVFQVVDHGVDTNLVEDMTRLARDFFALPPEEKLSFDMSGGKKGGFIVSSHLQGETVQDWREIVTYFSYPVRNRDYSRWPDKPEGWVKVTEEYSDKLMGLACKLLEVLSEAMGLEKEALTNACVDMDQKIVVNYYPKCPQPDLTLGLKRHTDPGTITLLLQDQVGGLQATRDDGKTWITVQPIEGAFVVNLGDHGHYLSNGRFKNADHQAVVNSNSSRLSIATFQNLAQDATVYPLKVREGEKPILEEPITFAEMYKRKMGKDLELARLKKLAKEENDQKLAKEEHDKNLAKEENDQKLAKEEHDQKLAKEEHDKSLAKEENYQKLAKDEHSHTEAVKRLGQILA

SEQ ID NO: 125 - DNA, 1545 bp, Thlaspi arvense
  source 1..1545: mol_type = genomic DNA
atggccactctcttactcacaatcctcctccccactttcctcttcctcctcgtcctcctcctttctctccgccgcaaccacaaccgcagtagccgtctcccaccaggcccaaaaccatggcccatcctcggaaacctccctcacatgggtcctaaaccccatcgaaccctagccgccatggtaaccacctacggtccaatcctccacctccgattagggttctccaacgtcgtggttgctgcgtctaaatccgtggccgaacagttcttcaaaatccatgatgccaatttcgctagccgaccaccaaattcaagagccaaacacatggcatataactatcaagatcttgtctttgcgccttacggacaacgatggagaatgttgaggaagattagttctgttcatttattttcagctaaagctcttgaagattacaagcatgttcggcaggaagaggtaggaacgctcacgcgcgagctagtggatgcaggcacgaaacccgtaaacttaggccagttggtgaacatgtgtgtggtcaacgcgcttggaagagagatgatcggacggcgtctgttcggcgccggagctgatcacaaagcggaggagtttcgatcgatggtgacggaaatgatggctctcgccggagtattcaacctcggagatttcgtgccggctctggattggttagatttacaaggcgttgctggtaaaatgaaacggctacacaaaagattcgacacttttctatcgtcgattttgaaggagcacgagatgatgcacggtcaagatcaaaagcataaagatatgctcagcactttaatctcgctcaagggaactgattttgacggtgacggcggaagcctaacggatactgagatcaaagccttgctcttgaacatgtttacggctggaactgacacgtcagcaagtacggtggactgggccatagctgaactgatacgacacccggatgcaatgaccagagcccaagaagaacttgattcagttgtaggccgcgataggcccattaacgagtcagacctttctcggcttccttatcttcaggcggttatcaaagagaatttcaggcttcatccgccgacaccactctcgttaccacacatcgcatcagagagctgtgagatcaacggctaccatatcccgaaaggatcgactcttttaacaaacatatgggccatagcccgtgacccggaacaatggtccgacccgttatcgtttcgacccgagagatttttacagggtggagaaaaatccggcgtcgatgtgaaaggaagcgatttcgagcttataccgttcggagccgggaggagaatctgcgctgggctcagtttagggctacggatgattcagttactgacggcgacgctggttcacggatttgattgggaattggccggaggaattccgccggagaagctgaatatggaggagacttatgggattactctgcaaagagcagttcctttggtggtgcatcctaagccaaggttggctcccagtgtttacgaactcgggtcgcgctaa

SEQ ID NO: 126 - protein, 514 aa, Thlaspi arvense
MATLLLTILLPTFLFLLVLLLSLRRNHNRSSRLPPGPKPWPILGNLPHMGPKPHRTLAAMVTTYGPILHLRLGFSNVVVAASKSVAEQFFKIHDANFASRPPNSRAKHMAYNYQDLVFAPYGQRWRMLRKISSVHLFSAKALEDYKHVRQEEVGTLTRELVDAGTKPVNLGQLVNMCVVNALGREMIGRRLFGAGADHKAEEFRSMVTEMMALAGVFNLGDFVPALDWLDLQGVAGKMKRLHKRFDTFLSSILKEHEMMHGQDQKHKDMLSTLISLKGTDFDGDGGSLTDTEIKALLLNMFTAGTDTSASTVDWAIAELIRHPDAMTRAQEELDSVVGRDRPINESDLSRLPYLQAVIKENFRLHPPTPLSLPHIASESCEINGYHIPKGSTLLTNIWAIARDPEQWSDPLSFRPERFLQGGEKSGVDVKGSDFELIPFGAGRRICAGLSLGLRMIQLLTATLVHGFDWELAGGIPPEKLNMEETYGITLQRAVPLVVHPKPRLAPSVYELGSR

SEQ ID NO: 127 - DNA, 1581 bp, Thlaspi arvense
  source 1..1581: mol_type = genomic DNA
atggatgaatcaagtatttttacggcaaagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaagacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 128 - protein, 526 aa, Thlaspi arvense
MDESSIFTAKKVIGAEKRELQGLLKAAVQSVEWTYSLFWQLCPQQRVLLWENGYYNGAIKTRKTTQPAEVTAEEAALERSQQLRELYEALLAGESSSEARACTALSPEDLTETEWFYLMCVSFSFPPPSGMPGKAYARRKHVWLCGANEVDSKIFSRAILAKSAKIQQTVVCIPMLDGVVELGTTNKVKEDIAFVELIKSFFHNHPKSNPKAALSEHSINEEHEEDEEQEEEEEEEVEEEMTMSEEIRLGSPDDDDVSNQNLLSDFHVESTHTLDTHMDMMNLMEEGGNYSQTVSTLLMSQPTSLFSDSVSTSSYIQSSFATWKADNFKEHQRVETKSTSSSQWMLKHIILRVPLLHDHTKEKRLPREELNHVVAERRRREKLNERFITLRSLVPFVTKMDKVSILGDTINYVNHLRNRVQELETNHHEQKHKRMRSCKGKTWEEVVEVSIIESDVLLEMRCEYRDGLLLDILQVLKEHGIETTAVHTAVNERDFEAEIRAMVRGKKPSIAEVKRAIHQTISNIKL

SEQ ID NO: 129 - DNA, 1581 bp, Thlaspi arvense
  source 1..1581: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaagacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatctaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 130 - protein, 316 aa, Thlaspi arvense
MDESSIFTAEKVIGAEKRELQGLLKAAVQSVEWTYSLFWQLCPQQRVLLWENGYYNGAIKTRKTTQPAEVTAEEAALERSQQLRELYEALLAGESSSEARACTALSPEDLTETEWFYLMCVSFSFPPPSGMPGKAYARRKHVWLCGANEVDSKIFSRAILAKSAKIQQTVVCIPMLDGVVELGTTNKVKEDIAFVELIKSFFHNHPKSNPKAALSEHSINEEHEEDEEQEEEEEEEVEEEMTMSEEIRLGSPDDDDVSNQNLLSDFHVESTHTLDTHMDMMNLMEEGGNYSQTVSTLLMSQPTSLFSDSVSTSSYI

SEQ ID NO: 131 - DNA, 1581 bp, Thlaspi arvense
  source 1..1581: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaagacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatgaaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 132 - protein, 322 aa, Thlaspi arvense
MDESSIFTAEKVIGAEKRELQGLLKAAVQSVEWTYSLFWQLCPQQRVLLWENGYYNGAIKTRKTTQPAEVTAEEAALERSQQLRELYEALLAGESSSEARACTALSPEDLTETEWFYLMCVSFSFPPPSGMPGKAYARRKHVWLCGANEVDSKIFSRAILAKSAKIQQTVVCIPMLDGVVELGTTNKVKEDIAFVELIKSFFHNHPKSNPKAALSEHSINEEHEEDEEQEEEEEEEVEEEMTMSEEIRLGSPDDDDVSNQNLLSDFHVESTHTLDTHMDMMNLMEEGGNYSQTVSTLLMSQPTSLFSDSVSTSSYIQSSFAT

SEQ ID NO: 133 - DNA, 1582 bp, Thlaspi arvense
  source 1..1582: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaagaacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 134 - protein, 99 aa, Thlaspi arvense
MDESSIFTAEKVIGAEKRELQGLLKAAVQSVEWTYSLFWQLCPQQRVLLWENGYYNGAIKNEEDNSAGGSDGGRGCVREESAAKGTLRGPFGRRVLIGS

SEQ ID NO: 135 - DNA, 1579 bp, Thlaspi arvense
  source 1..1579: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 136 - protein, 98 aa, Thlaspi arvense
MDESSIFTAEKVIGAEKRELQGLLKAAVQSVEWTYSLFWQLCPQQRVLLWENGYYNGAINEEDNSAGGSDGGRGCVREESAAKGTLRGPFGRRVLIGS

SEQ ID NO: 137 - DNA, 1582 bp, Thlaspi arvense
  source 1..1582: mol_type = genomic DNA
atggatgaatcaagtatttttacggcagagaaagtgatcggagctgagaaaagagagcttcaagggctgcttaaggcggcggtgcaatctgtggagtggacttatagtctcttctggcaactttgtcctcaacaaagggttttgctgtgggagaatggatactacaacggtgcaataaaggacgaggaagacaactcagccggcggaagtgacggcggaagaggctgcgttagagaggagtcagcagctaagggaactttacgaggcccttttggccggagagtcctcatcggaagctagggcatgcacggcattatcgccggaggatctgacggagactgaatggttttatctaatgtgtgtctctttctctttccctcctccttccgggatgccaggaaaggcgtatgcgaggaggaaacacgtatggctatgtggtgcaaatgaggttgacagtaaaatcttttctagggctattctcgcaaagagtgccaaaatccagcagacagtggtttgcattcccatgcttgatggcgttgtggaactaggcacaacgaacaaggtaaaagaagatatagcgtttgttgagctcataaagagttttttccataaccaccccaagtcaaacccaaaagctgctctttctgaacactccatcaacgaagagcacgaagaagacgaagaacaagaagaagaagaagaagaagaagtagaagaagaaatgacaatgtcagaggagataaggcttggctctcctgatgatgatgacgtctccaatcaaaacctactctctgatttccatgtagaatcaacccacactttagacacacacatggacatgatgaatctaatggaggagggtggaaactattctcagacagtatcaacacttcttatgtcacaacccacgagtcttttttcagattcagtttccacatcttcttacatccaatcatcatttgccacatggaaggctgataattttaaagagcatcagcgagtggaaactaaatcgacgtcgtcgtcgcaatggatgctcaaacacataatcttgagagttcctttactccacgaccacactaaagaaaagaggctgcctcgagaagagcttaatcacgtggtggcagagcgccgcaggagagagaagctgaatgagagattcataacactgagatcattggttccctttgtgaccaagatggataaagtctcaattcttggagacaccatcaactacgtaaaccatcttcgaaatagggtccaagagctggagactaatcatcacgaacaaaaacataagcggatgcgtagctgtaagggaaaaacgtgggaagaggtcgttgaggtttccatcatagagagtgatgttttgttagagatgagatgcgagtaccgagatggtctattgctcgacatccttcaggttcttaaggaacatggtatagagactactgcagttcataccgcggtgaacgagcgtgatttcgaggccgagataagggctatggtgagagggaagaaaccaagcattgctgaggtcaaaagagccatccatcaaactatatccaatattaaactatag

SEQ ID NO: 138 - protein, 99 aa, Thlaspi arvense; identical to SEQ ID NO: 134

SEQ ID NO: 139 - DNA, 1707 bp, Thlaspi arvense
  source 1..1707: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggaccagtgttaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttgatggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 140 - protein, 120 aa, Thlaspi arvense
MSQYSFFYFFLISLFLYENCIAYRYTFTVIEAPYSKLCSTKKILTVNGQFPGPVLRAYKGDTIYVNVRNQASENITLHWHGVEQPRNPWSDGPEYITQCPIQPGSDFTYKILLSIEDATV

SEQ ID NO: 141 - DNA, 1707 bp, Thlaspi arvense
  source 1..1707: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggaccagtgttaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcatttggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 142 - protein, 568 aa, Thlaspi arvense
MSQYSFFYFFLISLFLYENCIAYRYTFTVIEAPYSKLCSTKKILTVNGQFPGPVLRAYKGDTIYVNVRNQASENITLHWHGVEQPRNPWSDGPEYITQCPIQPGSDFTYKILLSIEDATVWWHAHSSWTRATVHGLIFVYPRPPDTLPFPEPDYEVPLVFGEWWKRDVREVVEDFMRNGGEPNVSDALTINGHPGFLYPCSQSDTFKLVVEKGKTYRIWMVNAAMNLILFFAIANHKLTVVAADGHYTKPLTASYITISPGQTLDLLLYADQSPESTYNMAARAYHSNPNVGFNNSTTVGILRYYSSNDAGTSSSERYPYLPGYNDTSAAFDFFTKIKGLYSRVAPAKVSRRIITTVSINLLKCPNDSCAGPNGSRLAASMNNISFVTPSHVDILRAYYLHINGVYGTRFPEFPPRIFNFTADDQPLFLQTPRLATEVKKFQYGETVEIVIQGTSLVGGGIDHPMHLHGFSFYVVGLGFGNFNARKDPSNYNLDDPPYRNTATVPRNGWIAIRFVADNPGVWFMHCHFDRHQTWGMNVVFIVKNGIKPNQKILPPPPGLPPCDQFENL

SEQ ID NO: 143 - DNA, 1708 bp, Thlaspi arvense
  source 1..1708: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggaccagtgtttaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 144 - protein, 60 aa, Thlaspi arvense
MSQYSFFYFFLISLFLYENCIAYRYTFTVIEAPYSKLCSTKKILTVNGQFPGPVFKGLQR

SEQ ID NO: 145 - DNA, 1708 bp, Thlaspi arvense
  source 1..1708: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggaccagtgattaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 146 - protein, 60 aa, Thlaspi arvense; identical to SEQ ID NO: 144

SEQ ID NO: 147 - DNA, 1700 bp, Thlaspi arvense
  source 1..1700: mol_type = genomic DNA
atgtcacaatattccttcttctatttcttcctaatctctcttttcctctacgaaaattgcattgcgtatcgctacacattcacggttattgaagctccatatagcaaactgtgtagcacgaagaagattttgaccgttaatggtcagtttcctggttaagggcttacaaaggtgacaccatttacgttaacgttcgtaaccaagctagtgaaaatatcacattgcattggcatggtgtagagcagccgagaaacccgtggtcagatggacccgaatacatcacacaatgcccgattcaacccgggtcagattttacgtacaaaattttactttccatcgaagacgcgactgtttggtggcatgcgcatagctcgtggacacgtgccaccgtacacggtctgattttcgtgtatcctcggcctcctgataccctgccttttccagaaccggactacgaagtccccttagtttttggagagtggtggaagagggatgtgagagaagtagtggaggatttcatgaggaacggaggtgaacctaatgtgtccgatgctttgactatcaatgggcatcctggtttcttgtatccttgctctcaatcagatacattcaagctcgtggtagagaagggcaaaacctaccgcattcggatggtaaacgccgcgatgaacctaattctcttcttcgccatcgcgaaccacaaactcaccgtggtcgccgccgatggccactacaccaaacctctaaccgctagttatatcaccatatctcctggccaaacgctagacctgttactatacgccgaccaaagtccagagagcacttataacatggcggccagagcttaccatagcaaccccaacgttgggttcaacaactctaccaccgtcgggatcttacgttactactcttcaaacgacgccggaacgtcttcatcagaacgttacccgtaccttcctggctacaatgacacctcagcagctttcgatttcttcacaaaaatcaaaggcttatactccagagtagctcccgccaaagtttcacgtaggataatcacgacggtttcgataaatctcctcaagtgtcccaacgactcgtgtgcaggcccaaacgggtcgaggttagcggcgagtatgaacaacatatcgttcgtcacaccgagccacgtggacatactaagagcttattaccttcacattaacggcgtttacggaacgcggtttccggagttcccaccgcggatattcaatttcacagcggacgaccaaccgctgtttttgcagactccgaggctggcgacggaggtaaagaagtttcagtacggggagacggttgagattgttatacaagggacgagtttggtaggtggtggaatcgatcatcctatgcatctccatggttttagcttctacgtggttggtttagggtttgggaattttaacgcacgtaaagatccctccaactataatctagacgatcctccttacagaaacacggcgactgtgcccaggaacggttggatcgctatcagattcgtagctgacaatccaggggtttggttcatgcactgtcactttgatagacatcaaacgtggggtatgaatgttgtcttcattgttaagaatggaataaaaccaaatcagaagattctacctccaccgcctggcttaccaccttgtgaccaatttgagaatctataa

SEQ ID NO: 148 - protein, 52 aa, Thlaspi arvense
MSQYSFFYFFLISLFLYENCIAYRYTFTVIEAPYSKLCSTKKILTVNGQFPG

SEQ ID NO: 149 - DNA, 1520 bp, Thlaspi arvense
  source 1..1520: mol_type = genomic DNA
atgagctccacggagacatatgagcctctattgagacggctccactcagattctcagttaaccgtaggttcttcaccggagatagaggagtttctcggccgtcgtagatccacggtgacgccacggtggtggctaaggctggccgtatgggaatcaaagcttctttggacgctctctggagcctccatagtggtctctgtgctgaattacatgctcagcttcgtcaccgttatgttcatcggccatcttggctctcttcagctcgccggcgcatccatcgccaccgtcggtatccaaggcctcgcttacggtatcatgttgggaatggcgagcgcggtccagacagtgtgtggtcaagcgtacggcgcgaggcagtactcatcaatgggaataatttgccaacgagccatggtcttgcacctcgcagctgcggtcctcctcacgttcctctactggtactcgggtccgatcctaaaggcgatgggccaatccgcagccatcgcacgcgagggtcaggtctttgcacgtgggattattccgcagatttatgcttttgccctcgcttgccctatgcagaggttcctccaggctcaaaaaattgtaaacccttttacatgtcactaggagttttcgtgctacacacgctactaacctggctggtaaccaacgtcctgcatttcggcttgctcggtgcagctctggtgctgagtttttcgtggtggcttctcgcggctgtgaatggtctgtatatcgtgatgagcccgagttgcaaggaaacttggaccgggttctcagctagggctttaagagggatttggccttacttcaagctcacgatagcttcagcagtcatgctatgtttggagatatggtacgtccaagggctagtgattatttccggtttactcaccaatcccacaattgccctagacgcaatttcgatttgcatgtattactggaattgggatatgcagttcatgcttggtctaagtgcggcaatcactgtccgagtgagcaacgagctaggagcgggaaacccacgagtggctaagttatcagtggtagtggttaacatcacgacggttgtcatcagcttattcctctgtgtcgttgtgctcgtgttccgcattggccttagtaaagccttcaccagcgacgcagaggttatagctgcagtctctgatctctttcccctgctcgccgtttccattttcttaaacggaatccaaccaattctctctggtgttgccattggaagtgggtggcaagcagtggtggcttatgtgaatcttgttacttactatgtcattggtcttcctattggctgtgttcttggcttcaaaaccagtcttggagttgcggggatctggtgggggatgattgcaggagttatacttcaaaccctaactttgattgttcttacactcagaactaactggaattccgaggtggagaatgcagctcataggttaaaagcttcagcaaatgagagtcaagaaatggctaccgaaggaatctaa

SEQ ID NO: 150 - protein, 207 aa, Thlaspi arvense
MSSTETYEPLLRRLHSDSQLTVGSSPEIEEFLGRRRSTVTPRWWLRLAVWESKLLWTLSGASIVVSVLNYMLSFVTVMFIGHLGSLQLAGASIATVGIQGLAYGIMLGMASAVQTVCGQAYGARQYSSMGIICQRAMVLHLAAAVLLTFLYWYSGPILKAMGQSAAIAREGQVFARGIIPQIYAFALACPMQRFLQAQKIVNPFTCH

SEQ ID NO: 151 - DNA, 1524 bp, Thlaspi arvense
  source 1..1524: mol_type = genomic DNA
atgagctccacggagacatatgagcctctattgagacggctccactcagattctcagttaaccgtaggttcttcaccggagatagaggagtttctcggccgtcgtagatccacggtgacgccacggtggtggctaaggctggccgtatgggaatcaaagcttctttggacgctctctggagcctccatagtggtctctgtgctgaattacatgctcagcttcgtcaccgttatgttcatcggccatcttggctctcttcagctcgccggcgcatccatcgccaccgtcggtatccaaggcctcgcttacggtatcatgttgggaatggcgagcgcggtccagacagtgtgtggtcaagcgtacggcgcgaggcagtactcatcaatgggaataatttgccaacgagccatggtcttgcacctcgcagctgcggtcctcctcacgttcctctactggtactcgggtccgatcctaaaggcgatgggccaatccgcagccatcgcacgcgagggtcaggtctttgcacgtgggattattccgcagatttatgcttttgccctcgcttgccctatgcagaggttcctccaggctcaaaaaattgtaaaccctttggcttacatgtcactaggagttttcgtgctacacacgctactaacctggctggtaaccaacgtcctgcatttcggcttgctcggtgcagctctggtgctgagtttttcgtggtggcttctcgcggctgtgaatggtctgtatatcgtgatgagcccgagttgcaaggaaacttggaccgggttctcagctagggctttaagagggatttggccttacttcaagctcacgatagcttcagcagtcatgctatgtttggagatatggtacgtccaagggctagtgattatttccggtttactcaccaatcccacaattgccctagacgcaatttcgatttgcatgtattactggaattgggatatgcagttcatgcttggtctaagtgcggcaatcactgtccgagtgagcaacgagctaggagcgggaaacccacgagtggctaagttatcagtggtagtggttaacatcacgacggttgtcatcagcttattcctctgtgtcgttgtgctcgtgttccgcattggccttagtaaagccttcaccagcgacgcagaggttatagctgcagtctctgatctctttcccctgctcgccgtttccattttcttaaacggaatccaaccaattctctctagtgttgccattggaagtgggtggcaagcagtggtggcttatgtgaatcttgttacttactatgtcattggtcttcctattggctgtgttcttggcttcaaaaccagtcttggagttgcggggatctggtgggggatgattgcaggagttatacttcaaaccctaactttgattgttcttacactcagaactaactggaattccgaggtggagaatgcagctcataggttaaaagcttcagcaaatgagagtcaagaaatggctaccgaaggaatctaa

SEQ ID NO: 152 - protein, 507 aa, Thlaspi arvense
MSSTETYEPLLRRLHSDSQLTVGSSPEIEEFLGRRRSTVTPRWWLRLAVWESKLLWTLSGASIVVSVLNYMLSFVTVMFIGHLGSLQLAGASIATVGIQGLAYGIMLGMASAVQTVCGQAYGARQYSSMGIICQRAMVLHLAAAVLLTFLYWYSGPILKAMGQSAAIAREGQVFARGIIPQIYAFALACPMQRFLQAQKIVNPLAYMSLGVFVLHTLLTWLVTNVLHFGLLGAALVLSFSWWLLAAVNGLYIVMSPSCKETWTGFSARALRGIWPYFKLTIASAVMLCLEIWYVQGLVIISGLLTNPTIALDAISICMYYWNWDMQFMLGLSAAITVRVSNELGAGNPRVAKLSVVVVNITTVVISLFLCVVVLVFRIGLSKAFTSDAEVIAAVSDLFPLLAVSIFLNGIQPILSSVAIGSGWQAVVAYVNLVTYYVIGLPIGCVLGFKTSLGVAGIWWGMIAGVILQTLTLIVLTLRTNWNSEVENAAHRLKASANESQEMATEGI

SEQ ID NO: 153 - DNA, 2214 bp, Thlaspi arvense
  source 1..2214: mol_type = genomic DNA
atgcttccattaatggcgataccacttgcgacttgcaggagcatcaactggtcagccacggaaaggattcctgtttcgcttctgttccggagtattcttctccaagacgacgaagtttgtagcgctgtgccactataccggatcctcgatcagaatgacgggcaacttggtcctataagtatggccgaggaatcagacaaaccattgctggatcctgatactctcaacagagaaggaattgacttgggtctgttgccattggaggaggtttttgaatacctaagaacatctccacgggggcttttatctggagatgctgaagaaagattgacgatatttggtcctaacagccttgaagagaaacgggagaacaagtttctgaagttcctaggttttatgtggaatcctttgtcatgggttatggaagctgcagcattgatggccatcgccctagcagatagtgaagtagagactatcagtcttttgctataccatttctgctcagtgctgaccggagaatcgctacctgtgaccaagaagaagggtgagcaagtcttctctggctctacttgtaagcaaggtgagatagaagctgttgtgatagccaccggttcgagcactttctttggtaaaacagcatctttggtggacagcacagatgcaactggacattttcagcaggttctaagcttgtgccagcagaaaaatgagattgcgcaaagagtttatgccatcataaatagatttgcagaaaaaggtttgaggtctcttgctgttgcttatcaggaaattccagagagaagcagcaacagtcctggaggaccatggttgttctgtggtctgttgccactgtttgatcctccaaggcatgacagtgctgaaaccatactgagagctcttaactttggagtttgtgttaagatgatcaccggtgatcagttggcgattgcaaaggagacaggaaggcgacttgggatgggaaccaatatgtatccttcttcctctttgttaggccacaacaacgatgatcacgaagccattccattggatgagcttattgaaatggcagatggatttgctggagtgttccctgaacacaagtatgagattgtaaagatattacaagaaaagaagcatgtggttggaatgaccggagatggtgtgaatgatgctcctgctctgaaaaaggctgacattggaatagctgtcgctgatgcaacagatgccgcaagaagttctgctgacattgtactaactgagcctggcttaagtgtaattatcagtgctgtcttgaccagcagagccattttccagcgtatgaagaactatacagtatatgcagtctcgatcaccatacgaatagtgctcggttttacacttttagcgttgatatgggaatacgactttccacctttcatggttttgataatcgcaatactcaatgacgggaccatcatgactatctctaaagatcgagtaaggccatctcctacacccgagagttggaagctcaaccagatatttgcgactggaattgtcattggaacataccttgcattggtcactgtcctattctactggatcattgtctctaccaccttcttcgagaaacacttccatgtaaaatcaatcggcaacaacagtgaacaagtctcatccgctctgtatctccaagtaagcatcatcagtcaagcactcatatttgtaacacgtagtcgaagctggtcttttcttgaacgtcccgggactctcctgattttcgccttccttgttgcccaacttgccgctacattgattgctgtctatgccaacatcagctttgctaacatcaccggcattggatggggatgggcaggtgttatatggttatacagtttgattttttacatacctcttgatattataaagttcttcttccactacgcattgagtggagatgcttggaaccttgtatttgaccgtaagacagcatttactaataagaaagattatagaaaagatgacggagcgtccaatgtaaccatctctcagagaagtcactctgcagaagaactcagtggaagtcgttctcgcgcatcttggatcgctgagcagaccagaaggcgtgcagaaaccgccaggctcttggagggacactcggtgtcaaggcatttggaatcagtaatgaagctcaaacaaattgaccccaagatgattcgtgcagacactgtctaa

SEQ ID NO: 154 - protein, 737 aa, Thlaspi arvense
MLPLMAIPLATCRSINWSATERIPVSLLFRSILLQDDEVCSAVPLYRILDQNDGQLGPISMAEESDKPLLDPDTLNREGIDLGLLPLEEVFEYLRTSPRGLLSGDAEERLTIFGPNSLEEKRENKFLKFLGFMWNPLSWVMEAAALMAIALADSEVETISLLLYHFCSVLTGESLPVTKKKGEQVFSGSTCKQGEIEAVVIATGSSTFFGKTASLVDSTDATGHFQQVLSLCQQKNEIAQRVYAIINRFAEKGLRSLAVAYQEIPERSSNSPGGPWLFCGLLPLFDPPRHDSAETILRALNFGVCVKMITGDQLAIAKETGRRLGMGTNMYPSSSLLGHNNDDHEAIPLDELIEMADGFAGVFPEHKYEIVKILQEKKHVVGMTGDGVNDAPALKKADIGIAVADATDAARSSADIVLTEPGLSVIISAVLTSRAIFQRMKNYTVYAVSITIRIVLGFTLLALIWEYDFPPFMVLIIAILNDGTIMTISKDRVRPSPTPESWKLNQIFATGIVIGTYLALVTVLFYWIIVSTTFFEKHFHVKSIGNNSEQVSSALYLQVSIISQALIFVTRSRSWSFLERPGTLLIFAFLVAQLAATLIAVYANISFANITGIGWGWAGVIWLYSLIFYIPLDIIKFFFHYALSGDAWNLVFDRKTAFTNKKDYRKDDGASNVTISQRSHSAEELSGSRSRASWIAEQTRRRAETARLLEGHSVSRHLESVMKLKQIDPKMIRADTV

SEQ ID NO: 155 - DNA, 2214 bp, Thlaspi arvense
  source 1..2214: mol_type = genomic DNA
atgcttccattaatggcgataccacttgcgacttgcaggagcatcaactggtcagccacggaaaggattcctgtttcgcttctgttccggagtattcttctccaagacgacgaagtttgtagcgctgtgccactataccggatcctcgatcagaatgacgggcaacttggtcctataagtatggccgaggaatcagacaaaccattgctggatcctgatactctcaacagagaaggaattgacttgggtctgttgccattggaggaggtttttgaatacctaagaacatctccacgggggcttttatctggagatgctgaagaaagattgacgatatttggtcctaacagccttgaagagaaacgggagaacaagtttctgaagttcctaggttttatgtggaatcctttgtcatgggttatggaagctgcagcattgatggccatcgccctagcagatagtgaagtagagactatcagtcttttgctataccatttctgctcagtgctgaccggagaatcgctacctgtgaccaagaagaagggtgagcaagtcttctctggctctacttgtaagcaaggtgagatagaagctgttgtgatagccaccggttcgagcactttctttggtaaaacagcatctttggtggacagcacagatgcaactggacattttcagcaggttctaagcttgtgccagcagaaaaatgagattgcgcaaagagtttatgccatcataaatagatttgcagaaaaaggtttgaggtctcttgctgttgcttatcaggaaattccagagagaagcagcaacagtcctggaggaccatggttgttctgtggtctgttgccactgtttgatcctccaaggcatgacagtgctgaaaccatactgagagctcttaaccttggagtttgtgttaagatgatcaccggtgatcagttggcgattgcaaaggagacaggaaggtgacttgggatgggaaccaatatgtatccttcttcctctttgttaggccacaacaacgatgatcacgaagccattccattggatgagcttattgaaatggcagatggatttgctggagtgttccctgaacacaagtatgagattgtaaagatattacaagaaaagaagcatgtggttggaatgaccggagatggtgtgaatgatgctcctgctctgaaaaaggctgacattggaatagctgtcgctgatgcaacagatgccgcaagaagttctgctgacattgtactaactgagcctggcttaagtgtaattatcagtgctgtcttgaccagcagagccattttccagcgtatgaagaactatacagtatatgcagtctcgatcaccatacgaatagtgctcggttttacacttttagcgttgatatgggaatacgactttccacctttcatggttttgataatcgcaatactcaatgacgggaccatcatgactatctctaaagatcgagtaaggccatctcctacacccgagagttggaagctcaaccagatatttgcgactggaattgtcattggaacataccttgcattggtcactgtcctattctactggatcattgtctctaccaccttcttcgagaaacacttccatgtaaaatcaatcggcaacaacagtgaacaagtctcatccgctctgtatctccaagtaagcatcatcagtcaagcactcatatttgtaacacgtagtcgaagctggtcttttcttgaacgtcccgggactctcctgattttcgccttccttgttgcccaacttgccgctacattgattgctgtctatgccaacatcagctttgctaacatcaccggcattggatggggatgggcaggtgttatatggttatacagtttgattttttacatacctcttgatattataaagttcttcttccactacgcattgagtggagatgcttggaaccttgtatttgaccgtaagacagcatttactaataagaaagattatagaaaagatgacggagcgtccaatgtaaccatctctcagagaagtcactctgcagaagaactcagtggaagtcgttctcgcgcatcttggatcgctgagcagaccagaaggcgtgcagaaaccgccaggctcttggagggacactcggtgtcaaggcatttggaatcagtaatgaagctcaaacaaattgaccccaagatgattcgtgcagacactgtctaa

SEQ ID NO: 156 - protein, 322 aa, Thlaspi arvense
MLPLMAIPLATCRSINWSATERIPVSLLFRSILLQDDEVCSAVPLYRILDQNDGQLGPISMAEESDKPLLDPDTLNREGIDLGLLPLEEVFEYLRTSPRGLLSGDAEERLTIFGPNSLEEKRENKFLKFLGFMWNPLSWVMEAAALMAIALADSEVETISLLLYHFCSVLTGESLPVTKKKGEQVFSGSTCKQGEIEAVVIATGSSTFFGKTASLVDSTDATGHFQQVLSLCQQKNEIAQRVYAIINRFAEKGLRSLAVAYQEIPERSSNSPGGPWLFCGLLPLFDPPRHDSAETILRALNLGVCVKMITGDQLAIAKETGR

SEQ ID NO: 157 - DNA, 2214 bp, Thlaspi arvense
  source 1..2214: mol_type = genomic DNA
atgcttccattaatggcgataccacttgcgacttgcaggagcatcaactggtcagccacggaaaggattcctgtttcgcttctgttccggagtattcttctccaagacgacgaagtttgtagcgctgtgccactataccggatcctcgatcagaatgacgggcaacttggtcctataagtatggccgaggaatcagacaaaccattgctggatcctgatactctcaacagagaaggaattgacttgggtctgttgccattggaggaggtttttgaatacctaagaacatctccacgggggcttttatctggagatgctgaagaaagattgacgatatttggtcctaacagccttgaagagaaacgggagaacaagtttctgaagttcctaggttttatgtggaatcctttgtcatgggttatggaagctgcagcattgatggccatcgccctagcagatagtgaagtagagactatcagtcttttgctataccatttctgctcagtgctgaccggagaatcgctacctgtgaccaagaagaagggtgagcaagtcttctctggctctacttgtaagcaaggtgagatagaagctgttgtgatagccaccggttcgagcactttctttggtaaaacagcatctttggtggacagcacagatgcaactggacattttcagcaggttctaagcttgtgccagcagaaaaatgagattgcgcaaagagtttatgccatcataaatagatttgcagaaaaaggtttgaggtctcttgctgttgcttatcaggaaattccagagagaagcagcaacagtcctggaggaccatggttgttctgtggtctgttgccactgtttgatcctccaaggcatgacagtgctgaaaccatactgagagctcttaaccttggagtttgtgttaagatgatcaccggtgatcagttggcgattgcaaaggagacaggaaggcgacttgggatgggaaccaatatgtatccttcttcctctttgttaggccacaacaacgatgatcacgaagccattccattggatgagcttattgaaatggcagatggatttgctggagtgttccctgaacacaagtatgagattgtaaagatattacaagaaaagaagcatgtggttggaatgaccagagatggtgtgaatgatgctcctgctctgaaaaaggctgacattggaatagctgtcgctgatgcaacagatgccgcaagaagttctgctgacattgtactaactgagcctggcttaagtgtaattatcagtgctgtcttgaccagcagagccattttccagcgtatgaagaactatacagtatatgcagtctcgatcaccatacgaatagtgctcggttttacacttttagcgttgatatgggaatacgactttccacctttcatggttttgataatcgcaatactcaatgacgggaccatcatgactatctctaaagatcgagtaaggccatctcctacacccgagagttggaagctcaaccagatatttgcgactggaattgtcattggaacataccttgcattggtcactgtcctattctactggatcattgtctctaccaccttcttcgagaaacacttccatgtaaaatcaatcggcaacaacagtgaacaagtctcatccgctctgtatctccaagtaagcatcatcagtcaagcactcatatttgtaacacgtagtcgaagctggtcttttcttgaacgtcccgggactctcctgattttcgccttccttgttgcccaacttgccgctacattgattgctgtctatgccaacatcagctttgctaacatcaccggcattggatggggatgggcaggtgttatatggttatacagtttgattttttacatacctcttgatattataaagttcttcttccactacgcattgagtggagatgcttggaaccttgtatttgaccgtaagacagcatttactaataagaaagattatagaaaagatgacggagcgtccaatgtaaccatctctcagagaagtcactctgcagaagaactcagtggaagtcgttctcgcgcatcttggatcgctgagcagaccagaaggcgtgcagaaaccgccaggctcttggagggacactcggtgtcaaggcatttggaatcagtaatgaagctcaaacaaattgaccccaagatgattcgtgcagacactgtctaa

SEQ ID NO: 158 - protein, 737 aa, Thlaspi arvense
MLPLMAIPLATCRSINWSATERIPVSLLFRSILLQDDEVCSAVPLYRILDQNDGQLGPISMAEESDKPLLDPDTLNREGIDLGLLPLEEVFEYLRTSPRGLLSGDAEERLTIFGPNSLEEKRENKFLKFLGFMWNPLSWVMEAAALMAIALADSEVETISLLLYHFCSVLTGESLPVTKKKGEQVFSGSTCKQGEIEAVVIATGSSTFFGKTASLVDSTDATGHFQQVLSLCQQKNEIAQRVYAIINRFAEKGLRSLAVAYQEIPERSSNSPGGPWLFCGLLPLFDPPRHDSAETILRALNLGVCVKMITGDQLAIAKETGRRLGMGTNMYPSSSLLGHNNDDHEAIPLDELIEMADGFAGVFPEHKYEIVKILQEKKHVVGMTRDGVNDAPALKKADIGIAVADATDAARSSADIVLTEPGLSVIISAVLTSRAIFQRMKNYTVYAVSITIRIVLGFTLLALIWEYDFPPFMVLIIAILNDGTIMTISKDRVRPSPTPESWKLNQIFATGIVIGTYLALVTVLFYWIIVSTTFFEKHFHVKSIGNNSEQVSSALYLQVSIISQALIFVTRSRSWSFLERPGTLLIFAFLVAQLAATLIAVYANISFANITGIGWGWAGVIWLYSLIFYIPLDIIKFFFHYALSGDAWNLVFDRKTAFTNKKDYRKDDGASNVTISQRSHSAEELSGSRSRASWIAEQTRRRAETARLLEGHSVSRHLESVMKLKQIDPKMIRADTV

SEQ ID NO: 159 - DNA, 733 bp, Thlaspi arvense
  source 1..733: mol_type = genomic DNA
atgggaagagggaagatagagataaagaagatagagaatcagacagcgaggcaagtgaccttctgcaagaggagaactggtcttatcaagaagactaatgagctctctgttctctgcgatgctcacattggtctcatcgtcttctcctccaccggaaagctctcgcagtactgttccgaacccctcaggatgcctcagctcattgaccgatacttgaagacccagtggaatgcgacttcctgatcctaatgacggccgggaggaattgtaccaagagatggaagtactaaaaagagagacatgtaagcttgagcttcgtctgcgtccataccatggacatgacttaacctcccttcctccacacgagctcgatggtctcgagcaacagctcgaacattctgtccttaaagtccgcgagcgtaagaatgagttgatgcagcaacagttggagaatctaagcagaaagaggcggatgctagaagaagataacaacaatatgtaccgtttgcttcatgagcatcgtaacgcggttgaatttcagcaagctgggatagagacgaaaccaggggagtatcaacagtttctagagcagcttcagtactataatgatcatcagcaacaaccaaacagtgttcttcagcttgctacgcttccttctgagattgatcctaattaccatctccagcttgctcagcctaatcttcaaaacgatccaacggccaagattgattag

SEQ ID NO: 160 - protein, 80 aa, Thlaspi arvense
MGRGKIEIKKIENQTARQVTFCKRRTGLIKKTNELSVLCDAHIGLIVFSSTGKLSQYCSEPLRMPQLIDRYLKTQWNATS

SEQ ID NO: 161 - DNA, 733 bp, Thlaspi arvense
  source 1..733: mol_type = genomic DNA
atgggaagagggaagatagagataaagaagatagagaatcagacagcgaggcaagtgaccttctgcaagaggagaactggtcttatcaagaagactaatgagctctctgttctctgcgatgctcacattggtctcatcgtcttctcctccaccggaaagctctcgcagtactgttccgaacccctcaggatgcctcagctcattgaccgatacttgaagacgcagtggaatgcgacttcctgatcctaatgacggccgggaggaattgtaccaagagatggaagtactaaaaagagagacatgtaagcttgagcttcgtctgcgtccataccatggacatgacttaacctcccttcctccacacgagctcgatggtctcgagcaacagctcgaacattctgtccttaaagtccgcgagcgtaagaatgagttgatgcagcaacagttggagaatctaagcagaaagaggcggatgctagaagaagataacaacaatatgtaccgtttgcttcatgagcatcgtaacgcggttgaatttcagcaagctgggatagagacgaaaccaggggagtatcaacagtttctagagcagcttcagtactataatgatcatcagcaacaaccaaacagtgttcttcagcttgctacgcttccttctgagattgatcctaattaccatctccagcttgctcagcctaatcttcaaaacgatccaacggccaagattgattag

SEQ ID NO: 162 - protein, 80 aa, Thlaspi arvense; identical to SEQ ID NO: 160

SEQ ID NO: 163 - DNA, 733 bp, Thlaspi arvense
  source 1..733: mol_type = genomic DNA
atgggaagagggaagatagagataaagaagatagagaatcagacagcgaggcaagtgaccttctgcaagaggagaactggtcttatcaagaagactaatgagctctctgttctctgcgatgctcacattggtctcatcgtcttctcctccaccggaaagctctcgcagtactgttccgaacccctcaggatgcctcagctcattgaccgatacttgaagactcagtggaatgcgacttcctgatcctaatgacggccgggaggaattgtaccaagagatggaagtactaaaaagagagacatgtaagcttgagcttcgtctgcgtccataccatggacatgacttaacctcccttcctccacacgagctcgatggtctcgagcaacagctcgaacattctgtccttaaagtccgcgagcgtaagaatgagttgatgcagcaacagttggagaatctaagcagaaagaggcggatgctagaagaagataacaacaatatgtaccgtttgcttcatgagcatcgtaacgcggttgaatttcagcaagctgggatagagacgaaaccaggggagtatcaacagtttctagagcagcttcagtactataatgatcatcagcaacaaccaaacagtgttcttcagcttgctacgcttccttctgagattgatcctaattaccatctccagcttgctcagcctaatcttcaaaacgatccaacggccaagattgattag

SEQ ID NO: 164 - protein, 80 aa, Thlaspi arvense; identical to SEQ ID NO: 160

SEQ ID NO: 165 - DNA, 1005 bp, Thlaspi arvense
  source 1..1005: mol_type = genomic DNA
atggataactcagctccggattctttgtccagatcggaaaccgccgtcacctacgattctccttacccactctacgccatggccttctcttccatcggtactccctccggccaccgaatcgccgtcggaagcttcctcgaggattacaacaaccgcatcgacattctctccttcgattccgactccatgacggtgaagcctctcccgaatctctccttcgatcatccttatcctcccacaaagctgatgttcagccctccctccctccgtcgtccctccgccggcgatctcctccgtttatgggagatcagtgaagattcctccaccgtcgagcctgtctcggtgctcaacaacagcaagacgagcgagttctgcgcgccgttgacctccttcgactggaacgacgtcgagccgaaacgcctgggaacctgcagcatcgacacgacctgcacgatttgggacatcgagaagtgcgtggtggagacgcagctcatagcgcacgataaggaggtccacgacatcgcttggggagaagctagggttttcgcgtcggtctccgccgacggatccgtcaggatcttcgatctacgcgacaaggagcattccaccatcatctacgagagtcctcagcccgatacgcctctcctaagactcgcctggaacaaacaagacctgaggtacatggcgacgatcctgatggattcgaataaggtcgtgattctcgacattcgctcgccgacgatgcctgtcgccgagctcgaacggcaccaggctagcgtcaacgccatagcttgggctccacagagctgcaaacacatctgctctgctggtgacgacacgcaggctctcatctgggagctcccgactgtagctggacccaacggcattgatccgatgtcggtttactcagccggctcggagataaaccagttgcagtggtcctcttcacagcctgattggattggcatcgctttcgctaacaaaatgcagctccttagagtttga

SEQ ID NO: 166 - protein, 334 aa, Thlaspi arvense
MDNSAPDSLSRSETAVTYDSPYPLYAMAFSSIGTPSGHRIAVGSFLEDYNNRIDILSFDSDSMTVKPLPNLSFDHPYPPTKLMFSPPSLRRPSAGDLLRLWEISEDSSTVEPVSVLNNSKTSEFCAPLTSFDWNDVEPKRLGTCSIDTTCTIWDIEKCVVETQLIAHDKEVHDIAWGEARVFASVSADGSVRIFDLRDKEHSTIIYESPQPDTPLLRLAWNKQDLRYMATILMDSNKVVILDIRSPTMPVAELERHQASVNAIAWAPQSCKHICSAGDDTQALIWELPTVAGPNGIDPMSVYSAGSEINQLQWSSSQPDWIGIAFANKMQLLRV

SEQ ID NO: 167 - DNA, 1026 bp, Thlaspi arvense
  source 1..1026: mol_type = genomic DNA
atggataactcagctccggattctttgtccagatcggaaaccgccgtcacctacgattctccttacccactctacgccatggccttctcttccatcggtactccctccggccaccgaatcgccgtcggaagcttcctcgaggattacaacaaccgcatcgacattctctccttcgattccgactccatgacggtgaagcctctcccgaatctctccttcgatcatccttatcctcccacaaagctgatgttcagccctccctccctccgtcgtccctccgccggcgatctcctcgcttccttcggcgatttcctccgtttatgggagatcagtgaagattcctccaccgtcgagcctgtctcggtgctcaacaacagcaagacgagcgagttctgcgcgccgttgacctccttcgactggaacgacgtcgagccgaaacgcctgggaacctgcagcatcgacacgacctgcacgatttgggacatcgagaagtgcgtggtggagacgcagctcatagcgcacgataaggaggtccacgacatcgcttggggagaagctagggttttcgcgtcggtctccgccgacggatccgtcaggatcttcgatctacgcgacaaggagcattccaccatcatctacgagagtcctcagcccgatacgcctctcctaagactcgcctggaacaaacaagacctgaggtacatggcgacgatcctgatggattcgaataaggtcgtgattctcgacattcgctcgccgacgatgcctgtcgccgagctcgaacggcaccaggctagcgtcaacgccatagcttgggctccacagagctgcaaacacatctgctctgctggtgacgacacgcaggctctcatctgggagctcccgactgtagctggacccaacggcattgatccgatgtcggtttactcagccggctcggagataaaccagttgcagtggtcctcttcacagcctgattggattggcatcgctttcgctaacaaaatgcagctccttagagtttga

SEQ ID NO: 168 - protein, 341 aa, Thlaspi arvense
MDNSAPDSLSRSETAVTYDSPYPLYAMAFSSIGTPSGHRIAVGSFLEDYNNRIDILSFDSDSMTVKPLPNLSFDHPYPPTKLMFSPPSLRRPSAGDLLASFGDFLRLWEISEDSSTVEPVSVLNNSKTSEFCAPLTSFDWNDVEPKRLGTCSIDTTCTIWDIEKCVVETQLIAHDKEVHDIAWGEARVFASVSADGSVRIFDLRDKEHSTIIYESPQPDTPLLRLAWNKQDLRYMATILMDSNKVVILDIRSPTMPVAELERHQASVNAIAWAPQSCKHICSAGDDTQALIWELPTVAGPNGIDPMSVYSAGSEINQLQWSSSQPDWIGIAFANKMQLLRV

SEQ ID NO: 169 - DNA, 1026 bp, Thlaspi arvense
  source 1..1026: mol_type = genomic DNA
atggataactcagctccggattctttgtccagatcggaaaccgccgtcacctacgattctccttacccactctacgccatggccttctcttccatcggtactccctccggccaccgaatcgccgtcggaagcttcctcgaggattacaacaaccgcatcgacattctctccttcgattccgactccatgacggtgaagcctctcccgaatctctccttcgatcatccttatcctcccacaaagctgatgttcagccctccctccctccgtcgtccctccgccggcgatctcctcgcttcctccggcgatttcctccgtttatgggagatcagtgaagattcctccaccgtcgagcctgtctcggtgctcaacaacagcaagacgagcgagttctgcgcgccgttgacctccttcgactggaacgacgtcgagccgaaacgcctgggaacctgcagcatcgacacgacctgcacgatttgggacatcgagaagtgcgtggtggagacgcagctcatagcgcacgataaggaggtccacgacatcgcttgggaagaagctagggttttcgcgtcggtctccgccgacggatccgtcaggatcttcgatctacgcgacaaggagcattccaccatcatctacgagagtcctcagcccgatacgcctctcctaagactcgcctggaacaaacaagacctgaggtacatggcgacgatcctgatggattcgaataaggtcgtgattctcgacattcgctcgccgacgatgcctgtcgccgagctcgaacggcaccaggctagcgtcaacgccatagcttgggctccacagagctgcaaacacatctgctctgctggtgacgacacgcaggctctcatctgggagctcccgactgtagctggacccaacggcattgatccgatgtcggtttactcagccggctcggagataaaccagttgcagtggtcctcttcacagcctgattggattggcatcgctttcgctaacaaaatgcagctccttagagtttga

SEQ ID NO: 170 - protein, 341 aa, Thlaspi arvense
MDNSAPDSLSRSETAVTYDSPYPLYAMAFSSIGTPSGHRIAVGSFLEDYNNRIDILSFDSDSMTVKPLPNLSFDHPYPPTKLMFSPPSLRRPSAGDLLASSGDFLRLWEISEDSSTVEPVSVLNNSKTSEFCAPLTSFDWNDVEPKRLGTCSIDTTCTIWDIEKCVVETQLIAHDKEVHDIAWEEARVFASVSADGSVRIFDLRDKEHSTIIYESPQPDTPLLRLAWNKQDLRYMATILMDSNKVVILDIRSPTMPVAELERHQASVNAIAWAPQSCKHICSAGDDTQALIWELPTVAGPNGIDPMSVYSAGSEINQLQWSSSQPDWIGIAFANKMQLLRV

SEQ ID NO: 171 - DNA, 1917 bp, Thlaspi arvense
  source 1..1917: mol_type = genomic DNA
atgatggatggaatggttactgaacaaaacagaatatctgttccggagaatctgaaaaaacagctcgcagtttcagttcgaagaattcaatggagttacgcaatcttctggtctatttctgcttctcagcctggagtgttagaatggggagatggatactataatggagatatcaaaacgaggaagacgattctagcatcggagatcaaagctgatcagcttggtttacgaaggagcgagcaacttagggagctttacgagtctctttctgtcgctgaatcttcttcctccggcgtcgccgccggatctcaggtcactagacgagcttctgccgccgcactctcgccggaagatctcgccgacacagagtggtactacttggtttgtatgtctttcgtcttcaacatcggtgaaggaatgcctggacgaacgtttgcgaacagtgaaccgatatggttgtgcaacgctcacaccgcagatagtaaagtctttagccgctctcttctggcaaaaagtgcttcggttgtgacagtgatttgcttcccgtttcttggaggagtcgttgagatcggtactacagaacatatcatagaggacatgaatgtaatacaatgcgtgaagacatcattcctcgaagctccagatccatacgcttcactgttaccaacaagatccgattatcacatcgacaacgttctcgatccgcaacagatcctaggtgacgagatttacgcgcctatgtttggtactgagacttttccggcgacttctcctagcagaactaccagcaggttcgatcctgaacacgagcaagtagcagaagatcatgactcgttcatggccgaaggaatcaatggaggagcttctcaggttcaaagctggcagttcatggacgaagagcttagtaactgcgttcaccaatcgcttaattccagtgattgcgtctctcaaacgtttgttaaagcagcggctggacgagtttcttgcaacgcaagaaaaggaagggctcaaaggttagggcatattcaagagcggcagagaaatgtgaaaatgttgtcttttgatccaagaaacgatgacgttcattaccaaagtgtgatctccacaatttttaagaccaaccatcagttaattctcggaccgcagttccggaattgcgataagcggtcaagcttcacgaggtggaagaaattatcgtcatcatcatctggaaccgcgtcaatcgtatcaccgtcacaaggaatgttaaagaagattcttttcgaggttcctcgagtgcaccagaaagagaagttgttgttggattcaccagggatcggggatgaaaccacgaaccacgcggttttagagaagaaacggcgtgagaaattgaacgaacggttcatgatcttgagatcaatcattccatcaatcaataagatcgataaagtatcaattcttgacgatacgatagagtatcttcaagaactggaaagacgtgttcaagaactagaatcttgcagagaatcaaccgatacagaaacacgtgggacaatgacagtgaagaggaagaaatctcacgacgcaggggaaagaacttcagccaactgcacaaacaatgaaaccggaaacgggaagagagttcaggttggagaaaccgagccggcagagaccgattataccggtttaactgataatttaaggatcggttcgtttggtaatgaggtcgttattgagcttagatgtgcttggagagaaggagtattgcttgagataatggatgtcattagtgatctcaacttagattctcattcggtacaatcctcgacaggagatggtttactctgcttaaccgtcaactgcaagcacaagggttcaaaaatgacgacaacaggaatgatcaaagaggcacttcaaaaggttgcatggatctgttga

SEQ ID NO: 172 - protein, 638 aa, Thlaspi arvense
MMDGMVTEQNRISVPENLKKQLAVSVRRIQWSYAIFWSISASQPGVLEWGDGYYNGDIKTRKTILASEIKADQLGLRRSEQLRELYESLSVAESSSSGVAAGSQVTRRASAAALSPEDLADTEWYYLVCMSFVFNIGEGMPGRTFANSEPIWLCNAHTADSKVFSRSLLAKSASVVTVICFPFLGGVVEIGTTEHIIEDMNVIQCVKTSFLEAPDPYASLLPTRSDYHIDNVLDPQQILGDEIYAPMFGTETFPATSPSRTTSRFDPEHEQVAEDHDSFMAEGINGGASQVQSWQFMDEELSNCVHQSLNSSDCVSQTFVKAAAGRVSCNARKGRAQRLGHIQERQRNVKMLSFDPRNDDVHYQSVISTIFKTNHQLILGPQFRNCDKRSSFTRWKKLSSSSSGTASIVSPSQGMLKKILFEVPRVHQKEKLLLDSPGIGDETTNHAVLEKKRREKLNERFMILRSIIPSINKIDKVSILDDTIEYLQELERRVQELESCRESTDTETRGTMTVKRKKSHDAGERTSANCTNNETGNGKRVQVGETEPAETDYTGLTDNLRIGSFGNEVVIELRCAWREGVLLEIMDVISDLNLDSHSVQSSTGDGLLCLTVNCKHKGSKMTTTGMIKEALQKVAWIC

SEQ ID NO: 173 - DNA, 3577 bp, Thlaspi arvense
  source 1..3577: mol_type = genomic DNA
atgatgtaaagagttgcaggtttttcttttctttttttgtttgctataattttaaccccaaaaaaaaaactcactttttctaaatcaggacaaaaatgaaattgatccttttgttttctttttccattttgtatggtttcatgaaaatttagggatggaatggttactgaacaaaacagaatatctgttccggagaatctgaaaaaacagctcgcagtttcagttcgaagaattcaatggagttacgcaatcttctggtctatttctgcttctcagcctgggtgagtttcttaatcactatccttgaaactgatcagttttgttatttttgaaggttcaaatctctactagggggaaaaaaatgaatttcgaattttcattgctaaagcctttgtctgaagctagaagtgcttttggttctgccttaaaaattattaaaactttgataaaaagattactgatttaatttgggaaattttgatttattctgcttctaagttccatcaatgaagttcttcaactcaagacaatgttctaagtcgatttgagttttaggttgatactgtcttagagattattagaaattttataagtggaaatagtgagaccttattagttacaaaaaaaactcaaaacaaagattaattatggttttttttttcttaaaattcaattttcttttgtcttttttttgtgtgtggataattcgggccggaaaccgattaatctcagagtgtgcaagttcgttgggttcttgaagttcttcattagtttcttactgattcgattcgattttttgttttttattgtttgaaccaaaagagtgttagaatggggagatggatactataatggagatatcaaaacgaggaagacgattctagcatcggagatcaaagctgatcagcttggtttacgaaggagcgagcaacttagggagctttacgagtctctttctgtcgctgaatcttcttcctccggcgtcgccgccggatctcaggtcactagacgagcttctgccgccgcactctcgccggaagatctcgccgacacagagtggtactacttggtttgtatgtctttcgtcttcaacatcggtgaagggtaattgcgtctatttatctttcggcattgacatttactatgtgcgtcgtcatgttcggattgatcttgtcagtgaaataaataaaaaaaagacaaaatacccttttattacatgtatgtctggatattagactttttggtaaaaaactaaaaagttttaactagaaaccaaaaattattattttccacaatcgtttgaaatttcttttttttttcttttttttttggtttggtttggttcagtttagggtttttttggattttcagatttttaggtcctggagaaaagaagtccaagatcggtttggttaagtcaaattttaaaatgatttggataattggtcttggtttggtttttaattaaaaattaatcaaaatgaaaatatataattagatttaatcattattataaattttgtaaatgcttaatgaaatgaatttcgatttattcagttctgttacaccaaatatgcgtttggttttgctttttatgcatggcatttccgtcttttcacgtatctagctattcattcacagaatgcctggacgaacgtttgcgaacagtgaaccgatatggttgtgcaacgctcacaccgcagatagtaaagtctttagccgctctcttctggcaaaagttagttgtattatattgtagttgctatataacatttctttttttgcgactgcaaacgcttaaattaatcgttttttttgtgtgtttgtgtgttttgtgcctgtttagagtgcttcggttgtgacagtgatttgcttcccgtttcttggaggagtcgttgagatcggtactacagaacatgtaaggcaaaatttccattttctgtcacaatttgagaatagttataaaacaaccactagttaagtttataacagtgcgaactaattcatcagaccgacgaattaaggtataaagttatgatacaaaaagtttaaagacttatcttgttgtgtgttttatcccgtagatcatagaggacatgaatgtaatacaatgcgtgaagacatcattcctcgaagctccagatccatacgcttcactgttaccaacaagatccgattatcacatcgacaacgttctcgatccgcaacagatcctaggtgacgagatttacgcgcctatgtttggtactgagacttttccggcgacttctcctagcagaactaccagcaggttcgatcctgaacacgagcaagtagcagaagatcatgactcgttcatggccgaaggaatcaatggaggagcttctcaggttcaaagctggcagttcatggacgaagagcttagtaactgcgttcaccaatcgcttaattccagtgattgcgtctctcaaacgtttgttaaagcagcggctggacgagtttcttgcaacgcaagaaaaggaagggctcaaaggttagggcatattcaagagcggcagagaaatgtgaaaatgttgtcttttgatccaagaaacgatgacgttcattaccaaagtgtgatctccacaatttttaagaccaaccatcagttaattctcggaccgcagttccggaattgcgataagcggtcaagcttcacgaggtggaagaaattatcgtcatcatcatctggaaccgcgtcaatcgtatcaccgtcacaaggaatgttaaagaagattcttttcgaggttcctcgagtgcaccagaaagagaagttgttgttggattcaccagggatcggggatgaaaccacgaaccacgcggttttagagaagaaacggcgtgagaaattgaacgaacggttcatgatcttgagatcaatcattccatcaatcaataaggtatatataaccgaatcttcaaaaattcaaattcatattatgtaaccagagttaactaattctaggtttgacattttcaatagatcgataaagtatcaattcttgacgatacgatagagtatcttcaagaactggaaagacgtgttcaagaactagaatcttgcagagaatcaaccgatacagaaacacgtgggacaatgacagtgaagaggaagaaatctcacgacgcaggggaaagaacttcagccaactgcacaaacaatgaaaccggaaacgggaagagagttcaggttggagaaaccgagccggcagagaccgattataccggtttaactgataatttaaggatcggttcgtttggtaatgaggtcgttattgagcttagatgtgcttggagagaaggagtattgcttgagataatggatgtcattagtgatctcaacttagattctcattcggtacaatcctcgacaggagatggtttactctgcttaaccgtcaactgcaaggtacagcctttaacctaatttttgcttaaccgggataaggttaacatggattgatatattgtgaaatgttggtttcagcacaagggttcaaaaatgacgacaacaggaatgatcaaagaggcacttcaaaaggttgcatggatctgttga

SEQ ID NO: 174 - DNA, 1917 bp, Thlaspi arvense
  source 1..1917: mol_type = genomic DNA
atgatggatggaatggttactgaacaaaacagaatatctgttccggagaatctgaaaaaacagctcgcagtttcagttcgaagaattcaatggagttacgcaatcttctggtctatttctgcttctcagcctggagtgttagaatggggagatggatactataatggagatatcaaaacgaggaagacgattctagcatcggagatcaaagctgatcagcttggtttacgaaggagcgagcaacttaggaagctttacgagtctctttctgtcgctgaatcttcttcctccggcgtcgccgccggatctcaggtcactagacgagcttctgccgccgcactctcgccggaagatctcgccgacacagagtggtactacttggtttgtatgtctttcgtcttcaacatcggtgaaggaatgcctggacgaacgtttgcgaacagtgaaccgatatggttgtgcaacgctcacaccgcagatagtaaagtctttagccgctctcttctggcaaaaagtgcttcggttgtgacagtgatttgcttcccgtttcttggaggagtcgttgagatcggtactacagaacatatcatagaggacatgaatgtaatacaatgcgtgaagacatcattcctcgaagctccagatccatacgcttcactgttaccaacaagatccgattatcacatcgacaacgttctcgatccgcaacagatcctaggtgacgagatttacgcgcctatgtttggtactgagacttttccggcgacttctcctagcagaactaccagcaggttcgatcctgaacacgagcaagtagcagaagatcatgactcgttcatggccgaaggaatcaatggaggagcttctcaggttcaaagctggcagttcatggacgaagagcttagtaactgcgttcaccaatcgcttaattccagtgattgcgtctctcaaacgtttgttaaagcagcggctggacgagtttcttgcaacgcaagaaaaggaagggctcaaaggttagggcatattcaagagcggcagagaaatgtgaaaatgttgtcttttgatccaagaaacgatgacgttcattaccaaagtgtgatctccacaatttttaagaccaaccatcagttaattctcggaccgcagttccggaattgcgataagcggtcaagcttcacgaggtggaagaaattatcgtcatcatcatctggaaccgcgtcaatcgtatcaccgtcacaaggaatgttaaagaagattcttttcgaggttcctcgagtgcaccagaaagagaagttgttgttggattcaccagggatcggggatgaaaccacgaaccacgcggttttagagaagaaacggcgtgagaaattgaacgaacggttcatgatcttgagatcaatcattccatcaatcaataagatcgataaagtatcaattcttgacgatacgatagagtatcttcaagaactggaaagacgtgttcaagaactagaatcttgcagagaatcaaccgatacagaaacacgtgggacaatgacagtgaagaggaagaaatctcacgacgcaggggaaagaacttcagccaactgcacaaacaatgaaaccggaaacgggaagagagttcaggttggagaaaccgagccggcagagaccgattataccggtttaactgataatttaaggatcggttcgtttggtaatgaggtcgttattgagcttagatgtgcttggagagaaggagtattgcttgagataatggatgtcattagtgatctcaacttagattctcattcggtacaatcctcgacaggagatggtttactctgcttaaccgtcaactgcaagcacaagggttcaaaaatgacgacaacaggaatgatcaaagaggcacttcaaaaggttgcatggatctgttga

SEQ ID NO: 175 - protein, 638 aa, Thlaspi arvense
MMDGMVTEQNRISVPENLKKQLAVSVRRIQWSYAIFWSISASQPGVLEWGDGYYNGDIKTRKTILASEIKADQLGLRRSEQLRKLYESLSVAESSSSGVAAGSQVTRRASAAALSPEDLADTEWYYLVCMSFVFNIGEGMPGRTFANSEPIWLCNAHTADSKVFSRSLLAKSASVVTVICFPFLGGVVEIGTTEHIIEDMNVIQCVKTSFLEAPDPYASLLPTRSDYHIDNVLDPQQILGDEIYAPMFGTETFPATSPSRTTSRFDPEHEQVAEDHDSFMAEGINGGASQVQSWQFMDEELSNCVHQSLNSSDCVSQTFVKAAAGRVSCNARKGRAQRLGHIQERQRNVKMLSFDPRNDDVHYQSVISTIFKTNHQLILGPQFRNCDKRSSFTRWKKLSSSSSGTASIVSPSQGMLKKILFEVPRVHQKEKLLLDSPGIGDETTNHAVLEKKRREKLNERFMILRSIIPSINKIDKVSILDDTIEYLQELERRVQELESCRESTDTETRGTMTVKRKKSHDAGERTSANCTNNETGNGKRVQVGETEPAETDYTGLTDNLRIGSFGNEVVIELRCAWREGVLLEIMDVISDLNLDSHSVQSSTGDGLLCLTVNCKHKGSKMTTTGMIKEALQKVAWIC

SEQ ID NO: 176 - DNA, 1917 bp, Thlaspi arvense
  source 1..1917: mol_type = genomic DNA
atgatggatggaatggttactgaacaaaacagaatatctgttccggagaatctgaaaaaacagctcgcagtttcagttcgaagaattcaatggagttacgcaatcttctggtctatttctgcttctcagcctggagtgttagaatggggagatggatactataatggagatatcaaaacgaggaagacgattctagcatcggagatcaaagctgatcagcttggtttacgaaggagcgagcaacttagggagctttacgagtctctttctgtcgctgaatcttcttcctccggcgtcgccgccggatctcaggtcactagacgagcttctgccgccgcactctcgccggaagatctcgccgacacaaagtggtactacttggtttgtatgtctttcgtcttcaacatcggtgaaggaatgcctggacgaacgtttgcgaacagtgaaccgatatggttgtgcaacgctcacaccgcagatagtaaagtctttagccgctctcttctggcaaaaagtgcttcggttgtgacagtgatttgcttcccgtttcttggaggagtcgttgagatcggtactacagaacatatcatagaggacatgaatgtaatacaatgcgtgaagacatcattcctcgaagctccagatccatacgcttcactgttaccaacaagatccgattatcacatcgacaacgttctcgatccgcaacagatcctaggtgacgagatttacgcgcctatgtttggtactgagacttttccggcgacttctcctagcagaactaccagcaggttcgatcctgaacacgagcaagtagcagaagatcatgactcgttcatggccgaaggaatcaatggaggagcttctcaggttcaaagctggcagttcatggacgaagagcttagtaactgcgttcaccaatcgcttaattccagtgattgcgtctctcaaacgtttgttaaagcagcggctggacgagtttcttgcaacgcaagaaaaggaagggctcaaaggttagggcatattcaagagcggcagagaaatgtgaaaatgttgtcttttgatccaagaaacgatgacgttcattaccaaagtgtgatctccacaatttttaagaccaaccatcagttaattctcggaccgcagttccggaattgcgataagcggtcaagcttcacgaggtggaagaaattatcgtcatcatcatctggaaccgcgtcaatcgtatcaccgtcacaaggaatgttaaagaagattcttttcgaggttcctcgagtgcaccagaaagagaagttgttgttggattcaccagggatcggggatgaaaccacgaaccacgcggttttagagaagaaacggcgtgagaaattgaacgaacggttcatgatcttgagatcaatcattccatcaatcaataagatcgataaagtatcaattcttgacgatacgatagagtatcttcaagaactggaaagacgtgttcaagaactagaatcttgcagagaatcaaccgatacagaaacacgtgggacaatgacagtgaagaggaagaaatctcacgacgcaggggaaagaacttcagccaactgcacaaacaatgaaaccggaaacgggaagagagttcaggttggagaaaccgagccggcagagaccgattataccggtttaactgataatttaaggatcggttcgtttggtaatgaggtcgttattgagcttagatgtgcttggagagaaggagtattgcttgagataatggatgtcattagtgatctcaacttagattctcattcggtacaatcctcgacaggagatggtttactctgcttaaccgtcaactgcaagcacaagggttcaaaaatgacgacaacaggaatgatcaaagaggcacttcaaaaggttgcatggatctgttga

SEQ ID NO: 177 - protein, 638 aa, Thlaspi arvense
MMDGMVTEQNRISVPENLKKQLAVSVRRIQWSYAIFWSISASQPGVLEWGDGYYNGDIKTRKTILASEIKADQLGLRRSEQLRELYESLSVAESSSSGVAAGSQVTRRASAAALSPEDLADTKWYYLVCMSFVFNIGEGMPGRTFANSEPIWLCNAHTADSKVFSRSLLAKSASVVTVICFPFLGGVVEIGTTEHIIEDMNVIQCVKTSFLEAPDPYASLLPTRSDYHIDNVLDPQQILGDEIYAPMFGTETFPATSPSRTTSRFDPEHEQVAEDHDSFMAEGINGGASQVQSWQFMDEELSNCVHQSLNSSDCVSQTFVKAAAGRVSCNARKGRAQRLGHIQERQRNVKMLSFDPRNDDVHYQSVISTIFKTNHQLILGPQFRNCDKRSSFTRWKKLSSSSSGTASIVSPSQGMLKKILFEVPRVHQKEKLLLDSPGIGDETTNHAVLEKKRREKLNERFMILRSIIPSINKIDKVSILDDTIEYLQELERRVQELESCRESTDTETRGTMTVKRKKSHDAGERTSANCTNNETGNGKRVQVGETEPAETDYTGLTDNLRIGSFGNEVVIELRCAWREGVLLEIMDVISDLNLDSHSVQSSTGDGLLCLTVNCKHKGSKMTTTGMIKEALQKVAWIC

SEQ ID NO: 178 - DNA, 1917 bp, Thlaspi arvense
  source 1..1917: mol_type = genomic DNA
atgatggatggaatggttactgaacaaaacagaatatctgttccggagaatctgaaaaaacagctcgcagtttcagttcgaagaattcaatggagttacgcaatcttctggtctatttctgcttctcagcctggagtgttagaatggggagatggatactataatggagatatcaaaacgaggaagacgattctagcatcggagatcaaagctgatcagcttggtttacgaaggagcgagcaacttagggagctttacgagtctctttctgtcgctgaatcttcttcctccggcgtcgccgccggatctcaggtcactagacgagcttctgccgccgcactctcgccggaagatctcgccgacacagagtggtactacttggtttgtatgtctttcgtcttcaacatcggtgaaggaatgcctggacgaacgtttgcgaacagtgaaccgatatggttgtgcaacgctcacaccgcagatagtaaagtctttagccgctctcttctggcaaaaagtgcttcggttgtgacagtgatttgcttcccgtttcttggaggagtcgttgagatcggtactacagaacatatcatagaggacatgaatgtaatacaatgcgtgaagacatcattcctcgaagctccagatccatacgcttcactgttaccaacaagatccgattatcacatcgacaacgttctcgatccgcaacagatcctaggtgacgagatttacgcgcctatgtttggtactgagacttttccggcgacttctcctagcagaactaccagcaggttcgatcctgaacacgagcaagtagcagaagatcatgactcgttcatggccgaaggaatcaatggaggagcttctcaggttcaaagctggcagttcatggacgaagagcttagtaactgcgttcaccaatcgcttaattccagtgattgcgtctctcaaacgtttgttaaagcagcggctggacgagtttcttgcaacgcaagaaaaggaagggctcaaaggttagggcatattcaagagcggcagagaaatgtgaaaatgttgtcttttgatccaagaaacgatgacgttcattaccaaagtgtgatctccacaatttttaagaccaaccatcagttaattctcggaccgcagttccggaattgcgataagcggtcaagcttcacgaggtggaagaaattatcgtcatcatcatctggaaccgcgtcaatcgtatcaccgtcacaaggaatgttaaagaagattcttttcgaggttcctcgagtgcaccagaaagagaagttgttgttggattcaccagggatcggggatgaaaccacgaaccacgcggttttagagaagaaacggcgtgagaaattgaacgaacggttcatgatcttgagatcaatcattccatcaatcaataagatcgataaagtatcaattcttgacgatacgatagagtatcttcaagaactggaaagacgtgttcaagaactagaatcttgcagagaatcaaccgatacagaaacacgtgggacaatgacagtgaagaggaagaaatctcacgacgcaggggaaagaacttcagccaactgcacaaacaatgaaaccggaaacgggaagagagttcaggttggagaaaccgagccggcagagaccgattataccggtttaactaataatttaaggatcggttcgtttggtaatgaggtcgttattgagcttagatgtgcttggagagaaggagtattgcttgagataatggatgtcattagtgatctcaacttagattctcattcggtacaatcctcgacaggagatggtttactctgcttaaccgtcaactgcaagcacaagggttcaaaaatgacgacaacaggaatgatcaaagaggcacttcaaaaggttgcatggatctgttga

SEQ ID NO: 179 - protein, 638 aa, Thlaspi arvense
MMDGMVTEQNRISVPENLKKQLAVSVRRIQWSYAIFWSISASQPGVLEWGDGYYNGDIKTRKTILASEIKADQLGLRRSEQLRELYESLSVAESSSSGVAAGSQVTRRASAAALSPEDLADTEWYYLVCMSFVFNIGEGMPGRTFANSEPIWLCNAHTADSKVFSRSLLAKSASVVTVICFPFLGGVVEIGTTEHIIEDMNVIQCVKTSFLEAPDPYASLLPTRSDYHIDNVLDPQQILGDEIYAPMFGTETFPATSPSRTTSRFDPEHEQVAEDHDSFMAEGINGGASQVQSWQFMDEELSNCVHQSLNSSDCVSQTFVKAAAGRVSCNARKGRAQRLGHIQERQRNVKMLSFDPRNDDVHYQSVISTIFKTNHQLILGPQFRNCDKRSSFTRWKKLSSSSSGTASIVSPSQGMLKKILFEVPRVHQKEKLLLDSPGIGDETTNHAVLEKKRREKLNERFMILRSIIPSINKIDKVSILDDTIEYLQELERRVQELESCRESTDTETRGTMTVKRKKSHDAGERTSANCTNNETGNGKRVQVGETEPAETDYTGLTNNLRIGSFGNEVVIELRCAWREGVLLEIMDVISDLNLDSHSVQSSTGDGLLCLTVNCKHKGSKMTTTGMIKEALQKVAWIC

SEQ ID NO: 180 - DNA, 1041 bp, Thlaspi arvense
  source 1..1041: mol_type = genomic DNA
atgacaatcttggatcagaccgttgtaacaaccggatcgaagaaggcttgtgtcatcggtggcacaggaaacttagcttctactctcatcaagcatttgcttcaaagtggctacaaagttaacactacagttagagatccagagaatgagaagaaaatggctcacttaagggtacttcaagaacttggggacctcaagatcttcaaggcggaatttactgatgaagagagtttcgattcaccagtttcgggctgtgattacgttttccatgtcgcaacgcctatcaactttacatctgaagatcccgagaaagacatgatcaagccaaggatacaaggagtgaccaatgtgttgaaatcttgcttaaaatcgaaatcagtcaagcgtgtgatctacacttcttcagctgctgcggtttccatcaacaatctttctggacctggacttgtgatgaacgaagaaaactggactgaccttgattatctcacaaaggagaagccgtttaactggggctacccagtgtcaaagatactagcagaaaaggcagcttgtaaatttgcggaagagaacaagatcgatctagttaccgtgattccggcactcatatccggaaaatctctcctctcggatcctcctccgagcagctcatttctctctatgtctttaatcaccgggaatgaaatgtatctgaaaggtctcaaggaaatgcagaagcaatctggctccatctcgttcagccacgtgaaggatttggctcgtgcccatttgtttcttgcggagaaagaaactgcgtctggtcgttacatttgctgtacttacaacacaagtgttccggagattgcagattttctcaggcagagatatcctaagtacaatgtgctgtctgaattcgaagagtgcttatcaagtgcgaagctgacgctatcttcggaaaaactcatcaatgaaggctttcgattcgaatatgggattaatgagatctatgatgagatgatagagcacttcgagtccaaaggattaatcaaagctaaagaatcttga

SEQ ID NO: 181 - protein, 346 aa, Thlaspi arvense
MTILDQTVVTTGSKKACVIGGTGNLASTLIKHLLQSGYKVNTTVRDPENEKKMAHLRVLQELGDLKIFKAEFTDEESFDSPVSGCDYVFHVATPINFTSEDPEKDMIKPRIQGVTNVLKSCLKSKSVKRVIYTSSAAAVSINNLSGPGLVMNEENWTDLDYLTKEKPFNWGYPVSKILAEKAACKFAEENKIDLVTVIPALISGKSLLSDPPPSSSFLSMSLITGNEMYLKGLKEMQKQSGSISFSHVKDLARAHLFLAEKETASGRYICCTYNTSVPEIADFLRQRYPKYNVLSEFEECLSSAKLTLSSEKLINEGFRFEYGINEIYDEMIEHFESKGLIKAKES

SEQ ID NO: 182 - DNA, 1188 bp, Thlaspi arvense
  source 1..1188: mol_type = genomic DNA
atggtgatgggtacacaaccgtcgttggaagagatcagaaaggcacagagagcggatggccccgcaggcatcttggggataggcacggccaaccctgcgaaccatgtgatccaggcagagtatccggactactacttccgcatcaccaacagtgagcacatgactgacctcaaggagaagttcaagcgcatgtgcgacaagtcgatgatacggaaacggcacatgcacctgacggaggagttcctgaaggagaatccggacatgtgcgcctacatggctccttctcttgatgtgaggcaggacatcgtggtggtcgaggtccctaagctagggaaagaggcggcagtgaaggccatcaaggagtggggtcagcccaagtccaagatcacccacgtcgtcttctgcactacatccggagttgacatgcctggtgctgactaccagctcaccaagctcctcggtcttcgcccttccgtcaagcgtctcatgatgtaccagcaaggttgctacgccggcggcactgtcctccgactcgccaaggacctcgctgagaataaccgtggtgctcgtgtccttgtcgtctgctccgagatcacagccgtcaccttccgtggcccctctgacacacacctcgactccctcgttggtcaggctctcttcagtgacggtgctgccgcgctcattgttggtgcggaccctgatgcctccgtgggagagaagcctatcttcgagatggtgtctgctgcacagaccatcctcccagactcggacggagccatagatggacacttgagggaagttgggctcaccttccatctcctcaaggacgtccctgggctcatctcgaagaacatagagaagagtctagaagaagcgtttaaaccgctcgggataagtgactggaactctctcttttggatagctcaccctgaaggtcctgcgatcctggaccaggttgagttaaagctaggactcaaggaagagaagatgagggccacgcgtcacgtgctgagcgagtacggaaacatgtcgagcgcgtgcgttctcttcattatggacgagatgaggaggaagtccaaggaggatggtgtggccacgacaggagaagggttggagtggggtgtcttgtttggtttcggaccaggtctcaccgtagagacagtcgtcttgcacagcgtccctgtttga

SEQ ID NO: 183 - protein, 395 aa, Thlaspi arvense
MVMGTQPSLEEIRKAQRADGPAGILGIGTANPANHVIQAEYPDYYFRITNSEHMTDLKEKFKRMCDKSMIRKRHMHLTEEFLKENPDMCAYMAPSLDVRQDIVVVEVPKLGKEAAVKAIKEWGQPKSKITHVVFCTTSGVDMPGADYQLTKLLGLRPSVKRLMMYQQGCYAGGTVLRLAKDLAENNRGARVLVVCSEITAVTFRGPSDTHLDSLVGQALFSDGAAALIVGADPDASVGEKPIFEMVSAAQTILPDSDGAIDGHLREVGLTFHLLKDVPGLISKNIEKSLEEAFKPLGISDWNSLFWIAHPEGPAILDQVELKLGLKEEKMRATRHVLSEYGNMSSACVLFIMDEMRRKSKEDGVATTGEGLEWGVLFGFGPGLTVETVVLHSVPV

(19) United States
(12) Patent Application Publication
(10) Pub. No.: US 2026/0293934 A1
(43) Pub. Date: Oct. 1, 2026

(54) LOW FIBER PENNYCRESS MEAL, SEEDS, AND METHODS OF MAKING

(71) Applicants: COVERCRESS INC., St. Louis, MO (US); BOARD OF TRUSTEES OF ILLINOIS STATE UNIVERSITY, Normal, IL (US); REGENTS OF THE UNIVERSITY OF MINNESOTA, Minneapolis, MN (US)

(72) Inventors: Tim Ulmasov, Chesterfield, MO (US); Gary Hartnell, St. Peters, MO (US); John C. Sedbrook, Bloomington, IL (US); Michael David Marks, Roseville, MN (US); Ratan Chopra, St. Louis, MO (US); Maliheh Esfahanian, San Diego, CA (US)

(21) Appl. No.: 19/701,911

(22) Filed: Jun. 9, 2026

Related U.S. Application Data
(63) Continuation of application No. 18/670,499, filed on May 21, 2024, now Pat. No. 12,667,119.
Application No. 18/670,499 is a division of application No. 18/185,195, filed on Mar. 16, 2023, now Pat. No. 12,022,848.
Application No. 18/185,195 is a division of application No. 17/643,730, filed on Dec. 10, 2021, now Pat. No. 11,647,765.
Application No. 17/643,730 is a division of application No. 16/893,636, filed on Jun. 5, 2020, now Pat. No. 11,224,237.
Application No. 16/893,636 is a division of application No. 16/131,633, filed on Sep. 14, 2018, now Pat. No. 10,709,151.
(60) Provisional application No. 62/559,122, filed on Sep. 15, 2017.

(51) Int. Cl. A23K 10/30; A23K 20/158

(52) U.S. Cl. CPC A23K 10/30; A23K 20/158

(57) ABSTRACT

Pennycress seed, seed lots, and seed meal having reduced fiber content and improved suitability for use in producing animal feed are provided.

### CROSS-REFERENCE TO RELATED APPLICATIONS

This is application is a continuation of U.S. Ser. No. 18/670,499, filed May 21, 2024, which is a divisional patent application of U.S. Ser. No. 18/185,195, filed Mar. 16, 2023, which is a divisional patent application of U.S. Ser. No. 17/643,730, filed Dec. 10, 2021, which is a divisional patent application of U.S. Ser. No. 16/893,636, filed Jun. 5, 2020, which is a divisional patent application of U.S. Ser. No. 16/131,633, filed Sep. 14, 2018, which claims the benefit of U.S. Provisional Patent Application Ser. No. 62/559,122, filed Sep. 15, 2017, all of which are incorporated herein by reference in their entireties.

### STATEMENT REGARDING FEDERAL FUNDING

This invention was made with government support under Grant Number 2014-67009-22305 and 2018-67009-27374 awarded by the National Institute of Food and Agriculture, USDA. The government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML format sequence listing, created on Jun. 8, 2026, is named “P13415US07.xml” and is 409,282 bytes in size.

### BACKGROUND

Different plants have seed contents that make them desirable for feed compositions. Examples are soybean, canola, rapeseed and sunflower. After crushing the seeds and recovering the oil, the resulting meal has a protein content making the meal useful as a feed ingredient for ruminants, monogastrics, poultry, and aquaculture. Nevertheless, there remains a desire for improved plant seeds that can provide additional sources of nutrition to animals.

Field Pennycress Thlaspi arvense L. (common names: fanweed, stinkweed, field pennycress), hereafter referred to as Pennycress or pennycress, is a winter cover crop that helps to protect soil from erosion, prevent the loss of farm-field nitrogen into water systems, and retain nutrients and residues to improve soil productivity. While it is well established that cover crops provide agronomic and ecological benefits to agriculture and environment, only 5% of farmers today are using them. One reason is economics—it requires on average ~$30-40/acre to grow a cover crop on the land that is otherwise idle between two seasons of cash crops such as corn and soy. In the last 5 years, it has been recognized that pennycress could be used as a novel cover crop, because in addition to providing cover crop benefits, it is an oilseed with its oil being useful as a biofuel. Extensive testing indicates that it can be interseeded over standing corn in early fall and harvested in spring prior to soybean planting (in appropriate climates). As such, its growth and development requires minimal incremental inputs (e.g., no/minimum tillage, no/low nitrogen, insecticides or herbicides). Pennycress also does not directly compete with existing crops when intercropped for energy production, and the recovered oil and meal can provide an additional source of income for farmers.

Pennycress is a winter annual belonging to the Brassicaceae (mustard) family. It's related to cultivated crops, rapeseed and canola, which are also members of the Brassicaceae family. Pennycress seeds are smaller than canola, but they are also high in oil content. They typically contain 36% oil, which is roughly twice the level found in soybean, and the oil has a very low saturated fat content (~4%). Pennycress represents a clear opportunity for sustainable optimization of agricultural systems. For example, in the US Midwest, ~35M acres that remain idle could be planted with pennycress after a corn crop is harvested and before the next soybean crop is planted. Pennycress can serve as an important winter cover crop working within the no/low-till corn and soybean rotation to guard against soil erosion and improve overall field soil nitrogen and pest management.

Pennycress has an oil content that makes it highly desirable as a biofuel, and potentially as a food oil. Once the oil is obtained from pennycress, either from mechanical expeller pressing or hexane extraction, the resulting meal has a high protein level with a favorable amino acid profile that could provide nutritional benefits to animals. However, studies of pennycress processing have consistently demonstrated that the meal produced has a high level of non-digestible fiber, and as a result, not enough metabolizable energy to be competitive with high-value products like soybean and canola meals as an animal feed.

### SUMMARY

Compositions comprising non-defatted pennycress seed meal comprising an acid detergent fiber (ADF) content of 5% to 20% by dry weight are provided herein.

Compositions comprising defatted pennycress seed meal comprising an acid detergent fiber (ADF) content of 7% to 25% by dry weight are provided herein.

Pennycress seed meals comprising an acid detergent fiber (ADF) content of 5% to 20% by dry weight, wherein the seed meal is non-defatted, are provided herein.

Pennycress seed meals comprising an acid detergent fiber (ADF) content of 7% to 25% by dry weight, wherein the seed meal is defatted, are provided herein.

Pennycress seed cakes comprising an acid detergent fiber (ADF) content of 7% to 25% by dry weight are provided herein.

In one embodiment, this disclosure provides a low fiber pennycress meal composition.

Seed lots comprising a population of pennycress seeds that comprise an acid detergent fiber (ADF) content of 5% to 20% by dry weight are provided herein.

Methods of making non-defatted pennycress seed meal comprising an acid detergent fiber (ADF) content of 5% to 20% by dry weight, comprising the step of grinding, macerating, extruding, and/or crushing the aforementioned seed lots, thereby obtaining the non-defatted seed meal, are provided herein.

Methods of making defatted pennycress seed meal comprising an acid detergent fiber (ADF) content of 7% to 25% by dry weight, comprising the step of solvent extracting the, separating the extracted seed meal from the solvent, thereby obtaining the defatted seed meal, are provided herein.

Methods of making pennycress seed cake comprising an acid detergent fiber (ADF) content of 7% to 25% by dry weight, comprising the step of crushing or expelling the seed of any of the aforementioned seed lots, thereby obtaining a seed cake, are provided herein.

Methods of making a pennycress seed lot comprising the steps of: (a) introducing at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof; (b) selecting germplasm that is homozygous for said loss-of-function mutation; and, (c) harvesting seed from the homozygous germplasm, thereby obtaining a seed lot, wherein said seed lot comprises an acid detergent fiber (ADF) content of 5% to 20% by dry weight, are provided herein.

Method of making a pennycress seed lot comprising the steps of: (a) introducing at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof into a pennycress plant genome; (b) selecting a transgenic plant line that comprises said transgene and (c) harvesting seed from the transgenic plant line, thereby obtaining a seed lot, wherein said seed lot comprises an acid detergent fiber (ADF) content of 5% to 20% by dry weight, are provided herein.

In one embodiment, this disclosure provides a method for producing low fiber pennycress seeds and meal. The method comprises genetically modifying pennycress seed (e.g., using gene editing or transgenic approach) to modify expression of one or more genes involved in seed coat development. Genetically altered seed lots with improved composition, such as lower fiber content, increased oil content, and increased protein content, all in comparison to control seed lots that lack the genetic alteration can be obtained by these methods.

### BRIEF DESCRIPTION OF THE DRAWINGS

The accompanying drawings, which are incorporated in and form a part of the specification, illustrate the embodiments of the present disclosure and together with the description, serve to explain the principles of the disclosure. In the drawings:

FIG. 1A, B, C illustrate mutant pennycress seeds with varying seed color. Dark seeds in the center are representative of a wild-type genetic background. The seeds of two pennycress seed isolates (Y1126 and Y1067), along with 7 pennycress M3-generation EMS mutants in the Spring 32 background are shown. All mutant seeds exhibit light-colored seed coats compared to the dark color of typical wild-type pennycress seeds (wild-type Spring 32 seeds shown as an example). Examples of dark and light-colored seed and meal (non-defatted) are also shown. Panel A: Spectrum of seed coat color ranging from dark to light in wild type and mutant pennycress seeds. Panel B: Pennycress meal produced from wild type (Beecher). Panel C: Pennycress meal produced from one of the light-colored seed lines (Y1126).

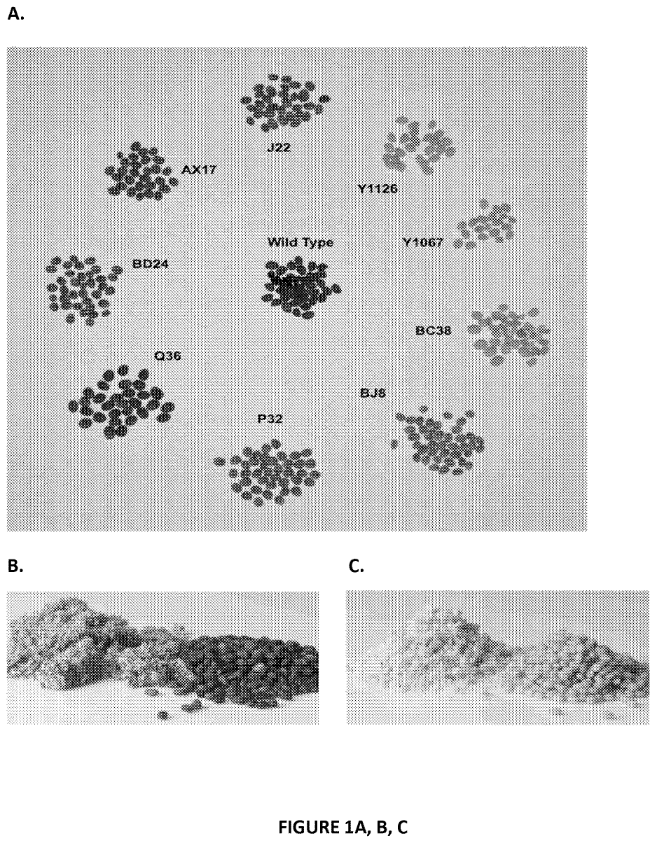

FIG. 2A, B illustrates pARV8 (SS51_Tt10), Agrobacterium CRISPR-Cas9 vector and its gene editing sgRNA cassette, for targeting pennycress homolog of Transparent testa 10 (Tt10) gene. Panel A: Plasmid map of pARV8 (SS51_Tt10). Panel B: sgRNA cluster in pARV8, targeting nucleotides 341-360 and 382-401 of SEQ ID NO: 33.

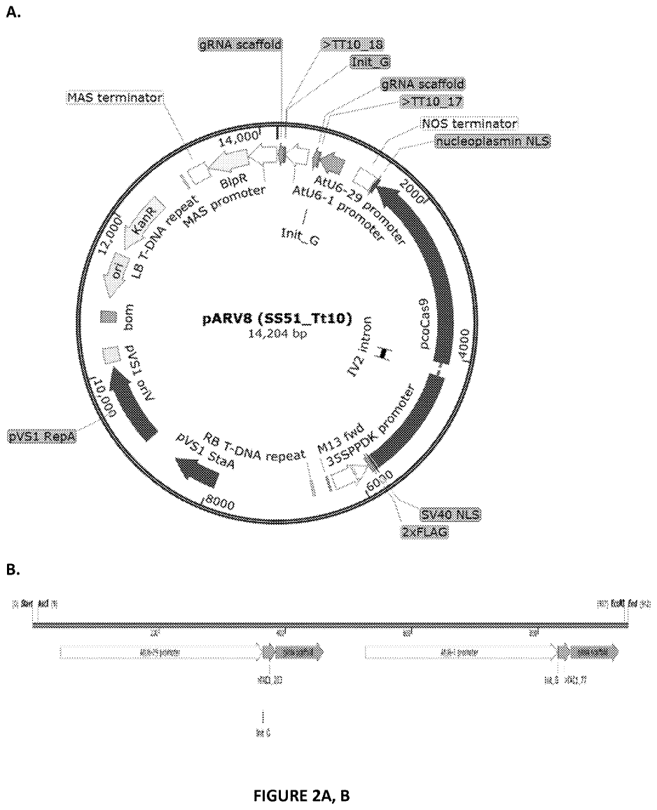

FIG. 3 illustrates pARV187, Agrobacterium CRISPR-FnCpf1 base vector for editing plant genome. gRNA cassette stuffers are inserted at the dual AarI site, replacing a small fragment of the vector with synthetic gRNA cassette.

FIG. 4 illustrates pARV191, Agrobacterium CRISPR-SmCsm1 base vector for editing plant genome. gRNA cassette stuffers are inserted at the dual AarI site, replacing a small fragment of the vector with synthetic gRNA cassette.

FIGS. 5A, B, C, D, E, F, G, gRNA cassettes targeting pennycress Transparent testa (Tt) genes. FIG. 5A illustrates a gRNA cassette stuffer, designed for insertion into the AarI-digested plant genome editing vector (such as pARV187 or pARV191) for targeting pennycress Tt1 gene, nucleotides 59-81 and 307-329 of SEQ ID NO: 27; FIG. 5B: gRNA cassette stuffer for targeting pennycress Tt2 gene, nucleotides 177-199 and 240-262 of SEQ ID NO: 1; FIG. 5C: gRNA cassette stuffer for targeting pennycress Tt8 gene, nucleotides 261-283 and 153-175 of SEQ ID NO: 69; FIG. 5D: gRNA cassette stuffer for targeting pennycress Tt8 gene, nucleotides 145-167 and 274-296 of SEQ ID NO: 69; FIG. 5E: gRNA cassette stuffer for targeting pennycress Tt10 gene, nucleotides 304-326 and 415-437 of SEQ ID NO: 33; FIG. 5F: gRNA cassette stuffer for targeting pennycress Tt12 gene, nucleotides 399-421 and 450-472 of SEQ ID NO: 36; FIG. 5G: gRNA cassette stuffer for targeting pennycress Tt15 gene, nucleotides 255-277 and 281-303 of SEQ ID NO: 42.

FIG. 6 illustrates total oil content in seeds of selected yellow-seeded pennycress mutants measured using GC-chromatography analysis.

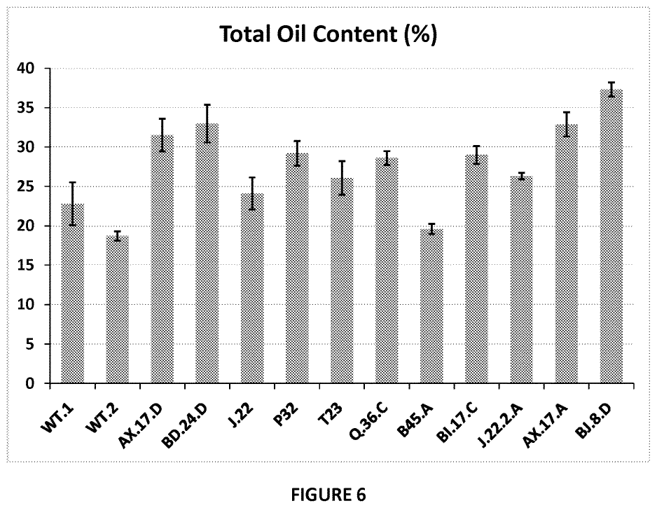

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Pennycress has value in both its oil and the resulting meal following the removal of oil. The meal is used for animal feed and is typically valued for its energy, protein and sometimes fiber. Fiber is usually delivered by forage elements (not protein supplements) and only a modest amount is desired. Fiber is measured by multiple measures including Crude Fiber (CF), Acid detergent Fiber (ADF) and Neutral detergent fiber (NDF). ADF is a useful determinant in estimating the energy available to animals. In certain embodiments, ADF can be measured gravimetrically using Association of Official Analytical Chemists (AOAC) Official Method 973.18 (1996): “Fiber (Acid Detergent) and Lignin in Animal Feed”. In certain embodiments, modifications of this method can include use of Sea Sand for filter aid as needed. NDF can be determined as disclosed in JAOAC 56, 1352-1356, 1973. In certain embodiments, fiber (ADF and/or NDF), protein, and/or oil content can be determined by Near-infrared (NIR) spectroscopy.

Defatted-pennycress seed meal having less fiber than defatted control pennycress seed meal obtained from wild type pennycress seed is provided herein. In certain embodiments, the ADF content of defatted pennycress seed meal and compositions comprising the same that are provided herein is reduced from about 1.25-, 1.5-, 2-, or 3-fold to about 4-, 5-, 6-, or 7-fold in comparison to control defatted pennycress seed meal and compositions comprising the same obtained from control wild-type pennycress seeds. Typically, the level of acid detergent fiber (ADF) in wild-type pennycress seed varies from about 25 to about 31% by dry weight. Defatted-pennycress meal is a product obtained from high-pressure crushing of seed, via mechanical pressing and/or expanding/extrusion, followed by a solvent extraction process, which removes oil from the whole seed. Solvents used in such extractions include, but are not limited to, hexane or mixed hexanes. The meal is the material that remains after most of the oil has been removed. During a typical oilseed processing procedure, extraction of the oil leads to concentration of fiber as a result of oil mass removal. The typical range of ADF in meal made from wild-type pennycress seed is 35-45%. To be useful as a high protein animal feed, and competitive with other protein feedstuffs, the level of ADF level in meal should be less than 20% by dry weight, less than 15% by dry weight, or less than 10% by dry weight of the meal. In certain embodiments, defatted pennycress seed meal having an ADF content of less than 25% by dry weight, less than 20% by dry weight, less than 15% by dry weight, less than 10% by dry weight, or less than 7% by dry weight of meal is provided herein. In certain embodiments, defatted pennycress seed meal having an ADF content of about 5%, 8%, or 10% to 15%, 18%, 20%, or 25% by dry weight is provided herein. Compositions comprising such defatted pennycress seed meal are also provided herein.

Non-defatted pennycress seed meal having less fiber than non-defatted control pennycress seed meal obtained from wild type pennycress seed is provided herein. In certain embodiments, the ADF content of non-defatted pennycress seed meal and compositions comprising the same that are provided herein is reduced from about 1.25-, 1.5-, 2-, or 3-fold to about 4-, 5-, 6-, or 7-fold in comparison to control non-defatted pennycress seed meal and compositions comprising the same obtained from control wild-type pennycress seeds. In certain embodiments, the non-defatted pennycress seed meal is obtained from pennycress seeds that have been crushed, ground, macerated, expelled, extruded, expanded, or any combination thereof. Typically, the level of acid detergent fiber (ADF) in wild-type pennycress seed and non-defatted seed meal obtained therefrom varies from about 20% to about 38% by dry weight. To be useful as a high protein animal feed, and competitive with other protein feedstuffs, the level of ADF level in non-defatted meal should be less than 20% by dry weight, less than 15% by dry weight, or less than 10% by dry weight of the meal. In certain embodiments, non-defatted pennycress seed meal having an ADF content of less than 20% by dry weight, less than 15% by dry weight, less than 10% by dry weight, or less than 7% by dry weight of the meal is provided herein. In certain embodiments, non-defatted pennycress seed meal having an ADF content of about 5%, 8%, or 10% to 15%, 18%, or 20% by dry weight is provided herein. Compositions comprising such non-defatted pennycress seed meal are also provided herein.

In certain embodiments, pennycress seed lots comprising a population of seed having reduced fiber content, reduced fiber content and increased protein content, reduced fiber content and increased oil content, or reduced fiber content and increased protein and oil content, all in comparison to fiber, protein, and oil content of the control seed lots of wild-type pennycress seed, are provided. In certain embodiments, the seed lots will comprise loss-of-function (LOF) mutations in one or more genes, coding sequences, and/or proteins that result in reduced fiber content, reduced fiber content and increased protein content, reduced fiber content and increased oil content, or reduced fiber content, increased protein, and increased oil content. Such LOF mutations include, but are not limited to, INDELS (insertions, deletions, and/or substitutions or any combination thereof), translocations, inversions, duplications, or any combination thereof in a promoter, a 5′ untranslated region, coding region, an intron of a gene, and/or a 3′ UTR of a gene. Such Indels can introduce one or more mutations including, but not limited to, frameshift mutations, missense mutations, pre-mature translation termination codons, splice donor and/or acceptor mutations, regulatory mutations, and the like that result in an LOF mutation. In certain embodiments, the LOF mutation will result in: (a) a reduction in the enzymatic or other biochemical activity associated with the encoded polypeptide in the plant comprising the LOF mutation in comparison to a wild-type control plant; or (b) both a reduction in the enzymatic or other biochemical activity and a reduction in the amount of a transcript (e.g., mRNA) in the plant comprising the LOF mutation in comparison to a wild-type control plant. Such reductions in activity or activity and transcript levels can, in certain embodiments, comprise a reduction of at least 50%, 60%, 70%, 75%, 80%, 85%, 90%, 95%, 98%, 99%, or 100% of activity or activity and transcript levels in the LOF mutant in comparison to the activity or transcript levels in a wild-type control plant. In certain embodiments, reductions in activity, specific activity, and/or transcript levels are provided by at least one LOF mutation in an endogenous wild-type pennycress gene, promoter, terminator, or protein set forth in Table 1. In certain embodiments, such aforementioned reductions in activity, specific activity and/or transcript levels are provided by at least one LOF mutation in an endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, allelic variants thereof, or any combination thereof. In certain embodiments, such aforementioned reductions in activity, specific activity, and/or transcript levels are provided by at least one LOF mutation in an endogenous wild-type pennycress gene, promoter, or terminator comprising a polynucleotide sequence selected from the group consisting of SEQ ID NO: 69, 71, 75, 77, 87, 88, allelic variants thereof, or any combination thereof. In certain embodiments, any of the aforementioned allelic variants of endogenous wild-type pennycress genes can have at least 80, at least 85, at least 90, at least 95, at least 98, or at least 99 percent sequence identity to SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, or 173. In certain embodiments, such aforementioned reductions in activity, specific activity, and/or transcript levels are provided by at least one LOF mutation in an endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, allelic variants thereof, or any combination thereof. In certain embodiments, such aforementioned reductions in activity or activity and transcript levels are provided by at least one LOF mutation in an endogenous wild-type pennycress gene encoding a polypeptide selected from the group consisting of SEQ ID NO: 70, 76, allelic variants thereof, or any combination thereof. In certain embodiments, an endogenous wild-type pennycress gene can encode a polypeptide allelic variant having at least 80, at least 85, at least 90, at least 95, at least 98, or at least 99 percent sequence identity to SEQ ID NO:2, 7, 10, 13, 16, 19, 22, 25, 28, 31, 34, 37, 40, 43, 46, 49, 52, 55, 58, 61, 64, 67, 70, 73, 76, 79, or 172. In certain embodiments, an endogenous wild-type pennycress gene can encode a polypeptide allelic variant having one or more (e.g., 2, 3, 4, 5, 6, 7, 8, 9, or 10) amino acid modifications (e.g., substitutions) relative to SEQ ID NO:2, 7, 10, 13, 16, 19, 22, 25, 28, 31, 34, 37, 40, 43, 46, 49, 52, 55, 58, 61, 64, 67, 70, 73, 76, 79, or 172. In certain embodiments, the seed lots will comprise one or more transgenes that suppress expression of one or more genes, coding sequences, and/or proteins, thus resulting in reduced fiber content, reduced fiber content and increased protein content, reduced fiber content and increased oil content, or reduced fiber content, increased protein content, and increased oil content, all in comparison to control or wild-type pennycress seed lots. Transgenes that can provide for such suppression include, but are not limited to, transgenes that produce artificial miRNAs targeting a given gene or gene transcript for suppression. In certain embodiments, the transgenes that suppress expression will result in: (a) a reduction in the enzymatic or other biochemical activity associated with the encoded polypeptide in the plant comprising the transgene in comparison to a wild-type control plant; or (b) both a reduction in the enzymatic or other biochemical activity and a reduction in the amount of a transcript (e.g., mRNA) in the plant comprising the transgene in comparison to a wild-type control plant. Such reductions in activity and transcript levels can in certain embodiments comprise a reduction of at least 50%, 60%, 70%, 75%, 80%, 85%, 90%, 95%, 98%, 99%, or 100% of activity and/or transcript levels in the transgenic plant in comparison to the activity or transcript levels in a wild-type control plant. In certain embodiments, certain genes, coding sequences, and/or proteins that can be targeted for introduction of LOF mutations or that are targeted for transgene-mediated suppression are provided in the following Table 1 and accompanying Sequence Listing. In certain embodiments, allelic variants of the wild-type genes, coding sequences, and/or proteins provided in Table 1 and the sequence listing are targeted for introduction of LOF mutations or are targeted for transgene-mediated suppression. Allelic variants found in distinct pennycress isolates or varieties that exhibit wild-type seed fiber, protein, and or oil content can be targeted for introduction of LOF mutations or are targeted for transgene-mediated suppression to obtain seed lots having reduced fiber content, reduced fiber content and increased protein content, reduced fiber content and increased oil content, or reduced fiber content, increased protein, and increased oil content, all in comparison to fiber, protein, and oil content of the control seed lots of wild-type pennycress. Such allelic variants can comprise polynucleotide sequences that have at least 80%, 85%, 90%, 95%, 96%, 97%, 98%, or 99% sequence identity across the entire length of the polynucleotide sequences of the wild-type coding regions or wild-type genes of Table 1 and the sequence listing. Such allelic variants can comprise polypeptide sequences that have at least 80%, 85%, 90%, 95%, 96%, 97%, 98%, or 99% sequence identity across the entire length of the polypeptide sequences of the wild-type proteins of Table 1 and the sequence listing. Pennycress seed lots having reduced seed coat fiber, lighter-colored seed coat due to reduced proanthocyanidins content, increased protein content, and/or higher seed oil content as described herein can comprise one or more LOF mutations in one or more genes that encode polypeptides involved in seed coat and embryo formation or can comprise transgenes that suppress expression of those genes. Polypeptides affecting these traits include, without limitation, TRANSPARENT TESTA1 (TT1) through TRANSPARENT TESTA19 (TT19) (e.g., TT1, TT2, TT3, TT4, TT5, TT6, TT7, TT8, TT9, TT10, TT12, TT13, TT15, TT16, TT18, and TT19), TRANSPARENT TESTA GLABRA1 and 2 (TTG1 and TTG2), GLABROUS 2 (GL2), GLABROUS 3 (GL3), ANR-BAN, and AUTOINHIBITED H+-ATPASE 10 (AHA10) disclosed in Table 1. In certain embodiments, pennycress seed lots provided herein can comprise LOF mutations in any of the aforementioned wild-type pennycress genes disclosed in Table 1 or any combination of mutations disclosed in Table 1. Compositions comprising defatted or non-defatted seed meal obtained from any of the aforementioned seed lots, defatted or non-defatted seed meal obtained from any of the aforementioned seed lots, and seed cakes obtained from any of the aforementioned seed lots are also provided herein. Methods of making any of the aforementioned seed lots, compositions, seed meals, or seed cakes are also provided herein. As used herein, the phrase “seed cake” refers to the material obtained after the seeds are crushed, ground, heated, and expeller pressed or extruded/expanded prior to solvent extraction.

In certain embodiments, reductions or increases in various features of seed lots, seed meal compositions, seed meal, or seed cake are in comparison to a control or wild-type seed lots, seed meal compositions, seed meal, or seed cake. Such controls include, but are not limited to, seed lots, seed meal compositions, seed meal, or seed cake obtained from control plants that lack the LOF mutations or transgene-mediated gene suppression. In certain embodiments, control plants that lack the LOF mutations or transgene-mediated gene suppression will be otherwise isogenic to the plants that contain the LOF mutations or transgene-mediated gene suppression.

In certain embodiments, the controls will comprise seed lots, seed meal compositions, seed meal, or seed cake obtained from plants that lack the LOF mutations or transgene-mediated gene suppression and that were grown in parallel with the plants having the LOF mutations or transgene-mediated gene suppression. Such features that can be compared to wild-type or control plants include, but are not limited to, ADF content, NDF fiber content, protein content, oil content, protein activity and/or transcript levels, and the like.

TABLE 1
Wild-type (WT) coding regions, encoded proteins, and genes that can be targeted
for introduction of LOF mutations or transgene-mediated suppression, their mutant variants
and representative genetic elements for achieving suppression of gene expression.

| | | | | Other Names Used |
|---|---|---|---|---|
| | | | | and Representative |
| | | | | Pennycress LOF |
| SEQ | Sequence | | | Mutants |
| ID NO: | Name | Type | Function/Nature of the mutation | Disclosed Herein |
| 1 | TT2 CDS | WT Coding | R2R3 MYB domain transcription | MYB123, |
| | | region | factor, a key determinant in | TRANSPARENT |
| 2 | TT2 ORF | WT Protein | proanthocyanidin accumulation | TESTA 2 (TT2) |
| 3 | TT2 Ta | WT Gene | | |
| | locus | | | |
| 4 | TT2 CDS- | Mutant Coding | Modified TT2 gene isolated from | tt2-1, tt2-2, BC38, |
| | Mut | region | an EMS-mutagenized population, | E5-547 |
| | | | GAACCATTGGAACTCAAAC | |
| | | | (nt 321-339 of SEQ ID NO: 1) → | |
| | | | GAACCATTGAAACTCAAAC | |
| | | | (nt 321-339 of SEQ ID NO: 4) | |
| 5 | TT2 Mut | Mutant Protein | Truncated protein, due to Trp | |
| | P1 | | (W) codon → Stop mutation | |
| 6 | ATS-KAN4 | WT Coding | Member of the KANADI family of | ABERRANT TESTA |
| | CDS | region | transcription factors, involved | SHAPE, ATS, KAN4, |
| 7 | ATS-KAN4 | WT Protein | in integument formation during | KANADI 4 |
| | ORF | | ovule development and expressed | |
| 8 | ATS-KAN4 | WT Gene | at the boundary between the | |
| | Ta locus | | inner and outer integuments. | |
| | | | Essential for directing laminar | |
| | | | growth of the inner integument | |
| 9 | BAN-ANR | WT Coding | Negative regulator of flavonoid | BAN, BANYULS, |
| | CDS | region | biosynthesis, putative | NAD(P)-binding |
| 10 | BAN-ANR | WT Protein | oxidoreductase. Mutants | Rossmann-fold |
| | ORF | | accumulate flavonoid pigments | superfamily protein |
| 11 | BAN-ANR | WT Gene | in seed coat. Putative ternary | |
| | Ta locus | | complex composed of TT2, TT8 | |
| | | | and TTG1 is believed to be | |
| | | | required for correct expression | |
| | | | of BAN in seed endothelium | |
| 12 | DTX35 CDS | WT Coding | Encodes a multidrug and toxin | Detoxifying Efflux |
| | | region | efflux family transporter. | Carrier 35, FFT, |
| 13 | DTX35 ORF | WT Protein | Involved in flavonoid | FLOWER FLAVONOID |
| 14 | DTX35 Ta | WT Gene | metabolism, affecting root | TRANSPORTER |
| | locus | | growth, seed development and | |
| | | | germination, pollen | |
| | | | development, release and | |
| | | | viability | |
| 15 | GL2 CDS | WT Coding | Glabra 2, a homeodomain protein | Glabra 2, HD-ZIP IV |
| | | region | affects epidermal cell identity | homeobox-leucine |
| 16 | GL2 ORF | WT Protein | including trichomes, root hairs, | zipper protein with |
| 17 | GL2 Ta | WT Gene | and seed coat. Abundantly | lipid-binding START |
| | locus | | expressed during early seed | domain |
| | | | development and in | |
| | | | atrichoblasts. Directly | |
| | | | regulated by WER | |
| 18 | MUM4_like | WT Coding | Encodes a putative NDP-L- | MUCILAGE-MODIFIED 4, |
| | 1 CDS | region | rhamnose synthase, an enzyme | RHAMNOSE |
| 19 | MUM4_like | WT Protein | required for the synthesis of | BIOSYNTHESIS 2, |
| | 1 ORF | | the pectin rhamnogalacturonan I, | RHM2, ATRHM2 |
| 20 | MUM4_like | WT Gene | major component of plant | |
| | 1 Ta locus | | mucilage. Involved in seed coat | |
| 21 | MUM4_like | WT Coding | mucilage cell development. | |
| | 2 CDS | region | Required for complete mucilage | |
| 22 | MUM4_like | WT Protein | synthesis, cytoplasmic | |
| | 2 ORF | | rearrangement and seed coat | |
| 23 | MUM4_like | WT Gene | development | |
| | 2 Ta locus | | | |
| 24 | MYB61 | WT Coding | Putative transcription factor. | MYB DOMAIN PROTEIN |
| | CDS | region | Mutants are deficient in | 61, ATMYB61 |
| 25 | MYB61 | WT Protein | mucilage extrusion from the | |
| | ORF | | seeds during imbibition, | |
| 26 | MYB61 Ta | WT Gene | resulting in reduced deposition | |
| | locus | | of mucilage during development | |
| | | | of the seed coat epidermis in | |
| | | | myb61 mutants | |
| 27 | TT1_like1 | WT Coding | Encodes a zinc finger protein; | WIP DOMAIN PROTEIN |
| | CDS | region | involved in photomorphogenesis, | 1, WIP1 |
| 28 | TT1_like1 | WT Protein | flavonoid biosynthesis, flower | |
| | ORF | | and seed development | |
| 29 | TT1_like1 | WT Gene | | |
| | Ta locus | | | |
| 30 | TT1_like2 | WT Coding | | |
| | CDS | region | | |
| 31 | TT1_like2 | WT Protein | | |
| | ORF | | | |
| 32 | TT1_like2 | WT Gene | | |
| | Ta locus | | | |
| 33 | TT10 CDS | WT Coding | Protein similar to laccase-like | ATLAC15, ATTT10, |
| | | region | polyphenol oxidases, with | LAC15 (LACCASE-LIKE |
| 34 | TT10 ORF | WT Protein | conserved copper binding | 15), TRANSPARENT |
| 35 | TT10 Ta | WT Gene | domains. Involved in lignin and | TESTA 10 (TT10) |
| | locus | | flavonoids biosynthesis. | |
| | | | Expressed in developing testa, | |
| | | | colocalizing with flavonoid end | |
| | | | products proanthocyanidins and | |
| | | | flavonols. Mutants exhibit delay | |
| | | | in developmentally determined | |
| | | | browning of the testa, | |
| | | | characterized by the pale brown | |
| | | | color of seed coat | |
| 36 | TT12 CDS | WT Coding | Proton antiporter, involved in | TRANSPARENT TESTA 12 |
| | | region | the transportation of | (TT12), ATTT12, |
| 37 | TT12 ORF | WT Protein | proanthocyanidin precursors | MATE efflux family |
| 38 | TT12 Ta | WT Gene | into the vacuole. Loss-of- | protein |
| | locus | | function mutation has strong | |
| | | | reduction of proanthocyanidin | |
| | | | deposition in vacuoles and | |
| | | | reduced dormancy. Expressed in | |
| | | | the endothelium of ovules and | |
| | | | in developing seeds | |
| 39 | TT13 CDS | WT Coding | Proton pump from the H⁺-ATPase | AHA10 |
| | | region | family, involved in | (AUTOINHIBITED H(+)- |
| 40 | TT13 ORF | WT Protein | proanthocyanidin biosynthesis. | ATPASE ISOFORM 10), |
| 41 | TT13 Ta | WT Gene | Mutations disturb vacuolar | TRANSPARENT TESTA |
| | locus | | biogenesis and acidification | 13 (TT13) |
| | | | process. The acidification of | |
| | | | the vacuole provides energy for | |
| | | | import of proanthocyanidins into | |
| | | | the vacuole | |
| 42 | TT15 CDS | WT Coding | Encodes a UDP-glucose:sterol- | TRANSPARENT TESTA 15 |
| | | region | glucosyltransferase. Mutants | (TT15), TRANSPARENT |
| 43 | TT15 ORF | WT Protein | produce pale greenish-brown | TESTA GLABROUS 15 |
| 44 | TT15 Ta | WT Gene | seeds with slightly reduced | (TTG15), UGT80B1, |
| | locus | | dormancy | UDP- |
| | | | | Glycosyltransferase |
| | | | | superfamily protein |
| 45 | TT16 CDS | WT Coding | MADS-box protein regulating | ABS, AGAMOUS-LIKE 32 |
| | | region | proanthocyanidin biosynthesis | (AGL32), ARABIDOPSIS |
| 46 | TT16 ORF | WT Protein | and cell shape in the inner- | BSISTER, TRANSPARENT |
| 47 | TT16 Ta | WT Gene | most cell layer of the seed | TESTA16 (TT16) |
| | locus | | coat. Required for determining | |
| | | | the identity of the endothelial | |
| | | | layer within the ovule. | |
| | | | Paralogous to GOA. Plays a | |
| | | | maternal role in fertilization | |
| | | | and seed development | |
| 48 | TT18 CDS | WT Coding | Encodes leucoanthocyanidin | ANS, ANTHOCYANIDIN |
| | | region | dioxygenase, which is involved | SYNTHASE, LDOX, |
| 49 | TT18 ORF | WT Protein | in proanthocyanin biosynthesis. | LEUCOANTHOCYANIDIN |
| 50 | TT18 Ta | WT Gene | Mutant analysis suggests that | DIOXYGENASE, TANNIN |
| | locus | | this gene is also involved in | DEFICIENT SEED 4 |
| | | | vacuole formation | (TDS4), TT18 |
| 51 | TT19 CDS | WT Coding | Encodes glutathione transferase | GLUTATHIONE S- |
| | | region | belonging to the phi class of | TRANSFERASE PHI 12, |
| 52 | TT19 ORF | WT Protein | GSTs. Mutants display no | ATGSTF12,GLUTATHIONE |
| 53 | TT19 Ta | WT Gene | pigments in the leaves or | S-TRANSFERASE 26 |
| | locus | | stems. Likely to function as a | (GST26), GLUTATHIONE |
| | | | carrier to transport anthocyanin | S-TRANSFERASE PHI |
| | | | from the cytosol to tonoplasts | 12, GSTF12, |
| | | | | TRANSPARENT |
| | | | | TESTA 19 (TT19) |
| 54 | TT3 CDS | WT Coding | Dihydroflavonol reductase. | DFR, DIHYDROFLAVONOL |
| | | region | Catalyzes conversion of | 4-REDUCTASE, M318, |
| 55 | TT3 ORF | WT Protein | dihydroquercetin to | TRANSPARENT TESTA |
| 56 | TT3 Ta | WT Gene | leucocyanidin in the | 3, (TT3) |
| | locus | | biosynthesis of anthocyanins | |
| 57 | TT4 CDS | WT Coding | Encodes chalcone synthase (CHS), | ATCHS, CHALCONE |
| | | region | a key enzyme in biosynthesis of | SYNTHASE, CHS, |
| 58 | TT4 ORF | WT Protein | flavonoids. Required for | TRANSPARENT TESTA |
| 59 | TT4 Ta | WT Gene | accumulation of purple | 4 (TT4) |
| | locus | | anthocyanins in leaves, stems | |
| | | | and seed coat. Also involved in | |
| | | | regulation of auxin transport | |
| | | | and root gravitropism | |
| 60 | TT5 CDS | WT Coding | Another key enzyme in | A11, ATCHI, CFI, |
| | | region | biosynthesis of flavonoids. | CHALCONE FLAVANONE |
| 61 | TT5 ORF | WT Protein | Catalyzes the conversion of | ISOMERASE, CHALCONE |
| 62 | TT5 Ta | WT Gene | chalcones into flavanones. | ISOMERASE, CHI, |
| | locus | | Required for the accumulation | TRANSPARENT |
| | | | of purple anthocyanins leaves, | TESTA 5 (TT5) |
| | | | stems and seed coat. Co- | |
| | | | expressed with CHS | |
| 63 | TT6 CDS | WT Coding | Encodes flavanone 3-hydroxylase, | F3′H, F3H, FLAVANONE |
| | | region | regulating flavonoid | 3-HYDROXYLASE, |
| 64 | TT6 ORF | WT Protein | biosynthesis. Coordinately | TRANSPARENT |
| 65 | TT6 Ta | WT Gene | expressed with chalcone synthase | TESTA 6 (TT6) |
| | locus | | and chalcone isomerases | |
| 66 | TT7 CDS | WT Coding | Required for flavonoid 3′- | F3′H CYP75B1, |
| | | region | hydroxylase activity. Enzyme | CYTOCHROME P450 |
| 67 | TT7 ORF | WT Protein | abundance relative to CHS | 75B1, D501, |
| 68 | TT7 Ta | WT Gene | determines Quercetin/Kaempferol | TRANSPARENT |
| | locus | | metabolite ratio | TESTA 7 (TT7) |
| 69 | TT8 CDS | WT Coding | TT8 is a transcription factor | ATTT8, BHLH42, |
| | | region | acting in concert with TT1, PAP1 | TRANSPARENT |
| 70 | TT8 ORF | WT Protein | and TTG1 on regulation of | TESTA 8, (TT8) |
| 71 | TT8 Ta | WT Gene | flavonoid pathways, namely | |
| | locus | | proanthocyanidin and anthocyanin | |
| | | | biosynthesis. Affects | |
| | | | dihydroflavonol 4-reductase gene | |
| | | | expression. It is believed that | |
| | | | a ternary complex composed of | |
| | | | TT2, TT8 and TTG1 is required | |
| | | | for correct expression of BAN in | |
| | | | seed endothelium. Interacts with | |
| | | | JAZ proteins to regulate | |
| | | | anthocyanin accumulation | |
| 72 | TT9 CDS | WT Coding | Encodes a peripheral membrane | GFS9, GREEN |
| | | region | protein localized at the Golgi | FLUORESCENT |
| 73 | TT9 ORF | WT Protein | apparatus. Involved in membrane | SEED 9, TRANSPARENT |
| 74 | TT9 Ta | WT Gene | trafficking, vacuole development | TESTA 9, TT9 |
| | locus | | and in flavonoid accumulation in | CLEC16A-like protein |
| | | | the seed coat. Mutant seed color | |
| | | | is pale brown | |
| 75 | TTG1 CDS | WT Coding | Part of a ternary complex | TTG1, TTG, URM23, |
| | | region | composed of TT2, TT8 and TTG1 | ATTTG1, Transducin/ |
| 76 | TTG1 ORF | WT Protein | necessary for correct expression | WD40-repeat- |
| 77 | TTG1 Ta | WT Gene | of BAN in seed endothelium. | containing protein |
| | locus | | Required for the accumulation | |
| | | | of purple anthocyanins in | |
| | | | leaves, stems and seed coat. | |
| | | | Controls epidermal cell fate | |
| | | | specification. Affects | |
| | | | dihydroflavonol 4-reductase gene | |
| | | | expression. TTG1 was shown to | |
| | | | act non-cell autonomously and | |
| | | | to move via plasmodesmata | |
| | | | between cells | |
| 78 | TTG2 CDS | WT Coding | Belongs to a family of WRKY | TRANSPARENT TESTA |
| | | region | transcription factors expressed | GLABRA 2 (TTG2), |
| 79 | TTG2 ORF | WT Protein | in seed integument and | AtWRKY44, DSL1 |
| 80 | TTG2 Ta | WT Gene | endosperm. Mutants are defective | (DR. STRANGELOVE 1) |
| | locus | | in proanthocyanidin synthesis | |
| | | | and seed mucilage deposition. | |
| | | | Seeds are yellow colored. Seed | |
| | | | size is also affected; seeds are | |
| | | | reduced in size but only when | |
| | | | the mutant allele is transmitted | |
| | | | through the female parent | |
| 81 | TT1 | Artificial | Artificial micro-RNA designed to | |
| | aMIR319a | miRNA | reduce expression of TT1 in | |
| | gene | | corresponding cell layer of | |
| | | | developing seed coat | |
| 82 | TT10 | Artificial | Artificial micro-RNA designed to | |
| | aMIR319a | miRNA | reduce expression of TT10 in | |
| | gene | | corresponding cell layer of | |
| | | | developing seed coat | |
| 83 | TT2 | Artificial | Artificial micro-RNA designed to | |
| | aMIR319a | miRNA | reduce expression of TT2 in | |
| | gene | | corresponding cell layer of | |
| | | | developing seed coat | |
| 84 | TT8 | Artificial | Artificial micro-RNA designed to | |
| | aMIR319a | miRNA | reduce expression of TT8 in | |
| | gene | | corresponding cell layer of | |
| | | | developing seed coat | |
| 85 | TT1 | Promoter | Genomic region of TT1 locus | |
| | Promoter | | upstream of TT1 start codon | |
| | | | containing TT1 promoter | |
| | | | regulatory elements | |
| 86 | TT1 | Transcriptional | Genomic region of TT1 locus | |
| | Terminator | terminator | downstream of TT1 stop codon | |
| | | | containing regulatory elements | |
| 87 | TT8 | Promoter | Genomic region of TT8 locus | |
| | Promoter | | upstream of TT8 start codon | |
| | | | containing TT8 promoter | |
| | | | regulatory elements | |
| 88 | TT8 | Transcriptional | Genomic region of TT8 locus | |
| | Terminator | terminator | downstream of TT8 stop codon | |
| | | | containing regulatory elements | |
| 89 | TT2_CRISPR- | Oligo- | TT2 CDS targeted for cleavage by | |
| | SpCAS9_F1 | nucleotide | SpCAS9 enzyme; part of gRNA | |
| | | | cassette | |
| 90 | TT2_CRISPR- | Oligo- | TT2 CDS targeted for cleavage by | |
| | SpCAS9_R1 | nucleotide | SpCAS9 enzyme; part of gRNA | |
| | | | cassette | |
| 91 | TT2_CRISPR- | Oligo- | TT2 CDS targeted for cleavage by | |
| | SaCAS9_F2 | nucleotide | SpCAS9 enzyme; part of gRNA | |
| | | | cassette | |
| 92 | TT2_CRISPR- | Oligo- | TT2 CDS targeted for cleavage by | |
| | SaCAS9_R2 | nucleotide | SpCAS9 enzyme; part of gRNA | |
| | | | cassette | |
| 93 | TT2_CRISPR- | Oligo- | TT2 CDS targeted for cleavage by | |
| | SaCAS9_F3 | nucleotide | SpCAS9 enzyme; part of gRNA | |
| | | | cassette | |
| 94 | TT2_CRISPR- | Oligo- | TT2 CDS targeted for cleavage by | |
| | SaCAS9_R3 | nucleotide | SpCAS9 enzyme; part of gRNA | |
| | | | cassette | |
| 95 | TT8_CRISPR- | Oligo- | TT8 CDS targeted for cleavage by | |
| | SpCAS9_F1 | nucleotide | SpCAS9 enzyme; part of gRNA | |
| | | | cassette | |
| 96 | TT8_CRISPR- | Oligo- | TT8 CDS targeted for cleavage by | |
| | SpCAS9_R1 | nucleotide | SpCAS9 enzyme; part of gRNA | |
| | | | cassette | |
| 97 | TT8_CRISPR- | Oligo- | TT8 CDS targeted for cleavage by | |
| | SpCAS9_F2 | nucleotide | SpCAS9 enzyme; part of gRNA | |
| | | | cassette | |
| 98 | TT8_CRISPR- | Oligo- | TT8 CDS targeted for cleavage by | |
| | SpCAS9_R2 | nucleotide | SpCAS9 enzyme; part of gRNA | |
| | | | cassette | |
| 99 | TT8_CRISPR- | Oligo- | TT8 CDS targeted for cleavage by | |
| | SpCAS9_F3 | nucleotide | SpCAS9 enzyme; part of gRNA | |
| | | | cassette | |
| 100 | TT8_CRISPR- | Oligo- | TT8 CDS targeted for cleavage by | |
| | SpCAS9_R3 | nucleotide | SpCAS9 enzyme; part of gRNA | |
| | | | cassette | |
| 101 | TT10_CRISPR- | Oligo- | TT10 CDS targeted for cleavage | |
| | SaCAS9_F1 | nucleotide | by SpCAS9 enzyme; part of gRNA | |
| | | | cassette | |
| 102 | TT10_CRISPR- | Oligo- | TT10 CDS targeted for cleavage | |
| | SaCAS9_R1 | nucleotide | by SpCAS9 enzyme; part of gRNA | |
| | | | cassette | |
| 103 | TT10_CRISPR- | Oligo- | TT10 CDS targeted for cleavage | |
| | SaCAS9_F2 | nucleotide | by SpCAS9 enzyme; part of gRNA | |
| | | | cassette | |
| 104 | TT10_CRISPR- | Oligo- | TT10 CDS targeted for cleavage | |
| | SaCAS9_R2 | nucleotide | by SpCAS9 enzyme; part of gRNA | |
| | | | cassette | |
| 105 | TT16_CRISPR- | Oligo- | TT16 CDS targeted for cleavage | |
| | SpCAS9_F1 | nucleotide | by SpCAS9 enzyme; part of gRNA | |
| | | | cassette | |
| 106 | TT16_CRISPR- | Oligo- | TT16 CDS targeted for cleavage | |
| | SpCAS9_R1 | nucleotide | by SpCAS9 enzyme; part of gRNA | |
| | | | cassette | |
| 107 | TT16_CRISPR- | Oligo- | TT16 CDS targeted for cleavage | |
| | SpCAS9_F2 | nucleotide | by SpCAS9 enzyme; part of gRNA | |
| | | | cassette | |
| 108 | TT16_CRISPR- | Oligo- | TT16 CDS targeted for cleavage | |
| | SpCAS9_R2 | nucleotide | by SpCAS9 enzyme; part of gRNA | |
| | | | cassette | |
| 109 | TT8_CRISPR- | Oligo- | TT8 CDS targeted for cleavage by | |
| | SpCAS9_F4 | nucleotide | SpCAS9 enzyme; part of gRNA | |
| | | | cassette | |
| 110 | TT8_CRISPR- | Oligo- | TT8 CDS targeted for cleavage by | |
| | SpCAS9_F5 | nucleotide | SpCAS9 enzyme; part of gRNA | |
| | | | cassette | |
| 111 | TT8_CRISPR- | Oligo- | TT8 CDS targeted for cleavage by | |
| | SaCAS9_F1 | nucleotide | SaCAS9 enzyme; part of gRNA | |
| | | | cassette | |
| 112 | TT8_CRISPR- | Oligo- | TT8 CDS targeted for cleavage by | |
| | SaCAS9_F2 | nucleotide | SaCAS9 enzyme; part of gRNA | |
| | | | cassette | |
| 113 | TTG1_CRISPR- | Oligo- | TTG1 CDS targeted for cleavage | |
| | SpCAS9_F1 | nucleotide | by SpCAS9 enzyme; part of gRNA | |
| | | | cassette | |
| 114 | TTG1_CRISPR- | Oligo- | TTG1 CDS targeted for cleavage | |
| | SpCAS9_F2 | nucleotide | by SpCAS9 enzyme; part of gRNA | |
| | | | cassette | |
| 115 | TTG1_CRISPR- | Oligo- | TTG1 CDS targeted for cleavage | |
| | SaCAS9_F1 | nucleotide | by SaCAS9 enzyme; part of gRNA | |
| | | | cassette | |
| 116 | TTG1_CRISPR- | Oligo- | TTG1 CDS targeted for cleavage | |
| | SaCAS9_F2 | nucleotide | by SaCAS9 enzyme; part of gRNA | |
| | | | cassette | |
| 117 | TT4-1 CDS- | Mutant Coding | GTCTGCTCCGAGATCACAG | tt4-1, A7-95 |
| | Mut | region | (nt 580-598 of SEQ ID NO: 57) → | |
| | | | GTCTGCTCCAAGATCACAG | |
| | | | (nt 580-598 of SEQ ID NO: 117) | |
| 118 | TT4 Mut P1 | Mutant Protein | Presumed LOF due to E→K aa | |
| | | | change | |
| 119 | TT4-2 CDS- | Mutant Coding | AAGTGACTGGAACTCTCTC | tt4-2, E5-549 |
| | Mut | region | (nt 894-912 of SEQ ID NO: 57) → | |
| | | | AAGTGACTGAAACTCTCTC | |
| | | | (nt 894-912 of SEQ ID NO: 119) | |
| 120 | TT4 Mut P2 | Mutant Protein | Truncated protein, W→Stop | |
| | | | change | |
| 121 | TT6-1 CDS- | Mutant Coding | GAGACTGTGCAAGATTGGA | tt6-1, AX17 |
| | Mut | region | (nt 364-382 of SEQ ID NO: 63) → | |
| | | | GAGACTGTGTAAGATTGGA | |
| | | | (nt 364-382 of SEQ ID NO: 121) | |
| 122 | TT6 Mut P1 | Mutant Protein | Truncated protein, Q→Stop | |
| | | | change | |
| 123 | TT6-2 CDS- | Mutant Coding | TTCAGAATCCGGCGCAGGA | tt6-2, Q36 |
| | Mut | region | (nt 872-890 of SEQ ID: 63) → | |
| | | | TTCAGAATCTGGCGCAGGA | |
| | | | (nt 872-890 of SEQ ID: 123) | |
| 124 | TT6 Mut P2 | Mutant Protein | Presumed LOF due to P→L aa | |
| | | | change | |
| 125 | TT7-1 CDS- | Mutant Coding | CCAAATTCAGGAGCCAAAC | tt7-1, A7-3, E5-586, |
| | Mut | region | (nt 304-322 of SEQ ID: 66) → | E5-484 P15, E5-484 |
| | | | CCAAATTCAAGAGCCAAAC | P5 |
| | | | (nt 304-322 of SEQ ID: 125) | |
| 126 | TT7-1 Mut | Mutant Protein | Presumed LOF due to G→R aa | |
| | P1 | | change | |
| 127 | TT8-1 CDS- | Mutant Coding | TTTACGGCAGAGAAAGTGA | tt8-1, D3-N10 P5 |
| | Mut | region | (nt 19-37 of SEQ ID: 69) → | |
| | | | TTTACGGCAAAGAAAGTGA | |
| | | | (nt 19-37 of SEQ ID: 127) | |
| 128 | TT8 Mut P1 | Mutant Protein | Presumed LOF due to E→K aa | |
| | | | change | |
| 129 | TT8-2 CDS- | Mutant Coding | TCTTACATCCAATCATCAT | tt8-2, D5-191, D3- |
| | Mut | region | (nt 940-958 of SEQ ID: 69) → | N25P1, E5-590, |
| | | | TCTTACATCTAATCATCAT | A7-191 |
| | | | (nt 940-958 of SEQ ID: 129) | |
| 130 | TT8 Mut P2 | Mutant Protein | Truncated protein, Q→Stop | |
| | | | change | |
| 131 | TT8-3 CDS- | Mutant Coding | TGCCACATGGAAGGCTGAT | tt8-3, I0193, E5- |
| | Mut | region | (nt 960-978 of SEQ ID: 69) → | 542, E5-548 |
| | | | TGCCACATGAAAGGCTGAT | |
| | | | (nt 960-978 of SEQ ID: 131) | |
| 132 | TT8 Mut P3 | Mutant Protein | Truncated protein, W→Stop | |
| | | | change | |
| 133 | TT8-11 | Mutant Coding | GCAATAAAGACGAGGAAGA | tt8-11 |
| | CDS-Mut | region | (nt 172-190 of SEQ ID: 69) → | |
| | | | GCAATAAAGAACGAGGAAGA | |
| | | | (nt 172-191 of SEQ ID: 133) | |
| 134 | TT8 Mut P4 | Mutant Protein | Frameshift caused by 1 bp | |
| | | | insertion | |
| 135 | TT8-12 | Mutant Coding | GCAATAAAGACGAGGAAGA | tt8-12 |
| | CDS-Mut | region | (nt 172-190 of SEQ ID: 69) → | |
| | | | GCAATAAA--CGAGGAAGA | |
| | | | (nt 172-188 of SEQ ID: 135) | |
| 136 | TT8 Mut P5 | Mutant Protein | Frameshift caused by 2 bp | |
| | | | deletion | |
| 137 | TT8-13 | Mutant Coding | GCAATAAAGACGAGGAAGA | tt8-13 |
| | CDS-Mut | region | (nt 172-190 of SEQ ID: 69) → | |
| | | | GCAATAAAGGACGAGGAAGA | |
| | | | (nt 172-191 of SEQ ID: 137) | |
| 138 | TT8 Mut P6 | Mutant Protein | Frameshift caused by 1 bp | |
| | | | insertion | |
| 139 | TT10-1 | Mutant Coding | GACTGTTTGGTGGCATGCG | tt10-1, E5-539, |
| | CDS-Mut | region | (nt 354-372 of SEQ ID: 33) → | E5-543 |
| | | | GACTGTTTGATGGCATGCG | |
| | | | (nt 354-372 of SEQ ID: 139) | |
| 140 | TT10 Mut | Mutant Protein | Truncated protein, W→Stop | |
| | P1 | | change | |
| 141 | TT10-2 | Mutant Coding | TACCGCATTCGGATGGTAA | tt10-2, E5-545 |
| | CDS-Mut | region | (nt 646-664 of SEQ ID: 33) → | |
| | | | TACCGCATTTGGATGGTAA | |
| | | | (nt 646-664 of SEQ ID: 141) | |
| 142 | TT10 Mut | Mutant Protein | Presumed LOF due to R→W aa | |
| | P2 | | change | |
| 143 | TT10-11 | Mutant Coding | GGACCAGTGTTAAGGGCT | tt10-11 |
| | CDS-Mut | region | (nt 154-171 of SEQ ID: 33) → | |
| | | | GGACCAGTGTTTAAGGGCT | |
| | | | (nt 154-172 of SEQ ID: 143) | |
| 144 | TT10 Mut | Mutant Protein | Frameshift caused by 1 bp | |
| | P3 | | insertion | |
| 145 | TT10-12 | Mutant Coding | GGACCAGTGTTAAGGGCT | tt10-12 |
| | CDS-Mut | region | (nt 154-171 of SEQ ID: 33) → | |
| | | | GGACCAGTGATTAAGGGCT | |
| | | | (nt 154-172 of SEQ ID: 145) | |
| 146 | TT10 Mut | Mutant Protein | Frameshift caused by 1 bp | |
| | P4 | | insertion | |
| 147 | TT10-13 | Mutant Coding | TCCTGGACCAGTGTTAAGG | tt10-13 |
| | CDS-Mut | region | (nt 150-168 of SEQ ID: 33) → | |
| | | | TCCTGG--------TTAAGG | |
| | | | (nt 150-161 of SEQ ID: 147) | |
| 148 | TT10 Mut | Mutant Protein | Frameshift caused by 7 bp | |
| | P5 | | deletion | |
| 149 | TT12-1 | Mutant Coding | AACCCTTTGGCTTACATGTC | tt12-1, A7-261 |
| | CDS-Mut | region | (nt 604-623 of SEQ ID: 36) → | |
| | | | AACCCTTT----TACATGTC | |
| | | | (nt 604-619 of SEQ ID: 149) | |
| 150 | TT12 Mut | Mutant Protein | Frameshift caused by 4 bp | |
| | P1 | | deletion | |
| 151 | TT12-2 | Mutant Coding | ATTCTCTCTGGTGTTGCCA | tt12-2, J22 |
| | CDS-Mut | region | (nt 1237-1255 of SEQ ID: 36) → | |
| | | | ATTCTCTCTAGTGTTGCCA | |
| | | | (nt 1237-1255 of SEQ ID: 151) | |
| 152 | TT12 Mut | Mutant Protein | Presumed LOF due to G→S aa | |
| | P2 | | change | |
| 153 | TT13-1 | Mutant Coding | GCTCTTAACCTTGGAGTTT | tt13-1, aha10-1, J22 |
| | CDS-Mut | region | (nt 895-913 of SEQ ID: 39) → | |
| | | | GCTCTTAACTTTGGAGTTT | |
| | | | (nt 895-913 of SEQ ID: 153) | |
| 154 | TT13 Mut | Mutant Protein | Truncated protein, L→F change | |
| | P1 | | | |
| 155 | TT13-2 | Mutant Coding | ACAGGAAGGCGACTTGGGA | tt13-2, P32 |
| | CDS-Mut | region | (nt 958-976 of SEQ ID: 39) → | |
| | | | ACAGGAAGGTGACTTGGGA | |
| | | | (nt 958-976 of SEQ ID: 155) | |
| 156 | TT13 Mut | Mutant Protein | Truncated protein, R→Stop | |
| | P2 | | change | |
| 157 | TT13-3 | Mutant Coding | GGAATGACCGGAGATGGTG | tt13-3, E5-540 |
| | CDS-Mut | region | (nt 1144-1162 of SEQ ID: 39) → | |
| | | | GGAATGACCAGAGATGGTG | |
| | | | (nt 1144-1162 of SEQ ID: 157) | |
| 158 | TT13 Mut | Mutant Protein | Truncated protein, G→R change | |
| | P3 | | | |
| 159 | TT16-1 | Mutant Coding | TACTTGAAGACCAGTGGAAT | tt16-1 |
| | CDS-Mut | region | (nt 211-230 of SEQ ID: 45) → | |
| | | | TACTTGAAGACCCAGTGGAAT | |
| | | | (nt 211-231 of SEQ ID: 159) | |
| 160 | TT16 Mut | Mutant Protein | Frameshift caused by 1 bp | |
| | P1 | | insertion | |
| 161 | TT16-2 | Mutant Coding | TACTTGAAGACCAGTGGAAT | tt16-2 |
| | CDS-Mut | region | (nt 211-230 of SEQ ID: 45) → | |
| | | | TACTTGAAGACGCAGTGGAAT | |
| | | | (nt 211-231 of SEQ ID: 161) | |
| 162 | TT16 Mut | Mutant Protein | Frameshift caused by 1 bp | |
| | P2 | | insertion | |
| 163 | TT16-3 | Mutant Coding | TACTTGAAGACCAGTGGAAT | tt16-3 |
| | CDS-Mut | region | (nt 211-230 of SEQ ID: 45) → | |
| | | | TACTTGAAGACTCAGTGGAAT | |
| | | | (nt 211-231 of SEQ ID: 163) | |
| 164 | TT16 Mut | Mutant Protein | Frameshift caused by 1 bp | |
| | P3 | | insertion | |
| 165 | TTG1 CDS- | Mutant Coding | GATCTCCTCGCTTCCTCCGGCGATTTCCT | Y1067, Y1126 |
| | Mut | region | (nt 286-314 of SEQ ID: 75) → | |
| | | | GATC---------------------TCCT | |
| | | | (nt 286-293 of SEQ ID: 165) | |
| 166 | TTG1 Mut | Mutant Protein | LOF caused by 21 bp/7 aa | |
| | P1 | | deletion | |
| 167 | TTG1-1 | Mutant Coding | TCGCTTCCTCCGGCGATTT | ttg1-1, E5-544 |
| | CDS-Mut | region | (nt 293-311 of SEQ ID: 75) → | |
| | | | TCGCTTCCTTCGGCGATTT | |
| | | | (nt 293-311 of SEQ ID: 167) | |
| 168 | TTG1 Mut | Mutant Protein | Presumed LOF due to S→F aa | |
| | P2 | | change | |
| 169 | TTG1-2 | Mutant Coding | TCGCTTGGGGAGAAGCTAG | ttg1-2, A7-187 |
| | CDS-Mut | region | (nt 542-560 of SEQ ID: 75) → | |
| | | | TCGCTTGGGAAGAAGCTAG | |
| | | | (nt 542-560 of SEQ ID: 169) | |
| 170 | TTG1 Mut | Mutant Protein | Presumed LOF due to G→E aa | |
| | P3 | | change | |
| 171 | GL3 CDS | WT Coding | Transcription activator of bHLH | GL3, MYC6.2 basic |
| | | region | superfamily involved in cell | helix-loop-helix |
| 172 | GL3 ORF | WT Protein | fate specification. In | protein |
| 173 | GL3 Ta | WT Gene | association with TTG1, promotes | |
| | locus | | trichome formation. Together | |
| | | | with MYB75/PAP1, plays a role | |
| | | | in the activation of anthocyanin | |
| | | | biosynthesis. Activates the | |
| | | | transcription of GL2. | |
| 174 | GL3-1 CDS- | Mutant Coding | CAACTTAGGGAGCTTTACG | gl3-1, E5-541, E5- |
| | Mut | region | (nt 241-259 of SEQ ID: 171) → | 559 |
| | | | CAACTTAGGAAGCTTTACG | |
| | | | (nt 241-259 of SEQ ID: 174) | |
| 175 | GL3 Mut P1 | Mutant Protein | Presumed LOF due to E→K aa | |
| | | | change | |
| 176 | GL3-2 CDS- | Mutant Coding | GCCGACACAGAGTGGTACT | gl3-2, A7-92, E5- |
| | Mut | region | (nt 358-376 of SEQ ID: 171) → | 444 |
| | | | GCCGACACAAAGTGGTACT | |
| | | | (nt 358-376 of SEQ ID: 176) | |
| 177 | GL3 Mut P2 | Mutant Protein | Presumed LOF due to E→K aa | |
| | | | change | |
| 178 | GL3-3 CDS- | Mutant Coding | GGTTTAACTGATAATTTAA | gl3-3, A7-229, E5- |
| | Mut | region | (nt 1663-1681 of SEQ ID: 171) → | 582 |
| | | | GGTTTAACTAATAATTTAA | |
| | | | (nt 1663-1681 of SEQ ID: 178) | |
| 179 | GL3 Mut P3 | Mutant Protein | Presumed LOF due to D→N aa | |
| | | | change | |
| 180 | BAN-1 | Mutant Coding | ATCAAGCCAGGGATACAAG | ban-1, BJ8, BJ8D |
| | CDS-Mut | region | (nt 319-337 of SEQ ID: 9) → | |
| | | | ATCAAGCCAAGGATACAAG | |
| | | | (nt 319-337 of SEQ ID: 9 and | |
| | | | SEQ ID: 180) | |
| 181 | BAN Mut | Mutant Protein | Presumed LOF due to G→R aa | |
| | P1 | | change | |
| 182 | TT4-3 CDS- | Mutant Coding | CTCACCCTGGAGGTCCTGC | tt4-3, A7-229, E5- |
| | Mut | region | (nt 923-941 of SEQ ID: 57) → | 582 |
| | | | CTCACCCTGAAGGTCCTGC | |
| | | | (nt 923-941 of SEQ ID: 182) | |
| 183 | TT4-3 Mut | Mutant Protein | Presumed LOF due to G→R aa | |
| | P1 | | change | |

In certain embodiments, pennycress plants having reduced seed coat fiber, lighter-colored seed coat, and/or higher seed oil content as described herein can be from the Y1067, Y1126, BC38, BJ8, P32, J22, Q36, BD24, AX17, E5-444, E5-540, E5-541, E5-542, E5-543, E5-544, E5-545, E5-547, E5-549, E5-582, E5-586, D3-N10 P5, D5-191, A7-95, A7-187, or A7-261 variant lines provided herein, or can be progeny derived from those lines.

A representative wild-type (WT) pennycress TT2 coding sequence is as shown in sequence listing (SEQ ID NO:1). In certain embodiments, a WT pennycress TT2 coding sequence can have a sequence that deviates from the coding sequence set forth above (e.g., SEQ ID NO: 1), and is referred to as an allelic variant sequence. In certain embodiments, a TT2 coding sequence allelic variant can have at least 80, at least 85, at least 90, at least 95, at least 98, or at least 99 percent sequence identity to SEQ ID NO: 1. A representative wild-type pennycress TT2 polypeptide is shown in sequence listing (SEQ ID NO:2). In certain embodiments, a WT pennycress TT2 polypeptide can have a sequence that deviates from the polypeptide sequence set forth above (SEQ ID NO:2) and is referred to as an allelic variant sequence.

In certain embodiments, a WT pennycress TT2 polypeptide can have a sequence that deviates from the polypeptide sequence set forth above (SEQ ID NO:2), referred to herein as an allelic variant sequence, provided the polypeptide maintains its wild-type function. For example, a TT2 polypeptide can have at least 80, at least 85, at least 90, at least 95, at least 98, or at least 99) percent sequence identity to SEQ ID NO:2. A TT2 polypeptide of an allelic variant can have one or more (e.g., 2, 3, 4, 5, 6, 7, 8, 9, or 10) amino acid modifications (e.g., substitutions) relative to SEQ ID NO:2.

In certain embodiments, pennycress seed lots having reduced seed coat fiber, lighter-colored seed coat due to reduced proanthocyanidins content, and/or higher seed oil content as described herein can include at least one loss-of-function modification in a TT2 gene (e.g., in a TT2 coding sequence, in a TT2 regulatory sequence including the promoter, 5′ UTR, intron, 3′ UTR, or in any combination thereof) or a transgene that suppresses expression of the TT2 gene. As used herein, a loss-of-function mutation in a TT2 gene can be any modification that is effective to reduce TT2 polypeptide expression or TT2 polypeptide function. In certain embodiments, reduced TT2 polypeptide expression and/or TT2 polypeptide function can be eliminated or reduced in comparison to a wild-type plant. Examples of genetic modifications that can provide for a loss-of-function mutation include, without limitation, deletions, insertions, substitutions, translocations, inversions, duplications, or any combination thereof.

In certain embodiments, pennycress seed lots having reduced seed coat fiber, lighter-colored seed coat, and/or higher seed oil and/or protein content as described herein can include a substitution (e.g., a single base-pair substitution) relative to the WT pennycress TT2 coding sequence. In certain embodiments, a modified TT2 coding sequence can include a single base-pair substitution of the cytosine (G) at nucleotide residue 330 in a WT pennycress TT2 coding sequence (e.g., SEQ ID NO: 1 or an allelic variant thereof). The G at nucleotide residue 330 can be substituted with any appropriate nucleotide (e.g., thymine (T), adenine (A), or cytosine (C)). For example, a single base-pair substitution can be a G to A substitution at nucleotide residue 330 in a WT pennycress TT2 coding sequence thereby producing a premature stop codon. A representative modified pennycress TT2 coding sequence having a loss-of-function single base pair substitution is presented in SEQ ID NO:4.

A modified pennycress TT2 coding sequence having a loss-of-function single base pair substitution (e.g., SEQ ID NO:4) can encode a modified TT2 polypeptide (e.g., a modified TT2 polypeptide having reduced TT2 polypeptide expression and/or reduced TT2 polypeptide function). For example, a modified pennycress TT2 coding sequence having a single base-pair substitution (e.g., SEQ ID NO:4) can encode a modified TT2 polypeptide. In certain embodiments, a modified TT2 polypeptide can include a truncation resulting from the introduction of a stop codon at codon position 110 within the TT2 open reading frame (e.g., SEQ ID NO: 4). A representative truncated pennycress TT2 polypeptide is presented in SEQ ID NO:5. Representative pennycress varieties having a mutation in the TT2 gene include the tt2-1, tt2-2, BC38, and E5-547 varieties.

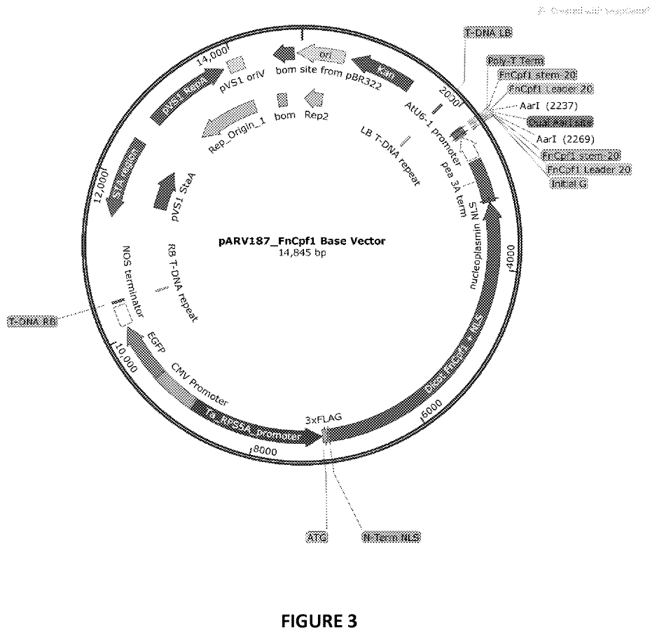

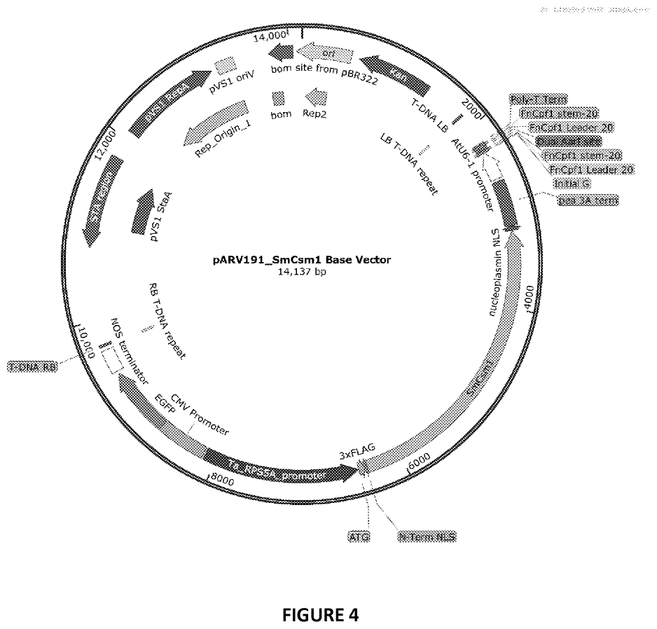

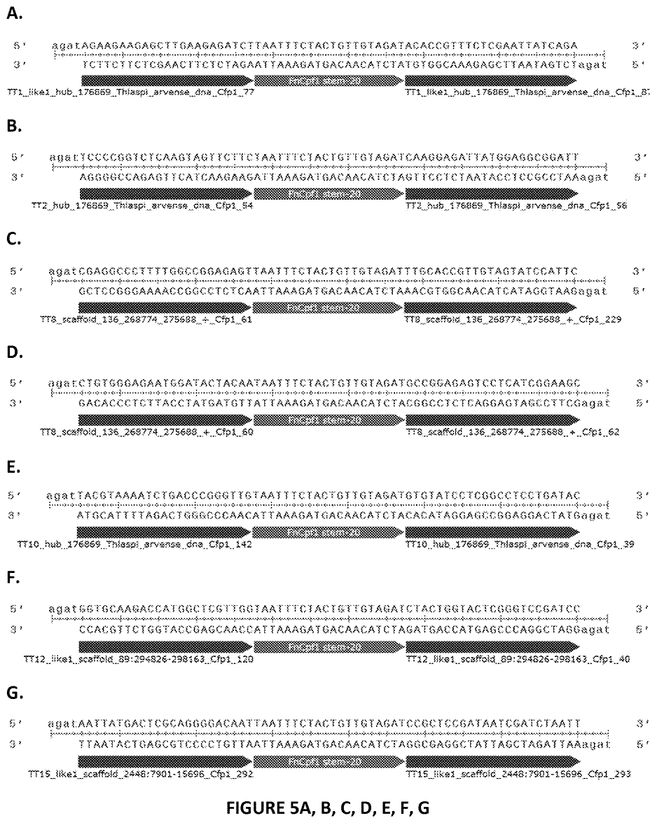

A representative WT pennycress TRANSPARENT TESTA8 (TT8) coding region is presented in SEQ ID NO:69. Two protospacer locations and adjacent protospacer-adjacent motif (PAM) sites that can be targeted by, for example, CRISPR-SpCAS9 correspond to nucleotides 164-183 and 287-306 (protospacers) or 184-186 and 284-286 (PAM sites). In another embodiment, two separate examples of alternative protospacer locations and adjacent protospacer-adjacent motifs (PAM) sites are provided in FIGS. 3-5. In each case, two protospacer locations can be targeted by, for example, CRISPR-FnCpf1, CRISPR-SmCsm1 or a similar enzyme, correspond to nucleotides 175-153 and 261-283 (protospacers) or 179-176 and 257-260 (PAM sites); and nucleotides 145-167 and 274-296 (protospacers) or 141-144 and 270-273 (PAM sites), all of SEQ ID NO:69.

In certain embodiments, a WT pennycress TT8 coding sequence can have a sequence that deviates from the coding sequence set forth above (e.g., SEQ ID NO:69), and is referred to as an allelic variant sequence. In certain embodiments, a TT8 coding sequence allelic variant can have at least 80, at least 85, at least 90, at least 95, at least 98, or at least 99 percent sequence identity to SEQ ID NO:69. A representative WT pennycress TT8 polypeptide is presented in SEQ ID NO:70.

In certain embodiments, a WT pennycress TT8 polypeptide can have a sequence that deviates from the polypeptide sequence set forth above (SEQ ID NO:70) and is referred to as an allelic variant sequence. For example, a TT8 polypeptide can have at least 80, at least 85, at least 90, at least 95, at least 98, or at least 99 percent sequence identity to SEQ ID NO:70. ATT8 polypeptide can have one or more (e.g., 2, 3, 4, 5, 6, 7, 8, 9, or 10) amino acid modifications (e.g., substitutions) relative to SEQ ID NO:70.

In certain embodiments, pennycress seed lots having reduced fiber content as described herein can include a loss-of-function modification in a TT8 gene (e.g., in a TT8 coding sequence) or a transgene that suppresses expression of the TT8 gene. As used herein, a loss-of-function mutation in a TT8 gene can be any modification that is effective to reduce TT8 polypeptide expression or TT8 polypeptide function. In certain embodiments, reduced TT8 polypeptide expression and/or TT8 polypeptide function can be eliminated or reduced. Examples of genetic modifications include, without limitation, deletions, insertions, substitutions, translocations, inversions, duplications, and any combination thereof. Representative TT8 gene mutations include the mutations shown in SEQ ID NO:127, 129, 131, 133, 135, and 137 that result in the TT8 mutant polypeptides of SEQ ID NO:128, 130, 132, 134, 136, and 138, respectively. Representative pennycress varieties with TT8 gene mutations include the tt4-2 tt8-1, tt8-2, tt8-3, tt8-11, tt8-12, tt8-12, tt8-13, 10193, E5-542, E5-548, D5-191, D3-N25P1, E5-590, A7-191, and D3-N10 P5 varieties.

In certain embodiments, a WT pennycress TT1 coding sequence can have a sequence that deviates from the coding sequence set forth above (e.g., SEQ ID NO:27 or 30), and is referred to as an allelic variant sequence. In certain embodiments, a TT1 coding sequence allelic variant can have at least 80, at least 85, at least 90, at least 95, at least 98, or at least 99 percent sequence identity to SEQ ID NO:27 or 30. In certain embodiments, a WT pennycress TT1 polypeptide can have a sequence that deviates from the polypeptide sequence set forth above (SEQ ID NO:28 or 31), and is referred to as an allelic variant sequence. For example, a TT1 polypeptide allelic variant can have at least 80, at least 85, at least 90, at least 95, at least 98, or at least 99 percent sequence identity to SEQ ID NO:28 or 31. A TT1 polypeptide allelic variant can have one or more (e.g., 2, 3, 4, 5, 6, 7, 8, 9, or 10) amino acid modifications (e.g., substitutions) relative to SEQ ID NO:28 or 31.

In certain embodiments, pennycress seed lots having reduced fiber as described herein can include a loss-of-function modification in a TT1 encoding gene or a transgene that suppresses expression of the TT1 gene. As used herein, a loss-of-function mutation in a TT1 gene can be any modification that is effective to reduce TT1 polypeptide expression or TT1 polypeptide function. In certain embodiments, reduced TT1 polypeptide expression and/or TT1 polypeptide function can be eliminated or reduced. Examples of genetic modifications include, without limitation, deletions, insertions, substitutions, translocations, inversions, duplications, and any combination thereof.

In certain embodiments, pennycress seed lots having reduced fiber as described herein can include a loss-of-function modification in a TT1 encoding gene, a promoter thereof, or a terminator, thereof, or a transgene that suppresses expression of the TT1 gene. As used herein, a loss-of-function mutation in a TT1 gene can be any modification that is effective to reduce TT1 polypeptide expression or TT1 polypeptide function. In certain embodiments, reduced TT1 polypeptide expression and/or TT1 polypeptide function can be eliminated or reduced. Examples of genetic modifications include, without limitation, deletions, insertions, substitutions, translocations, inversions, duplications, and any combination thereof.

In certain embodiments, a WT pennycress TT4 coding sequence can have a sequence that deviates from the coding sequence set forth above (e.g., SEQ ID NO:57), and is referred to as an allelic variant sequence. In certain embodiments, a TT4 coding sequence allelic variant can have at least 80, at least 85, at least 90, at least 95, at least 98, or at least 99 percent sequence identity to SEQ ID NO:57. In certain embodiments, a WT pennycress TT4 polypeptide can have a sequence that deviates from the polypeptide sequence set forth above (SEQ ID NO:58), and is referred to as an allelic variant sequence. For example, a TT4 polypeptide allelic variant can have at least 80, at least 85, at least 90, at least 95, at least 98, or at least 99 percent sequence identity to SEQ ID NO:58. A TT4 polypeptide allelic variant can have one or more (e.g., 2, 3, 4, 5, 6, 7, 8, 9, or 10) amino acid modifications (e.g., substitutions) relative to SEQ ID NO:58.

In certain embodiments, pennycress seed lots having reduced fiber as described herein can include a loss-of-function modification in a TT4 encoding gene or a transgene that suppresses expression of the TT4 gene. As used herein, a loss-of-function mutation in a TT4 gene can be any modification that is effective to reduce TT4 polypeptide expression or TT4 polypeptide function. In certain embodiments, reduced TT4 polypeptide expression and/or TT4 polypeptide function can be eliminated or reduced. Examples of genetic modifications include, without limitation, deletions, insertions, substitutions, translocations, inversions, duplications, and any combination thereof. Representative TT4 gene mutations include the mutation shown in SEQ ID NO: 119 that results in the truncated TT4 mutant polypeptide of SEQ ID NO:120. Representative TT4 gene mutations also include the mutations shown in SEQ ID NO: 117 and 182 that result in the TT4 mutant polypeptides of SEQ ID NO: 118 and 183, respectively. Representative pennycress varieties with TT4 gene mutations include the tt4-1, tt4-2, tt4-3, A7-229, E5-582 and E5-549 varieties.

In certain embodiments, a WT pennycress TT5, TT9, TT15, TT18, or TT19 coding sequence can have a sequence that deviates from the coding sequence set forth above (e.g., SEQ ID NO: 60, 72, 42, 48, or 51, respectively), and is referred to as an allelic variant sequence. In certain embodiments, a TT5, TT9, TT15, TT18, or TT19 coding sequence allelic variant can have at least 80, at least 85, at least 90, at least 95, at least 98, or at least 99 percent sequence identity to SEQ ID NO:60, 72, 42, 48, or 51, respectively. In certain embodiments, a WT pennycress TT5, TT9, TT15, TT18, or TT19 polypeptide can have a sequence that deviates from the polypeptide sequence set forth above (SEQ ID NO:61, 73, 43, 49, or 52, respectively), and is referred to as an allelic variant sequence. For example, a TT5, TT9, TT15, TT18, or TT19 polypeptide allelic variant can have at least 80, at least 85, at least 90, at least 95, at least 98, or at least 99 percent sequence identity to SEQ ID NO:61, 73, 43, 49, or 52, respectively. A TT5, TT9, TT15, TT18, or TT19 polypeptide allelic variant can have one or more (e.g., 2, 3, 4, 5, 6, 7, 8, 9, or 10) amino acid modifications (e.g., substitutions) relative to SEQ ID NO:61, 73, 43, 49, or 52, respectively.

In certain embodiments, pennycress seed lots having reduced fiber as described herein can include a loss-of-function modification in a TT5, TT9, TT15, TT18, or TT19 encoding gene or a transgene that suppresses expression of the TT5, TT9, TT15, TT18, or TT19 gene. As used herein, a loss-of-function mutation in a TT5 gene can be any modification that is effective to reduce TT5, TT9, TT15, TT18, or TT19 polypeptide expression or TT5, TT9, TT15, TT18, or TT19 polypeptide function. In certain embodiments, TT5, TT9, TT15, TT18, or TT19 polypeptide expression and/or TT5, TT9, TT15, TT18, or TT19 polypeptide function can be eliminated or reduced. Examples of genetic modifications include, without limitation, deletions, insertions, substitutions, translocations, inversions, duplications, and any combination thereof.

In certain embodiments, a WT pennycress TT6 coding sequence can have a sequence that deviates from the coding sequence set forth above (e.g., SEQ ID NO:63), and is referred to as an allelic variant sequence. In certain embodiments, a TT6 coding sequence allelic variant can have at least 80, at least 85, at least 90, at least 95, at least 98, or at least 99 percent sequence identity to SEQ ID NO:63. In certain embodiments, a WT pennycress TT6 polypeptide can have a sequence that deviates from the polypeptide sequence set forth above (SEQ ID NO:64), and is referred to as an allelic variant sequence. For example, a TT6 polypeptide allelic variant can have at least 80, at least 85, at least 90, at least 95, at least 98, or at least 99 percent sequence identity to SEQ ID NO:64. A TT6 polypeptide allelic variant can have one or more (e.g., 2, 3, 4, 5, 6, 7, 8, 9, or 10) amino acid modifications (e.g., substitutions) relative to SEQ ID NO:64.

In certain embodiments, pennycress seed lots having reduced fiber as described herein can include a loss-of-function modification in a TT6 encoding gene or a transgene that suppresses expression of the TT6 gene. As used herein, a loss-of-function mutation in a TT6 gene can be any modification that is effective to reduce TT6 polypeptide expression or TT6 polypeptide function. In certain embodiments, reduced TT6 polypeptide expression and/or TT6 polypeptide function can be eliminated or reduced. Examples of genetic modifications include, without limitation, deletions, insertions, substitutions, translocations, inversions, duplications, and any combination thereof. Representative TT6 gene mutations include the mutation shown in SEQ ID NO:121 that results in the TT6 mutant polypeptide of SEQ ID NO:122. Representative pennycress varieties with TT6 gene mutations mutants include the tt6-1 and AX17 varieties. Representative TT6 gene mutations also include the mutation shown in SEQ ID NO:123 that results in the TT6 mutant polypeptide of SEQ ID NO:124. Representative pennycress varieties with TT6 gene mutations mutants also include the tt6-1, tt6-2 and Q36 varieties.

In certain embodiments, a WT pennycress TT7 coding sequence can have a sequence that deviates from the coding sequence set forth above (e.g., SEQ ID NO:66), and is referred to as an allelic variant sequence. In certain embodiments, a TT7 coding sequence allelic variant can have at least 80, at least 85, at least 90, at least 95, at least 98, or at least 99 percent sequence identity to SEQ ID NO:66. In certain embodiments, a WT pennycress TT7 polypeptide can have a sequence that deviates from the polypeptide sequence set forth above (SEQ ID NO:67), and is referred to as an allelic variant sequence. For example, a TT7 polypeptide allelic variant can have at least 80, at least 85, at least 90, at least 95, at least 98, or at least 99 percent sequence identity to SEQ ID NO:67. A TT7 polypeptide allelic variant can have one or more (e.g., 2, 3, 4, 5, 6, 7, 8, 9, or 10) amino acid modifications (e.g., substitutions) relative to SEQ ID NO:67.

In certain embodiments, pennycress seed lots having reduced fiber as described herein can include a loss-of-function modification in a TT7 encoding gene or a transgene that suppresses expression of the TT7 gene. As used herein, a loss-of-function mutation in a TT7 gene can be any modification that is effective to reduce TT7 polypeptide expression or TT7 polypeptide function. In certain embodiments, reduced TT7 polypeptide expression and/or TT7 polypeptide function can be eliminated or reduced. Examples of genetic modifications include, without limitation, deletions, insertions, substitutions, translocations, inversions, duplications, and any combination thereof. Representative TT7 gene mutations include the mutation shown in SEQ ID NO:125 that results in the TT7 mutant polypeptide of SEQ ID NO:126. Representative pennycress varieties with TT7 gene mutations include the tt7-1, A7-3, E5-586, E5-484 P15, and E5-484 P5 varieties.

In certain embodiments, a WT pennycress TTG1 coding sequence can have a sequence that deviates from the coding sequence set forth above (e.g., SEQ ID NO:75), and is referred to as an allelic variant sequence. In certain embodiments, a TTG1 coding sequence allelic variant can have at least 80, at least 85, at least 90, at least 95, at least 98, or at least 99 percent sequence identity to SEQ ID NO:75. In certain embodiments, a WT pennycress TTG1 polypeptide can have a sequence that deviates from the polypeptide sequence set forth above (SEQ ID NO:76), and is referred to as an allelic variant sequence. For example, a TTG1 polypeptide allelic variant can have at least 80, at least 85, at least 90, at least 95, at least 98, or at least 99 percent sequence identity to SEQ ID NO:28 or 31. A TTG1 polypeptide allelic variant can have one or more (e.g., 2, 3, 4, 5, 6, 7, 8, 9, or 10) amino acid modifications (e.g., substitutions) relative to SEQ ID NO: 76.

In certain embodiments, pennycress seed lots having reduced fiber as described herein can include a loss-of-function (LOF) modification in a TTG1 encoding gene or a transgene that suppresses expression of the TTG1 gene. As used herein, a loss-of-function mutation in a TTG1 gene can be any modification that is effective to reduce TTG1 polypeptide expression or TTG1 polypeptide function. In certain embodiments, reduced TTG1 polypeptide expression and/or TTG1 polypeptide function can be eliminated or reduced. Examples of genetic modifications include, without limitation, deletions, insertions, substitutions, translocations, inversions, duplications, and any combination thereof. In certain embodiments, a LOF mutation in a TTG1 gene can comprise a 21 bp deletion in the TTG1 coding sequence as shown in SEQ ID NO:165. In other embodiments, a LOF mutation in a TTG1 gene can comprise ttg1-1 and ttg1-2 mutant alleles having single nucleotide substitutions that result in the substitution of a conserved amino acid residue in the TTG protein (SEQ ID NOs: 167-170). Representative TTG1 gene mutations thus include the mutations shown in SEQ ID NO: 165, 167, and 169 that result in the TTG1 mutant polypeptides of SEQ ID NO:166, 1268, and 170, respectively. Representative pennycress varieties with TTG1 gene mutations include the Y1067, Y1126, ttg1-1, E5-544, ttg1-2, and A7-187 varieties.

In certain embodiments, a WT pennycress TT10 coding sequence can have a sequence that deviates from the coding sequence set forth above (e.g., SEQ ID NO:33), and is referred to as an allelic variant sequence. In certain embodiments, a TT10 coding sequence allelic variant can have at least 80, at least 85, at least 90, at least 95, at least 98, or at least 99 percent sequence identity to SEQ ID NO:33. In certain embodiments, a WT pennycress TT10 polypeptide can have a sequence that deviates from the polypeptide sequence set forth above (SEQ ID NO:34), and is referred to as an allelic variant sequence. For example, a TT10 polypeptide allelic variant can have at least 80, at least 85, at least 90, at least 95, at least 98, or at least 99 percent sequence identity to SEQ ID NO:34. A TT10 polypeptide allelic variant can have one or more (e.g., 2, 3, 4, 5, 6, 7, 8, 9, or 10) amino acid modifications (e.g., substitutions) relative to SEQ ID NO:34.

In certain embodiments, pennycress seed lots having reduced fiber as described herein can include a loss-of-function modification in a TT10 encoding gene or a transgene that suppresses expression of the TT10 gene. As used herein, a loss-of-function mutation in a TT10 gene can be any modification that is effective to reduce TT10 polypeptide expression or TT10 polypeptide function. In certain embodiments, reduced TT10 polypeptide expression and/or TT10 polypeptide function can be eliminated or reduced. Examples of genetic modifications include, without limitation, deletions, insertions, substitutions, translocations, inversions, duplications, and any combination thereof.

In certain embodiments, pennycress seed lots having reduced fiber as described herein can include a loss-of-function modification in a TT10 encoding gene or a transgene that suppresses expression of the TT10 gene. As used herein, a loss-of-function mutation in a TT10 gene can be any modification that is effective to reduce TT10 polypeptide expression or TT10 polypeptide function. In certain embodiments, reduced TT10 polypeptide expression and/or TT10 polypeptide function can be eliminated or reduced. Examples of genetic modifications include, without limitation, deletions, insertions, substitutions, translocations, inversions, duplications, and any combination thereof. Representative TT10 gene mutations include the mutations shown in SEQ ID NO:139, 141, 143, 145, or 147 that result in the TT10 mutant polypeptides of SEQ ID NO: 140, 142, 144, 146, or 148, respectively. Representative pennycress varieties with TT10 gene mutations include the tt10-1, tt10-2, tt10-1, tt10-12, tt10-13, E5-539, E5-543, and E5-545 varieties.

In certain embodiments, a WT pennycress TT12 coding sequence can have a sequence that deviates from the coding sequence set forth above (e.g., SEQ ID NO:36), and is referred to as an allelic variant sequence. In certain embodiments, a TT12 coding sequence allelic variant can have at least 80, at least 85, at least 90, at least 95, at least 98, or at least 99 percent sequence identity to SEQ ID NO:36. In certain embodiments, a WT pennycress TT12 polypeptide can have a sequence that deviates from the polypeptide sequence set forth above (SEQ ID NO:37), and is referred to as an allelic variant sequence. For example, a TT12 polypeptide allelic variant can have at least 80, at least 85, at least 90, at least 95, at least 98, or at least 99 percent sequence identity to SEQ ID NO:37. A TT12 polypeptide allelic variant can have one or more (e.g., 2, 3, 4, 5, 6, 7, 8, 9, or 10) amino acid modifications (e.g., substitutions) relative to SEQ ID NO:37.

In certain embodiments, pennycress seed lots having reduced fiber as described herein can include a loss-of-function modification in a TT12 encoding gene or a transgene that suppresses expression of the TT12 gene. As used herein, a loss-of-function mutation in a TT12 gene can be any modification that is effective to reduce TT12 polypeptide expression or TT12 polypeptide function. In certain embodiments, reduced TT12 polypeptide expression and/or TT12 polypeptide function can be eliminated or reduced. Examples of genetic modifications include, without limitation, deletions, insertions, substitutions, translocations, inversions, duplications, and any combination thereof.

In certain embodiments, pennycress seed lots having reduced fiber as described herein can include a loss-of-function modification in a TT12 encoding gene or a transgene that suppresses expression of the TT12 gene. As used herein, a loss-of-function mutation in a TT12 gene can be any modification that is effective to reduce TT12 polypeptide expression or TT12 polypeptide function. In certain embodiments, reduced TT12 polypeptide expression and/or TT12 polypeptide function can be eliminated or reduced. Examples of genetic modifications include, without limitation, deletions, insertions, substitutions, translocations, inversions, duplications, and any combination thereof. Representative TT12 gene mutations include the mutations shown in SEQ ID NO: 149 or 151 that result in the TT12 mutant polypeptides of SEQ ID NO: 150 or 152, respectively. Representative pennycress varieties with TT12 gene mutations include the tt12-1, tt12-2, A7-261, and J22 varieties.

In certain embodiments, a WT pennycress TT13 coding sequence can have a sequence that deviates from the coding sequence set forth above (e.g., SEQ ID NO:39), and is referred to as an allelic variant sequence. In certain embodiments, a TT13 coding sequence allelic variant can have at least 80, at least 85, at least 90, at least 95, at least 98, or at least 99 percent sequence identity to SEQ ID NO:39. In certain embodiments, a WT pennycress TT13 polypeptide can have a sequence that deviates from the polypeptide sequence set forth above (SEQ ID NO:40), and is referred to as an allelic variant sequence. For example, a TT13 polypeptide allelic variant can have at least 80, at least 85, at least 90, at least 95, at least 98, or at least 99 percent sequence identity to SEQ ID NO:40. A TT13 polypeptide allelic variant can have one or more (e.g., 2, 3, 4, 5, 6, 7, 8, 9, or 10) amino acid modifications (e.g., substitutions) relative to SEQ ID NO:40.

In certain embodiments, pennycress seed lots having reduced fiber as described herein can include a loss-of-function modification in a TT13 encoding gene or a transgene that suppresses expression of the TT13 gene. As used herein, a loss-of-function mutation in a TT13 gene can be any modification that is effective to reduce TT13 polypeptide expression or TT13 polypeptide function. In certain embodiments, reduced TT13 polypeptide expression and/or TT13 polypeptide function can be eliminated or reduced. Examples of genetic modifications include, without limitation, deletions, insertions, substitutions, translocations, inversions, duplications, and any combination thereof. Representative TT13 gene mutations include the mutations shown in SEQ ID NO:153, 155, or 157 that result in the TT13 mutant polypeptides of SEQ ID NO:154, 156, or 158, respectively. Representative pennycress varieties with TT13 gene mutations include the tt13-1, tt13-2, tt13-3, aha10-1, J22, and P32 E5-540 varieties.

In certain embodiments, a WT pennycress TT16 coding sequence can have a sequence that deviates from the coding sequence set forth above (e.g., SEQ ID NO:45), and is referred to as an allelic variant sequence. In certain embodiments, a TT16 coding sequence allelic variant can have at least 80, at least 85, at least 90, at least 95, at least 98, or at least 99 percent sequence identity to SEQ ID NO:45. In certain embodiments, a WT pennycress TT16 polypeptide can have a sequence that deviates from the polypeptide sequence set forth above (SEQ ID NO:46), and is referred to as an allelic variant sequence. In certain embodiments, a TT16 polypeptide allelic variant can have at least 80, at least 85, at least 90, at least 95, at least 98, or at least 99 percent sequence identity to SEQ ID NO:46. A TT16 polypeptide allelic variant can have one or more (e.g., 2, 3, 4, 5, 6, 7, 8, 9, or 10) amino acid modifications (e.g., substitutions) relative to SEQ ID NO:46.

In certain embodiments, pennycress seed lots having reduced fiber as described herein can include a loss-of-function modification in a TT16 encoding gene or a transgene that suppresses expression of the TT16 gene. As used herein, a loss-of-function mutation in a TT16 gene can be any modification that is effective to reduce TT16 polypeptide expression or TT16 polypeptide function. In certain embodiments, reduced TT16 polypeptide expression and/or TT16 polypeptide function can be eliminated or reduced. Examples of genetic modifications include, without limitation, deletions, insertions, substitutions, translocations, inversions, duplications, and any combination thereof.

In certain embodiments, pennycress seed lots having reduced fiber as described herein can include a loss-of-function modification in a TT16 encoding gene or a transgene that suppresses expression of the TT16 gene. As used herein, a loss-of-function mutation in a TT16 gene can be any modification that is effective to reduce TT16 polypeptide expression or TT16 polypeptide function. In certain embodiments, reduced TT16 polypeptide expression and/or TT16 polypeptide function can be eliminated or reduced. Examples of genetic modifications include, without limitation, deletions, insertions, substitutions, translocations, inversions, duplications, and any combination thereof. Representative TT16 gene mutations include the mutations shown in SEQ ID NO:159, 161, or 163 that result in the TT16 mutant polypeptides of SEQ ID NO:160, 162, or 164, respectively. Representative pennycress varieties with TT16 gene mutations include the tt16-1, tt16-2, and tt16-3 varieties.

In certain embodiments, a genome editing system such as a CRISPR-Cas9 system can be used to introduce one or more loss-of-function mutations into genes such as the TRANSPARENT TESTA (TT) and related genes provided herewith in Table 1 and the sequence listing that are associated with agronomically-relevant seed traits including reduced seed coat fiber, lighter-colored seed coat due to reduced proanthocyanidins content, increased protein content, and/or higher seed oil content. For example, a CRISPR-Cas9 vector can include at least one guide sequence specific to a pennycress TT2 sequence (see, e.g., SEQ ID NO:1) and/or at least one guide sequence specific to a pennycress TT8 sequence (see, e.g., SEQ ID NO:5). A Cas9 enzyme will bind to and cleave within the gene when the target site is followed by a PAM sequence. For example, the canonical SpCAS9 PAM site is the sequence 5′-NGG-3′, where Nis any nucleotide followed by two guanine (G) nucleotides. The Cas9 component of a CRISPR-Cas9 system designed to introduce one or more loss-of-function modifications described herein can be any appropriate Cas9. In certain embodiments, the Cas9 of a CRISPR-Cas9 system described herein can be a Streptococcus pyogenes Cas9 (SpCas9). One example of an SpCas9 is described in (Fauser et al., 2014).

In certain embodiments, a WT pennycress GL3 coding sequence can have a sequence that deviates from the coding sequence set forth above (e.g., SEQ ID NO:171), and is referred to as an allelic variant sequence. In certain embodiments, a GL3 coding sequence allelic variants can have at least 80, at least 85, at least 90, at least 95, at least 98, or at least 99 percent sequence identity to SEQ ID NO:171. In certain embodiments, a WT pennycress GL3 polypeptide can have a sequence that deviates from the polypeptide sequence set forth above (SEQ ID NO:172), and is referred to as an allelic variant sequence. For example, a GL3 polypeptide allelic variant can have at least 80, at least 85, at least 90, at least 95, at least 98, or at least 99 percent sequence identity to SEQ ID NO:160. A GL3 polypeptide allelic variant can have one or more (e.g., 2, 3, 4, 5, 6, 7, 8, 9, or 10) amino acid modifications (e.g., substitutions) relative to SEQ ID NO:172.

In certain embodiments, pennycress seed lots having reduced fiber as described herein can include a loss-of-function modification in a GL3 encoding gene or a transgene that suppresses expression of the GL3 gene. As used herein, a loss-of-function mutation in a GL3 gene can be any modification that is effective to reduce GL3 polypeptide expression or GL3 polypeptide function. In certain embodiments, GL3 polypeptide expression and/or GL3 polypeptide function can be eliminated or reduced. Examples of genetic modifications include, without limitation, deletions, insertions, substitutions, translocations, inversions, duplications, and any combination thereof. In certain embodiments, the GL3 mutation can comprise the coding sequence mutations of SEQ ID NO:174, 176, 178 and/or the protein sequence mutation of SEQ ID NO:175, 177, 180. Representative pennycress varieties with GL3 gene mutations include the gl3-1, gl3-2, gl3-3, E5-541, E5-559, A7-92, E5-444, A7-229, and E5-582 varieties.

In certain embodiments, a WT pennycress BAN-ANR (or BAN) coding sequence can have a sequence that deviates from the coding sequence set forth above (e.g., SEQ ID NO:9), and is referred to as an allelic variant sequence. In certain embodiments, a BAN coding sequence allelic variant can have at least 80, at least 85, at least 90, at least 95, at least 98, or at least 99 percent sequence identity to SEQ ID NO:9. In certain embodiments, a WT pennycress BAN polypeptide can have a sequence that deviates from the polypeptide sequence set forth above (SEQ ID NO:10), and is referred to as an allelic variant sequence. For example, a BAN polypeptide allelic variant can have at least 80, at least 85, at least 90, at least 95, at least 98, or at least 99 percent sequence identity to SEQ ID NO:10. A BAN polypeptide allelic variant can have one or more (e.g., 2, 3, 4, 5, 6, 7, 8, 9, or 10) amino acid modifications (e.g., substitutions) relative to SEQ ID NO:10.

In certain embodiments, pennycress seed lots having reduced fiber as described herein can include a loss-of-function modification in a BAN encoding gene or a transgene that suppresses expression of the BAN gene. As used herein, a loss-of-function mutation in a BAN gene can be any modification that is effective to reduce BAN polypeptide expression and/or BAN polypeptide function. In certain embodiments, BAN polypeptide expression and/or BAN polypeptide function can be eliminated or reduced. Examples of genetic modifications include, without limitation, deletions, insertions, substitutions, translocations, inversions, duplications, and any combination thereof. In certain embodiments, the BAN mutation can comprise the coding sequence mutation of SEQ ID NO:180 and/or the protein sequence mutation of SEQ ID NO: 181. Representative pennycress varieties with BAN gene mutations include the ban-1, BJ8, and BJ8D varieties.

In certain embodiments, pennycress seeds or seed lots having reduced fiber, as well as pennycress seed meal obtained therefrom (including both defatted and non-defatted seed meal), as described herein can include a loss-of-function mutation in more than one of the genes or coding sequences set forth in Table 1. In certain embodiments, pennycress seeds or seed lots having reduced fiber can have a LOF mutation in the gene(s) and/or coding sequences of any combination of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and/or any allelic variants thereof. In certain embodiments, pennycress seed meal, including de-fatted and non-defatted forms) and having reduced fiber can comprise a detectable amount of any combination of nucleic acids having a LOF mutation in the gene(s) and/or coding sequences of any combination of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and/or any allelic variants thereof.

The LOF mutations in any of the genes or coding sequences of Table 1 can be introduced by a variety of methods. Methods for introduction of the LOF mutations include, but are not limited to, traditional mutagenesis (e.g., with EMS or other mutagens), TILLING, meganucleases, zinc finger nucleases, transcription activator-like effector nucleases, clustered regularly interspaced short palindromic repeat (CRISPR)-associated nuclease (e.g., S. pyogenes Cas9 and its variants, S. aureus Cas9 and its variants, eSpCas9, Cpf1, Cms1 and their variants) targetrons, and the like. Various tools that can be used to introduce mutations into genes have been disclosed in Guha et al. Comput Struct Biotechnol J. 2017; 15:146-160. Methods for modifying genomes by use of Cpf1 or Csm1 nucleases are disclosed in US Patent Application Publication 20180148735, which is incorporated herein by reference in its entirety, and can be adapted for introduction of the LOF mutations disclosed herein. Methods for modifying genomes by use of CRISPR/CAS systems are disclosed in US Patent Application Publication 20180179547, which is incorporated herein by reference in its entirety, and can be adapted for introduction of the LOF mutations disclosed herein. The genome editing reagents described herein can be introduced into a pennycress plant by any appropriate method. In certain embodiments, nucleic acids encoding the genome editing reagents can be introduced into a plant cell using Agrobacterium or Ensifer mediated transformation, particle bombardment, liposome delivery, nanoparticle delivery, electroporation, polyethylene glycol (PEG) transformation, or any other method suitable for introducing a nucleic acid into a plant cell. In certain embodiments, the Site-Specific Nuclease (SSN) or other expressed gene editing reagents can be delivered as RNAs or as proteins to a plant cell and the RT, if one is used, can be delivered as DNA.

The disclosure will be further described in the following examples, which do not limit the scope of the disclosure described in the claims.

### EXAMPLES

### Example 1: Meal Made from Wild Type Pennycress Plants is High in Fiber, but Low in Metabolizable Energy

Higher dietary fiber results in lower net energy for swine (Kil et al., 2013) and poultry (Meloche et al., 2013). It was also reported that hemicellulose displayed the strongest correlation with apparent metabolizable energy (AMEn), followed by neutral detergent fiber (NDF), total dietary fiber (TDF), and crude fiber (CF) in broilers fed corn co-products (Rochelle et al., 2011). Thus, a reduction in fiber will result in increased available energy to pigs and poultry.

When comparing mechanically expeller-pressed meals made from two USDA-developed pennycress varieties (Beecher and Ruby II) to mechanically expeller-pressed canola meal, the various fiber fractions when analyzed as crude fiber (CF), acid detergent fiber (ADF), neutral detergent fiber (NDF) and total dietary fiber (TDF) were 1.5-2 times the levels in canola meal (Table 2). Similar levels were observed when comparing different lots of pennycress meal with canola meal (Table 3). Analysis conducted by Arvegenix at University of Georgia showed similar results (Table 4).

TABLE 2
Nutrient composition of mechanically expeller-pressed canola
and pennycress meals produced at Dairyland by Arvegenix in
August 2015. All numbers are in percent dry weight (% DW).

| | Expeller- | Pennycress | Pennycress |
|---|---|---|---|
| Meal | Pressed | Meal | Meal |
| Constituent | Canola Meal | (Beecher) | (Ruby II) |
| Crude Protein | 38.7 | 31.3 | 31.1 |
| Either extract | 11.2 | 10.1 | 10.6 |
| Crude fiber | 10.9 | 27.1 | 27.9 |
| ADF | 18.1 | 35.6 | 33.8 |
| NDF | 22.7 | 40.5 | 36.8 |
| Total | 29.5 | 43.3 | 37.8 |
| Dietary Fiber | | | |

TABLE 3
Lot variation in proximate values in mechanically expeller-pressed pennycress
meal, composite mechanically expeller-pressed pennycress meal blend (all produced
by Arvegenix), and commercially available mechanically expeller-pressed canola
(ME Canola). All numbers represent the average of duplicate analytical runs
for mean and standard error measured in percent dry weight (% DW).

| | Meal Constituent Processing Date(s) | | | | | |
|---|---|---|---|---|---|---|
| | | | | | | ME |
| | Lot 1 | Lot 2 | Lot 3 | Lot 4 | Blend* | Canola |
| | 22 Jul. 2015 | 23 Jul. 2015 | 23 Jul. 2015 | 23 Jul. 2015 | 22-27 Jul. 2015 | N/A |
| Moisture (% FW) | 2.12 ± 0.08 | 6.10 ± 0.1 | 5.20 ± 0.01 | 4.06 ± 0.08 | 3.36 ± 0.05 | 4.41 ± 0.13 |
| Ash Content | 7.32 ± 0.06 | 7.24 ± 0.1 | 7.13 ± 0.01 | 7.17 ± 0.02 | 5.62 ± 2.38 | 6.88 ± 0.02 |
| Carbohydrates | 51.4 ± 0.07 | 50.9 ± 0.7 | 50.9 ± 0.14 | 49.7 ± 0.07 | 49.8 ± 2.26 | 40.7 ± 1.3 |
| Crude Fat | 8.99 ± 0.03 | 10.3 ± 0.01 | 10.6 ± 0.14 | 11.1 ± 0.01 | 11.6 ± 0.01 | 13.5 ± 1.5 |
| Crude Protein | 32.2 ± 0.1 | 31.6 ± 0.7 | 31.4 ± 0.1 | 32.0 ± 0.01 | 33.1 ± 0.1 | 38.9 ± 0.2 |
| Crude Fiber | 28.7 ± 1.2 | 29.5 ± 2.1 | 30.3 ± 0.2 | 28.0 ± 0.1 | 26.4 ± 0.6 | 10.9 ± 0.5 |
| Acid Detergent | 37.9 ± 0.5 | 38.7 ± 0.1 | 36.7 ± 2.8 | 36.8 ± 0.5 | 32.1 ± 0.8 | 18.25 ± 0.1 |
| Fiber | | | | | | |
| Neutral Detergent | 39.8 ± 0.6 | 39.9 ± 0.1 | 39.5 ± 0.8 | 38.5 ± 0.6 | 34.8 ± 2.0 | 23.3 ± 0.2 |
| Fiber | | | | | | |
| Total Dietary | 41.6 ± 1.2 | 41.2 ± 1.2 | 41.0 ± 1.0 | 39.0 ± 0.1 | 42.2 ± 7.4 | 29.7 ± 1.3 |
| Fiber | | | | | | |

*The Blend sample, consisting of Lots 1-4 (~66% by weight) and Lot 5 (~33% by weight), was blended and analyzed for nutrition studies.

TABLE 4
Proximate compositions (% as is) for canola
meal (CM) and pennycress meal samples.

| | CM ¹ | PM ² |
|---|---|---|
| Crude Protein | 36.7 | 32.0 |
| Fat | 11.4 | 8.61 |
| Crude Fiber | 9.27 | 19.9 |
| ADF ³ | 18.3 | 39.6 |
| NDF ⁴ | 22.7 | 43.0 |
| Ash | 6.51 | 7.57 |
| Dry Matter | 94.1 | 94.4 |

Total Metabolizable Energy (TMEn) corrected for nitrogen was measured in mechanically expeller-pressed pennycress meal and canola meal. TMEn was found to be 18.2% or 18.9% less in the pennycress meal as compared to the canola meal when fed to chickens due to the higher fiber content (Table 5) and Metabolizable Energy (ME) was 16% less in pennycress meal as compared to the canola meal when fed to pigs due to the higher fiber content (Table 6).

TABLE 5
Total metabolizable energy corrected for nitrogen
(TMEn) for mechanically expeller-pressed canola
and pennycress meal when fed to chickens.

| | Mech Pennycress | Mech | Difference, |
|---|---|---|---|
| | Meal (Beecher) | Canola Meal | % |
| Energy | Parsons 2015 | Parsons 2006 | |
| TMEn (kcal/g DM) | 2.455 | 3 | −18.17 |

TABLE 6
Concentration of digestible energy (DE) and metabolizable
energy (ME) in pennycress expeller and canola expellers when fed
to pigs (data¹ produced at University of Illinois).

| | Ingredients | | | |
|---|---|---|---|---|
| | Pennycress | Canola | | |
| Item | expellers | expellers | SEM | P - value |
| DE, kcal/kg | 3,191 | 3,582 | 92.18 | 0.009 |
| DE, kcal/kg of DM | 3,536 | 3,833 | 99.43 | 0.053 |
| ME, kcal/kg | 2,652 | 3,269 | 143.98 | 0.009 |
| ME, kcal/kg of DM | 2,938 | 3,499 | 158.17 | 0.025 |

¹Data are means of 8 observations per treatment. SEM abbreviation stands for standard error of the mean. DM abbreviation is for Dry Matter.

In summary, Beecher and Ruby II varieties of pennycress meal contain between 1.5× to 2× the fiber content as compared to similarly processed canola meal resulting in 18-19% less energy when fed to chickens and pigs. Reduction in the fiber content of pennycress to levels of those in canola should result in a significant increase in value and energy to poultry and pigs.

### Example 2: Selection of Mutant Pennycress Plants Low in Fiber, High in Oil and Protein from Cultivated Isolates

About 850 wildtype pennycress seed samples exhibited a dark-brown seed coat were collected. These wildtype samples were then cultivated as independent lines for over two seasons in over 10,000 unique and managed plots. Upon careful analysis of the harvests from these dark type plantings, a few individual seeds which were yellow in color were identified in only two of the 850 cultivated lines (Table 2) and selected for further propagation and breeding. Certain selected pennycress variant lines Y1067 and Y1126 were isolated from a cultivated field in Grantfork IL. Certain selected pennycress Y1126 lines were isolated from a cultivated field in Macomb IL in 2015. As no yellow pennycress seeds were reported to date, initially, the isolates were first assumed to be weed seeds from a species other than pennycress. However, upon careful evaluations of plants grown from these seeds in the greenhouse, they were positively identified as pennycress using visual (plant morphology) and molecular (PCR/sequencing) inspections. The selected Y1067 and Y1126 lines were then carefully grown as single seed isolates to produce progeny lines which consisted of 100% yellow seeds. The yellow seed coat trait in the selected Y1067 and Y1126 lines has now been confirmed to be stable for several generations in both greenhouse and field environments.

Seeds from the yellow-seeded lines (Y1067 and Y1126) were carefully bulked up and sent to an analytical lab (Dairyland Laboratories) for analysis. Upon removal of the oil using standard defatting procedure, a small amount of yellow pennycress meal was produced and determined to have an ADF level (adjusted for oil content) of 15.5% and 11.5% vs. 27.5% in wild type, demonstrating 43-58% reduction in ADF fiber. Other measurements of fiber content such as NDF and CF were also significantly (29-55%) lower in the yellow-seeded lines relative to wild type, while the protein level was significantly (~50%) higher. The composition of yellow and dark brown seeds is listed in Table 7. The yellow Y1067 and Y1126 lines have since been crossed with “regular” dark brown-seeded pennycress and demonstrated a non-reciprocal pattern of inheritance indicating that yellow seed coat is a maternally inherited trait.

TABLE 7
The composition of meal (adjusted for oil content)
made from yellow and dark brown seeds (Dairyland
Laboratories, Arcadia, Wisconsin).

| Pennycress | Seed coat | % | ADF | NDF | Crude | |
|---|---|---|---|---|---|---|
| line | color | moisture | fiber | fiber | fiber | Protein |
| Y1067 | yellow | 6.63 | 15.5 | 22.3 | 15.5 | 32.4 |
| Y1126 | yellow | 6.38 | 11.5 | 15.2 | 9.9 | 31.9 |
| 1063 | dark brown | 7.39 | 27.2 | 30.6 | 22.6 | 21.3 |
| 1067 | dark brown | 7.29 | 26.6 | 29.8 | 19.9 | 19.8 |
| 1126 | dark brown | 6.43 | 28.4 | 33.7 | 24.7 | 24.6 |
| 1139 | dark brown | 6.50 | 26.4 | 29.8 | 19.9 | 22.4 |
| 1204 | dark brown | 6.58 | 26.3 | 28.9 | 18.7 | 20.9 |
| 1228 | dark brown | 6.30 | 28.8 | 33.8 | 25.4 | 22.1 |
| 1326 | dark brown | 6.47 | 29.2 | 32.6 | 23.4 | 21.7 |
| 2032 | dark brown | 6.16 | 24.7 | 28.8 | 17.6 | 22.1 |
| 2084 | dark brown | 6.89 | 26.0 | 29.0 | 19.4 | 22.2 |
| 2116 | dark brown | 7.16 | 30.4 | 36.2 | 24.4 | 20.1 |
| 2133 | dark brown | 6.64 | 29.6 | 34.4 | 25.0 | 21.5 |
| 2206 | dark brown | 6.69 | 25.5 | 29.4 | 18.1 | 20.7 |
| 2229 | dark brown | 6.61 | 27.1 | 32.5 | 23.0 | 21.9 |
| 2253 | dark brown | 6.42 | 24.0 | 28.3 | 17.8 | 22.5 |
| 2288 | dark brown | 6.28 | 26.6 | 33.0 | 25.5 | N/A |
| 2329 | dark brown | 6.57 | 26.6 | 31.9 | 18.8 | 20.8 |
| 2369 | dark brown | 6.05 | 23.1 | 26.7 | 17.9 | 23.2 |
| 2458 | dark brown | 6.39 | 25.4 | 29.8 | 18.8 | 22.2 |
| 2460 | dark brown | 6.49 | 30.6 | 36.3 | 26.7 | 21.2 |
| 2369 | light brown | 6.50 | 36.9 | 45.8 | 32.1 | 19.1 |
| Average | yellow | 6.51 | 13.5 | 18.7 | 12.7 | 32.2 |
| Average | dark brown | 6.59 | 27.5 | 32.1 | 22.0 | 21.6 |
| % change | yellow | Y1067 | −43% | −30% | −29% | 50% |
| % change | yellow | Y1126 | −58% | −53% | −55% | 48% |

### Example 3: Identification of Mutated Gene in Pennycress Plants Low in Fiber, High in Oil and Protein from Cultivated Isolates

In order to determine molecular nature of the mutations responsible for the low fiber, high oil/high protein phenotype in Y1067 and Y1126 lines, a combination of a genetic method called bulk segregant analysis (Michelmore et. al., 1991) and a next generation sequencing (NGS) method was used. In brief, for each of the yellow-seeded lines, a genetically close black-seeded relative line was identified and 200 individuals from each population were grown. They were harvested in bulk and used for DNA isolation that was subsequently used for preparation of NGS libraries and sequencing using standard Illumina technology. It was determined that Y1067 and Y1126 lines carry the same 21 bp deletion in TTG1 gene (Seq ID No. 165) by analyzing the sequencing data through comparative bioinformatics techniques. Comparative bioinformatics tools that were used in part to analyze the data are disclosed in Magwene et. al., 2011. This mutation results in a deletion of 7 amino acids in the conserved area of TTG1 protein, likely leading to a complete loss of function. The definitive nature of this 21 bp deletion was confirmed in heterologous (black ♀×yellow ♂) crosses, where only the progeny of F2 segregants carrying the described deletion displayed the yellow-seeded phenotype.

### Example 4: Generation and Characterization of EMS-Mutagenized Light-Colored Seed Coat Mutant Lines BC38, BJ8, P32, J22, Q36, BD24, AX17, E5-444, E5-540, E5-541, E5-542, E5-543, E5-545, E5-547, E5-549, E5-582, E5-586, D3-N10 P5, D5-191, A7-95, A7-187 and A7-261

In addition to mutants carrying domestication enabling traits selected from natural isolates, light colored pennycress mutants were isolated from a mutant population created using chemical mutagen (EMS) using the protocol described in the Materials and Methods section below.

To identify useful domestication genes in pennycress plants, pennycress seeds were mutagenized with several different mutagens, including ethyl methanesulfonate (EMS), fast neutrons (FN) and gamma rays (□ rays). Treatment of dry plant seeds with mutagens results in the generation of distinct sets of mutations in a variety of cells in the seed. The fate of many of these cells can be followed when a mutation in one of these cells results in a visible phenotype creating a marked plant sector.

Pennycress plants exhibiting domestication enabling traits such as reduced seed coat fiber, lighter-colored seed coat due to reduced proanthocyanidins content, and/or higher seed oil content were analyzed and loss of function mutations in domestication genes were identified.

### Materials and Methods

### Solutions

| | | |
|---|---|---|
| A) | 0.2M sodium phosphate monobasic | 6.9 g/250 mL |
| | (NaH₂PO₄*H₂O) | |
| B) | 0.2M sodium phosphate dibasic | 7.1 g/250 mL |
| | (NaH₂PO₄ anhydrous) | |

| For 50 mL of 0.1M sodium phosphate buffer at pH 7: | |
|---|---|
| 9.75 mL | A |
| 15.25 mL | B |
| 25.0 mL | dH₂O |

0.2% EMS in buffer:
20 mL 0.1M Sodium Phosphate Buffer, pH 7
40 μL EMS liquid (Sigma #M0880-5G)
0.1M sodium thiosulfate at pH 7.3:
12.4 g sodium thiosulfate in 500 mL

### Primary Seed Surface Sterilization

Wild-type pennycress (Thlaspi arvense) seeds (Spring 32 ecotype) were surface sterilized for 10 minutes in a 30% bleach, 0.05% SDS solution before being rinsed 3× with sterile water. Sterilized seeds were immediately subjected to EMS treatment.

### Ethyl Methane Sulfonate (EMS) Treatment of Pennycress Seeds

Sterilized pennycress seeds (41 g) were agitated in distilled water overnight. Four 250 mL Erlenmeyer flasks with 10 g seed each, and 1 g in a separate small flask as a control, were agitated. The water was decanted.

25 mLs of 0.2% EMS in 0.1M sodium phosphate buffer (pH 7) was added. The control received only phosphate buffer with no EMS. The flasks were shaken in fume hood for 18 hours. The EMS solution was decanted off into an EMS waste bottle.

To rinse the seeds, 25 ml of dH₂O was added to each flask, and the flasks were shaken for 20 minutes. The rinse water was decanted into the EMS waste bottle.

To deactivate the EMS, seeds were washed for 20 minutes in 0.1M sodium thiosulfate (pH 7.3), rinsed 4 with dH₂O for 15 minutes, suspended in 0.1% agarose, and germinated directly in autoclaved Reddiearth soil at a density of approximately 10 seeds per 4-inch pot.

### Plant Growth Conditions

EMS-treated pennycress seeds were germinated and grown in an environmental growth chamber at 21° C., 16:8 6400K fluorescent light/dark, 50% humidity. Approximately 14 days after planting, plants were thinned and transplanted to a density of 4 plants per 4-inch pot. These M₁-generation plants showed telltale chlorotic leaf sectors that are indicative of a successful mutagenesis.

After dry down, these M₁-generation plants were catalogued and harvested. The M₂- and M₃-generation seeds were surface sterilized, planted and grown according to the protocols previously described.

### Identification and Characterization of Light-Colored Seed Coat Mutant Lines

Light-colored seed coat mutants in the M₃-generation were identified as those having mature seed coats of a lighter color relative to that of wild type. Seeds (M₃-generation) from putative M₂-generation mutants were planted and grown in potting soil-containing 4-inch pots in a growth chamber and the seed coat color phenotype re-assessed upon plant senescence.

Near infrared (NIR) spectroscopic analysis was used to determine the fiber content of selected seed lines to compare the obtained values to the range of fiber in control dark brown seeds. The results are presented in Table 8 of Example 5 (five light-colored lines mentioned above vs. almost one hundred control dark brown seed lines). These results indicate that ADF and NDF fiber levels in certain selected light-colored seed lines are significantly lower and are outside of the corresponding ranges found in control dark-colored seeds, while oil and protein levels are often higher and are also outside of their corresponding ranges found in dark-colored control seeds.

EMS mutagenesis typically introduces single-nucleotide transition mutations (e.g. G to A, or C to T) into plant genomes. To identify the causative mutations in selected light seed colored plants, DNA was extracted from mutant and wild-type leaf tissue and used for NGS and comparative bioinformatics analysis as described in Example 3. Underlying gene and protein mutations were identified (Table 1, SEQ ID NO: 117-132, 139-142, 149-158, 167-170 and 174-181) and confirmed using standard Sanger sequencing and genetic segregation analyses.

### Example 5: Generation of Transgenic Pennycress Lines Harboring the CRISPR-Cas9 or CRISPR-Cpf1 or CRISPR-Cms1 Constructs

### Materials and Methods

Construction of the Thlaspi arvense (Pennycress) TT1, TT2, TT8, TT10, and TT16 Gene-Specific CRISPR Genome-Editing Vectors.

The constructs and cloning procedures for generation of the Thlaspi arvense (pennycress) TT2-, TT8-, TT10-, and TT16-specific CRISPR-SpCas9, CRISPR-SaCas9, CRISPR-Cpf1 and CRISPR-Cms1 constructs are described in Fauser et. al., 2014, Steinert et. al., 2015 and Begemann et. al., 2017.

The plant selectable markers (formerly NPT) in the original pDe-SpCas9 and pDe-SaCas9 binary vectors were swapped for hygromycin resistance (Hygromycin phosphotransferase (HPT) gene.

Complementary oligo pairs described in Table 1 (Seq ID NO: 89-116) were synthesized, annealed to create the 20-mer protospacers specific to the designated pennycress genes and used for construction of gene-editing binary vectors as described (Fauser et. al., 2014, Steinert et. al., 2015 and Begemann et. al., 2017).

Vector Transformation into Agrobacterium

The pDe-SpCas9_Hyg and pDe-SaCas9_Hyg and related vectors containing the CRISPR nuclease and guide RNA cassettes with the corresponding sequence-specific protospacers were transformed into Agrobacterium tumefaciens strain GV3101 using the freeze/thaw method (Holsters et al, 1978).

The transformation product was plated on 1% agar Luria Broth (LB) plates with gentamycin (50 μg/ml) rifampicin (50 μg/ml) and spectinomycin (75 μg/ml). Single colonies were selected after two days of growth at 28° C.

### Plant Transformation—Pennycress Floral Dip

DAY ONE: 5 mL of LB+5 uL with appropriate antibiotics (Rifampin (50), Spectinomycin (75), and/or Gentamycin (50)) were inoculated with Agrobacterium. The cultures were allowed to grow, with shaking, overnight at 28° C.

DAY TWO (early morning): 25 mL of Luria Broth+25 μL appropriate antibiotics (Rifampin (50), Spectinomycin (75), and/or Gentamycin (50)) were inoculated with the initial culture from day one. The cultures were allowed to grow, with shaking, overnight at 28° C.

DAY TWO (late afternoon): 250 mL of Luria Broth+250 μL appropriate antibiotic (Rifampin (50), Spectinomycin (75), and/or Gentamycin (50)) were inoculated with 25 mL culture. The cultures were allowed to grow, with shaking, overnight at 28° C.

DAY THREE: When the culture had grown to an OD₆₀₀ of ~1.0, the culture was decanted into large centrifuge tubes and spun at 3,500 RPM at room temperature for 10 minutes to pellet cells. The supernatant was decanted off. The pelleted cells were resuspended in a solution of 5% sucrose and 0.02% Silwet L-77. The suspension was poured into clean beakers and placed in a vacuum chamber.

Newly flowering inflorescences of pennycress were fully submerged into the beakers and subjected to a negative vacuum pressure of 25-30 PSI for 10 minutes.

After pennycress plants were dipped, they were covered loosely with Saran wrap to maintain humidity and kept in the dark overnight before being uncovered and placed back in the environmental growth chamber.

### Screening Transgenic Plants and Growth Condition

Pennycress seeds were surface sterilized by first rinsing in 70% ethanol then incubating 10 minutes in a 30% bleach, 0.05% SDS solution before being rinsed two times with sterile water and plated on selective plates (0.8% agar/one half-strength Murashige and Skoog salts with hygromycin B selection (40 U/ml) or glufosinate (18 μg/ml). Plates were wrapped in parafilm and kept in an environmental growth chamber at 21° C., 16:8 day/night for 8 days until antibiotic or herbicide selection was apparent.

Surviving hygromycin or glufosinate-resistant T₁-generation seedlings were transplanted into autoclaved Reddiearth soil mix and grown in an environmental growth chamber set to 16-hour days/8-hour nights at 21° C. and 50% humidity. T₂-generation seeds were planted, and ~1.5 mg of leaf tissue from each T₂-generation plant was harvested with a 3-mm hole punch, then processed using the Thermo Scientific™ Phire™ Plant Direct PCR Kit as per manufacturer's instructions. Subsequently, PCR reactions for genotyping (20 μl volume) were performed.

Gene editing using Cas9, Cpf1 and Cms1 nucleases typically introduces a double-stranded break into a targeted genome area in close proximity to the nuclease's PAM site. During non-homologous end-joining process (NHEJ), these double-stranded breaks are repaired, often resulting in introduction of indel-type mutations into targeted genomes. To identify plants with small indels in genes of interest, standard Sanger sequencing or T7 endonuclease assay (Guschin et. al., 2010) were employed. Sequence analysis revealed that multiple guide RNAs/CRISPR nuclease combinations were effective in generating loss-of-function (LOF) mutations in targeted genes, as described in Table 1 (Seq ID Nos. 133-138, 143-148, 159-164). Plants carrying LOF mutations were grown to homozygosity, and the phenotypes were confirmed using visual and analytical assessments.

### Example 6. Selected Yellow-Seeded Pennycress Mutants Demonstrate Significant Reductions in Fiber and Fiber Components

Homozygous light seed coat-colored mutants obtained from screening EMS populations or from gene editing were bulked up in the greenhouse or in the fields and their fiber composition was assessed using standard methods below at Dairyland Laboratories (Arcadia, Wisconsin).

### ADF (Acid Detergent Fiber)

Fiber (Acid Detergent) and Lignin in Animal Feed: AOAC Official Method 973.18 (1996) (Modification includes use of Sea Sand for filter aid as needed).

### Crude Fiber

Fiber (Crude) in Animal Feed and Pet Food (Fritted Glass Crucible Method): AOAC Official Method 978.10 ch4 p28 (1979) (Modification includes use of Sea Sand for filter aid as needed).

### Lignin

Fiber (Acid Detergent) and Lignin in Animal Feed: AOAC Official Method 973.18 (1996) (Modification includes use of Sea Sand for filter aid as needed, use of Whatman GF/C filter paper to collect residue, and holding crucibles in beakers to cover fiber with 72% sulfuric acid for full time required).

### NDF (Neutral Detergent Fiber)

Amylase-Treated Neutral Detergent Fiber in Feeds AOAC Official Method 2002.04 2005 (Modification includes use of Sea Sand for filter aid and Whatman GF/C filter paper for residue collection).

The results presented in Table 8 indicate that majority of the light-colored mutants have 35-60% less fiber and its components relative to WT plants (MN106 and Beecher).

TABLE 8
Composition of sixteen selected light-colored pennycress mutants vs. two wild type
pennycress accessions measured using wet chemistry methods at Dairyland Laboratories
(Arcadia, Wisconsin). The numbers represent percent of dry matter (% DM).

| | | Mutated | Seed | | Crude | | | Crude |
|---|---|---|---|---|---|---|---|---|
| No. | Name/ID | Gene/Allele | Coat | Moisture | Protein | ADF | aNDF | fiber |
| 1 | Y1126 | ttg1 | light | 7.6 | 28.1 | 13.9 | 16.6 | 9.6 |
| 2 | E5-543 | tt10-1 | light | 7.4 | 26.5 | 15.3 | 19.7 | 14.4 |
| 3 | E5-542 | tt8 | light | 7.5 | 30.6 | 9.1 | 17.5 | 13.8 |
| 4 | E5-547 | tt2-1 | light | 6.7 | 28.1 | 12.8 | 17.2 | 12.1 |
| 5 | A7-63 | N/A | light | 6.9 | 28.7 | 14.6 | 20.5 | 11.8 |
| 6 | A7-187 | ttg1-2 | light | 7.5 | 29.2 | 12.9 | 17.8 | 13.1 |
| 7 | E5-559 | gl3-1 | light | 7.0 | 26.3 | 21.8 | 32.5 | 22.5 |
| 8 | E5-539 | tt10-1 | light | 7.5 | 27.3 | 13.9 | 17.6 | 12.0 |
| 9 | A7-261 | tt12-1 | light | 6.6 | 27.2 | 14.9 | 19.5 | 13.6 |
| 10 | E5-549 | tt4-2 | light | 7.4 | 26.5 | 16.2 | 22.3 | 12.7 |
| 11 | E5-444 | gl3-2 | light | 7.8 | 27.7 | 14.6 | 17.5 | 10.8 |
| 12 | D5-191 | tt8-2 | light | 6.5 | 26.6 | 13.3 | 17.9 | 13.0 |
| 13 | E5-586 | tt7-1 | light | 7.4 | 27.9 | 12.6 | 17.2 | 11.3 |
| 14 | E5-542 | tt8-3 | light | 6.9 | 26.0 | 13.5 | 19.9 | 16.2 |
| 15 | E5-541 | gl3-1 | light | 6.8 | 27.2 | 15.1 | 19.2 | 13.2 |
| 16 | E5-545 | tt10-2 | light | 6.7 | 24.5 | 14.8 | 18.5 | 12.9 |
| 17 | MN106 | WT | dark | 6.7 | 25.2 | 22.7 | 25.8 | 16.1 |
| 18 | Beecher | WT | dark | 6.5 | 25.6 | 21.1 | 23.9 | 15.4 |
| 19 | MIN of | light-colored | % of | 6.5 | 24.5 | 9.1 | 16.6 | 9.6 |
| | | | DM | | | | | |
| 20 | MAX of | light-colored | % of | 7.8 | 30.6 | 21.8 | 32.5 | 22.5 |
| | | | DM | | | | | |
| 21 | MIN of | light-colored | % of | 97% | 97% | 40% | 64% | 60% |
| | | | WT | | | | | |

### Example 7. Selected Yellow-Seeded Pennycress Mutants Demonstrate Significant Increases in Protein and Oil Composition

TABLE 9
Composition of five selected light-colored pennycress mutants vs.
95 wild type pennycress accessions harvested at various locations
across USA and measured using NIR spectroscopy analysis.

| | | | | % | % | | % | % | |
|---|---|---|---|---|---|---|---|---|---|
| | | | % | Erucic | Total | Sinigrin | ADF | NDF | % |
| No. | Accession | Color | Moisture | Acid | Oil | μmol/g | Fiber | Fiber | Protein |
| 1 | Y1067 | Yellow | 7.2 | 25.1 | 37.6 | 149.1 | 15.5 | 16.2 | 32.5 |
| 2 | Y1126 | Yellow | 8.3 | 31.1 | 43.3 | 49.9 | 11.5 | 14.9 | 31.8 |
| 3 | P32 | Light | 6.0 | 39.5 | 36.4 | 180.2 | 13.5 | 18.0 | 29.1 |
| | | brown | | | | | | | |
| 4 | Q36.C | Brown | 6.1 | 22.8 | 33.0 | 196.2 | 19.7 | 24.1 | 25.0 |
| 5 | BJ.8 | Tan | 7.0 | 39.0 | 49.0 | 107.4 | 10.0 | 13.1 | 33.6 |
| 6 | 1126 | Dark | 10.2 | 33.7 | 30.8 | 59.2 | 27.6 | 31.2 | 22.2 |
| | | brown | | | | | | | |
| 7 | Spring32 | Dark | 8.6 | 34.8 | 30.6 | 116.0 | 27.6 | 32.2 | 22.0 |
| | (WT) | brown | | | | | | | |
| 8 | 1069 | Dark | 8.8 | 32.9 | 29.4 | 103.4 | 37.8 | 35.1 | 22.6 |
| | | brown | | | | | | | |
| 9 | 1096 | Dark | 8.4 | 31.3 | 26.0 | 128.7 | 32.9 | 34.2 | 20.1 |
| | | brown | | | | | | | |
| 10 | 2139 | Dark | 8.7 | 29.6 | 23.1 | 147.0 | 29.0 | 33.9 | 20.4 |
| | | brown | | | | | | | |
| 11 | 2057 | Dark | 8.2 | 31.0 | 23.7 | 157.6 | 31.5 | 33.8 | 18.7 |
| | | brown | | | | | | | |
| 12 | 1126 | Dark | 7.8 | 29.2 | 30.6 | 117.4 | 34.7 | 31.1 | 20.8 |
| | | brown | | | | | | | |
| 13 | 2066 | Dark | 8.7 | 36.8 | 35.2 | 83.0 | 26.2 | 29.1 | 22.4 |
| | | brown | | | | | | | |
| 14 | 2142 | Dark | 8.9 | 32.6 | 32.5 | 85.5 | 29.8 | 32.7 | 20.4 |
| | | brown | | | | | | | |
| 15 | 2170 | Dark | 8.8 | 31.8 | 29.4 | 118.4 | 30.6 | 31.3 | 22.3 |
| | | brown | | | | | | | |
| 16 | 2055 | Dark | 8.7 | 30.8 | 27.6 | 87.1 | 36.1 | 34.0 | 21.1 |
| | | brown | | | | | | | |
| 17 | 2065 | Dark | 9.0 | 27.8 | 29.7 | 127.6 | 30.0 | 33.9 | 19.7 |
| | | brown | | | | | | | |
| 18 | 2110 | Dark | 9.0 | 27.3 | 31.4 | 85.3 | 35.4 | 33.1 | 20.5 |
| | | brown | | | | | | | |
| 19 | 2154 | Dark | 8.7 | 32.0 | 34.6 | 58.1 | 33.2 | 32.2 | 20.1 |
| | | brown | | | | | | | |
| 20 | 2195 | Dark | 8.6 | 32.3 | 34.3 | 61.6 | 29.2 | 32.5 | 19.1 |
| | | brown | | | | | | | |
| 21 | 1311 | Dark | 8.3 | 34.8 | 30.1 | 126.6 | 26.7 | 28.4 | 25.0 |
| | | brown | | | | | | | |
| 22 | 2003 | Dark | 8.3 | 33.4 | 25.4 | 79.5 | 29.6 | 29.6 | 20.7 |
| | | brown | | | | | | | |
| 23 | 1065 | Dark | 8.7 | 34.2 | 29.6 | 112.5 | 29.2 | 31.7 | 23.5 |
| | | brown | | | | | | | |
| 24 | 2045 | Dark | 8.8 | 33.9 | 25.3 | 122.0 | 33.0 | 31.9 | 22.4 |
| | | brown | | | | | | | |
| 25 | 2128 | Dark | 8.5 | 34.6 | 29.5 | 129.3 | 23.4 | 27.2 | 25.2 |
| | | brown | | | | | | | |
| 26 | 2182 | Dark | 8.4 | 32.7 | 33.7 | 81.6 | 28.2 | 29.6 | 22.2 |
| | | brown | | | | | | | |
| 27 | 2030 | Dark | 7.7 | 31.3 | 33.2 | 105.8 | 24.0 | 27.7 | 20.3 |
| | | brown | | | | | | | |
| 28 | 2034 | Dark | 8.1 | 32.4 | 29.6 | 116.9 | 26.6 | 30.0 | 22.9 |
| | | brown | | | | | | | |
| 29 | 2072 | Dark | 8.2 | 30.2 | 27.8 | 97.3 | 30.8 | 31.0 | 21.3 |
| | | brown | | | | | | | |
| 30 | 2145 | Dark | 8.2 | 33.1 | 29.7 | 119.0 | 23.3 | 28.6 | 24.1 |
| | | brown | | | | | | | |
| 31 | 1027 | Dark | 8.0 | 29.4 | 30.6 | 110.6 | 30.5 | 29.1 | 23.4 |
| | | brown | | | | | | | |
| 32 | 1323 | Dark | 8.5 | 31.2 | 28.2 | 115.3 | 33.0 | 32.2 | 23.3 |
| | | brown | | | | | | | |
| 33 | 1340 | Dark | 8.0 | 32.3 | 29.2 | 129.8 | 28.5 | 29.4 | 22.9 |
| | | brown | | | | | | | |
| 34 | 2129 | Dark | 8.0 | 33.1 | 29.6 | 109.4 | 21.5 | 27.4 | 24.1 |
| | | brown | | | | | | | |
| 35 | 2167 | Dark | 8.5 | 28.6 | 34.8 | 71.8 | 34.4 | 31.7 | 21.5 |
| | | brown | | | | | | | |
| 36 | 2171 | Dark | 8.0 | 33.4 | 28.6 | 108.1 | 24.5 | 28.5 | 20.7 |
| | | brown | | | | | | | |
| 37 | 1054 | Dark | 8.3 | 34.0 | 29.0 | 128.4 | 29.4 | 31.3 | 22.2 |
| | | brown | | | | | | | |
| 38 | 1092 | Dark | 8.3 | 36.6 | 29.8 | 131.6 | 27.2 | 30.1 | 22.6 |
| | | brown | | | | | | | |
| 39 | 2196 | Dark | 9.2 | 32.4 | 32.5 | 113.1 | 22.7 | 30.7 | 21.2 |
| | | brown | | | | | | | |
| 40 | 2183 | Dark | 8.1 | 33.4 | 28.0 | 111.7 | 27.0 | 30.0 | 21.2 |
| | | brown | | | | | | | |
| 41 | 2020 | Dark | 8.5 | 32.5 | 31.9 | 128.1 | 22.5 | 29.0 | 21.4 |
| | | brown | | | | | | | |
| 42 | 2123 | Dark | 8.5 | 34.9 | 30.9 | 122.3 | 22.7 | 27.1 | 25.3 |
| | | brown | | | | | | | |
| 43 | 1296 | Dark | 8.0 | 36.2 | 30.6 | 113.3 | 25.9 | 28.3 | 23.7 |
| | | brown | | | | | | | |
| 44 | 2062 | Dark | 8.8 | 31.6 | 26.7 | 117.5 | 29.5 | 31.7 | 22.2 |
| | | brown | | | | | | | |
| 45 | 1167 | Dark | 8.0 | 34.0 | 28.3 | 121.0 | 31.7 | 30.4 | 22.3 |
| | | brown | | | | | | | |
| 46 | 1359 | Dark | 7.7 | 33.4 | 29.4 | 125.9 | 25.2 | 27.2 | 22.9 |
| | | brown | | | | | | | |
| 47 | 1265 | Dark | 8.4 | 34.6 | 32.2 | 78.0 | 29.6 | 30.7 | 22.8 |
| | | brown | | | | | | | |
| 48 | 1331 | Dark | 8.0 | 37.6 | 29.0 | 112.3 | 27.0 | 28.3 | 23.1 |
| | | brown | | | | | | | |
| 49 | 2002 | Dark | 7.9 | 33.1 | 27.4 | 59.8 | 28.6 | 30.0 | 20.6 |
| | | brown | | | | | | | |
| 50 | 2009 | Dark | 7.4 | 35.9 | 32.3 | 67.1 | 26.7 | 26.9 | 22.7 |
| | | brown | | | | | | | |
| 51 | 2079 | Dark | 8.0 | 37.5 | 29.3 | 126.2 | 21.0 | 28.3 | 22.5 |
| | | brown | | | | | | | |
| 52 | 2092 | Dark | 9.1 | 32.3 | 33.4 | 89.7 | 27.6 | 33.4 | 21.0 |
| | | brown | | | | | | | |
| 53 | 2107 | Dark | 8.8 | 35.8 | 29.7 | 103.4 | 21.3 | 28.8 | 21.5 |
| | | brown | | | | | | | |
| 54 | 2113 | Dark | 8.8 | 31.9 | 33.7 | 83.4 | 28.5 | 30.3 | 23.0 |
| | | brown | | | | | | | |
| 55 | 2117 | Dark | 8.2 | 30.8 | 26.6 | 99.0 | 23.7 | 29.5 | 20.9 |
| | | brown | | | | | | | |
| 56 | 2132 | Dark | 8.0 | 36.1 | 29.2 | 121.4 | 25.1 | 27.9 | 23.4 |
| | | brown | | | | | | | |
| 57 | 2137 | Dark | 7.9 | 32.9 | 28.8 | 115.6 | 27.7 | 28.8 | 22.2 |
| | | brown | | | | | | | |
| 58 | 2140 | Dark | 8.7 | 32.0 | 27.5 | 103.9 | 24.7 | 31.2 | 20.7 |
| | | brown | | | | | | | |
| 59 | 2008 | Dark | 7.7 | 35.0 | 29.7 | 75.5 | 23.8 | 26.3 | 22.1 |
| | | brown | | | | | | | |
| 60 | 2102 | Dark | 7.9 | 18.3 | 24.0 | 193.8 | 35.2 | 32.3 | 16.4 |
| | | brown | | | | | | | |
| 61 | 2021 | Dark | 9.0 | 30.5 | 28.1 | 127.7 | 26.4 | 33.3 | 19.7 |
| | | brown | | | | | | | |
| 62 | 2114 | Dark | 9.4 | 30.6 | 30.1 | 114.7 | 27.1 | 32.2 | 20.3 |
| | | brown | | | | | | | |
| 63 | 1022 | Dark | 8.7 | 33.8 | 28.4 | 137.0 | 26.6 | 30.8 | 22.3 |
| | | brown | | | | | | | |
| 64 | 2051 | Dark | 9.4 | 34.8 | 31.7 | 73.9 | 30.1 | 32.7 | 21.3 |
| | | brown | | | | | | | |
| 65 | 2073 | Dark | 9.8 | 33.5 | 27.6 | 132.3 | 27.3 | 34.0 | 20.2 |
| | | brown | | | | | | | |
| 66 | 2078 | Dark | 7.6 | 37.1 | 29.2 | 74.5 | 22.3 | 27.4 | 22.0 |
| | | brown | | | | | | | |
| 67 | 2209 | Dark | 8.1 | 31.0 | 28.4 | 104.2 | 27.3 | 29.2 | 22.1 |
| | | brown | | | | | | | |
| 68 | 2210 | Dark | 8.6 | 32.5 | 33.4 | 86.3 | 24.9 | 29.4 | 20.5 |
| | | brown | | | | | | | |
| 69 | 1332 | Dark | 7.9 | 36.5 | 30.1 | 113.4 | 24.1 | 26.9 | 23.8 |
| | | brown | | | | | | | |
| 70 | 2095 | Dark | 8.6 | 31.0 | 27.4 | 114.6 | 30.7 | 31.2 | 22.8 |
| | | brown | | | | | | | |
| 71 | 2143 | Dark | 9.0 | 29.1 | 33.1 | 97.8 | 23.7 | 32.3 | 21.5 |
| | | brown | | | | | | | |
| 72 | 2156 | Dark | 8.1 | 35.5 | 28.5 | 144.4 | 22.1 | 28.7 | 23.7 |
| | | brown | | | | | | | |
| 73 | 1235 | Dark | 8.1 | 32.7 | 27.8 | 148.3 | 27.4 | 28.4 | 23.0 |
| | | brown | | | | | | | |
| 74 | 2058 | Dark | 8.2 | 31.1 | 26.1 | 142.6 | 26.3 | 28.8 | 23.4 |
| | | brown | | | | | | | |
| 75 | 2151 | Dark | 8.7 | 29.5 | 33.2 | 68.4 | 37.3 | 34.1 | 20.4 |
| | | brown | | | | | | | |
| 76 | 1002 | Dark | 8.1 | 29.2 | 26.8 | 141.7 | 28.7 | 31.1 | 22.1 |
| | | brown | | | | | | | |
| 77 | 1218 | Dark | 8.0 | 23.9 | 26.6 | 120.2 | 37.9 | 34.9 | 18.3 |
| | | brown | | | | | | | |
| 78 | 1345 | Dark | 8.0 | 36.1 | 32.5 | 99.1 | 27.4 | 27.9 | 24.5 |
| | | brown | | | | | | | |
| 79 | 1366 | Dark | 8.0 | 36.5 | 31.3 | 115.1 | 26.9 | 28.2 | 22.4 |
| | | brown | | | | | | | |
| 80 | 2185 | Dark | 9.1 | 32.9 | 31.7 | 97.0 | 28.1 | 32.4 | 21.5 |
| | | brown | | | | | | | |
| 81 | 2221 | Dark | 7.7 | 35.8 | 29.9 | 123.2 | 23.3 | 26.9 | 23.2 |
| | | brown | | | | | | | |
| 82 | 2332 | Dark | 8.2 | 30.6 | 28.7 | 70.4 | 34.0 | 31.9 | 20.9 |
| | | brown | | | | | | | |
| 83 | 1149 | Dark | 8.2 | 31.7 | 29.8 | 114.2 | 30.5 | 31.0 | 23.1 |
| | | brown | | | | | | | |
| 84 | 1001 | Dark | 7.7 | 30.4 | 30.7 | 124.6 | 29.6 | 28.2 | 23.7 |
| | | brown | | | | | | | |
| 85 | 1082 | Dark | 8.1 | 30.8 | 30.7 | 85.6 | 33.3 | 30.2 | 22.4 |
| | | brown | | | | | | | |
| 86 | 2286 | Dark | 8.5 | 34.2 | 34.3 | 74.7 | 27.2 | 30.7 | 22.8 |
| | | brown | | | | | | | |
| 87 | 2298 | Dark | 8.0 | 33.6 | 27.5 | 106.8 | 25.2 | 30.6 | 20.8 |
| | | brown | | | | | | | |
| 88 | 2304 | Dark | 7.6 | 33.5 | 29.7 | 108.0 | 23.8 | 26.9 | 23.0 |
| | | brown | | | | | | | |
| 89 | 2308 | Dark | 8.7 | 36.0 | 29.0 | 113.9 | 27.0 | 30.0 | 22.8 |
| | | brown | | | | | | | |
| 90 | 2318 | Dark | 9.2 | 31.4 | 32.5 | 90.6 | 28.8 | 32.3 | 21.5 |
| | | brown | | | | | | | |
| 91 | 2319 | Dark | 9.0 | 27.4 | 32.2 | 71.6 | 31.1 | 35.1 | 20.2 |
| | | brown | | | | | | | |
| 92 | 2332 | Dark | 8.8 | 25.0 | 22.9 | 169.3 | 26.7 | 31.5 | 17.0 |
| | | brown | | | | | | | |
| 93 | 2338 | Dark | 8.0 | 24.5 | 24.1 | 145.7 | 20.8 | 30.9 | 15.3 |
| | | brown | | | | | | | |
| 94 | 2346 | Dark | 8.3 | 31.7 | 27.6 | 140.9 | 27.6 | 30.4 | 22.8 |
| | | brown | | | | | | | |
| 95 | 2347 | Dark | 8.8 | 31.0 | 34.4 | 78.9 | 27.8 | 30.5 | 22.9 |
| | | brown | | | | | | | |
| 96 | 2349 | Dark | 9.6 | 31.2 | 32.3 | 88.0 | 26.6 | 32.2 | 21.7 |
| | | brown | | | | | | | |
| 97 | 2354 | Dark | 8.3 | 28.9 | 27.2 | 84.5 | 30.4 | 30.1 | 21.7 |
| | | brown | | | | | | | |
| 98 | 2359 | Dark | 7.6 | 29.3 | 27.7 | 101.4 | 28.2 | 30.2 | 20.3 |
| | | brown | | | | | | | |
| 99 | 2362 | Dark | 8.7 | 30.5 | 28.6 | 86.7 | 30.1 | 31.3 | 22.7 |
| | | brown | | | | | | | |
| 100 | 2364 | Dark | 9.2 | 31.4 | 32.2 | 89.6 | 28.9 | 34.4 | 21.6 |
| | | brown | | | | | | | |

| | | | % | % | | % | % | |
|---|---|---|---|---|---|---|---|---|
| | | % | Erucic | Total | Sinigrin | ADF | NDF | % |
| | Color | Moisture | Acid | Oil | μmol/g | Fiber | Fiber | Protein |
| Minimum | Light | 6.0 | 22.8 | 33.0 | 49.9 | 10.0 | 13.1 | 25.0 |
| Minimum | Dark | 7.4 | 18.3 | 22.9 | 58.1 | 20.8 | 26.3 | 15.3 |
| Maximum | Light | 8.3 | 39.5 | 49 | 196.2 | 19.7 | 24.1 | 33.6 |
| Maximum | Dark | 10.2 | 37.6 | 35.2 | 193.8 | 37.9 | 35.1 | 25.3 |

### Example 8. Composition and Performance of Pennycress Meal Produced from Y1126 Yellow-Seeded Mutant is Superior Relative to Meal Made from Black-Seeded Pennycress and is Similar to Canola Meal

Approximately 13 lbs each of cleaned Y1126 yellow-seeded mutant and regular black-seeded pennycress seed were processed into oil and hexane-extracted meal at the Texas A&M Engineering Experiment Station's Process Engineering Research & Development Center (College Station, TX). The material was conditioned using a single deck of the French cooker for approximately 5 minutes at 100° F.+10° F. Conditioned seed was processed using a Ferrel Ross flaking rolls to yield flakes with a thickness of approximately 0.012 inches or thinner.

The flakes were loaded into a cooker with the objective of inactivating lipases, myrosinases, and other hydrolytic enzymes to facilitate pre-pressing. Maximum steam was used to get the flakes to 190° F. without lingering to avoid activation of such enzymes. This was achieved in 10-15 minutes. The press (Rosedowns Mini 200) was fed from a Wenger metered feeder with flake at a rate of 3.5-4 pounds per minute. The press operated best at 50-55 Hz, which corresponds to 38-40 RPM.

The presscake was extracted in stainless batch cans using commercial hexane at a temperature of 110-140° F.±10° F. Solvent was added and drained sequentially in 6 rounds of incubation, each of which was approximately 12 minutes. To remove residual hexane and yield desolventized meal, a batch-type desolventizer/toaster (DT) was heated, which showed a product temperature of 150-175° F. under vacuum. Crude oil was made by desolventizing using a Precision Scientific Evaporator. The hexane extracted meal was air dried overnight.

Samples of the hexane extracted meal were sent to Dairyland and DairyOne Laboratories for analysis. A sample of commercial canola meal was acquired from a feed plant in Wisconsin, which was also sent to DairyOne for comparison.

TABLE 10
The meal produced from Y1126 yellow-seeded pennycress mutant is significantly more valuable
(lower in fiber, higher in protein and available energy and nutrients) than regular pennycress
meal and is closer in composition and predicted performance to canola meal.

| | | | | | | Yellow | |
|---|---|---|---|---|---|---|---|
| | | | | Desired | | seed | |
| Meal Component | | Type | Unit | Change | Pennycress | (Y1126) | Canola |
| CP | Crude Protein | Protein | % Dry | Increased | 31.9 | 40.5 | 41.4 |
| | | | Matter | | | | |
| RUP | Rumen Undegraded | Protein | % CP | No change | 41.45 | 42 | 55 |
| | Protein | | | | | | |
| Fat | Oil | Oil | % Dry | No change | 1.17 | 1.69 | 3.6 |
| | | | Matter | | | | |
| ADF | Acid Detergent Fiber | Fiber | % Dry | Reduce | 41.7 | 20.6 | 22.9 |
| | | | Matter | | | | |
| NDF | Neutral Detergent Fiber | Fiber | % Dry | Reduce | 45.5 | 27.2 | 34.3 |
| | | | Matter | | | | |
| Lignin | indigestible cell wall | Fiber | % Dry | Reduce | 24.3 | 7.7 | 10 |
| | material | | Matter | | | | |
| Starch | Starch | Starch | % Dry | No change | 0.5 | 0.5 | 0.3 |
| | | | Matter | | | | |
| Sugar | Sugar | Sugar | % Dry | No change | 6.5 | 9.5 | 8 |
| | | | Matter | | | | |
| IVTD | 24 hour In Vitro Total | Energy | % Dry | Increase | 65 | 89 | 82 |
| 24 | Digestibility | | Matter | | | | |
| TDN | Total Digestible Nutrients | Energy | % Dry | Increase | 53 | 68.5 | 67 |
| | | | Matter | | | | |
| ME, 1X | Calculated Metabolizable | Energy | Mcal/lb | Increase | 0.93 | 1.33 | 1.33 |
| | Energy, 1X maintenance | | | | | | |
| NEL, | Calculated Net Energy | Energy | Mcal/lb | Increase | 1.08 | 1.52 | 1.55 |
| 1X | Lactation, 1X | | | | | | |
| | maintenance | | | | | | |
| NEG, | Calculated Net Energy | Energy | Mcal/lb | Increase | 0.32 | 0.91 | 0.93 |
| 1X | Gain, 1X maintenance | | | | | | |
| NEM, | Calculated Net Energy | Energy | Mcal/lb | Increase | 0.86 | 1.5 | 1.52 |
| 1X | Maintenance, 1X | | | | | | |
| | maintenance | | | | | | |

Samples of the meal made from Y1126 yellow-seeded mutant, regular black-seeded pennycress and commercial canola meal were sent to the University of Illinois (Urbana-Champaign, IL) for Total Metabolizable Energy corrected for nitrogen (TMEn) and digestible amino acid analysis. The University of Illinois utilized the cecectomized rooster assay to measure TMEn and the digestibility of amino acids.

TABLE 11
Y1126 yellow-seed mutant had increased TMEn as compared to
the black-seeded pennycress and was comparable to canola.

| | Dry Matter (DM) | TMEn |
|---|---|---|
| Feed | % | Kcal/g DM |
| Pennycress | 97.0 | 1.68 |
| Yellow Seed (Y1126) | 97.6 | 2.02 |
| Canola | 89.1 | 2.14 |

TABLE 12
Y1126 yellow-seeded mutant has increased true amino acid
digestibility as compared to the black-seeded pennycress
and was as digestible or more so than canola.

| | Amino | | | Yellow Seed | |
|---|---|---|---|---|---|
| No. | Acid | Unit | Canola | Y1126 | Pennycress |
| 1 | ASP | % | 77.6 | 84.8 | 79.6 |
| 2 | THR | % | 77.0 | 79.2 | 73.6 |
| 3 | SER | % | 76.7 | 81.8 | 81.8 |
| 4 | GLU | % | 87.5 | 90.0 | 82.6 |
| 5 | PRO | % | 76.0 | 82.2 | 66.0 |
| 6 | ALA | % | 76.9 | 82.4 | 76.1 |
| 7 | CYS | % | 76.6 | 71.0 | 63.7 |
| 8 | VAL | % | 75.5 | 81.3 | 72.9 |
| 9 | MET | % | 85.9 | 84.9 | 75.8 |
| 10 | ILE | % | 77.2 | 82.2 | 75.7 |
| 11 | LEU | % | 81.5 | 86.1 | 79.1 |
| 12 | TYR | % | 77.1 | 83.8 | 78.2 |
| 13 | PHE | % | 81.6 | 87.1 | 80.4 |
| 14 | LYS | % | 73.5 | 76.7 | 68.9 |
| 15 | HIS | % | 83.4 | 86.6 | 70.1 |
| 16 | ARG | % | 87.0 | 93.0 | 83.6 |
| 17 | TRP | % | 95.4 | 93.2 | 89.2 |

### REFERENCES

Kil, D. J., B. G. Kim, and H. H. Stein. (2013). Feed energy evaluation for growing pigs. Asian-Austrs. J. Animal. Sci. 26 (9): 1205-1217.
Meloche, K. J., B. J. Kerr, G. C. Shurson, and W. A. Dozier, III. (2013). Apparent metabolizable energy and prediction equations for reduced-oil corn distillers fried grains with solubles in broiler chicks. Poultry Science 92 (12): 3176-3183.
Rochelle, S. J., B. J. Kerr, and W. A. Dozier III. (2011). Energy determination of corn co-products fed to broiler chicks from 15 to 24 days of age and use of composition analysis to predict nitrogen-corrected apparent metabolizable energy. Poultry Science 90:1999-2007.
Slominski B A, Simbaya J, Campbell L D, Rakow G, Guenter W (1999) Nutritive value for broilers of meals derived from newly developed varieties of yellow-seeded canola. Anim Feed Sci Technol 78:249-262.
Chauhan, Y. S. and Kumar, K. (1987). Genetics of seed colour in mustard (Brassica juncea L. Czern and Coss), Cruciferae Newsletter 12, 22-23.
Appelhagen I, Lu G H, Huep G, Schmelzer E, Weisshaar B, Sagasser M. (2011) TRANSPARENT TESTA1 interacts with R2R3-MYB factors and affects early and late steps of flavonoid biosynthesis in the endothelium of Arabidopsis thaliana seeds. Plant J. 67:406-419.
Appelhagen I, Thiedig K, Nordholt N, Schmidt N, Huep G, Sagasser M, Weisshaar B. (2014) Update on Transparent testa mutants from Arabidopsis thaliana: characterisation of new alleles from an isogenic collection. Planta 240:955-970.
Baudry A, Heim M A, Dubreucq B, Caboche M, Weisshaar B, Lepiniec L. (2004) TT2, TT8, and TTG1 synergistically specify the expression of BANYULS and proanthocyanidin biosynthesis in Arabidopsis thaliana. Plant J. 39:366-380.
Begemann M B, Gray B N, January E, Gordon G C, He Y, Liu H, Wu X, Brutnell T P, Mockler T C, Oufattole M. (2017) Precise insertion and guided editing of higher plant genomes using Cpf1 CRISPR nucleases. Scientific reports 7:11606.
Begemann M B, Gray B N, January E, Singer A, Kesler D C, He Y, Liu H, Guo H, Jordan A, Brutnell T P, Mockler T C. (2017) Characterization and Validation of a Novel Group of Type V, Class 2 Nucleases for in vivo Genome Editing. bioRxiv. 2017:192799.
Chen M, Wang Z, Zhu Y, Li Z, Hussain N, Xuan L, Guo W, Zhang G, Jiang L. (2012) The effect of TRANSPARENT TESTA2 on seed fatty acid biosynthesis and tolerance to environmental stresses during young seedling establishment in Arabidopsis. Plant Physiol. 160:1023-1036.
Chen M, Xuan L, Wang Z, Zhou L, Li Z, Du X, Ali E, Zhang G, Jiang L. (2014)

TRANSPARENT TESTA8 inhibits seed fatty acid accumulation by targeting several seed development regulators in Arabidopsis. Plant Physiol 165:905-916.
Debeaujon I, Peeters A J, Léon-Kloosterziel K M, Koornneef M. (2001) The TRANSPARENT TESTA12 gene of Arabidopsis encodes a multidrug secondary transporter-like protein required for flavonoid sequestration in vacuoles of the seed coat endothelium. Plant Cell 13:853-871.
Fauser F, Schiml S, Puchta H (2014) Both CRISPR/Cas-based nucleases and nickases can be used efficiently for genome engineering in Arabidopsis thaliana. Plant J 79:348-359.
Guschin D Y, Waite A J, Katibah G E, Miller J C, Holmes M C, Rebar E J. (2010) A rapid and general assay for monitoring endogenous gene modification. In: Engineered zinc finger proteins: 247-256. Humana Press, Totowa, NJ.
Holsters, M., De Waele, D., Depicker, A., Messens, E., Van Montagu, M., & Schell, J. (1978). Transfection and transformation of Agrobacterium tumefaciens. Molecular and General Genetics (MGG), 163 (2), 181-187.
Li X, Chen L, Hong M, Zhang Y, Zu F, Wen J, Yi B, Ma C, Shen J, Tu J, Fu T. (2012) A large insertion in bHLH transcription factor BrTT8 resulting in yellow seed coat in Brassica rapa. PLOS One 7: e44145.
Lian J, Lu X, Yin N, Ma L, Lu J, Liu X, Li J, Lu J, Lei B, Wang R, Chai Y. (2017) Silencing of BnTTI family genes affects seed flavonoid biosynthesis and alters seed fatty acid composition in Brassica napus. Plant Sci. 254:32-47.
Liang M, Davis E, Gardner D, Cai X, Wu Y. (2006) Involvement of AtLAC15 in lignin synthesis in seeds and in root elongation of Arabidopsis. Planta 224:1185-1196.
Michelmore R W, Paran I, Kesseli R V. (1991) Identification of markers linked to disease-resistance genes by bulked segregant analysis: a rapid method to detect markers in specific genomic regions by using segregating populations. Proceedings of the National Academy of Sciences 88:9828-9832.
Magwene P M, Willis J H, Kelly J K. (2011) The statistics of bulk segregant analysis using next generation sequencing. PLoS computational biology 7:11.
Nesi N, Debeaujon I, Jond C, Pelletier G, Caboche M, Lepiniec L. (2000) The TT8 gene encodes a basic helix-loop-helix domain protein required for expression of DFR and BAN genes in Arabidopsis siliques. Plant Cell 12:1863-1878.
Nesi N, Debeaujon I, Jond C, Stewart A J, Jenkins G I, Caboche M, Lepiniec L. (2002) The TRANSPARENT TESTA16 locus encodes the ARABIDOPSIS BSISTER MADS domain protein and is required for proper development and pigmentation of the seed coat. Plant Cell 14:2463-2479.
Nesi N, Jond C, Debeaujon I, Caboche M, Lepiniec L. (2001) The Arabidopsis TT2 gene encodes an R2R3 MYB domain protein that acts as a key determinant for proanthocyanidin accumulation in developing seed. Plant Cell 13:2099-2114.
Pourcel L, Routaboul J M, Kerhoas L, Caboche M, Lepiniec L, Debeaujon I. (2005) TRANSPARENT TESTA10 encodes a laccase-like enzyme involved in oxidative polymerization of flavonoids in Arabidopsis seed coat. Plant Cell 17:2966-2980.
Sagasser M, Lu G H, Hahlbrock K, Weisshaar B. (2002) A. thaliana TRANSPARENT TESTA 1 is involved in seed coat development and defines the WIP subfamily of plant zinc finger proteins. Genes Dev 16:138-149.
Steinert J, Schiml S, Fauser F, Puchta H (2015) Highly efficient heritable plant genome engineering using Cas9 orthologues from Streptococcus thermophilus and Staphylococcus aureus. Plant J 84:1295-305.
Zhang J, Lu Y, Yuan Y, Zhang X, Geng J, Chen Y, Cloutier S, McVetty P B, Li G. (2008) Map-based cloning and characterization of a gene controlling hairiness and seed coat color traits in Brassica rapa. Plant Mol Biol. 69:553-563.

### OTHER EMBODIMENTS

It is to be understood that while certain embodiments have been described in conjunction with the detailed description thereof, the foregoing description is intended to illustrate and not limit the scope of the disclosure. Other aspects, advantages, and modifications are within the scope of the following embodiments and claims.

Embodiment 1. A composition comprising non-defatted pennycress seed meal comprising an acid detergent fiber (ADF) content of 5%, 8%, or 10% to 15%, 18%, or 20% by dry weight.

Embodiment 2. The composition of embodiment 1, wherein said composition has a protein content of 28%, 30%, 32%, or 34% to 38% or 40% by dry weight.

Embodiment 3. The composition of embodiment 1, wherein said composition has an oil content of 30%, 32%, or 34% to 40%, 42%, 46%, 48%, or 50% by dry weight.

Embodiment 4. The composition of embodiment 1, wherein said composition has a neutral detergent fiber (NDF) content of 10%, 12%, 14%, or 16% to 20%, 22%, 24%, or 25% by dry weight.

Embodiment 5. The composition of embodiment 1, wherein said composition has a protein content of 28%, 30%, 32%, or 34% to 38% or 40% by dry weight and an oil content of 30% to 50% by dry weight.

Embodiment 6. A composition comprising defatted pennycress seed meal comprising an acid detergent fiber (ADF) content of 7%, 8%, 10%, or 12% to 20%, 22%, 24%, or 25% by dry weight.

Embodiment 7. The composition of embodiment 6, wherein said composition has a protein content of 30%, 35%, 40%, or 45% to 55%, 60%, 65%, or 70% by dry weight.

Embodiment 8. The composition of embodiment 6, wherein said composition has an oil content of 0%, 2%, or 4% to 8%, 10%, or 12% by dry weight.

Embodiment 9. The composition of embodiment 6, wherein said composition has a neutral detergent fiber (NDF) content of 10%, 12%, or 15% to 20%, 25%, 28%, or 30% by dry weight.

Embodiment 10. The composition of embodiment 6, wherein said composition has a protein content of 30%, 35%, 40%, or 45% to 55%, 60%, 65%, or 70% by dry weight and an oil content of 0%, 2%, or 4% to 8%, 10%, or 12% by dry weight.

Embodiment 11. The composition of embodiment 6, wherein said composition has a protein content of 30%, 35%, 40%, or 45% to 55%, 60%, 65%, or 70% by dry weight and a neutral detergent fiber (NDF) content of 10%, 12%, or 15% to 20%, 25%, 28%, or 30% by dry weight.

Embodiment 12. The composition of any one of embodiments 1-11, wherein said composition further comprises a preservative, a dust preventing agent, a bulking agent, a flowing agent, or any combination thereof.

Embodiment 13. The composition of any one of embodiments 1-12, wherein said pennycress seed meal is obtained from pennycress seeds that have been crushed, ground, macerated, expelled, extruded, expanded, or any combination thereof.

Embodiment 14. The composition of any one of embodiments 1-13, wherein said pennycress seed meal is obtained from a population of pennycress seeds comprising seeds having at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 15. The composition of any one of embodiments 1-14, wherein said pennycress seed meal is obtained from a population of pennycress seeds comprising seeds having at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

Embodiment 16. The composition of any one of embodiments 1-15, wherein said composition comprises a detectable amount of a polynucleotide comprising at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 17. The composition of any one of embodiments 1-16, wherein said pennycress seed meal comprises: (i) pennycress variety Y1067, Y1126, BC38, BJ8, P32, J22, Q36, BD24, AX17, E5-444, E5-540, E5-541, E5-542, E5-543, E5-544, E5-545, E5-547, E5-549, E5-582, E5-586, D3-N10 P5, D5-191, A7-95, A7-187 or A7-261 seed meal; (ii) seed meal of hybrids of the varieties; (iii) seed meal from progeny of the varieties; (iv) seed meal from seed comprising germplasm from the varieties that provides seed comprising an acid detergent fiber (ADF) content of 5% to 20% by dry weight; or (v) seed meal of any combination of said varieties, hybrid varieties, progeny of said varieties, or seed comprising the germplasm.

Embodiment 18. The composition of any one of embodiments 1-17, wherein said pennycress seed meal comprises seed meal obtained from the seed lot of anyone of embodiments 43 to 62, or any combination thereof.

Embodiment 19. The composition of any one of embodiments 1 to 18, wherein the composition exhibits a lighter-color in comparison to a control composition comprising wild-type pennycress seed meal.

Embodiment 20. Pennycress seed meal comprising an acid detergent fiber (ADF) content of 5%, 8%, or 10% to 15%, 18%, or 20% by dry weight, wherein the seed meal is non-defatted.

Embodiment 21. The seed meal of embodiment 20, wherein said seed meal has a protein content of 28%, 30%, 32%, or 34% to 38% or 40% by dry weight.

Embodiment 22. The seed meal of embodiment 21, wherein said seed meal has an oil content of 30%, 32%, or 34% to 40%, 42%, 46%, 48%, or 50% by dry weight.

Embodiment 23. The seed meal of embodiment 21, wherein said seed meal has a neutral detergent fiber (NDF) content of 10%, 12%, 14%, or 16% to 20%, 22%, 24%, or 25% by dry weight.

Embodiment 24. The seed meal of embodiment 21, wherein said seed meal has a protein content of 28%, 30%, 32%, or 34% to 38% or 40% by dry weight and an oil content of 30%, 32%, or 34% to 40%, 42%, 46%, 48%, or 50% by dry weight.

Embodiment 25. Pennycress seed meal comprising an acid detergent fiber (ADF) content of 7%, 8%, 10%, or 12% to 20%, 22%, 24%, or 25% by dry weight, wherein the seed meal is defatted.

Embodiment 26. The seed meal of embodiment 25, wherein said seed meal has a protein content of 30%, 35%, 40%, or 45% to 55%, 60%, 65%, or 70% by dry weight.

Embodiment 27. The seed meal of embodiment 25, wherein said seed meal has an oil content of 0%, 2%, or 4% to 8%, 10%, or 12% by dry weight.

Embodiment 27. The seed meal of embodiment 25, wherein said seed meal has a neutral detergent fiber (NDF) content of 10%, 12%, or 15% to 20%, 25%, 28%, or 30% by dry weight.

Embodiment 28. The seed meal of embodiment 25, wherein said seed meal has a protein content of 30%, 35%, 40%, or 45% to 55%, 60%, 65%, or 70% by dry weight and an oil content of 0%, 2%, or 4% to 8%, 10%, or 12% by dry weight.

Embodiment 29. The pennycress seed meal of any one of embodiments 20-28, wherein the meal comprises ground and/or macerated seed of the seed lot of any one of embodiments 43 to 62.

Embodiment 30. The pennycress seed meal of any one of embodiments 20-29, wherein said meal comprises a detectable amount of a polynucleotide comprising at least one loss-of-function mutation in at least one endogenous wild-type pennycress gene comprising a polynucleotide sequence selected from the group consisting of SEQ ID NO:1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 31. The pennycress seed meal of any one of embodiments 20-30, wherein said meal comprises ground and/or macerated seed of a population of pennycress seeds comprising seeds having at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 32. The pennycress seed meal of any one of embodiments 20-31, wherein said meal comprises ground and/or macerated seed of a population of pennycress seeds comprising seeds having at least one loss-of-function mutation in at least one endogenous pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172 and allelic variants thereof.

Embodiment 33. The pennycress seed meal of any one of embodiments 20-32, wherein said meal comprises ground and/or macerated seed of a population of pennycress seeds comprising seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

Embodiment 34. The pennycress seed meal of any one of embodiments 20-33, wherein the meal exhibits a lighter-color in comparison to a control pennycress seed meal prepared from wild-type pennycress seed.

Embodiment 35. Pennycress seed cake comprising an acid detergent fiber (ADF) content of 7%, 8%, 10%, or 12% to 20%, 22%, 24%, or 25% by dry weight.

Embodiment 36. The seed cake of embodiment 35, wherein said seed meal has a protein content of 30%, 35%, 40%, or 45% to 55%, 60%, 65%, or 70% by dry weight.

Embodiment 37. The seed cake of embodiment 35, wherein said seed meal has an oil content of 0%, 2%, or 4% to 8%, 10%, or 12% by dry weight.

Embodiment 38. The seed cake of embodiment 35, wherein said seed meal has a neutral detergent fiber (NDF) content of 10%, 12%, or 15% to 20%, 25%, 28%, or 30% by dry weight.

Embodiment 39. The seed cake of embodiment 35, wherein said seed meal has a protein content of 30%, 35%, 40%, or 45% to 55%, 60%, 65%, or 70% by dry weight and an oil content of 0%, 2%, or 4% to 8%, 10%, or 12% by dry weight.

Embodiment 40. The pennycress seed cake of any one of embodiments 35 to 39, wherein the cake comprises crushed or expelled seed of the seed lot of any one of embodiments 43 to 62.

Embodiment 41. The pennycress seed cake of any one of embodiments 35 to 40, wherein the cake comprises a detectable amount of a polynucleotide comprising at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 42. The pennycress seed meal or pennycress seed meal cake of any one of embodiments 36 to 41, wherein the cake exhibits a lighter-color in comparison to a control pennycress seed meal cake prepared from wild-type pennycress seed.

Embodiment 43. A seed lot comprising a population of pennycress seeds that comprise an acid detergent fiber (ADF) content of 5%, 8%, or 10% to 15%, 18%, or 20% by dry weight.

Embodiment 44. The seed lot of embodiment 43, wherein said seed has a protein content of 28%, 30%, 32%, or 34% to 38% or 40% by dry weight.

Embodiment 45. The seed lot of embodiment 43, wherein said seed has an oil content of 30%, 32%, or 34% to 40%, 42%, 46%, 48%, or 50% by dry weight.

Embodiment 46. The seed lot embodiment 43, wherein said seed has a neutral detergent fiber (NDF) content of 10%, 12%, 14%, or 16% to 20%, 22%, 24%, or 25% by dry weight.

Embodiment 47. The seed lot of embodiment 43, wherein said seed has a protein content of 28%, 30%, 32%, or 34% to 38% or 40% by dry weight and an oil content of 30%, 32%, or 34% to 40%, 42%, 46%, 48%, or 50% by dry weight.

Embodiment 48. The seed lot of any one of embodiments 43 to 47, wherein the population comprises at least 10, 20, 50, 100, 500, or 1,000 seeds comprising said ADF content.

Embodiment 49. The seed lot of any one of embodiments 43 to 48, wherein at least 95% of the pennycress seeds in the seed lot are seeds comprising said ADF content and said protein content.

Embodiment 50. The seed lot of any one of embodiments 43 to 49, wherein less than 5% of the seeds in said seed lot have an ADF content of greater than 20% by dry weight.

Embodiment 51. The seed lot of any one of embodiments 43 to 50, wherein said seeds further comprise an agriculturally acceptable excipient or adjuvant.

Embodiment 52. The seed lot of any one of embodiments 43 to 51, wherein said seeds further comprise a fungicide, a safener, or any combination thereof.

Embodiment 53. The seed lot of any one of embodiments 43 to 52, wherein said population of pennycress seeds comprise seeds having at least one loss-of-function mutation in at least one endogenous pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof or comprise seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

Embodiment 54. The seed lot of any one of embodiments 43 to 53, wherein said population of pennycress seeds comprise seeds having at least one loss-of-function mutation in an endogenous wild-type pennycress gene that encodes SEQ ID NO:2, 70, 76, or an allelic variant thereof.

Embodiment 55. The seed lot of embodiment 54, wherein the loss-of-function mutation in the gene encoding SEQ ID NO:2, 70, 76, or the allelic variant thereof comprises an insertion, deletion, or substitution of one or more nucleotides.

Embodiment 56. The seed lot of embodiment 54, wherein the loss-of-function mutation in the gene encoding SEQ ID NO:2 or the allelic variant thereof comprises a mutation that introduces a pre-mature stop codon or frameshift mutation at codon positions 1-108 of SEQ ID NO: 1 or an allelic variant thereof, wherein the loss-of-function mutation in the gene encoding SEQ ID NO:70 or the allelic variant thereof comprises a mutation set forth in SEQ ID NO:127, 129, 131, 133, 135, or 137, or wherein the loss-of-function mutation in the gene encoding SEQ ID NO: 76 or the allelic variant thereof comprises a mutation set forth in SEQ ID NO: 165, 167, or 170.

Embodiment 57. The seed lot of any one of embodiments 54-56, wherein the loss-of-function mutation in the gene encoding SEQ ID NO:2 or the allelic variant thereof comprises a substitution of a guanine residue at nucleotide 491 of SEQ ID NO: 1 with an adenine residue or a substitution of a guanine residue a nucleotide equivalent to nucleotide 491 of SEQ ID NO: 1 in the allelic variant thereof with an adenine residue.

Embodiment 58. The seed lot of any one of embodiments 43 to 57, wherein said population of pennycress seeds comprise seeds having at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

Embodiment 59. The seed lot of any one of embodiments 43 to 58, wherein said population of pennycress seeds comprising seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

Embodiment 60. The seed lot of any one of embodiments 43 to 59, wherein said population of pennycress seeds comprise: (i) pennycress variety Y1067, Y1126, BC38, BJ8, P32, J22, Q36, BD24, AX17, AX17, E5-444, E5-540, E5-541, E5-542, E5-543, E5-544, E5-545, E5-547, E5-549, E5-582, E5-586, D3-N10 P5, D5-191, A7-95, A7-187 or A7-261 seed; (ii) hybrid seed of said varieties; (iii) seed from progeny of said varieties; (iv) seed comprising germplasm from said varieties that provides seed having an acid detergent fiber (ADF) content of 10% to 20% by dry weight; or (v) any combination of said seed, hybrid seed, seed from progeny of said varieties, or seed comprising said germplasm.

Embodiment 61. The seed lot of any one of embodiments 43 to 60, wherein the seeds in the population exhibit a lighter-colored seed coat in comparison to a wild-type pennycress seed.

Embodiment 62. A method of making non-defatted pennycress seed meal comprising an acid detergent fiber (ADF) content of 5%, 8%, or 10% to 15%, 18%, or 20% by dry weight, comprising the step of grinding, macerating, extruding, and/or crushing the seed lot of any one of embodiments 43 to 62, thereby obtaining the non-defatted seed meal.

Embodiment 63. The method of embodiment 62, wherein the seed meal has a protein content of 28%, 30%, 32%, or 34% to 38% or 40% by dry weight, or the combination thereof.

Embodiment 64. The method of embodiment 62, wherein said seed meal has an oil content of 30%, 32%, or 34% to 40%, 42%, 46%, 48%, or 50% by dry weight.

Embodiment 65. The method of embodiment 62, wherein said seed meal has a neutral detergent fiber (NDF) content of 10%, 12%, 14%, or 16% to 20%, 22%, 24%, or 25% by dry weight.

Embodiment 66. The method of embodiment 62, wherein said seed meal has a protein content of 28%, 30%, 32%, or 34% to 38% or 40% by dry weight and an oil content of 30%, 32%, or 34% to 40%, 42%, 46%, 48%, or 50% by dry weight.

Embodiment 67. A method of making defatted pennycress seed meal comprising an acid detergent fiber (ADF) content of 7%, 8%, 10%, or 12% to 20%, 22%, 24%, or 25% by dry weight, comprising the step of solvent extracting the seed lot of any one of embodiments 43 to 62, separating the extracted seed meal from the solvent, thereby obtaining the defatted seed meal.

Embodiment 68. The method of embodiment 67, wherein the seed meal has a protein content of 30%, 35%, 40%, or 45% to 55%, 60%, 65%, or 70% by dry weight.

Embodiment 69. The method of embodiment 67, wherein said seed meal has an oil content of 0%, 2%, or 4% to 8%, 10%, or 12% by dry weight.

Embodiment 70. The method of embodiment 67, wherein said seed meal has a neutral detergent fiber (NDF) content of 10% to 30% by dry weight.

Embodiment 71. The method of embodiment 67 wherein said seed meal has a protein content of 30%, 35%, 40%, or 45% to 55%, 60%, 65%, or 70% by dry weight and an oil content of 0%, 2%, or 4% to 8%, 10%, or 12% by dry weight.

Embodiment 72. The method of any one of embodiments 67 to 71, wherein the solvent is hexane or mixed hexanes.

Embodiment 73. A method of making pennycress seed cake comprising an acid detergent fiber (ADF) content of 7%, 8%, 10%, or 12% to 20%, 22%, 24%, or 25% by dry weight, comprising the step of crushing or expelling the seed of the seed lot any one of embodiments 43 to 62, thereby obtaining a seed cake.

Embodiment 74. The method of embodiment 73, wherein the seed cake has a protein content of 30%, 35%, 40%, or 45% to 55%, 60%, 65%, or 70% by dry weight.

Embodiment 75. The method of embodiment 74, wherein the seed cake has an oil content of 0%, 2%, or 4% to 8%, 10%, or 12% by dry weight.

Embodiment 76. A method of making a pennycress seed lot comprising the steps of:
(a) introducing at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof;
(b) selecting germplasm that is homozygous for said loss-of-function mutation; and,
(c) harvesting seed from the homozygous germplasm, thereby obtaining a seed lot, wherein said seed lot comprises an acid detergent fiber (ADF) content of 5%, 8%, or 10% to 15%, 18%, or 20% by dry weight.

Embodiment 77. The method of embodiment 76, wherein said seed lot comprise the seed lot of any one of embodiments 43 to 61.

Embodiment 78. A method of making a pennycress seed lot comprising the steps of:
(a) introducing at least one transgene that suppresses expression of at least one endogenous wild-type pennycress gene encoding a polypeptide selected from the group consisting of SEQ ID NO: 2, 7, 10, 13, 16, 19, 22, 25, 28, 31, 34, 37, 40, 43, 46, 49, 52, 55, 58, 61, 64, 67, 70, 73, 76, 79, 172, and allelic variants thereof into a pennycress plant genome;
(b) selecting a transgenic plant line that comprises said transgene; and,
(c) harvesting seed from the transgenic plant line, thereby obtaining a seed lot, wherein said seed lot comprises an acid detergent fiber (ADF) content of 5%, 8%, or 10% to 15%, 18%, or 20% by dry weight.

Embodiment 79. The method of embodiment 78, wherein said harvested seed comprise a seed lot of any one of embodiments 43 to 61.

What is claimed is:

1. A composition comprising non-defatted pennycress seed meal comprising an acid detergent fiber (ADF) content of 5% to 20% by dry weight.

2. The composition of claim 1, wherein said composition has a protein content of 28% to 40% by dry weight.

3. The composition of claim 1, wherein said composition has an oil content of 30% to 50% by dry weight.

4. The composition of claim 1, wherein said composition has a neutral detergent fiber (NDF) content of 10% to 25% by dry weight.

5. The composition of claim 1, wherein said composition has a protein content of 28% to 40% by dry weight and an oil content of 30% to 50% by dry weight.

6. A composition comprising defatted pennycress seed meal comprising an acid detergent fiber (ADF) content of 7% to 25% by dry weight.

7. The composition of claim 6, wherein said composition has a protein content of 30% to 70% by dry weight.

8. The composition of claim 6, wherein said composition has an oil content of 0% to 12% by dry weight.

9. The composition of claim 6, wherein said composition has a neutral detergent fiber (NDF) content of 10% to 30% by dry weight.

10. The composition of claim 6, wherein said composition has a protein content of 30% to 70% by dry weight and an oil content of 0% to 12% by dry weight.

11. The composition of claim 6, wherein said composition has a protein content of 30%, 35%, 40%, or 45% to 55%, 60%, 65%, or 70% by dry weight and a neutral detergent fiber (NDF) content of 10%, 12%, or 15% to 20%, 25%, 28%, or 30% by dry weight.

12. The composition of any one of claims 1 to 11, wherein said composition further comprises a preservative, a dust preventing agent, a bulking agent, a flowing agent, or any combination thereof.

13. The composition of any one of claims 1 to 11, wherein said pennycress seed meal is obtained from pennycress seeds that have been crushed, ground, macerated, expelled, extruded, expanded, or any combination thereof.

14. The composition of any one of claims 1 to 11, wherein said pennycress seed meal is obtained from a population of pennycress seeds comprising seeds having at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

15. The composition of any one of claims 1 to 11, wherein said pennycress seed meal is obtained from a population of pennycress seeds comprising seeds having at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

16. The composition of any one of claims 1 to 11, wherein said composition comprises a detectable amount of a polynucleotide comprising at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

17. The composition of any one of claims 1 to 11, wherein said pennycress seed meal comprises: (i) pennycress variety Y1067, Y1126, BC38, BJ8, P32, J22, Q36, BD24, AX17, E5-444, E5-540, E5-541, E5-542, E5-543, E5-544, E5-545, E5-547, E5-549, E5-582, E5-586, D3-N10 P5, D5-191, A7-95, A7-187, or A7-261 seed meal; (ii) seed meal of hybrids of the varieties; (iii) seed meal from progeny of the varieties; (iv) seed meal from seed comprising germplasm from the varieties that provides seed comprising an acid detergent fiber (ADF) content of 5% to 20% by dry weight; or (v) seed meal of any combination of said varieties, hybrid varieties, progeny of said varieties, or seed comprising the germplasm.

18. The composition of any one of claims 1 to 11, wherein said pennycress seed meal comprises seed meal obtained from a seed lot comprising a population of pennycress seeds that comprise an acid detergent fiber (ADF) content of 5% to 20% by dry weight.

19. The composition of any one of claims 1 to 11, wherein the composition exhibits a lighter-color in comparison to a control composition comprising wild-type pennycress seed meal.

20. Pennycress seed meal comprising an acid detergent fiber (ADF) content of 5% to 20% by dry weight, wherein the seed meal is non-defatted.

21. The seed meal of claim 20, wherein said seed meal has a protein content of 28% to 40% by dry weight.

22. The seed meal of claim 20, wherein said seed meal has an oil content of 30% to 50% by dry weight.

23. The seed meal of claim 20, wherein said seed meal has a neutral detergent fiber (NDF) content of 10% to 25% by dry weight.

24. The seed meal of claim 20, wherein said seed meal has a protein content of 28% to 40% by dry weight and an oil content of 30% to 50% by dry weight.

25. Pennycress seed meal comprising an acid detergent fiber (ADF) content of 7% to 25% by dry weight, wherein the seed meal is defatted.

26. The seed meal of claim 25, wherein said seed meal has a protein content of 30% to 70% by dry weight.

27. The seed meal of claim 25, wherein said seed meal has an oil content of 0% to 12% by dry weight.

28. The seed meal of claim 25, wherein said seed meal has a neutral detergent fiber (NDF) content of 10% to 30% by dry weight.

29. The seed meal of claim 25, wherein said seed meal has a protein content of 30% to 70% by dry weight and an oil content of 0% to 12% by dry weight.

30. The pennycress seed meal of claim 20 or 25, wherein the meal comprises ground and/or macerated seed of a seed lot comprising a population of pennycress seeds that comprise an acid detergent fiber (ADF) content of 5% to 20% by dry weight.

31. The pennycress seed meal of claim 20 or 25, wherein said meal comprises a detectable amount of a polynucleotide comprising at least one loss-of-function mutation in at least one endogenous wild-type pennycress gene comprising a polynucleotide sequence selected from the group consisting of SEQ ID NO:1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

32. The pennycress seed meal of claim 20 or 25, wherein said meal comprises ground and/or macerated seed of a population of pennycress seeds comprising seeds having at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

33. The pennycress seed meal of claim 20 or 25, wherein said meal comprises ground and/or macerated seed of a population of pennycress seeds comprising seeds having at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

34. The pennycress seed meal of claim 20 or 25, wherein said meal comprises ground and/or macerated seed of a population of pennycress seeds comprising seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

35. The pennycress seed meal of claim 20 or 25, wherein the meal exhibits a lighter-color in comparison to a control pennycress seed meal prepared from wild-type pennycress seed.

36. Pennycress seed cake comprising an acid detergent fiber (ADF) content of 7% to 25% by dry weight.

37. The seed cake of claim 36, wherein said seed meal has a protein content of 30% to 70% by dry weight.

38. The seed cake of claim 36, wherein said seed meal has an oil content of 0% to 16% by dry weight.

39. The seed cake of claim 36, wherein said seed meal has a neutral detergent fiber (NDF) content of 10% to 30% by dry weight.

40. The seed cake of claim 36, wherein said seed meal has a protein content of 30% to 70% by dry weight and an oil content of 0% to 16% by dry weight.

41. The pennycress seed cake of claim 36, wherein the cake comprises crushed or expelled seed of a seed lot comprising a population of pennycress seeds that comprise an acid detergent fiber (ADF) content of 5% to 20% by dry weight.

42. The pennycress seed cake of any one of claims 36 to 41, wherein the cake comprises a detectable amount of a polynucleotide comprising at least one loss-of-function mutation in at least one endogenous wild-type pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

43. The pennycress seed meal or pennycress seed meal cake of any one of claims 36 to 41, wherein the cake exhibits a lighter-color in comparison to a control pennycress seed meal cake prepared from wild-type pennycress seed.

44. A seed lot comprising a population of pennycress seeds that comprise an acid detergent fiber (ADF) content of 5% to 20% by dry weight.

45. The seed lot of claim 44, wherein said seeds have a protein content of 28% to 40% by dry weight.

46. The seed lot of claim 44, wherein said seeds have an oil content of 30% to 50% by dry weight.

47. The seed lot claim 44, wherein said seeds have a neutral detergent fiber (NDF) content of 10% to 25% by dry weight.

48. The seed lot of claim 44, wherein said seeds have a protein content of 28% to 40% by dry weight and an oil content of 30% to 50% by dry weight.

49. The seed lot of any one of claims 44 to 48, wherein the population comprises at least 10 seeds comprising said ADF content.

50. The seed lot of any one of claims 44 to 48, wherein at least 95% of the pennycress seeds in the seed lot are seeds comprising said ADF content and said protein content.

51. The seed lot of any one of claims 44 to 48, wherein less than 5% of the seeds in said seed lot have an ADF content of greater than 20% by dry weight.

52. The seed lot of any one of claims 44 to 48, wherein said seeds further comprise an agriculturally acceptable excipient or adjuvant.

53. The seed lot of any one of claims 44 to 48, wherein said seeds further comprise a fungicide, a safener, or any combination thereof.

54. The seed lot of any one of claims 44 to 48, wherein said population of pennycress seeds comprise seeds having at least one loss-of-function mutation in at least one endogenous wild-type pennycress gene encoding a polypeptide selected from the group consisting of SEQ ID NO: 2, 7, 10, 13, 16, 19, 22, 25, 28, 31, 34, 37, 40, 43, 46, 49, 52, 55, 58, 61, 64, 67, 70, 73, 76, 79, and allelic variants thereof or comprise seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

55. The seed lot of any one of claims 44 to 48, wherein said population of pennycress seeds comprise seeds having at least one loss-of-function mutation in an endogenous wild-type pennycress gene that encodes SEQ ID NO:2, 70, 76, or an allelic variant thereof.

56. The seed lot of claim 55, wherein the loss-of-function mutation in the gene encoding SEQ ID NO: 2, 70, 76, or the allelic variant thereof comprises an insertion, deletion, or substitution of one or more nucleotides.

57. The seed lot of claim 55, wherein the loss-of-function mutation in the gene encoding SEQ ID NO: 2 or the allelic variant thereof comprises a mutation that introduces a pre-mature stop codon or frameshift mutation at codon positions 1-108 of SEQ ID NO:1 or an allelic variant thereof, wherein the loss-of-function mutation in the gene encoding SEQ ID NO:70 or the allelic variant thereof comprises a mutation set forth in SEQ ID NO: 127, 129, 131, 133, 135, or 137, or wherein the loss-of-function mutation in the gene encoding SEQ ID NO:76 or the allelic variant thereof comprises a mutation set forth in SEQ ID NO:165, 167, or 170.

58. The seed lot of claim 55, wherein the loss-of-function mutation in the gene encoding SEQ ID NO: 2 or the allelic variant thereof comprises a substitution of a guanine residue at nucleotide 491 of SEQ ID NO: 1 with an adenine residue or a substitution of a guanine residue a nucleotide equivalent to nucleotide 491 of SEQ ID NO: 1 in the allelic variant thereof with an adenine residue.

59. The seed lot of any one of claims 44 to 48, wherein said population of pennycress seeds comprise seeds having at least one loss-of-function mutation in at least one endogenous pennycress gene comprising a polynucleotide sequence selected from the group consisting of SEQ ID NO: 1, 3, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, 63, 65, 66, 68, 69, 71, 72, 74, 75, 77, 78, 80, 171, 173, and allelic variants thereof.

60. The seed lot of any one of claims 44 to 48, wherein said population of pennycress seeds comprising seeds having at least one transgene that suppresses expression of at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof.

61. The seed lot of any one of claims 44 to 48, wherein said population of pennycress seeds comprise: (i) pennycress variety Y1067, Y1126, BC38, BJ8, P32, J22, Q36, BD24, AX17, E5-444, E5-540, E5-541, E5-542, E5-543, E5-544, E5-545, E5-547, E5-549, E5-582, E5-586, D3-N10 P5, D5-191, A7-95, A7-187, or A7-261 seed; (ii) hybrid seed of said varieties; (iii) seed from progeny of said varieties; (iv) seed comprising germplasm from said varieties that provides seed having an acid detergent fiber (ADF) content of 5% to 20% by dry weight; or (v) any combination of said seed, hybrid seed, seed from progeny of said varieties, or seed comprising said germplasm.

62. The seed lot of any one of claims 44 to 48, wherein the seeds in the population exhibit a lighter-colored seed coat in comparison to a wild-type pennycress seed.

63. A method of making non-defatted pennycress seed meal comprising an acid detergent fiber (ADF) content of 5% to 20% by dry weight, comprising the step of grinding, macerating, extruding, expanding, and/or crushing the seed lot of any one of claims 44 to 48 thereby obtaining the non-defatted seed meal.

64. The method of claim 63, wherein the seed meal has a protein content of 28% to 40% by dry weight, or the combination thereof.

65. The method of claim 63, wherein said seed meal has an oil content of 30% to 50% by dry weight.

66. The method of claim 63, wherein said seed meal has a neutral detergent fiber (NDF) content of 10% to 25% by dry weight.

67. The method of claim 63, wherein said seed meal has a protein content of 28% to 40% by dry weight and an oil composition of 30% to 50% by dry weight.

68. A method of making defatted pennycress seed meal comprising an acid detergent fiber (ADF) content of 7% to 25% by dry weight, comprising the step of solvent extracting the seed lot of any one of claims 44 to 48, separating the extracted seed meal from the solvent, thereby obtaining the defatted seed meal.

69. The method of claim 68, wherein the seed meal has a protein content of 30% to 70% by dry weight.

70. The method of claim 68, wherein said seed meal has an oil content of 0% to 12% by dry weight.

71. The method of claim 68, wherein said seed meal has a neutral detergent fiber (NDF) content of 10% to 30% by dry weight.

72. The method of claim 68, wherein said seed meal has a protein content of 30% to 70% by dry weight and an oil content of 0% to 12% by dry weight.

73. The method of claim 68, wherein the solvent is hexane or mixed hexanes.

74. A method of making pennycress seed cake comprising an acid detergent fiber (ADF) content of 7% to 25% by dry weight, comprising the step of crushing or expelling the seed of the seed lot any one of claims 44 to 62, thereby obtaining a seed cake.

75. The method of claim 74, wherein the seed cake has a protein content of 30% to 70% by dry weight.

76. The method of claim 74, wherein the seed cake has an oil content of 0% to 12% by dry weight.

77. A method of making a pennycress seed lot comprising the steps of:
(a) introducing at least one loss-of-function mutation in at least one endogenous wild-type pennycress gene encoding a polypeptide selected from the group consisting of SEQ ID NO:2, 7, 10, 13, 16, 19, 22, 25, 28, 31, 34, 37, 40, 43, 46, 49, 52, 55, 58, 61, 64, 67, 70, 73, 76, 79, 172, and allelic variants thereof;
(b) selecting germplasm that is homozygous for said loss-of-function mutation; and,
(c) harvesting seed from the homozygous germplasm, thereby obtaining a seed lot, wherein said seed lot comprises an acid detergent fiber (ADF) content of 5% to 20% by dry weight.

78. The method of claim 77, wherein said seed lot comprise the seed lot of any one of claims 44 to 48.

79. A method of making a pennycress seed lot comprising the steps of:
(a) introducing at least one transgene that suppresses expression of at least one endogenous wild-type pennycress gene encoding a polypeptide selected from the group consisting of SEQ ID NO: 2, 7, 10, 13, 16, 19, 22, 25, 28, 31, 34, 37, 40, 43, 46, 49, 52, 55, 58, 61, 64, 67, 70, 73, 76, 79, 172, and allelic variants thereof into a pennycress plant genome;
(b) selecting a transgenic plant line that comprises said transgene; and,
(c) harvesting seed from the transgenic plant line, thereby obtaining a seed lot, wherein said seed lot comprises an acid detergent fiber (ADF) content of 5% to 20% by dry weight.

80. The method of claim 79, wherein said harvested seed comprise a seed lot of any one of claims 44 to 48.
